(19) United States
(12) Patent Application Publication
(10) Pub. No.: US 2026/0232658 A1
(43) Pub. Date: Aug. 13, 2026

(54) AZETIDINYL BENZOXAZOLE COMPOUNDS AND THEIR USE AS MER AND AXL INHIBITORS

(71) Applicant: DONG-A ST CO., LTD., Seoul (KR)

(72) Inventors: Robin R. FREY, Libertyville, IL (US); Zhiqin JI, Libertyville, IL (US); Yujia DAI, Gurnee, IL (US); Julie M. MIYASHIRO, Morton Grove, IL (US); Thomas D. PENNING, Elmhurst, IL (US)

(21) Appl. No.: 19/122,199

(22) PCT Filed: Oct. 20, 2023
(86) PCT No.: PCT/IB2023/060601

§ 371 Date: Apr. 17, 2025

Related U.S. Application Data
(60) Provisional application No. 63/380,302, filed on Oct. 20, 2022.

(51) Int. Cl. A61K 31/497; A61K 31/454; A61K 31/501; A61K 31/5377; A61K 31/675; A61P 35/00; C07D 413/14; C07D 417/14; C07F 9/653

(52) U.S. Cl. CPC A61K 31/497; A61K 31/454; A61K 31/501; A61K 31/5377; A61K 31/675; A61P 35/00; C07D 413/14; C07D 417/14; C07F 9/65324

(57) ABSTRACT

Compounds of formula (I)

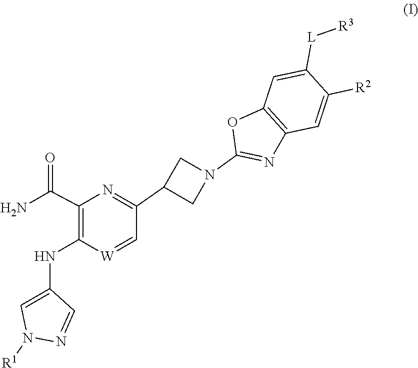

and an enantiomer, a diastereomer, a racemate, a tautomer, a prodrug, a hydrate, a solvate and pharmaceutically acceptable salts thereof, wherein L, R¹, R², R³ and W, are as defined in the specification, are useful in the treatment of diseases and conditions influenced by inhibition of Mer kinase, including cancer.

### TECHNICAL FIELD

Compounds of formula (I) and methods for inhibiting Mer and Axl kinases are disclosed. Additionally, the present disclosure relates to compositions containing compounds of the present disclosure and methods for their use.

### BACKGROUND ART

Receptor tyrosine kinases (RTKs) are enzymes that can phosphorylate specific tyrosine residues in target proteins, using ATP and share a highly conserved catalytic domain. The conservation makes it hard to develop a selective tyrosine kinase inhibitor (TKI). The TAM receptor family consists of Tyro3, Axl, and Mer, which play important roles in cancer, hemostasis and inflammation. Mer is, especially, a key regulator of macrophage and dendritic cells activation. Mer activation promotes apoptotic cell clearance by macrophages and additional cell types such as retinal pigmented epithelial cells. Moreover, Mer is abnormally overexpressed in human cancers such as AML, ALL, lung cancer, glioma, melanoma, prostate cancer, schwannoma, mantle cell lymphoma and rhabdomyoscaroma. Axl is more broadly expressed in a variety of cell types and tissues and participates in diverse cellular processes such as angiogenesis and tumorigenesis. Axl overexpression is critical for the development, growth, and spread of tumors, including proliferation, invasiveness and immune modulation. Axl has been implicated in chemoresistance as well as resistance to targeted therapy of oncogenic drivers, such as BRAF and EGFR. Its overexpression in resistant tumors is correlated with poor survival in a number of aggressive tumors including triple-negative breast cancer, acute myeloid leukemia, non-small-cell lung cancer, pancreatic cancer and ovarian cancer. Much less is known about Tyro3 but its main function is involved in innate immune response, platelet aggregation and neuronal signaling.

Since ATP-binding site is similar for all protein kinases, it is challenging to find an inhibitor that is specific for Mer and Axl kinases. Compound-52, a 2,6,9-trisubstituted purine that competitively binds to the ATP binding pocket, was actually the first molecule that was found to be successful in inhibiting Mer (J Struct Biol. 2009 February; 165(2): 88-96). This inhibitor has, however, limited potency and lack of selectivity. Several compounds have been discovered mostly by modifying Compound-52 including UNC-569, UNC-1062, and UNC-2025 (ACS Med Chem Lett. 2012 Feb. 9; 3(2):129-134, Eur J Med Chem. 2013 July; 65:83-93, J Med Chem. 2014 Aug. 28; 57(16):7031-41). More recently, highly potent and selective Mer kinase inhibitors were disclosed in U.S. Pat. No. 10,125,118 and WO2018071343.

It is an object of the invention to provide reagents and methods of modulating receptor tyrosines kinases Mer and Axl. This and other objects of the invention are provided by one or more of the embodiments described below.

### DISCLOSURE

### Technical Problem

The present disclosure is directed to compounds, enantiomers, diastereomers, racemates, tautomers, prodrugs, hydrates, solvates or pharmaceutically acceptable salts thereof, having a structure of formula (I):

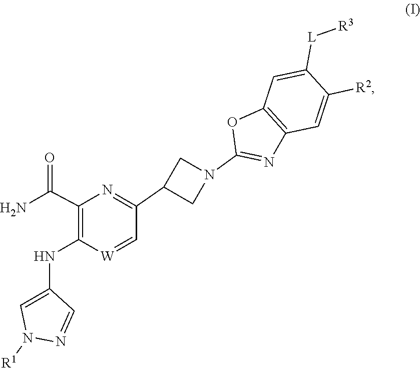

where
W is N or CH;
L is selected from the group consisting of a bond, —CH═CHCR^{a}R^{b}—, —(CR^{a}R^{b})ₙ—O—, —(CR^{a}R^{b})ₙ—C(O)—, —O—, —O—(CR^{a}R^{b})ₙ—, —O—(CR^{a}R^{b})ₙ—O—, —OCH₂C(O)—, and —C(O)—;
each R^{a} is independently selected from the group consisting of hydrogen, C₁-C₄ alkyl, and C₁-C₂ hydroxyalkyl;
each R^{b} is independently selected from the group consisting of hydrogen and C₁-C₄ alkyl;
where R^{a} and R^{b} optionally combine to form a C₃₋₆ cycloalkyl or 3-6 membered heterocycle;
R¹ is selected from the group consisting of H, C₁-C₆ alkyl, and 5-6 membered heterocyclyl; wherein the C₁-C₆ alkyl of R¹ is optionally substituted with 1 or 2 of R⁴;
R² is selected from the group consisting of hydrogen, fluoro, chloro, bromo, and C₁-C₄ alkyl;
R³ is selected from the group consisting of hydrogen, fluoro, chloro, bromo, —CN, C₁-C₄ alkyl, C₁-C₄ haloalkyl, —OH, —OR^{3a}, —NR^{3b}R^{3c}, —P(O)(OR^{3d})₂, —C(O)CHR^{3e}NR^{3b}R^{3c}, —C(O)OH, —C(O)OR^{3a}, —C(O)NR^{3b}R^{3c}, C₃-C₈ cycloalkyl, C₅-C₇ cycloalkenyl, 3-6 membered heterocyclyl, C₅-C₇ heterocycloalkenyl, C₆ aryl, 5-6 membered heteroaryl and

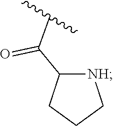 [SMILES: *C(=O)C1CCCN1]

wherein the C₃-C₈ cycloalkyl, C₆ aryl, 3-6 membered heterocyclyl, and 5-6 membered heteroaryl of R³, are optionally substituted with 1 or 2 of R⁵;
R^{3a} is C₁-C₄ alkyl;
R^{3b} and R^{3c} are each independently selected from the group consisting of hydrogen, C₁-C₄ alkyl, C₃-C₆ cycloalkyl, and —CO₂R^{3f};
R^{3d} is selected from the group consisting of hydrogen, C₁-C₄ alkyl, and C₂-C₄ alkenyl;
R^{3e} is selected from the group consisting of hydrogen and C₁-C₄ alkyl; wherein the C₁-C₄ alkyl of R^{3d} is optionally substituted with 1 R^{3g};
R^{3f} is C₁-C₄ alkyl;
R^{3g} selected from the group consisting of —OH, —SH, —SCH₃, —NH₂, —C(O)OH, —C(O)NH₂, —NHC(NH)NH₂, C₆ aryl, and 5-9 membered heteroaryl; wherein the R^{3g} C₆ aryl of R^{3g} is optionally substituted with 1 of —OH;
R⁴ is selected from the group consisting of —OH, —OR^{4a}, —C(O)NR^{4b}R^{4c}, and 5-6 membered heterocyclyl; wherein the 5-6 membered heterocyclyl of R⁴ is optionally substituted with 1 or 2 of C₁-C₄ alkyl;
R^{4a} is C₁-C₄ alkyl; wherein the C₁-C₄ alkyl of R^{4a} is optionally substituted with 1 of —Si(R^{4d})₃;
R^{4b} and R^{4c} are each independently selected from the group consisting of hydrogen and C₁-C₄ alkyl;
R^{4d} is C₁-C₄ alkyl;
R⁵ is selected from the group consisting of fluoro, —CN, —OH, —OR^{5a}, =O, —CH₂CO₂R^{5b}, C₁-C₄ alkyl, C₁-C₄ haloalkyl, C₁-C₄ hydroxyalkyl, C₁-C₄ hydroxyalkoxy, and C₂-C₆ alkoxyalkyl;
R^{5a} is C₁-C₄ alkyl; and
R^{5b} is C₁-C₄ alkyl;
n is an integer of 1 to 3.

In certain embodiments, the present disclosure is directed to compounds of formula (I), enantiomers, diastereomers, racemates, tautomers, prodrugs, hydrates, solvates or pharmaceutically acceptable salts thereof, wherein L is —O—(CR^{a}R^{b})ₙO—.

In certain embodiments, the present disclosure is directed to compounds of formula (I), enantiomers, diastereomers, racemates, tautomers, prodrugs, hydrates, solvates or pharmaceutically acceptable salts thereof, wherein
L is a bond; and
R³ is selected from the group consisting of hydrogen, fluoro, chloro, bromo, —CN, C₁-C₄ alkyl, C₁-C₄ haloalkyl, —OH, —OR^{3a}, —NR^{3b}R^{3c}, —C(O)OH, —C(O)OR^{3a}, —C(O)NR^{3b}R^{3c}, C₃-C₈ cycloalkyl, C₅-C₇ cycloalkenyl, 3-6 membered heterocyclyl, C₅-C₇ heterocycloalkenyl, C₆ aryl, and 5-6 membered heteroaryl;
wherein the C₃-C₈ cycloalkyl, C₆ aryl, 3-6 membered heterocyclyl, and 5-6 membered heteroaryl of R³, are optionally substituted with 1 or 2 of R⁵.

In certain embodiments, the present disclosure is directed to compounds of formula (I), enantiomers, diastereomers, racemates, tautomers, prodrugs, hydrates, solvates or pharmaceutically acceptable salts thereof, wherein
R¹ is C₁-C₆ alkyl; wherein the C₁-C₆ alkyl of R¹ is optionally substituted with 1 or 2 of R⁴;
L is a bond; and
R³ is selected from the group consisting of hydrogen, fluoro, chloro, bromo, —CN, C₁-C₄ alkyl, C₁-C₄ haloalkyl, —OH, —OR^{3a}, —NR^{3b}R^{3c}, —C(O)OH, —C(O)OR^{3a}, —C(O)NR^{3b}R^{3c}, C₃-C₈ cycloalkyl, C₅-C₇ cycloalkenyl, 3-6 membered heterocyclyl, C₅-C₇ heterocycloalkenyl, C₆ aryl, and 5-6 membered heteroaryl;
wherein the C₃-C₈ cycloalkyl, C₆ aryl, 3-6 membered heterocyclyl, and 5-6 membered heteroaryl of R³, are optionally substituted with 1 or 2 of R⁵; and
R⁴ is selected from the group consisting of —OH, —OR^{4a}, —C(O)NR^{4b}R^{4c}, and 5-6 membered heterocyclyl. In certain embodiments, R¹ is methyl.

In certain embodiments, the present disclosure is directed to compounds of formula (I), enantiomers, diastereomers, racemates, tautomers, prodrugs, hydrates, solvates or pharmaceutically acceptable salts thereof, wherein
R¹ is methyl;
L is a bond; and
R³ is selected from the group consisting of hydrogen, C₁-C₄ alkyl, C₆ aryl, and 5-6 membered heteroaryl; wherein the C₆ aryl, and 5-6 membered heteroaryl of R³, are optionally substituted with 1 or 2 of R⁵; and
R⁵ is selected from the group consisting of —OH, —OR^{5a}, C₁-C₄ alkyl, C₁-C₄ hydroxyalkyl, C₁-C₄ hydroxyalkoxy, and C₂-C₆ alkoxyalkyl.

In certain embodiments, the present disclosure is directed to compounds of formula (I), enantiomers, diastereomers, racemates, tautomers, prodrugs, hydrates, solvates or pharmaceutically acceptable salts thereof, wherein
L is —O—(CR^{a}R^{b})ₙ—;
R³ is selected from the group consisting of —CN, C₃-C₈ cycloalkyl, 3-6 membered heterocyclyl, C₆ aryl, and 5-6 membered heteroaryl; wherein the C₃-C₈ cycloalkyl, C₆ aryl, 3-6 membered heterocyclyl, and 5-6 membered heteroaryl of R³, are optionally substituted with 1 or 2 R⁵; and
R⁵ is selected from the group consisting of —OH, —CH₂CO₂R^{5b}, C₁-C₄ alkyl, and C₁-C₄ hydroxyalkyl.

In certain embodiments, the present disclosure is directed to compounds of formula (II), enantiomers, diastereomers, racemates, tautomers, prodrugs, hydrates, solvates or pharmaceutically acceptable salts thereof, wherein

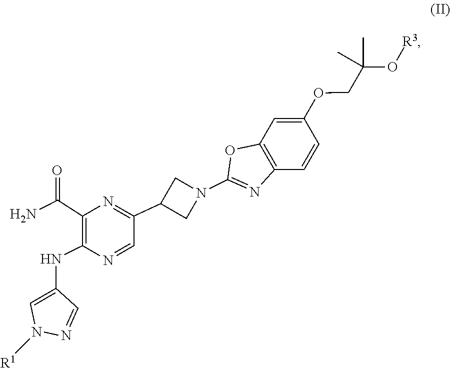

where
R¹ is selected from the group consisting of hydrogen, C₁-C₆ alkyl, and 5-6 membered heterocyclyl; wherein the R¹ C₁-C₆ alkyl is optionally substituted with 1 or 2 R⁴;
R³ is selected from the group consisting of hydrogen, C₁-C₄ alkyl, C₁-C₄ haloalkyl, —P(O)(OR^{3d})₂, —C(O)CHR^{3e}NR^{3b}R^{3c}, —C(O)OH, —C(O)OR^{3a}, —C(O)NR^{3b}R^{3c}, C₃-C₈ cycloalkyl, C₅-C₇ cycloalkenyl, 3-6 membered heterocyclyl, C₅-C₇ heterocycloalkenyl, C₆ aryl, and 5-6 membered heteroaryl;
wherein the C₃-C₈ cycloalkyl, C₆ aryl, 3-6 membered heterocyclyl, and 5-6 membered heteroaryl of R³, are optionally substituted with 1 or 2 of R⁵;
R^{3a} is C₁-C₄ alkyl;
R^{3b} and R^{3c} are each independently selected from the group consisting of hydrogen, C₁-C₄ alkyl, C₃-C₆ cycloalkyl, and —CO₂R^{3f};
R^{3d} is selected from the group consisting of hydrogen, C₁-C₄ alkyl, and C₂-C₄ alkenyl;
R^{3e} is selected from the group consisting of hydrogen and C₁-C₄ alkyl; wherein the C₁-C₄ alkyl of R^{3d} is optionally substituted with 1 of R^{3g};
R^{3f} is C₁-C₄ alkyl;
R^{3g} selected from the group consisting of —OH, —SH, —SCH₃, —NH₂, —C(O)OH, —C(O)NH₂, —NHC(NH)NH₂, C₆ aryl, and 5-9 membered heteroaryl; wherein the C₆ aryl of R^{3g} is optionally substituted with 1 of —OH;
R⁴ is selected from the group consisting of —OH, —OR^{4a}, —C(O)NR^{4b}R^{4c}, and 5-6 membered heterocyclyl; wherein the 3-10 membered heterocyclyl of R⁴ is optionally substituted with 1 or 2 C₁-C₄ alkyl;
R^{4a} is C₁-C₄ alkyl; wherein the C₁-C₄ alkyl of R^{4a} is optionally substituted with 1-Si(R^{4d})₃;
R^{4b} and R^{4c} are each independently selected from the group consisting of H and C₁-C₄ alkyl;
R^{4d} is C₁-C₄ alkyl;
R⁵ is selected from the group consisting of fluoro, —CN, —OH, —OR^{1a}, ═O, —CH₂CO₂R^{5b}, C₁-C₄ alkyl, C₁-C₄ haloalkyl, C₁-C₄ hydroxyalkyl, C₁-C₄ hydroxyalkoxy, and C₂-C₆ alkoxyalkyl;
R^{5a} is C₁-C₄ alkyl; and
R^{5b} is C₁-C₄ alkyl;
n is an integer of 1 to 3.

In certain embodiments, the present disclosure is directed at compounds of formula (II), enantiomers, diastereomers, racemates, tautomers, prodrugs, hydrates, solvates or pharmaceutically acceptable salts thereof, wherein R³ is selected from the group consisting of hydrogen, C₁-C₄ alkyl, —P(O)(OR^{3d})₂, and —C(O)CHR^{3e}NR^{3b}R^{3c}.

In certain embodiments, the present disclosure is directed at compounds of formula (II), enantiomers, diastereomers, racemates, tautomers, prodrugs, hydrates, solvates or pharmaceutically acceptable salts thereof, wherein
R¹ is selected from the group consisting of hydrogen and C₁-C₆ alkyl; wherein the C₁-C₆ alkyl of R¹ is optionally substituted with 1 or 2 of R⁴;
R³ is selected from the group consisting of hydrogen, C₁-C₄ alkyl, —P(O)(OR^{3d})₂, and —C(O)CHR^{3e}NR^{3b}R^{3c}; and
R⁴ is selected from the group consisting of —OH and —OR^{4a}.

In certain embodiments, the compound of the formula (I) or (II) is

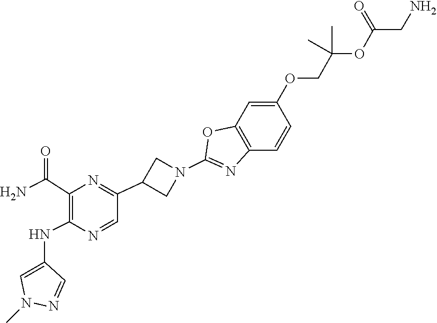 [SMILES: Cn1cc(Nc2ncc(C3CN(c4nc5ccc(OCC(C)(C)OC(=O)CN)cc5o4)C3)nc2C(N)=O)cn1]

In certain embodiments, the compound, or pharmaceutically acceptable salt is also provided.

In certain embodiments, the compound of the formula (I) or (II) is

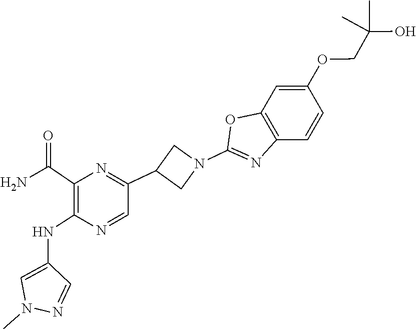 [SMILES: Cn1cc(Nc2ncc(C3CN(c4nc5ccc(OCC(C)(C)O)cc5o4)C3)nc2C(N)=O)cn1]

In certain embodiments, the compound, or pharmaceutically acceptable salt is also provided.

In certain embodiments, the compound of the formula (I) or (II), enantiomers, diastereomers, racemates, tautomers, prodrugs, hydrates, solvates or pharmaceutically acceptable salts thereof, is selected from the group consisting of 6-[1-(6-hydroxy-1,3-benzoxazol-2-yl)azetidin-3-yl]-3-[(1-{[2-(trimethylsilyl)ethoxy]methyl}-1H-pyrazol-4-yl)amino]pyrazine-2-carboxamide; 6-[1-(1,3-benzoxazol-2-yl)azetidin-3-yl]-3-[(1-methyl-1H-pyrazol-4-yl)amino]pyrazine-2-carboxamide; 6-[1-(6-chloro-1,3-benzoxazol-2-yl)azetidin-3-yl]-3-[(1-methyl-1H-pyrazol-4-yl)amino]pyrazine-2-carboxamide; 6-[1-(6-methyl-1,3-benzoxazol-2-yl)azetidin-3-yl]-3-[(1-methyl-1H-pyrazol-4-yl)amino]pyrazine-2-carboxamide; 6-[1-(5,6-dimethyl-1,3-benzoxazol-2-yl)azetidin-3-yl]-3-[(1-methyl-1H-pyrazol-4-yl)amino]pyrazine-2-carboxamide; 6-[1-(6-ethyl-1,3-benzoxazol-2-yl)azetidin-3-yl]-3-[(1-methyl-1H-pyrazol-4-yl)amino]pyrazine-2-carboxamide; 6-{1-[6-(cyclohex-1-en-1-yl)-1,3-benzoxazol-2-yl]azetidin-3-yl}-3-[(1-methyl-1H-pyrazol-4-yl)amino]pyrazine-2-carboxamide; 6-[1-(6-bromo-1,3-benzoxazol-2-yl)azetidin-3-yl]-3-[(1-methyl-1H-pyrazol-4-yl)amino]pyrazine-2-carboxamide; 6-[1-(6-cyclohexyl-1,3-benzoxazol-2-yl)azetidin-3-yl]-3-[(1-methyl-1H-pyrazol-4-yl)amino]pyrazine-2-carboxamide; 3-[(1-methyl-1H-pyrazol-4-yl)amino]-6-[1-(6-phenyl-1,3-benzoxazol-2-yl)azetidin-3-yl]pyrazine-2-carboxamide; 6-[1-(6-methoxy-1,3-benzoxazol-2-yl)azetidin-3-yl]-3-[(1-methyl-1H-pyrazol-4-yl)amino]pyrazine-2-carboxamide; 6-[1-(6-cyclopentyl-1,3-benzoxazol-2-yl)azetidin-3-yl]-3-[(1-methyl-1H-pyrazol-4-yl)amino]pyrazine-2-carboxamide; 6-{1-[6-(cyclopent-1-en-1-yl)-1,3-benzoxazol-2-yl]azetidin-3-yl}-3-[(1-methyl-1H-pyrazol-4-yl)amino]pyrazine-2-carboxamide; 6-{1-[6-(3,6-dihydro-2H-pyran-4-yl)-1,3-benzoxazol-2-yl]azetidin-3-yl}-3-[(1-methyl-1H-pyrazol-4-yl)amino]pyrazine-2-carboxamide; 3-[(1-methyl-1H-pyrazol-4-yl)amino]-6-{1-[6-(trifluoromethyl)-1,3-benzoxazol-2-yl]azetidin-3-yl}pyrazine-2-carboxamide; methyl 2-(3-{6-carbamoyl-5-[(1-methyl-1H-pyrazol-4-yl)amino]pyrazin-2-yl}azetidin-1-yl)-1,3-benzoxazole-6-carboxylate; 6-[1-(1,3-benzoxazol-2-yl)azetidin-3-yl]-3-[(1H-pyrazol-4-yl)amino]pyrazine-2-carboxamide; 3-[(1-methyl-1H-pyrazol-4-yl)amino]-6-{1-[6-(oxolan-3-yl)-1,3-benzoxazol-2-yl]azetidin-3-yl}pyrazine-2-carboxamide; 6-{1-[6-(2,5-dihydrofuran-3-yl)-1,3-benzoxazol-2-yl]azetidin-3-yl}-3-[(1-methyl-1H-pyrazol-4-yl)amino]pyrazine-2-carboxamide; 6-{1-[6-(hydroxymethyl)-1,3-benzoxazol-2-yl]azetidin-3-yl}-3-[(1-methyl-1H-pyrazol-4-yl)amino]pyrazine-2-carboxamide; 6-(1-{6-[(1E)-3-hydroxy-3-methylbut-1-en-1-yl]-1,3-benzoxazol-2-yl}azetidin-3-yl)-3-[(1-methyl-1H-pyrazol-4-yl)amino]pyrazine-2-carboxamide; 2-(3-{6-carbamoyl-5-[(1-methyl-1H-pyrazol-4-yl)amino]pyrazin-2-yl}azetidin-1-yl)-N,N-dimethyl-1,3-benzoxazole-6-carboxamide; 2-(3-{6-carbamoyl-5-[(1-methyl-1H-pyrazol-4-yl)amino]pyrazin-2-yl}azetidin-1-yl)-1,3-benzoxazole-6-carboxylic acid; 6-{1-[6-(2-hydroxypropan-2-yl)-1,3-benzoxazol-2-yl]azetidin-3-yl}-3-[(1-methyl-1H-pyrazol-4-yl)amino]pyrazine-2-carboxamide; 6-[1-(6-hydroxy-1,3-benzoxazol-2-yl)azetidin-3-yl]-3-[(1-methyl-1H-pyrazol-4-yl)amino]pyrazine-2-carboxamide; 6-{1-[6-(3-hydroxy-3-methylbutyl)-1,3-benzoxazol-2-yl]azetidin-3-yl}-3-[(1-methyl-1H-pyrazol-4-yl)amino]pyrazine-2-carboxamide; 6-(1-{6-[(1E)-3-methoxyprop-1-en-1-yl]-1,3-benzoxazol-2-yl}azetidin-3-yl)-3-[(1-methyl-1H-pyrazol-4-yl)amino]pyrazine-2-carboxamide; 6-{1-[6-(cyclopropylmethoxy)-1,3-benzoxazol-2-yl]azetidin-3-yl}-3-[(1-methyl-1H-pyrazol-4-yl)amino]pyrazine-2-carboxamide; 6-{1-[6-(cyanomethoxy)-1,3-benzoxazol-2-yl]azetidin-3-yl}-3-[(1-methyl-1H-pyrazol-4-yl)amino]pyrazine-2-carboxamide; 6-(1-{6-[3-hydroxy-2-(hydroxymethyl)propyl]-1,3-benzoxazol-2-yl}azetidin-3-yl)-3-[(1-methyl-1H-pyrazol-4-yl)amino]pyrazine-2-carboxamide; ethyl {[2-(3-{6-carbamoyl-5-[(1-methyl-1H-pyrazol-4-yl)amino]pyrazin-2-yl}azetidin-1-yl)-1,3-benzoxazol-6-yl]oxy}acetate; 6-[1-(6-fluoro-1,3-benzoxazol-2-yl)azetidin-3-yl]-3-{[1-(oxan-4-yl)-1H-pyrazol-4-yl]amino}pyrazine-2-carboxamide; 6-[1-(6-fluoro-1,3-benzoxazol-2-yl)azetidin-3-yl]-3-{[1-(2-hydroxy-2-methylpropyl)-1H-pyrazol-4-yl]amino}pyrazine-2-carboxamide; 6-[1-(1,3-benzoxazol-2-yl)azetidin-3-yl]-3-{[1-(2-methoxyethyl)-1H-pyrazol-4-yl]amino}pyrazine-2-carboxamide; 6-[1-(1,3-benzoxazol-2-yl)azetidin-3-yl]-3-{[1-(2-hydroxy-2-methylpropyl)-1H-pyrazol-4-yl]amino}pyrazine-2-carboxamide; 6-{1-[6-(3-hydroxy-3-methylbutoxy)-1,3-benzoxazol-2-yl]azetidin-3-yl}-3-[(1-methyl-1H-pyrazol-4-yl)amino]pyrazine-2-carboxamide; 6-{1-[6-(3-hydroxypropyl)-1,3-benzoxazol-2-yl]azetidin-3-yl}-3-[(1-methyl-1H-pyrazol-4-yl)amino]pyrazine-2-carboxamide; 6-(1-{6-[(1E)-3-hydroxyprop-1-en-1-yl]-1,3-benzoxazol-2-yl}azetidin-3-yl)-3-[(1-methyl-1H-pyrazol-4-yl)amino]pyrazine-2-carboxamide; 6-{1-[6-(3-methoxypropyl)-1,3-benzoxazol-2-yl]azetidin-3-yl}-3-[(1-methyl-1H-pyrazol-4-yl)amino]pyrazine-2-carboxamide; 6-[1-(1,3-benzoxazol-2-yl)azetidin-3-yl]-3-({1-[2-(dimethylamino)-2-oxoethyl]-1H-pyrazol-4-yl}amino)pyrazine-2-carboxamide; 6-{1-[6-(2-methoxyethoxy)-1,3-benzoxazol-2-yl]azetidin-3-yl}-3-[(1-methyl-1H-pyrazol-4-yl)amino]pyrazine-2-carboxamide; 3-{[1-(2,3-dihydroxypropyl)-1H-pyrazol-4-yl]amino}-6-[1-(6-fluoro-1,3-benzoxazol-2-yl)azetidin-3-yl]pyrazine-2-carboxamide; 6-[1-(6-fluoro-1,3-benzoxazol-2-yl)azetidin-3-yl]-3-[(1H-pyrazol-4-yl)amino]pyrazine-2-carboxamide; 3-({1-[(2,2-dimethyl-1,3-dioxolan-4-yl)methyl]-1H-pyrazol-4-yl}amino)-6-[1-(6-fluoro-1,3-benzoxazol-2-yl)azetidin-3-yl]pyrazine-2-carboxamide; 6-{1-[6-(2-amino-2-oxoethoxy)-1,3-benzoxazol-2-yl]azetidin-3-yl}-3-[(1-methyl-1H-pyrazol-4-yl)amino]pyrazine-2-carboxamide; 6-{1-[6-(2-hydroxy-2-methylpropoxy)-1,3-benzoxazol-2-yl]azetidin-3-yl}-3-[(1-methyl-1H-pyrazol-4-yl)amino]pyrazine-2-carboxamide; 6-(1-{6-[2-(dimethylamino)-2-oxoethoxy]-1,3-benzoxazol-2-yl}azetidin-3-yl)-3-[(1-methyl-1H-pyrazol-4-yl)amino]pyrazine-2-carboxamide; 6-{1-[6-(2-hydroxyethoxy)-1,3-benzoxazol-2-yl]azetidin-3-yl}-3-[(1-methyl-1H-pyrazol-4-yl)amino]pyrazine-2-carboxamide; 3-[(1-methyl-1H-pyrazol-4-yl)amino]-6-(1-{6-[(oxetan-3-yl)methoxy]-1,3-benzoxazol-2-yl}azetidin-3-yl)pyrazine-2-carboxamide; 6-(1-{6-[(3-hydroxycyclobutyl)methoxy]-1,3-benzoxazol-2-yl}azetidin-3-yl)-3-[(1-methyl-1H-pyrazol-4-yl)amino]pyrazine-2-carboxamide; 6-{1-[6-(2,3-dihydroxypropoxy)-1,3-benzoxazol-2-yl]azetidin-3-yl}-3-[(1-methyl-1H-pyrazol-4-yl)amino]pyrazine-2-carboxamide; 6-(1-{6-[(1-hydroxycyclobutyl)methoxy]-1,3-benzoxazol-2-yl}azetidin-3-yl)-3-[(1-methyl-1H-pyrazol-4-yl)amino]pyrazine-2-carboxamide; 6-(1-{6-[2-(3-hydroxyoxetan-3-yl)ethyl]-1,3-benzoxazol-2-yl}azetidin-3-yl)-3-[(1-methyl-1H-pyrazol-4-yl)amino]pyrazine-2-carboxamide; 3-[(1-methyl-1H-pyrazol-4-yl)amino]-6-{1-[6-(morpholine-4-carbonyl)-1,3-benzoxazol-2-yl]azetidin-3-yl}pyrazine-2-carboxamide; 6-(1-{6-[2-(dimethylamino)-2-oxoethyl]-1,3-benzoxazol-2-yl}azetidin-3-yl)-3-[(1-methyl-1H-pyrazol-4-yl)amino]pyrazine-2-carboxamide; 6-[1-(6-{[(2R)-1,4-dioxan-2-yl]methoxy}-1,3-benzoxazol-2-yl)azetidin-3-yl]-3-[(1-methyl-1H-pyrazol-4-yl)amino]pyrazine-2-carboxamide; 3-[(1-methyl-1H-pyrazol-4-yl)amino]-6-(1-{6-[2-oxo-2-(pyrrolidin-1-yl)ethoxy]-1,3-benzoxazol-2-yl}azetidin-3-yl)pyrazine-2-carboxamide; 6-(1-{6-[2-(methylamino)-2-oxoethyl]-1,3-benzoxazol-2-yl}azetidin-3-yl)-3-[(1-methyl-1H-pyrazol-4-yl)amino]pyrazine-2-carboxamide; 6-(1-{6-[3-hydroxy-2-(hydroxymethyl)propoxy]-1,3-benzoxazol-2-yl}azetidin-3-yl)-3-[(1-methyl-1H-pyrazol-4-yl)amino]pyrazine-2-carboxamide; 6-(1-{6-[(2,2-dimethyl-1,3-dioxan-5-yl)methoxy]-1,3-benzoxazol-2-yl}azetidin-3-yl)-3-[(1-methyl-1H-pyrazol-4-yl)amino]pyrazine-2-carboxamide; N-tert-butyl-2-(3-{6-carbamoyl-5-[(1-methyl-1H-pyrazol-4-yl)amino]pyrazin-2-yl}azetidin-1-yl)-1,3-benzoxazole-6-carboxamide; 2-(3-{6-carbamoyl-5-[(1-methyl-1H-pyrazol-4-yl)amino]pyrazin-2-yl}azetidin-1-yl)-N-cyclopropyl-1,3-benzoxazole-6-carboxamide; 3-{[1-(2-hydroxyethyl)-1H-pyrazol-4-yl]amino}-6-[1-(6-methyl-1,3-benzoxazol-2-yl)azetidin-3-yl]pyrazine-2-carboxamide; 3-{[1-(2-methoxyethyl)-1H-pyrazol-4-yl]amino}-6-[1-(6-methyl-1,3-benzoxazol-2-yl)azetidin-3-yl]pyrazine-2-carboxamide; 6-[1-(6-{[3-(hydroxymethyl)cyclobutyl]methoxy}-1,3-benzoxazol-2-yl)azetidin-3-yl]-3-[(1-methyl-1H-pyrazol-4-yl)amino]pyrazine-2-carboxamide; 6-(1-{6-[(3-hydroxyoxetan-3-yl)methoxy]-1,3-benzoxazol-2-yl}azetidin-3-yl)-3-[(1-methyl-1H-pyrazol-4-yl)amino]pyrazine-2-carboxamide; 6-[1-(6-{[(1S,2R)-2-(hydroxymethyl)cyclopropyl]methoxy}-1,3-benzoxazol-2-yl)azetidin-3-yl]-3-[(1-methyl-1H-pyrazol-4-yl)amino]pyrazine-2-carboxamide; 6-(1-{6-[(2R)-2-(hydroxymethyl)pyrrolidine-1-carbonyl]-1,3-benzoxazol-2-yl}azetidin-3-yl)-3-[(1-methyl-1H-pyrazol-4-yl)amino]pyrazine-2-carboxamide; 3-[(1-methyl-1H-pyrazol-4-yl)amino]-6-{1-[6-(3-phenylpropoxy)-1,3-benzoxazol-2-yl]azetidin-3-yl}pyrazine-2-carboxamide; 6-{1-[6-(3-hydroxy-2,2-dimethylpropoxy)-1,3-benzoxazol-2-yl]azetidin-3-yl}-3-[(1-methyl-1H-pyrazol-4-yl)amino]pyrazine-2-carboxamide; 6-(1-{6-[3-(dimethylamino)-3-oxopropyl]-1,3-benzoxazol-2-yl}azetidin-3-yl)-3-[(1-methyl-1H-pyrazol-4-yl)amino]pyrazine-2-carboxamide; 3-[(1-methyl-1H-pyrazol-4-yl)amino]-6-(1-{6-[3-oxo-3-(pyrrolidin-1-yl)propyl]-1,3-benzoxazol-2-yl}azetidin-3-yl)pyrazine-2-carboxamide; 6-(1-{6-[(2R)-2-(methoxymethyl)pyrrolidine-1-carbonyl]-1,3-benzoxazol-2-yl}azetidin-3-yl)-3-[(1-methyl-1H-pyrazol-4-yl)amino]pyrazine-2-carboxamide; 3-[(1-methyl-1H-pyrazol-4-yl)amino]-6-(1-{6-[2-oxo-2-(pyrrolidin-1-yl)ethyl]-1,3-benzoxazol-2-yl}azetidin-3-yl)pyrazine-2-carboxamide; [2-(3-{6-carbamoyl-5-[(1-methyl-1H-pyrazol-4-yl)amino]pyrazin-2-yl}azetidin-1-yl)-1,3-benzoxazol-6-yl]acetic acid; 6-[1-(6-{2-[(2S)-2-(hydroxymethyl)pyrrolidin-1-yl]-2-oxoethyl}-1,3-benzoxazol-2-yl)azetidin-3-yl]-3-[(1-methyl-1H-pyrazol-4-yl)amino]pyrazine-2-carboxamide; 6-{1-[6-(2-hydroxy-2-methylpropyl)-1,3-benzoxazol-2-yl]azetidin-3-yl}-3-[(1-methyl-1H-pyrazol-4-yl)amino]pyrazine-2-carboxamide; 6-(1-{6-[(2R)-2-hydroxypropoxy]-1,3-benzoxazol-2-yl}azetidin-3-yl)-3-[(1-methyl-1H-pyrazol-4-yl)amino]pyrazine-2-carboxamide; 3-[(1-methyl-1H-pyrazol-4-yl)amino]-6-[1-(6-{[(3S)-oxolan-3-yl]oxy}-1,3-benzoxazol-2-yl)azetidin-3-yl]pyrazine-2-carboxamide; 6-[1-(6-{2-[(3S)-3-methoxypyrrolidin-1-yl]-2-oxoethyl}-1,3-benzoxazol-2-yl)azetidin-3-yl]-3-[(1-methyl-1H-pyrazol-4-yl)amino]pyrazine-2-carboxamide; 6-[1-(6-{2-[(2R)-2-(methoxymethyl)pyrrolidin-1-yl]-2-oxoethyl}-1,3-benzoxazol-2-yl)azetidin-3-yl]-3-[(1-methyl-1H-pyrazol-4-yl)amino]pyrazine-2-carboxamide; 6-(1-{6-[(1-methyl-2-oxopyrrolidin-3-yl)oxy]-1,3-benzoxazol-2-yl}azetidin-3-yl)-3-[(1-methyl-1H-pyrazol-4-yl)amino]pyrazine-2-carboxamide; 6-(1-{6-[2-(azetidin-1-yl)-2-oxoethoxy]-1,3-benzoxazol-2-yl}azetidin-3-yl)-3-[(1-methyl-1H-pyrazol-4-yl)amino]pyrazine-2-carboxamide; {[2-(3-{6-carbamoyl-5-[(1-methyl-1H-pyrazol-4-yl)amino]pyrazin-2-yl}azetidin-1-yl)-1,3-benzoxazol-6-yl]oxy}acetic acid; 6-[1-(6-methyl-1,3-benzoxazol-2-yl)azetidin-3-yl]-3-[(1H-pyrazol-4-yl)amino]pyrazine-2-carboxamide; 6-(1-{6-[(2R)-2-(2-hydroxypropan-2-yl)pyrrolidine-1-carbonyl]-1,3-benzoxazol-2-yl}azetidin-3-yl)-3-[(1-methyl-1H-pyrazol-4-yl)amino]pyrazine-2-carboxamide; 6-(1-{6-[(1-methyl-2-oxopiperidin-3-yl)oxy]-1,3-benzoxazol-2-yl}azetidin-3-yl)-3-[(1-methyl-1H-pyrazol-4-yl)amino]pyrazine-2-carboxamide; 4-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylbutan-2-yl dihydrogen phosphate; 4-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylbutan-2-yl diprop-2-en-1-yl phosphate; 6-[1-(6-methyl-1,3-benzoxazol-2-yl)azetidin-3-yl]-3-[(1-methyl-1H-pyrazol-4-yl)amino]pyridine-2-carboxamide; 6-(1-{6-[(2-hydroxycyclopentyl)oxy]-1,3-benzoxazol-2-yl}azetidin-3-yl)-3-[(1-methyl-1H-pyrazol-4-yl)amino]pyrazine-2-carboxamide; 3-[(1-methyl-1H-pyrazol-4-yl)amino]-6-(1-{6-[(1-methyl-1H-pyrazol-3-yl)methoxy]-1,3-benzoxazol-2-yl}azetidin-3-yl)pyrazine-2-carboxamide; 3-[(1-methyl-1H-pyrazol-4-yl)amino]-6-[1-(6-{2-oxo-2-[(2R)-2-(trifluoromethyl)pyrrolidin-1-yl]ethyl}-1,3-benzoxazol-2-yl)azetidin-3-yl]pyrazine-2-carboxamide; 3-[(1-methyl-1H-pyrazol-4-yl)amino]-6-(1-{6-[(5-methyl-1,3-thiazol-2-yl)methoxy]-1,3-benzoxazol-2-yl}azetidin-3-yl)pyrazine-2-carboxamide; 6-(1-{6-[2-(dimethylamino)-2-oxoethoxy]-1,3-benzoxazol-2-yl}azetidin-3-yl)-3-[(1-methyl-1H-pyrazol-4-yl)amino]pyridine-2-carboxamide; 6-{1-[6-(3-hydroxy-3-methylbutoxy)-1,3-benzoxazol-2-yl]azetidin-3-yl}-3-[(1-methyl-1H-pyrazol-4-yl)amino]pyridine-2-carboxamide; 6-[1-(6-hydroxy-1,3-benzoxazol-2-yl)azetidin-3-yl]-3-[(1-methyl-1H-pyrazol-4-yl)amino]pyridine-2-carboxamide; 6-{1-[6-(2-hydroxy-2-methylpropoxy)-1,3-benzoxazol-2-yl]azetidin-3-yl}-3-[(1-methyl-1H-pyrazol-4-yl)amino]pyridine-2-carboxamide; 6-(1-{6-[(5-methyl-1,3,4-oxadiazol-2-yl)methoxy]-1,3-benzoxazol-2-yl}azetidin-3-yl)-3-[(1-methyl-1H-pyrazol-4-yl)amino]pyrazine-2-carboxamide; 6-{1-[6-(2-cyclopropyl-2-oxoethoxy)-1,3-benzoxazol-2-yl]azetidin-3-yl}-3-[(1-methyl-1H-pyrazol-4-yl)amino]pyrazine-2-carboxamide; 6-{1-[6-(4-hydroxyphenyl)-1,3-benzoxazol-2-yl]azetidin-3-yl}-3-[(1-methyl-1H-pyrazol-4-yl)amino]pyrazine-2-carboxamide; 6-(1-{6-[4-(2-hydroxyethoxy)phenyl]-1,3-benzoxazol-2-yl}azetidin-3-yl)-3-[(1-methyl-1H-pyrazol-4-yl)amino]pyrazine-2-carboxamide; 6-(1-{6-[1-(2-hydroxyethyl)-1H-pyrazol-4-yl]-1,3-benzoxazol-2-yl}azetidin-3-yl)-3-[(1-methyl-1H-pyrazol-4-yl)amino]pyrazine-2-carboxamide; 6-(1-{6-[1-(2-methoxyethyl)-1H-pyrazol-4-yl]-1,3-benzoxazol-2-yl}azetidin-3-yl)-3-[(1-methyl-1H-pyrazol-4-yl)amino]pyrazine-2-carboxamide; 3-[(1-methyl-1H-pyrazol-4-yl)amino]-6-{1-[6-(1-methyl-1H-pyrazol-4-yl)-1,3-benzoxazol-2-yl]azetidin-3-yl}pyrazine-2-carboxamide; 3-[(1-methyl-1H-pyrazol-4-yl)amino]-6-(1-{6-[(1H-pyrazol-4-yl)methoxy]-1,3-benzoxazol-2-yl}azetidin-3-yl)pyrazine-2-carboxamide; 3-[(1-tert-butyl-1H-pyrazol-4-yl)amino]-6-{1-[6-(3-hydroxy-3-methylbutoxy)-1,3-benzoxazol-2-yl]azetidin-3-yl}pyrazine-2-carboxamide; 4-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylbutan-2-yl glycinate; 4-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylbutan-2-yl [(tert-butoxycarbonyl)amino]acetate; 6-{1-[6-(3-hydroxy-3-methylbutoxy)-1,3-benzoxazol-2-yl]azetidin-3-yl}-3-{[1-(2-hydroxy-2-methylpropyl)-1H-pyrazol-4-yl]amino}pyrazine-2-carboxamide; 6-{1-[6-(3-hydroxy-3-methylbutoxy)-1,3-benzoxazol-2-yl]azetidin-3-yl}-3-[(1-{[2-(trimethylsilyl)ethoxy]methyl}-1H-pyrazol-4-yl)amino]pyrazine-2-carboxamide; 6-{1-[6-(3-hydroxy-3-methylbutoxy)-1,3-benzoxazol-2-yl]azetidin-3-yl}-3-[(1H-pyrazol-4-yl)amino]pyrazine-2-carboxamide; 3-[(1-methyl-1H-pyrazol-4-yl)amino]-6-(1-{6-[(1-methyl-1H-pyrazol-4-yl)methoxy]-1,3-benzoxazol-2-yl}azetidin-3-yl)pyrazine-2-carboxamide; 6-(1-{6-[(1-methyl-1H-imidazol-4-yl)methoxy]-1,3-benzoxazol-2-yl}azetidin-3-yl)-3-[(1-methyl-1H-pyrazol-4-yl)amino]pyrazine-2-carboxamide; 3-[(1-methyl-1H-pyrazol-4-yl)amino]-6-{1-[6-(2-methyl-1,3-thiazol-5-yl)-1,3-benzoxazol-2-yl]azetidin-3-yl}pyrazine-2-carboxamide; 6-(1-{6-[4-(2-methoxyethoxy)phenyl]-1,3-benzoxazol-2-yl}azetidin-3-yl)-3-[(1-methyl-1H-pyrazol-4-yl)amino]pyrazine-2-carboxamide; 6-{1-[6-(2-ethoxypropan-2-yl)-1,3-benzoxazol-2-yl]azetidin-3-yl}-3-[(1-methyl-1H-pyrazol-4-yl)amino]pyrazine-2-carboxamide; 6-(1-{6-[(5-methoxypyrimidin-2-yl)oxy]-1,3-benzoxazol-2-yl}azetidin-3-yl)-3-[(1-methyl-1H-pyrazol-4-yl)amino]pyrazine-2-carboxamide; 3-[(1-methyl-1H-pyrazol-4-yl)amino]-6-(1-{6-[(pyridazin-3-yl)oxy]-1,3-benzoxazol-2-yl}azetidin-3-yl)pyridine-2-carboxamide; 6-[1-(6-{[1-(2-hydroxyethyl)-1H-pyrazol-4-yl]methoxy}-1,3-benzoxazol-2-yl)azetidin-3-yl]-3-[(1-methyl-1H-pyrazol-4-yl)amino]pyrazine-2-carboxamide; ethyl [4-({[2-(3-{6-carbamoyl-5-[(1-methyl-1H-pyrazol-4-yl)amino]pyrazin-2-yl}azetidin-1-yl)-1,3-benzoxazol-6-yl]oxy}methyl)-1H-pyrazol-1-yl]acetate; 6-{1-[6-(1-methyl-1H-imidazol-4-yl)-1,3-benzoxazol-2-yl]azetidin-3-yl}-3-[(1-methyl-1H-pyrazol-4-yl)amino]pyrazine-2-carboxamide; 1-{[2-(3-{6-carbamoyl-5-[(1-methyl-1H-pyrazol-4-yl)amino]pyridin-2-yl}azetidin-1-yl)-1,3-benzoxazol-6-yl]oxy}-2-methylpropan-2-yl dihydrogen phosphate; 6-{1-[6-(2-cyano-2-methylpropoxy)-1,3-benzoxazol-2-yl]azetidin-3-yl}-3-[(1-methyl-1H-pyrazol-4-yl)amino]pyrazine-2-carboxamide; 1-{[2-(3-{6-carbamoyl-5-[(1-methyl-1H-pyrazol-4-yl)amino]pyridin-2-yl}azetidin-1-yl)-1,3-benzoxazol-6-yl]oxy}-2-methylpropan-2-yl glycinate; 4-{[2-(3-{6-carbamoyl-5-[(1-methyl-1H-pyrazol-4-yl)amino]pyridin-2-yl}azetidin-1-yl)-1,3-benzoxazol-6-yl]oxy}-2-methylbutan-2-yl dihydrogen phosphate; 4-{[2-(3-{6-carbamoyl-5-[(1-methyl-1H-pyrazol-4-yl)amino]pyridin-2-yl}azetidin-1-yl)-1,3-benzoxazol-6-yl]oxy}-2-methylbutan-2-yl glycinate; 6-{1-[6-(1-hydroxy-2-methylpropan-2-yl)-1,3-benzoxazol-2-yl]azetidin-3-yl}-3-[(1-methyl-1H-pyrazol-4-yl)amino]pyrazine-2-carboxamide; ethyl 2-[2-(3-{6-carbamoyl-5-[(1-methyl-1H-pyrazol-4-yl)amino]pyrazin-2-yl}azetidin-1-yl)-1,3-benzoxazol-6-yl]-2-methylpropanoate; 1-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylpropan-2-yl dihydrogen phosphate; and 1-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylpropan-2-yl glycinate.

In certain embodiments, the compound or pharmaceutically acceptable salts of 6-{1-[6-(2-hydroxy-2-methylpropoxy)-1,3-benzoxazol-2-yl]azetidin-3-yl}-3-[(1-methyl-1H-pyrazol-4-yl)amino]pyrazine-2-carboxamide are provided. In certain embodiments, the compound 6-{1-[6-(2-hydroxy-2-methylpropoxy)-1,3-benzoxazol-2-yl]azetidin-3-yl}-3-[(1-methyl-1H-pyrazol-4-yl)amino]pyrazine-2-carboxamide is provided.

In certain embodiments, the compound, or pharmaceutically acceptable salts of 1-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylpropan-2-yl glycinate are provided. In certain embodiments, the compound 1-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylpropan-2-yl glycinate is provided.

Certain embodiments of the present disclosure relate to pharmaceutical compositions comprising compounds of the present disclosure enantiomers, diastereomers, racemates, tautomers, prodrugs, hydrates, solvates or pharmaceutically acceptable salts thereof, and in combination with a pharmaceutically acceptable carrier.

### Technical Solution

The present invention provides novel compounds capable of selectively inhibiting Mer and Axl kinases, which compounds are useful for the prevention and/or the treatment of cancer and other immune-related disease such as infection and sepsis Highly potent and selective receptor tyrosine kinase Mer and Axl inhibitors based on an aminopyridine scaffold are described. Such compounds have the following formula (I).

Disclosed herein are compounds of formula (I), or a pharmaceutically acceptable salt thereof,

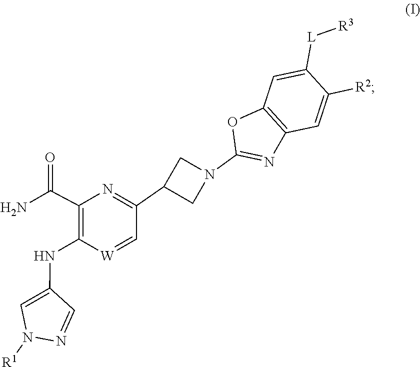

wherein L, R¹, R², R³, and W are defined above in the Summary and below in the Detailed Description Further, composition comprising such compounds are also disclosed.

Compounds disclosed herein may contain one or more variable(s) that occur more than one time in any substituent or in the Formula herein. Definition of a variable on each occurrence is independent of its definition at another occurrence. Further, combinations of substituents are permissible only if such combinations result in stable compounds. Stable compounds are compounds, which can be isolated from a reaction mixture.

### Definitions

Certain terms as used in the specification and claims are intended to refer to the following definitions, as detailed below.

It is noted that, as used in this specification and the intended claims, the singular form “a,” “an,” and “the” include plural referents unless the context clearly dictates otherwise. Thus, for example, reference to “a compound” includes a single compound as well as one or more of the same or different compounds. Reference to “a pharmaceutically acceptable carrier” means a single pharmaceutically acceptable carrier as well as one or more pharmaceutically acceptable carriers.

As used in the specification and the appended claims, unless specified to the contrary, the following terms have the meaning indicated:

The term “alkenyl,” as used herein, refers to a straight or branched hydrocarbon chain radical containing at least one carbon-carbon double bond. Representative examples of alkenyl include, but are not limited to, ethenyl, 2-propenyl, 3-butenyl, 4-pentenyl, 5-hexenyl, 2-heptenyl, 2-methyl-1-heptenyl, and 3-decenyl, and the like.

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy, and the like.

The term “alkoxyalkyl,” as used herein, refers to an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl, and the like.

The term “alkyl,” as used herein, refers to a saturated, straight or branched hydrocarbon chain radical. Representative examples of alkyl include, but are not limited to, methyl, ethyl, n-propyl, isopropyl, n-butyl, sec-butyl, isobutyl, tert-butyl, n-pentyl, isopentyl, neopentyl, n-hexyl, 1-methylbutyl, 2-methylbutyl, 3-methylbutyl, 3,3-dimethylbutyl, 1,1-dimethylpropyl, 1,2-dimethylpropyl, 2,2-dimethylpropyl, 1-methylpropyl, 2-methylpropyl, 1-ethylpropyl, and 1,2,2-trimethylpropyl, and the like.

The term “aryl,” as used herein, refers to a monocyclic, bicyclic, or a tricyclic fused hydrocarbon ring system radical wherein one or more of the hydrocarbon rings is aromatic. The bicyclic aryl is naphthyl, or a phenyl fused to a cycloalkyl, or a phenyl fused to a cycloalkenyl. The bicyclic aryl and tricyclic aryl are attached to the parent molecular moiety through any carbon atom contained within the ring system. Representative examples of the aryl groups include, but are not limited to, phenyl, dihydroindenyl, indenyl, naphthyl, dihydronaphthalenyl, tetrahydronaphthalenyl, and the like.

The term “cycloalkenyl,” as used herein, refers to a radical of a non-aromatic monocyclic or a bicyclic hydrocarbon ring system containing at least one double bond. The “monocyclic cycloalkenyl” has five, six, or seven carbon atoms. The five and six membered ring systems have one or two double bonds, and the seven membered ring systems has one, two or three double bonds. Representative examples of monocyclic cycloalkenyl groups include, but are not limited to, cyclopentenyl, cyclohexenyl, and cycloheptenyl, and the like.

The term “cycloalkyl,” as used herein, refers to a saturated hydrocarbon ring radical containing carbon ring atoms. The cycloalkyl may be a monocyclic, a bicyclic, or a spirocyclic cycloalkyl. The monocyclic cycloalkyl is a carbocyclic ring system containing three to eight carbon atoms, zero heteroatoms and zero double bonds. Examples of monocyclic ring systems include cyclopropyl, cyclobutyl, cyclopentyl, cyclohexyl, cycloheptyl, and cyclooctyl. The bicyclic cycloalkyl is a monocyclic cycloalkyl fused to a monocyclic cycloalkyl ring, or a bridged monocyclic ring system in which two non-adjacent carbon atoms of the monocyclic ring are linked by an alkylene bridge containing one, two, three, or four carbon atoms. Representative examples of bicyclic ring systems include, but are not limited to, bicyclo[3.1.1]heptanyl, bicyclo[2.2.1]heptanyl, bicyclo[2.2.2]octanyl, and the like. Spirocyclic cycloalkyl is exemplified by a monocyclic or a bicyclic cycloalkyl, wherein two of the substituents on the same carbon atom of the ring, together with said carbon atom, form a 4-, 5-, or 6-membered monocyclic cycloalkyl. An example of a spirocyclic cycloalkyl is spiro[2.5]octanyl.

In a fused ring system, the two rings share one common bond.

The term “halo” or “halogen,” as used herein, means Cl, Br, I, and F.

The term “haloalkyl,” as used herein, refers to an alkyl group, as defined herein, in which one or more hydrogen atoms are replaced by halogen. Representative examples of haloalkyl include, but are not limited to, chloromethyl, 2-fluoroethyl, 2,2-difluoroethyl, fluoromethyl, 2,2,2-trifluoroethyl, trifluoromethyl, difluoromethyl, pentafluoroethyl, 2-chloro-3-fluoropentyl, trifluorobutyl, and trifluoropropyl, and the like.

The term “heteroaryl,” as used herein, refers to an aromatic ring radical containing one or more heteroatoms or a ring system containing one or more heteroaryl rings. The monocyclic heteroaryl is a four-, five- or six-membered ring. The five-membered ring contains two double bonds and one or more heteroatoms selected from O, S, and N. The six-membered ring contains three double bonds and one, two, three or four nitrogen atoms. Representative examples of monocyclic heteroaryl include, but are not limited to, furanyl, imidazolyl, isoxazolyl, isothiazolyl, oxadiazolyl, 1,3-oxazolyl, pyridinyl, pyridazinyl, pyrimidinyl, pyrazinyl, pyrazolyl, pyrrolyl, tetrazolyl, thiadiazolyl, 1,3-thiazolyl, thienyl, triazolyl, and triazinyl, and the like.

The term “heteroatom,” as used herein, means a nitrogen, oxygen, or sulfur atom.

The term “heterocycle” or “heterocyclyl” as used herein, refers to a hydrocarbon ring radical wherein at least one carbon atom is replaced by a heteroatom independently selected from the group consisting of O, N, and S. Monocyclic ring systems are exemplified by any 3- or 4-membered ring containing a heteroatom independently selected from oxygen, nitrogen and sulfur; or a 5-, or 6-membered ring containing one, two or three heteroatoms wherein the heteroatoms are independently selected from nitrogen, oxygen and sulfur. The 5-membered ring has from 0-2 double bonds and the 6-membered ring has from 0-3 double bonds. Representative examples of heterocyclyl monocyclic ring systems include, but are not limited to 1,3-dioxolanyl, dioxanyl, dithianyl, furanyl (furyl), imidazolyl, imidazolinyl, imidazolidinyl, isothiazolyl, isothiazolinyl, isothiazolidinyl, isoxazolyl, isoxazolinyl, isoxazolidinyl, morpholinyl, oxadiazolyl, oxadiazolinyl, oxadiazolidinyl, oxazolyl, oxazolinyl, oxazolidinyl, piperazinyl, piperidinyl, pyranyl, pyrazinyl, pyrazolyl, pyrazolinyl, pyrazolidinyl, pyridinyl, pyrimidinyl, pyridazinyl, pyrrolyl, pyrrolinyl, pyrrolidinyl, tetrahydrofuranyl, tetrahydrothienyl, tetrazinyl, tetrazolyl, thiadiazolyl, thiadiazolinyl, thiadiazolidinyl, thiazolyl, thiazolinyl, thiazolidinyl, thienyl, thiomorpholinyl, thiopyranyl, triazinyl, triazolyl, trithianyl, and the like.

The term “heterocycloalkenyl” refers to a non-aromatic partially unsaturated monocyclic or polycyclic heterocycloalkene radical containing carbon ring atoms and 1 or more ring heteroatoms independently selected from S, N or O. A heterocycloalkenyl may be a single (monocyclic) ring. Examples of monocyclic heterocycloalkenyls include 2,5-dihydrofuranyl, 3,6-dihydro-2H-pyranyl, 2,3,4,5-tetrahydro-1H-azepinyl, 3,4,5,6-tetrahydro-2H-oxocinyl, 1,2,3,6-tetrahydropyridinyl, and 4,5-dihydro-1H-imidazolyl. A heterocycloalkenyl may alternatively be polycyclic (contain more than one ring). Examples of polycyclic heterocycloalkenyls include bridged, fused, and spirocyclic heterocycloalkenyls in which at least one ring is a heterocycloalkenyl and the others are heterocycloalkenyl, heterocycloalkyl, cycloalkenyl or cycloalkyl rings. Alternatively, a polycyclic heterocycloalkenyl may consist of one or more heterocycloalkyl rings and one or more cycloalkenyl rings.

The term “hydroxyalkoxy,” as used herein, refers to a hydroxy group, as defined herein, appended to the parent molecular moiety through an alkoxy group, as defined herein. Representative examples of hydroxyalkoxy include, but are not limited to, hydroxymethoxy, hydroxyethoxy, hydroxypropoxy, and the like.

The term “hydroxyalkyl,” as used herein, refers to a hydroxy group, as defined herein, appended to the parent molecular moiety through an alkyl group, as defined herein. Representative examples of hydroxyalkyl include, but are not limited to, hydroxymethyl, 2-hydroxyethyl, 3-hydroxypropyl, 2-hydroxypropan-2-yl, and the like.

In some instances, the number of carbon atoms in a moiety is indicated by the prefix “Cₓ-C_{y}”, wherein x is the minimum and y is the maximum number of carbon atoms in the substituent. Thus, for example, “C₁-C₆ alkyl” means an alkyl substituent containing from 1 to 6 carbon atoms and “C₁-C₃ alkyl” means an alkyl substituent containing from 1 to 3 carbon atoms. Also, for example, “C₆-C₁₀ aryl” as used herein, means phenyl or a bicyclic aryl with 6 to 10 carbon atoms.

In some instances, the number of ring atoms in a moiety is indicated by the prefix “x-y membered”, wherein x is the minimum and y is the maximum number of ring atoms in the substituent. Thus, for example, the term “5- to 6-membered heteroaryl” means a heteroaryl containing 5 to 6 ring atoms.

If a moiety is described as being “optionally substituted,” the moiety may be either (1) not substituted or (2) substituted. If a moiety is described as being optionally substituted with up to a particular number of non-hydrogen radicals, that moiety may be either (1) not substituted; or (2) substituted by up to that particular number of non-hydrogen radicals or by up to the maximum number of substitutable positions on the moiety, whichever is less. Thus, for example, if a moiety is described as a heteroaryl optionally substituted with up to 3 non-hydrogen radicals, then any heteroaryl with less than 3 substitutable positions would be optionally substituted by up to only as many non-hydrogen radicals as the heteroaryl has substitutable positions. To illustrate, tetrazolyl (which has only one substitutable position) would be optionally substituted with up to one non-hydrogen radical. To illustrate further, if an amino nitrogen is described as being optionally substituted with up to 2 non-hydrogen radicals, then a primary amino nitrogen will be optionally substituted with up to 2 non-hydrogen radicals, whereas a secondary amino nitrogen will be optionally substituted with up to only 1 non-hydrogen radical.

With reference to the use of the words “comprise” or “comprises” or “comprising” in this patent application (including the claims), Applicants note that unless the context requires otherwise, those words are used on the basis and clear understanding that they are to be interpreted inclusively, rather than exclusively, and that Applicants intend each of those words to be so interpreted in construing this patent application, including the claims below.

The phrase “pharmaceutical composition” refers to a composition suitable for administration in medical or veterinary use.

The phrase “pharmaceutically acceptable salt” refers to those salts which are, within the scope of sound medical judgement, suitable for use in contact with the tissues of humans and lower animals without undue toxicity, irritation, allergic response and the like and are commensurate with a reasonable benefit/risk ratio.

The terms “prevent,” “preventing,” and “prevention” refer to a method of preventing the onset of a disease and/or its attendant symptoms or barring a subject from acquiring a disease. As used herein, “prevent,” “preventing,” and “prevention” also include delaying the onset of a disease and/or its attendant symptoms and reducing a subject's risk of acquiring or developing a disease or disorder.

The term “subject,” as used herein, refers to animals such as mammals, including, but not limited to, primates (e.g., humans), cows, sheep, goats, pigs, horses, dogs, cats, rabbits, rats, mice and the like. In one embodiment, the subject is a human. The terms “human,” “patient,” and “subject” are used interchangeably herein.

If a moiety is described as “substituted,” a non-hydrogen radical is in the place of hydrogen radical of any substitutable atom of the moiety. Thus, for example, a substituted heterocycle moiety is a heterocycle moiety in which at least one non-hydrogen radical is in the place of a hydrogen radical on the heterocycle. It should be recognized that if there are more than one substitution on a moiety, each non-hydrogen radical may be identical or different (unless otherwise stated).

The phrase “therapeutically effective amount” refers to an amount of a compound, or a pharmaceutically acceptable salt thereof, sufficient to prevent the development of or to alleviate to some extent one or more of the symptoms of the condition or disorder being treated when administered alone or in conjunction with one or more therapeutic agents for treatment in a particular subject or subject population. The “therapeutically effective amount” may vary depending on the compound, the disease and its severity, and the age, weight, health, etc., of the subject to be treated. For example in a human or other mammal, a therapeutically effective amount may be determined experimentally in a laboratory or clinical setting, or may be the amount required by the guidelines of the United States Food and Drug Administration, or equivalent foreign agency, for the particular disease and subject being treated.

The terms “treat,” “treating,” and “treatment,” as used herein, refer to a method of alleviating or abrogating a disease and/or its attendant symptoms.

### Compounds

Compounds of the present disclosure have the general formula (I) as described above in the Summary and Detailed Description.

In certain embodiments, compounds of the present disclosure are represented by formula (I), and enantiomers, diastereomers, racemates, tautomers, prodrugs, hydrates, solvates and pharmaceutically acceptable salts thereof, where

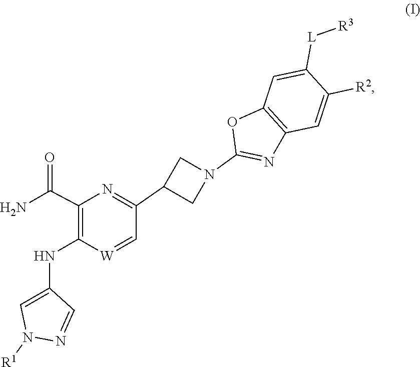

where
W is N or CH;
L is selected from the group consisting of a bond, —CH═CHCR^{a}R^{b}—, —(CR^{a}R^{b})ₙ—O—, —(CR^{a}R^{b})ₙ—C(O)—, —O—, —O—(CR^{a}R^{b})ₙ—, —O—(CR^{a}R^{b})ₙ—O—, —OCH₂C(O)—, and —C(O)—;
each R^{a} is independently selected from the group consisting of hydrogen, C₁-C₄ alkyl, and C₁-C₂ hydroxyalkyl;
each R^{b} is independently selected from the group consisting of hydrogen and C₁-C₄ alkyl;
where R^{a} and R^{b} optionally combine to form a C₃₋₆ cycloalkyl or 3-6 membered heterocycle;
R¹ is selected from the group consisting of H, C₁-C₆ alkyl, and 5-6 membered heterocyclyl; wherein the C₁-C₆ alkyl of R¹ is optionally substituted with 1 or 2 of R⁴;
R² is selected from the group consisting of hydrogen, fluoro, chloro, bromo, and C₁-C₄ alkyl;
R³ is selected from the group consisting of hydrogen, fluoro, chloro, bromo, —CN, C₁-C₄ alkyl, C₁-C₄ haloalkyl, —OH, —OR^{3a}, —NR^{3b}R^{3c}, —P(O)(OR^{3d})₂, —C(O)CHR^{3e}NR^{3b}R^{3c}, —C(O)OH, —C(O)OR^{3a}, —C(O)NR^{3b}R^{3c}, C₃-C₈ cycloalkyl, C₅-C₇ cycloalkenyl, 3-6 membered heterocyclyl, C₅-C₇ heterocycloalkenyl, C₆ aryl, 5-6 membered heteroaryl and

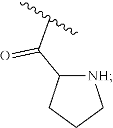 [SMILES: *C(=O)C1CCCN1]

wherein the C₃-C₈ cycloalkyl, C₆ aryl, 3-6 membered heterocyclyl, and 5-6 membered heteroaryl of R³, are optionally substituted with 1 or 2 R⁵;
R^{3a} is C₁-C₄ alkyl;
R^{3b} and R^{3c} are each independently selected from the group consisting of hydrogen, C₁-C₄ alkyl, C₃-C₆ cycloalkyl, and —CO₂R^{3f};
R^{3d} is selected from the group consisting of hydrogen, C₁-C₄ alkyl, and C₂-C₄ alkenyl;
R^{3c} is selected from the group consisting of hydrogen and C₁-C₄ alkyl; wherein the C₁-C₄ alkyl of R^{3d} is optionally substituted with 1 of R^{3g};
R^{3f} is C₁-C₄ alkyl;
R^{3g} selected from the group consisting of —OH, —SH, —SCH₃, —NH₂, —C(O)OH, —C(O)NH₂, —NHC(NH)NH₂, C₆ aryl, and 5-9 membered heteroaryl; wherein the C₆ aryl of R^{3g} is optionally substituted with 1 of —OH;
R⁴ is selected from the group consisting of —OH, —OR^{4a}, —C(O)NR^{4b}R^{4c}, and 5-6 membered heterocyclyl; wherein the 5-6 membered heterocyclyl of R⁴ is optionally substituted with 1 or 2 of C₁-C₄ alkyl;
R^{4a} is C₁-C₄ alkyl; wherein the C₁-C₄ alkyl of R^{4a} is optionally substituted with 1 of —Si(R^{4d})₃;
R^{4b} and R^{4c} are each independently selected from the group consisting of hydrogen and C₁-C₄ alkyl;
R^{4d} is C₁-C₄ alkyl;
R⁵ is selected from the group consisting of fluoro, —CN, —OH, —OR^{5a}, ═O, —CH₂CO₂R^{5b}, C₁-C₄ alkyl, C₁-C₄ haloalkyl, C₁-C₄ hydroxyalkyl, C₁-C₄ hydroxyalkoxy, and C₂-C₆ alkoxyalkyl;
R^{5a} is C₁-C₄ alkyl; and
R^{5b} is C₁-C₄ alkyl;
n is an integer of 1 to 3.

In certain embodiments of formula (I), or enantiomers, diastereomers, racemates, tautomers, prodrugs, hydrates, solvates or pharmaceutically acceptable salts thereof, R^{3c} is selected from the group consisting of hydrogen, —CH₃, —CH(CH₃)₂, —CH(CH₃)CH₂CH₃, —CH₂CH(CH₃)₂, —CH₂OH, —CH(OH)CH₃, —CH₂CH₂SCH₃, —CH₂SH, —CH₂CH₂CH₂CH₂NH₂, —CH₂C(O)OH, —CH₂CH₂C(O)OH, —CH₂C(O)NH₂, —CH₂CH₂C(O)NH₂, —CH₂CH₂CH₂NHC(NH)NH₂, —CH₂C₆H₅, —CH₂C₆H₄(OH), —CH₂(indol-3-yl), and —CH₂(imidazol-4-yl); and the remaining variables are as defined for formula (I).

In certain embodiments of formula (I), or enantiomers, diastereomers, racemates, tautomers, prodrugs, hydrates, solvates or pharmaceutically acceptable salts thereof, L is —O—(CR^{a}R^{b})ₙ—; and the remaining variables are as defined for formula (I).

In certain embodiments of formula (I), or enantiomers, diastereomers, racemates, tautomers, prodrugs, hydrates, solvates or pharmaceutically acceptable salts thereof, R³ is selected from the group consisting of hydrogen, C₁-C₄ alkyl, —P(O)(OR^{3d})₂, —C(O)CHR^{3e}NR^{3b}R^{3c}; L is —O—(CR^{a}R^{b})ₙ—; and the remaining variables are as defined for formula (I).

In certain embodiments of formula (I), or enantiomers, diastereomers, racemates, tautomers, prodrugs, hydrates, solvates or pharmaceutically acceptable salts thereof, R³ is —C(O)CHR^{3e}NR^{3b}R^{3c}; L is —O—(CR^{a}R^{b})ₙ—; and the remaining variables are as defined for formula (I).

In certain embodiments of formula (I), or enantiomers, diastereomers, racemates, tautomers, prodrugs, hydrates, solvates or pharmaceutically acceptable salts thereof, R³ is —C(O)CHR^{3e}NR^{3b}R^{3c}; R³⁰ is selected from the group consisting of hydrogen, CH₃, CH(CH₃)₂, CH(CH₃)CH₂CH₃, CH₂CH(CH₃)₂, CH₂OH, CH(OH)CH₃, CH₂CH₂SCH₃, CH₂SH, —CH₂CH₂CH₂CH₂NH₂, CH₂C(O)OH, CH₂CH₂C(O)OH, CH₂C(O)NH₂, CH₂CH₂C(O)NH₂, —CH₂CH₂CH₂NHC(NH)NH₂, CH₂C₆H₅, CH₂C₆H₄(OH), CH₂(indol-3-yl), and CH₂(imidazol-4-yl); L is —O—(CR^{a}R^{b})ₙ—; and the remaining variables are as defined for formula (I).

In certain embodiments of formula (I), or enantiomers, diastereomers, racemates, tautomers, prodrugs, hydrates, solvates or pharmaceutically acceptable salts thereof, R¹ is selected from the group consisting of hydrogen and C₁-C₆ alkyl; wherein the C₁-C₆ alkyl of R¹ is optionally substituted with 1 or 2 of R⁴; R³ is —C(O)CHR^{3e}NR^{3b}R^{3c}; R^{3e} is selected from the group consisting of hydrogen, CH₃, CH(CH₃)₂, CH(CH₃)CH₂CH₃, CH₂CH(CH₃)₂, —CH₂OH, CH(OH)CH₃, CH₂CH₂SCH₃, CH₂SH, CH₂CH₂CH₂CH₂NH₂, CH₂C(O)OH, —CH₂CH₂C(O)OH, CH₂C(O)NH₂, CH₂CH₂C(O)NH₂, CH₂CH₂CH₂NHC(NH)NH₂, CH₂C₆H₅, CH₂C₆H₄(OH), CH₂(indol-3-yl), and CH₂(imidazol-4-yl); R⁴ is selected from the group consisting of OH and OR^{4a}; L is —O—(CR^{a}R^{b})ₙ—; and the remaining variables are as defined for formula (I).

In certain embodiments of formula (I), or enantiomers, diastereomers, racemates, tautomers, prodrugs, hydrates, solvates or pharmaceutically acceptable salts thereof, R¹ is methyl; R³ is —C(O)CHR^{3e}NR^{3b}R^{3c}; R^{3e} is selected from the group consisting of hydrogen, —CH₃, —CH(CH₃)₂, —CH(CH₃)CH₂CH₃, —CH₂CH(CH₃)₂, —CH₂OH, —CH(OH)CH₃, —CH₂CH₂SCH₃, —CH₂SH, —CH₂CH₂CH₂CH₂NH₂, —CH₂C(O)OH, —CH₂CH₂C(O)OH, —CH₂C(O)NH₂, —CH₂CH₂C(O)NH₂, —CH₂CH₂CH₂NHC(NH)NH₂, —CH₂C₆H₅, —CH₂C₆H₄(OH), —CH₂(indol-3-yl), and —CH₂(imidazol-4-yl); L is —O—(CR^{a}R^{b})ₙ—; and the remaining variables are as defined for formula (I).

In certain embodiments of formula (I), or enantiomers, diastereomers, racemates, tautomers, prodrugs, hydrates, solvates or pharmaceutically acceptable salts thereof, R¹ is selected from the group consisting of hydrogen and C₁-C₆ alkyl; wherein the R¹ C₁-C₆ alkyl is optionally substituted with 1 or 2 of R⁴; R³ is selected from the group consisting of hydrogen, C₁-C₄ alkyl, —P(O)(OR^{3d})₂, —C(O)CHR^{3e}NR^{3b}R^{3c}; R⁴ is selected from the group consisting of —OH and —OR^{4a}; L is —O—(CR^{a}R^{b})ₙ—; and the remaining variables are as defined for formula (I).

In certain embodiments of formula (I), or enantiomers, diastereomers, racemates, tautomers, prodrugs, hydrates, solvates or pharmaceutically acceptable salts thereof, R¹ is methyl; R³ is selected from the group consisting of hydrogen, C₁-C₄ alkyl, —P(O)(OR^{3d})₂, —C(O)CHR^{3e}NR^{3b}R^{3c}; L is —O—(CR^{a}R^{b})ₙ—; and the remaining variables are as defined for formula (I).

In certain embodiments of formula (I), or enantiomers, diastereomers, racemates, tautomers, prodrugs, hydrates, solvates or pharmaceutically acceptable salts thereof, L is -a bond; R³ is selected from the group consisting of hydrogen, fluoro, chloro, bromo, —CN, C₁-C₄ alkyl, C₁-C₄ haloalkyl, —OH, —OR^{3a}, —NR^{3b}R^{3c}, —C(O)OH, —C(O)OR^{3a}, —C(O)NR^{3b}R^{3c}, C₃-C₈ cycloalkyl, C₅-C₇ cycloalkenyl, 3-6 membered heterocyclyl, C₅-C₇ heterocycloalkenyl, C₆ aryl, and 5-6 membered heteroaryl; wherein the C₃-C₈ cycloalkyl, C₆ aryl, 3-6 membered heterocyclyl, and 5-6 membered heteroaryl of R³, are optionally substituted with 1 or 2 of R⁵; and the remaining variables are as defined for formula (I).

In certain embodiments of formula (I), or enantiomers, diastereomers, racemates, tautomers, prodrugs, hydrates, solvates or pharmaceutically acceptable salts thereof, L is -a bond; R¹ is C₁-C₆ alkyl; wherein the C₁-C₆ alkyl of R¹ is optionally substituted with 1 or 2 R⁴; R⁴ selected from the group consisting of —OH, —OR^{4a}, —C(O)NR^{4b}R^{4c}, and 5-6 membered heterocyclyl; R³ is selected from the group consisting of hydrogen, fluoro, chloro, bromo, —CN, C₁-C₄ alkyl, C₁-C₄ haloalkyl, —OH, —OR^{3a}, —NR^{3b}R^{3c}, —C(O)OH, —C(O)OR^{3a}, —C(O)NR^{3b}R^{3c}, C₃-C₈ cycloalkyl, C₅-C₇ cycloalkenyl, 3-6 membered heterocyclyl, C₅-C₇ heterocycloalkenyl, C₆ aryl, and 5-6 membered heteroaryl; wherein the C₃-C₈ cycloalkyl, C₆ aryl, 3-6 membered heterocyclyl, and 5-6 membered heteroaryl of R³, are optionally substituted with 1 or 2 of R⁵; R⁵ is selected from the group consisting of fluoro, —CN, —OH, —OR^{1a}, ═O, —CH₂CO₂R^{5b}, C₁-C₄ alkyl, C₁-C₄ haloalkyl, C₁-C₄ hydroxyalkyl, C₁-C₄ hydroxyalkoxy, and C₂-C₆ alkoxyalkyl; and the remaining variables are as defined for formula (I).

In certain embodiments of formula (I), or enantiomers, diastereomers, racemates, tautomers, prodrugs, hydrates, solvates or pharmaceutically acceptable salts thereof, L is -a bond; R¹ is methyl; R³ is selected from the group consisting of hydrogen, fluoro, chloro, bromo, —CN, C₁-C₄ alkyl, C₁-C₄ haloalkyl, —OH, —OR^{3a}, —NR^{3b}R^{3c}, —C(O)OH, —C(O)OR^{3a}, —C(O)NR^{3b}R^{3c}, C₃-C₈ cycloalkyl, C₅-C₇ cycloalkenyl, 3-6 membered heterocyclyl, C₅-C₇ heterocycloalkenyl, C₆ aryl, and 5-6 membered heteroaryl; wherein the C₃-C₈ cycloalkyl, C₆ aryl, 3-6 membered heterocyclyl, and 5-6 membered heteroaryl of R³, are optionally substituted with 1 or 2 R⁵; R⁵ is selected from the group consisting of fluoro, —CN, —OH, —OR^{5a}, ═O, —CH₂CO₂R^{5b}, C₁-C₄ alkyl, C₁-C₄ haloalkyl, C₁-C₄ hydroxyalkyl, C₁-C₄ hydroxyalkoxy, and C₂-C₆ alkoxyalkyl; and the remaining variables are as defined for formula (I).

In certain embodiments of formula (I), or enantiomers, diastereomers, racemates, tautomers, prodrugs, hydrates, solvates or pharmaceutically acceptable salts thereof, L is -a bond; R¹ is methyl; R³ is selected from the group consisting of hydrogen, C₁-C₄ alkyl, C₆ aryl, and 5-6 membered heteroaryl; wherein the R³, C₆ aryl, and 5-6 membered heteroaryl, are optionally substituted with 1 or 2 of R⁵; R⁵ is selected from the group consisting of —OH, —OR^{5a}, C₁-C₄ alkyl, C₁-C₄ hydroxyalkyl, C₁-C₄ hydroxyalkoxy, and C₂-C₆ alkoxyalkyl; and the remaining variables are as defined for formula (I).

In certain embodiments of formula (I), or enantiomers, diastereomers, racemates, tautomers, prodrugs, hydrates, solvates or pharmaceutically acceptable salts thereof, L is —O—(CR^{a}R^{b})ₙ—; R³ is selected from the group consisting of —CN, C₃-C₈ cycloalkyl, 3-6 membered heterocyclyl, C₆ aryl, and 5-6 membered heteroaryl; wherein the C₃-C₈ cycloalkyl, C₆ aryl, 3-6 membered heterocyclyl, and 5-6 membered heteroaryl of R³, are optionally substituted with 1 or 2 R⁵; R⁵ is selected from the group consisting of —OH, —CH₂CO₂R^{5b}, C₁-C₄ alkyl, and C₁-C₄ hydroxyalkyl; and the remaining variables are as defined for formula (I).

In certain embodiments of formula (I), or enantiomers, diastereomers, racemates, tautomers, prodrugs, hydrates, solvates or pharmaceutically acceptable salts thereof, L is —O—(CR^{a}R^{b})ₙ—; R¹ is methyl; R³ is selected from the group consisting of —CN, C₃-C₈ cycloalkyl, 3-6 membered heterocyclyl, C₆ aryl, and 5-6 membered heteroaryl; wherein the C₃-C₈ cycloalkyl, C₆ aryl, 3-6 membered heterocyclyl, and 5-6 membered heteroaryl of R³, are optionally substituted with 1 or 2 of R⁵; R⁵ is selected from the group consisting of —OH, —CH₂CO₂R^{5b}, C₁-C₄ alkyl, and C₁-C₄ hydroxyalkyl; and the remaining variables are as defined for formula (I).

In certain embodiments of formula (I), or enantiomers, diastereomers, racemates, tautomers, prodrugs, hydrates, solvates or pharmaceutically acceptable salts thereof, L is —O—(CR^{a}R^{b})ₙ—; R¹ is methyl; R³ is 5-6 membered heteroaryl; and the remaining variables are as defined for formula (I).

In certain embodiments of formula (I), or enantiomers, diastereomers, racemates, tautomers, prodrugs, hydrates, solvates or pharmaceutically acceptable salts thereof, L is —O—(CR^{a}R^{b})ₙ—; R³ is selected from the group consisting of hydrogen and C₁-C₄ alkyl; and the remaining variables are as defined for formula (I).

In certain embodiments of formula (I), or enantiomers, diastereomers, racemates, tautomers, prodrugs, hydrates, solvates or pharmaceutically acceptable salts thereof, L is —O—(CR^{a}R^{b})ₙ—; R³ is hydrogen; and the remaining variables are as defined for formula (I).

In certain embodiments of formula (I), or enantiomers, diastereomers, racemates, tautomers, prodrugs, hydrates, solvates or pharmaceutically acceptable salts thereof, L is —O—(CR^{a}R^{b})ₙ—; R¹ is methyl; R³ is hydrogen; and the remaining variables are as defined for formula (I).

In certain embodiments of formula (I), or enantiomers, diastereomers, racemates, tautomers, prodrugs, hydrates, solvates or pharmaceutically acceptable salts thereof, L is —(CR^{a}R^{b})ₙ—C(O)—; R³ is selected from the group consisting of —OH, —OR^{3a}, —NR^{3b}R^{3c} and 3-6 membered heterocyclyl; wherein the 3-6 membered heterocyclyl of R³ is optionally substituted with 1 or 2 R⁵; R⁵ is selected from the group consisting —OH, —OR^{5a}, C₁-C₄ haloalkyl, C₁-C₄ hydroxyalkyl, and C₂-C₆ alkoxyalkyl; and the remaining variables are as defined for formula (I).

In certain embodiments of formula (I), or enantiomers, diastereomers, racemates, tautomers, prodrugs, hydrates, solvates or pharmaceutically acceptable salts thereof, L is —(CR^{a}R^{b})ₙ—C(O)—; R³ is 3-6 membered heterocyclyl is optionally substituted with 1 or 2 R⁵; R⁵ is selected from the group consisting —OH, —OR^{5a}, C₁-C₄ haloalkyl, C₁-C₄ hydroxyalkyl, and C₂-C₆ alkoxyalkyl; and the remaining variables are as defined for formula (I).

In certain embodiments of formula (I), or enantiomers, diastereomers, racemates, tautomers, prodrugs, hydrates, solvates or pharmaceutically acceptable salts thereof, L is —(CR^{a}R^{b})ₙ—C(O)—; R¹ is methyl; R³ is 3-6 membered heterocyclyl is optionally substituted with 1 or 2 R⁵; R⁵ is selected from the group consisting —OH, —OR^{5a}, C₁-C₄ haloalkyl, C₁-C₄ hydroxyalkyl, and C₂-C₆ alkoxyalkyl; and the remaining variables are as defined for formula (I).

In certain embodiments of formula (I), or enantiomers, diastereomers, racemates, tautomers, prodrugs, hydrates, solvates or pharmaceutically acceptable salts thereof, L is —C(O)—; R³ is selected from the group consisting of —OH, —OR^{3c}, —NR^{3b}R^{3c} and 3-6 membered heterocyclyl; wherein the R³ 3-6 membered heterocyclyl, is optionally substituted with 1 or 2 R⁵; R⁵ is selected from the group consisting of C₁-C₄ hydroxyalkyl and C₂-C₆ alkoxyalkyl; and the remaining variables are as defined for formula (I).

In certain embodiments of formula (I), or enantiomers, diastereomers, racemates, tautomers, prodrugs, hydrates, solvates or pharmaceutically acceptable salts thereof, L is —C(O)—; R³ is 3-6 membered heterocyclyl optionally substituted with 1 or 2 of R⁵; R⁵ is selected from the group consisting of C₁-C₄ hydroxyalkyl and C₂-C₆ alkoxyalkyl; and the remaining variables are as defined for formula (I).

In certain embodiments of formula (I), or enantiomers, diastereomers, racemates, tautomers, prodrugs, hydrates, solvates or pharmaceutically acceptable salts thereof, L is —C(O)—; R¹ is methyl; R³ is 3-6 membered heterocyclyl optionally substituted with 1 or 2 of R⁵; R⁵ is selected from the group consisting of C₁-C₄ hydroxyalkyl and C₂-C₆ alkoxyalkyl; and the remaining variables are as defined for formula (I).

In certain embodiments of formula (I), or enantiomers, diastereomers, racemates, tautomers, prodrugs, hydrates, solvates or pharmaceutically acceptable salts thereof, L is —O—; R³ is selected from the group consisting of hydrogen, C₁-C₄ alkyl, C₃-C₈ cycloalkyl, 3-6 membered heterocyclyl, and 5-6 membered heteroaryl; wherein the R³ C₃-C₈ cycloalkyl, 3-6 membered heterocyclyl, and 5-6 membered heteroaryl, is optionally substituted with 1 or 2 of R⁵; R⁵ is selected from the group consisting of —OH, —OR^{5a}, ═O, and C₁-C₄ alkyl; and the remaining variables are as defined for formula (I).

In certain embodiments of formula (I), or enantiomers, diastereomers, racemates, tautomers, prodrugs, hydrates, solvates or pharmaceutically acceptable salts thereof, L is —OCH₂C(O)—; R³ is selected from the group consisting of —OH, —OR³, —NR^{3b}R^{3c}, C₃-C₈ cycloalkyl, and 3-6 membered heterocyclyl; and the remaining variables are as defined for formula (I).

In certain embodiments of formula (I), or enantiomers, diastereomers, racemates, tautomers, prodrugs, hydrates, solvates or pharmaceutically acceptable salts thereof, L is —OCH₂C(O)—; R¹ is methyl; R³ is selected from the group consisting of —OH, —OR^{3a}, —NR^{3b}R^{3c}, C₃-C₈ cycloalkyl, and 3-6 membered heterocyclyl; and the remaining variables are as defined for formula (I).

In certain embodiments of formula (I), or enantiomers, diastereomers, racemates, tautomers, prodrugs, hydrates, solvates or pharmaceutically acceptable salts thereof, L is —OCH₂C(O)—; R¹ is methyl; R³ is —NR^{3b}R^{3c}; and the remaining variables are as defined for formula (I).

In certain embodiments, compounds of the present disclosure are represented by formula (II), or enantiomers, diastereomers, racemates, tautomers, prodrugs, hydrates, solvates or pharmaceutically acceptable salts thereof,

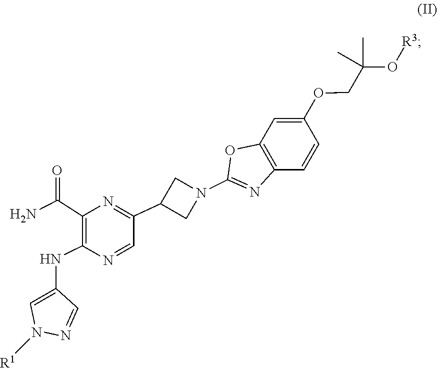

where
R¹ is selected from the group consisting of hydrogen, C₁-C₆ alkyl, and 5-6 membered heterocyclyl; wherein the C₁-C₆ alkyl of R¹ is optionally substituted with 1 or 2 R⁴;
R³ is selected from the group consisting of hydrogen, C₁-C₄ alkyl, C₁-C₄ haloalkyl, —P(O)(OR^{3d})₂, —C(O)CHR^{3e}NR^{3b}R^{3c}, —C(O)OH, —C(O)OR^{3a}, —C(O)NR^{3b}R^{3c}, C₃-C₈ cycloalkyl, C₅-C₇ cycloalkenyl, 3-6 membered heterocyclyl, C₅-C₇ heterocycloalkenyl, C₆ aryl, and 5-6 membered heteroaryl;
wherein the C₃-C₈ cycloalkyl, C₆ aryl, 3-6 membered heterocyclyl, and 5-6 membered heteroaryl of R³, are optionally substituted with 1 or 2 R⁵;
R^{3a} is C₁-C₄ alkyl;
R^{3b} and R^{3c} are each independently selected from the group consisting of hydrogen, C₁-C₄ alkyl, C₃-C₆ cycloalkyl, and —CO₂R^{3f};
R^{3d} is selected from the group consisting of hydrogen, C₁-C₄ alkyl, and C₂-C₄ alkenyl;
R^{3e} is selected from the group consisting of hydrogen and C₁-C₄ alkyl; wherein the C₁-C₄ alkyl of R^{3e} is optionally substituted with 1 of R^{3g};
R^{3f} is C₁-C₄ alkyl;
R^{3g} selected from the group consisting of —OH, —SH, —SCH₃, —NH₂, —C(O)OH, —C(O)NH₂, —NHC(NH)NH₂, C₆ aryl, and 5-9 membered heteroaryl; wherein the C₆ aryl of R^{3g} is optionally substituted with 1-OH;
R⁴ is selected from the group consisting of —OH, —OR^{4a}, —C(O)NR^{4b}R^{4c}, and 5-6 membered heterocyclyl; wherein the 3-10 membered heterocyclyl of R⁴ is optionally substituted with 1 or 2 C₁-C₄ alkyl;
R^{4a} is C₁-C₄ alkyl; wherein the R^{4a} is C₁-C₄ alkyl is optionally substituted with 1-Si(R^{4d})₃;
R^{4b} and R^{4c} are each independently selected from the group consisting of H and C₁-C₄ alkyl;
R^{4d} is C₁-C₄ alkyl;
R⁵ is selected from the group consisting of fluoro, —CN, —OH, —OR^{5a}, ═O, —CH₂CO₂R^{5b}, C₁-C₄ alkyl, C₁-C₄ haloalkyl, C₁-C₄ hydroxyalkyl, C₁-C₄ hydroxyalkoxy, and C₂-C₆ alkoxyalkyl;
R^{5a} is C₁-C₄ alkyl; and
R^{5b} is C₁-C₄ alkyl.

In certain embodiments of formula (II), or enantiomers, diastereomers, racemates, tautomers, prodrugs, hydrates, solvates or pharmaceutically acceptable salts thereof, R³ is selected from the group consisting of hydrogen, C₁-C₄ alkyl, —P(O)(OR^{3d})₂, and —C(O)CHR^{3e}NR^{3b}R^{3c}, and the remaining variables are as defined for formula (I).

In certain embodiments of formula (II), or enantiomers, diastereomers, racemates, tautomers, prodrugs, hydrates, solvates or pharmaceutically acceptable salts thereof, R¹ is selected from the group consisting of hydrogen and C₁-C₆ alkyl; wherein the R¹ C₁-C₆ alkyl is optionally substituted with 1 or 2 of R⁴; R⁴ is selected from the group consisting of —OH and —OR^{4a}; R³ is selected from the group consisting of hydrogen, C₁-C₄ alkyl, —P(O)(OR^{3d})₂, and —C(O)CHR^{3e}NR^{3b}R^{3c}, and the remaining variables are as defined for formula (I).

In certain embodiments the compound or enantiomers, diastereomers, racemates, tautomers, prodrugs, hydrates, solvates or pharmaceutically acceptable salts thereof, is

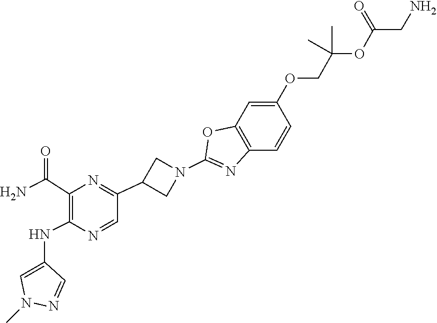 [SMILES: Cn1cc(Nc2ncc(C3CN(c4nc5ccc(OCC(C)(C)OC(=O)CN)cc5o4)C3)nc2C(N)=O)cn1]

In certain embodiments the compound or enantiomers, diastereomers, racemates, tautomers, prodrugs, hydrates, solvates or pharmaceutically acceptable salts thereof, is

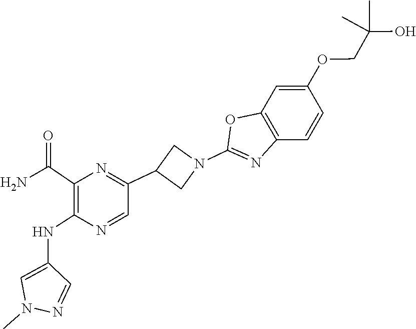 [SMILES: Cn1cc(Nc2ncc(C3CN(c4nc5ccc(OCC(C)(C)O)cc5o4)C3)nc2C(N)=O)cn1]

Compound names are assigned by using Name 2019 by Advanced Chemical Development (ACD)/ChemSketch 2019.1.1 naming algorithm.

In one embodiment, the compound of Formula (I) is selected from the group consisting of 6-[1-(6-hydroxy-1,3-benzoxazol-2-yl)azetidin-3-yl]-3-[(1-{[2-(trimethylsilyl)ethoxy]methyl}-1H-pyrazol-4-yl)amino]pyrazine-2-carboxamide; 6-[1-(1,3-benzoxazol-2-yl)azetidin-3-yl]-3-[(1-methyl-1H-pyrazol-4-yl)amino]pyrazine-2-carboxamide; 6-[1-(6-chloro-1,3-benzoxazol-2-yl)azetidin-3-yl]-3-[(1-methyl-1H-pyrazol-4-yl)amino]pyrazine-2-carboxamide; 6-[1-(6-methyl-1,3-benzoxazol-2-yl)azetidin-3-yl]-3-[(1-methyl-1H-pyrazol-4-yl)amino]pyrazine-2-carboxamide; 6-[1-(5,6-dimethyl-1,3-benzoxazol-2-yl)azetidin-3-yl]-3-[(1-methyl-1H-pyrazol-4-yl)amino]pyrazine-2-carboxamide; 6-[1-(6-ethyl-1,3-benzoxazol-2-yl)azetidin-3-yl]-3-[(1-methyl-1H-pyrazol-4-yl)amino]pyrazine-2-carboxamide; 6-{1-[6-(cyclohex-1-en-1-yl)-1,3-benzoxazol-2-yl]azetidin-3-yl}-3-[(1-methyl-1H-pyrazol-4-yl)amino]pyrazine-2-carboxamide; 6-[1-(6-bromo-1,3-benzoxazol-2-yl)azetidin-3-yl]-3-[(1-methyl-1H-pyrazol-4-yl)amino]pyrazine-2-carboxamide; 6-[1-(6-cyclohexyl-1,3-benzoxazol-2-yl)azetidin-3-yl]-3-[(1-methyl-1H-pyrazol-4-yl)amino]pyrazine-2-carboxamide; 3-[(1-methyl-1H-pyrazol-4-yl)amino]-6-[1-(6-phenyl-1,3-benzoxazol-2-yl)azetidin-3-yl]pyrazine-2-carboxamide; 6-[1-(6-methoxy-1,3-benzoxazol-2-yl)azetidin-3-yl]-3-[(1-methyl-1H-pyrazol-4-yl)amino]pyrazine-2-carboxamide; 6-[1-(6-cyclopentyl-1,3-benzoxazol-2-yl)azetidin-3-yl]-3-[(1-methyl-1H-pyrazol-4-yl)amino]pyrazine-2-carboxamide; 6-{1-[6-(cyclopent-1-en-1-yl)-1,3-benzoxazol-2-yl]azetidin-3-yl}-3-[(1-methyl-1H-pyrazol-4-yl)amino]pyrazine-2-carboxamide; 6-{1-[6-(3,6-dihydro-2H-pyran-4-yl)-1,3-benzoxazol-2-yl]azetidin-3-yl}-3-[(1-methyl-1H-pyrazol-4-yl)amino]pyrazine-2-carboxamide; 3-[(1-methyl-1H-pyrazol-4-yl)amino]-6-{1-[6-(trifluoromethyl)-1,3-benzoxazol-2-yl]azetidin-3-yl}pyrazine-2-carboxamide; methyl 2-(3-{6-carbamoyl-5-[(1-methyl-1H-pyrazol-4-yl)amino]pyrazin-2-yl}azetidin-1-yl)-1,3-benzoxazole-6-carboxylate; 6-[1-(1,3-benzoxazol-2-yl)azetidin-3-yl]-3-[(1H-pyrazol-4-yl)amino]pyrazine-2-carboxamide; 3-[(1-methyl-1H-pyrazol-4-yl)amino]-6-{1-[6-(oxolan-3-yl)-1,3-benzoxazol-2-yl]azetidin-3-yl}pyrazine-2-carboxamide; 6-{1-[6-(2,5-dihydrofuran-3-yl)-1,3-benzoxazol-2-yl]azetidin-3-yl}-3-[(1-methyl-1H-pyrazol-4-yl)amino]pyrazine-2-carboxamide; 6-{1-[6-(hydroxymethyl)-1,3-benzoxazol-2-yl]azetidin-3-yl}-3-[(1-methyl-1H-pyrazol-4-yl)amino]pyrazine-2-carboxamide; 6-(1-{6-[(1E)-3-hydroxy-3-methylbut-1-en-1-yl]-1,3-benzoxazol-2-yl}azetidin-3-yl)-3-[(1-methyl-1H-pyrazol-4-yl)amino]pyrazine-2-carboxamide; 2-(3-{6-carbamoyl-5-[(1-methyl-1H-pyrazol-4-yl)amino]pyrazin-2-yl}azetidin-1-yl)-N,N-dimethyl-1,3-benzoxazole-6-carboxamide; 2-(3-{6-carbamoyl-5-[(1-methyl-1H-pyrazol-4-yl)amino]pyrazin-2-yl}azetidin-1-yl)-1,3-benzoxazole-6-carboxylic acid; 6-{1-[6-(2-hydroxypropan-2-yl)-1,3-benzoxazol-2-yl]azetidin-3-yl}-3-[(1-methyl-1H-pyrazol-4-yl)amino]pyrazine-2-carboxamide; 6-[1-(6-hydroxy-1,3-benzoxazol-2-yl)azetidin-3-yl]-3-[(1-methyl-1H-pyrazol-4-yl)amino]pyrazine-2-carboxamide; 6-{1-[6-(3-hydroxy-3-methylbutyl)-1,3-benzoxazol-2-yl]azetidin-3-yl}-3-[(1-methyl-1H-pyrazol-4-yl)amino]pyrazine-2-carboxamide; 6-(1-{6-[(1E)-3-methoxyprop-1-en-1-yl]-1,3-benzoxazol-2-yl}azetidin-3-yl)-3-[(1-methyl-1H-pyrazol-4-yl)amino]pyrazine-2-carboxamide; 6-{1-[6-(cyclopropylmethoxy)-1,3-benzoxazol-2-yl]azetidin-3-yl}-3-[(1-methyl-1H-pyrazol-4-yl)amino]pyrazine-2-carboxamide; 6-{1-[6-(cyanomethoxy)-1,3-benzoxazol-2-yl]azetidin-3-yl}-3-[(1-methyl-1H-pyrazol-4-yl)amino]pyrazine-2-carboxamide; 6-(1-{6-[3-hydroxy-2-(hydroxymethyl)propyl]-1,3-benzoxazol-2-yl}azetidin-3-yl)-3-[(1-methyl-1H-pyrazol-4-yl)amino]pyrazine-2-carboxamide; ethyl {[2-(3-{6-carbamoyl-5-[(1-methyl-1H-pyrazol-4-yl)amino]pyrazin-2-yl}azetidin-1-yl)-1,3-benzoxazol-6-yl]oxy}acetate; 6-[1-(6-fluoro-1,3-benzoxazol-2-yl)azetidin-3-yl]-3-{[1-(oxan-4-yl)-1H-pyrazol-4-yl]amino}pyrazine-2-carboxamide; 6-[1-(6-fluoro-1,3-benzoxazol-2-yl)azetidin-3-yl]-3-{[1-(2-hydroxy-2-methylpropyl)-1H-pyrazol-4-yl]amino}pyrazine-2-carboxamide; 6-[1-(1,3-benzoxazol-2-yl)azetidin-3-yl]-3-{[1-(2-methoxyethyl)-1H-pyrazol-4-yl]amino}pyrazine-2-carboxamide; 6-[1-(1,3-benzoxazol-2-yl)azetidin-3-yl]-3-{[1-(2-hydroxy-2-methylpropyl)-1H-pyrazol-4-yl]amino}pyrazine-2-carboxamide; 6-{1-[6-(3-hydroxy-3-methylbutoxy)-1,3-benzoxazol-2-yl]azetidin-3-yl}-3-[(1-methyl-1H-pyrazol-4-yl)amino]pyrazine-2-carboxamide; 6-{1-[6-(3-hydroxypropyl)-1,3-benzoxazol-2-yl]azetidin-3-yl}-3-[(1-methyl-1H-pyrazol-4-yl)amino]pyrazine-2-carboxamide; 6-(1-{6-[(1E)-3-hydroxyprop-1-en-1-yl]-1,3-benzoxazol-2-yl}azetidin-3-yl)-3-[(1-methyl-1H-pyrazol-4-yl)amino]pyrazine-2-carboxamide; 6-{1-[6-(3-methoxypropyl)-1,3-benzoxazol-2-yl]azetidin-3-yl}-3-[(1-methyl-1H-pyrazol-4-yl)amino]pyrazine-2-carboxamide; 6-[1-(1,3-benzoxazol-2-yl)azetidin-3-yl]-3-({1-[2-(dimethylamino)-2-oxoethyl]-1H-pyrazol-4-yl}amino)pyrazine-2-carboxamide; 6-{1-[6-(2-methoxyethoxy)-1,3-benzoxazol-2-yl]azetidin-3-yl}-3-[(1-methyl-1H-pyrazol-4-yl)amino]pyrazine-2-carboxamide; 3-{[1-(2,3-dihydroxypropyl)-1H-pyrazol-4-yl]amino}-6-[1-(6-fluoro-1,3-benzoxazol-2-yl)azetidin-3-yl]pyrazine-2-carboxamide; 6-[1-(6-fluoro-1,3-benzoxazol-2-yl)azetidin-3-yl]-3-[(1H-pyrazol-4-yl)amino]pyrazine-2-carboxamide; 3-({1-[(2,2-dimethyl-1,3-dioxolan-4-yl)methyl]-1H-pyrazol-4-yl}amino)-6-[1-(6-fluoro-1,3-benzoxazol-2-yl)azetidin-3-yl]pyrazine-2-carboxamide; 6-{1-[6-(2-amino-2-oxoethoxy)-1,3-benzoxazol-2-yl]azetidin-3-yl}-3-[(1-methyl-1H-pyrazol-4-yl)amino]pyrazine-2-carboxamide; 6-{1-[6-(2-hydroxy-2-methylpropoxy)-1,3-benzoxazol-2-yl]azetidin-3-yl}-3-[(1-methyl-1H-pyrazol-4-yl)amino]pyrazine-2-carboxamide; 6-(1-{6-[2-(dimethylamino)-2-oxoethoxy]-1,3-benzoxazol-2-yl}azetidin-3-yl)-3-[(1-methyl-1H-pyrazol-4-yl)amino]pyrazine-2-carboxamide; 6-{1-[6-(2-hydroxyethoxy)-1,3-benzoxazol-2-yl]azetidin-3-yl}-3-[(1-methyl-1H-pyrazol-4-yl)amino]pyrazine-2-carboxamide; 3-[(1-methyl-1H-pyrazol-4-yl)amino]-6-(1-{6-[(oxetan-3-yl)methoxy]-1,3-benzoxazol-2-yl}azetidin-3-yl)pyrazine-2-carboxamide; 6-(1-{6-[(3-hydroxycyclobutyl)methoxy]-1,3-benzoxazol-2-yl}azetidin-3-yl)-3-[(1-methyl-1H-pyrazol-4-yl)amino]pyrazine-2-carboxamide; 6-{1-[6-(2,3-dihydroxypropoxy)-1,3-benzoxazol-2-yl]azetidin-3-yl}-3-[(1-methyl-1H-pyrazol-4-yl)amino]pyrazine-2-carboxamide; 6-(1-{6-[(1-hydroxycyclobutyl)methoxy]-1,3-benzoxazol-2-yl}azetidin-3-yl)-3-[(1-methyl-1H-pyrazol-4-yl)amino]pyrazine-2-carboxamide; 6-(1-{6-[2-(3-hydroxyoxetan-3-yl)ethyl]-1,3-benzoxazol-2-yl}azetidin-3-yl)-3-[(1-methyl-1H-pyrazol-4-yl)amino]pyrazine-2-carboxamide; 3-[(1-methyl-1H-pyrazol-4-yl)amino]-6-{1-[6-(morpholine-4-carbonyl)-1,3-benzoxazol-2-yl]azetidin-3-yl}pyrazine-2-carboxamide; 6-(1-{6-[2-(dimethylamino)-2-oxoethyl]-1,3-benzoxazol-2-yl}azetidin-3-yl)-3-[(1-methyl-1H-pyrazol-4-yl)amino]pyrazine-2-carboxamide; 6-[1-(6-{[(2R)-1,4-dioxan-2-yl]methoxy}-1,3-benzoxazol-2-yl)azetidin-3-yl]-3-[(1-methyl-1H-pyrazol-4-yl)amino]pyrazine-2-carboxamide; 3-[(1-methyl-1H-pyrazol-4-yl)amino]-6-(1-{6-[2-oxo-2-(pyrrolidin-1-yl)ethoxy]-1,3-benzoxazol-2-yl}azetidin-3-yl)pyrazine-2-carboxamide; 6-(1-{6-[2-(methylamino)-2-oxoethyl]-1,3-benzoxazol-2-yl}azetidin-3-yl)-3-[(1-methyl-1H-pyrazol-4-yl)amino]pyrazine-2-carboxamide; 6-(1-{6-[3-hydroxy-2-(hydroxymethyl)propoxy]-1,3-benzoxazol-2-yl}azetidin-3-yl)-3-[(1-methyl-1H-pyrazol-4-yl)amino]pyrazine-2-carboxamide; 6-(1-{6-[(2,2-dimethyl-1,3-dioxan-5-yl)methoxy]-1,3-benzoxazol-2-yl}azetidin-3-yl)-3-[(1-methyl-1H-pyrazol-4-yl)amino]pyrazine-2-carboxamide; N-tert-butyl-2-(3-{6-carbamoyl-5-[(1-methyl-1H-pyrazol-4-yl)amino]pyrazin-2-yl}azetidin-1-yl)-1,3-benzoxazole-6-carboxamide; 2-(3-{6-carbamoyl-5-[(1-methyl-1H-pyrazol-4-yl)amino]pyrazin-2-yl}azetidin-1-yl)-N-cyclopropyl-1,3-benzoxazole-6-carboxamide; 3-{[1-(2-hydroxyethyl)-1H-pyrazol-4-yl]amino}-6-[1-(6-methyl-1,3-benzoxazol-2-yl)azetidin-3-yl]pyrazine-2-carboxamide; 3-{[1-(2-methoxyethyl)-1H-pyrazol-4-yl]amino}-6-[1-(6-methyl-1,3-benzoxazol-2-yl)azetidin-3-yl]pyrazine-2-carboxamide; 6-[1-(6-{[3-(hydroxymethyl)cyclobutyl]methoxy}-1,3-benzoxazol-2-yl)azetidin-3-yl]-3-[(1-methyl-1H-pyrazol-4-yl)amino]pyrazine-2-carboxamide; 6-(1-{6-[(3-hydroxyoxetan-3-yl)methoxy]-1,3-benzoxazol-2-yl}azetidin-3-yl)-3-[(1-methyl-1H-pyrazol-4-yl)amino]pyrazine-2-carboxamide; 6-[1-(6-{[(1S,2R)-2-(hydroxymethyl)cyclopropyl]methoxy}-1,3-benzoxazol-2-yl)azetidin-3-yl]-3-[(1-methyl-1H-pyrazol-4-yl)amino]pyrazine-2-carboxamide; 6-(1-{6-[(2R)-2-(hydroxymethyl)pyrrolidine-1-carbonyl]-1,3-benzoxazol-2-yl}azetidin-3-yl)-3-[(1-methyl-1H-pyrazol-4-yl)amino]pyrazine-2-carboxamide; 3-[(1-methyl-1H-pyrazol-4-yl)amino]-6-{1-[6-(3-phenylpropoxy)-1,3-benzoxazol-2-yl]azetidin-3-yl}pyrazine-2-carboxamide; 6-{1-[6-(3-hydroxy-2,2-dimethylpropoxy)-1,3-benzoxazol-2-yl]azetidin-3-yl}-3-[(1-methyl-1H-pyrazol-4-yl)amino]pyrazine-2-carboxamide; 6-(1-{6-[3-(dimethylamino)-3-oxopropyl]-1,3-benzoxazol-2-yl}azetidin-3-yl)-3-[(1-methyl-1H-pyrazol-4-yl)amino]pyrazine-2-carboxamide; 3-[(1-methyl-1H-pyrazol-4-yl)amino]-6-(1-{6-[3-oxo-3-(pyrrolidin-1-yl)propyl]-1,3-benzoxazol-2-yl}azetidin-3-yl)pyrazine-2-carboxamide; 6-(1-{6-[(2R)-2-(methoxymethyl)pyrrolidine-1-carbonyl]-1,3-benzoxazol-2-yl}azetidin-3-yl)-3-[(1-methyl-1H-pyrazol-4-yl)amino]pyrazine-2-carboxamide; 3-[(1-methyl-1H-pyrazol-4-yl)amino]-6-(1-{6-[2-oxo-2-(pyrrolidin-1-yl)ethyl]-1,3-benzoxazol-2-yl}azetidin-3-yl)pyrazine-2-carboxamide; [2-(3-{6-carbamoyl-5-[(1-methyl-1H-pyrazol-4-yl)amino]pyrazin-2-yl}azetidin-1-yl)-1,3-benzoxazol-6-yl]acetic acid; 6-[1-(6-{2-[(2S)-2-(hydroxymethyl)pyrrolidin-1-yl]-2-oxoethyl}-1,3-benzoxazol-2-yl)azetidin-3-yl]-3-[(1-methyl-1H-pyrazol-4-yl)amino]pyrazine-2-carboxamide; 6-{1-[6-(2-hydroxy-2-methylpropyl)-1,3-benzoxazol-2-yl]azetidin-3-yl}-3-[(1-methyl-1H-pyrazol-4-yl)amino]pyrazine-2-carboxamide; 6-(1-{6-[(2R)-2-hydroxypropoxy]-1,3-benzoxazol-2-yl}azetidin-3-yl)-3-[(1-methyl-1H-pyrazol-4-yl)amino]pyrazine-2-carboxamide; 3-[(1-methyl-1H-pyrazol-4-yl)amino]-6-[1-(6-{[(3S)-oxolan-3-yl]oxy}-1,3-benzoxazol-2-yl)azetidin-3-yl]pyrazine-2-carboxamide; 6-[1-(6-{2-[(3S)-3-methoxypyrrolidin-1-yl]-2-oxoethyl}-1,3-benzoxazol-2-yl)azetidin-3-yl]-3-[(1-methyl-1H-pyrazol-4-yl)amino]pyrazine-2-carboxamide; 6-[1-(6-{2-[(2R)-2-(methoxymethyl)pyrrolidin-1-yl]-2-oxoethyl}-1,3-benzoxazol-2-yl)azetidin-3-yl]-3-[(1-methyl-1H-pyrazol-4-yl)amino]pyrazine-2-carboxamide; 6-(1-{6-[(1-methyl-2-oxopyrrolidin-3-yl)oxy]-1,3-benzoxazol-2-yl}azetidin-3-yl)-3-[(1-methyl-1H-pyrazol-4-yl)amino]pyrazine-2-carboxamide; 6-(1-{6-[2-(azetidin-1-yl)-2-oxoethoxy]-1,3-benzoxazol-2-yl}azetidin-3-yl)-3-[(1-methyl-1H-pyrazol-4-yl)amino]pyrazine-2-carboxamide; {[2-(3-{6-carbamoyl-5-[(1-methyl-1H-pyrazol-4-yl)amino]pyrazin-2-yl}azetidin-1-yl)-1,3-benzoxazol-6-yl]oxy}acetic acid; 6-[1-(6-methyl-1,3-benzoxazol-2-yl)azetidin-3-yl]-3-[(1H-pyrazol-4-yl)amino]pyrazine-2-carboxamide; 6-(1-{6-[(2R)-2-(2-hydroxypropan-2-yl)pyrrolidine-1-carbonyl]-1,3-benzoxazol-2-yl}azetidin-3-yl)-3-[(1-methyl-1H-pyrazol-4-yl)amino]pyrazine-2-carboxamide; 6-(1-{6-[(1-methyl-2-oxopiperidin-3-yl)oxy]-1,3-benzoxazol-2-yl}azetidin-3-yl)-3-[(1-methyl-1H-pyrazol-4-yl)amino]pyrazine-2-carboxamide; 4-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylbutan-2-yl dihydrogen phosphate; 4-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylbutan-2-yl diprop-2-en-1-yl phosphate; 6-[1-(6-methyl-1,3-benzoxazol-2-yl)azetidin-3-yl]-3-[(1-methyl-1H-pyrazol-4-yl)amino]pyridine-2-carboxamide; 6-(1-{6-[(2-hydroxycyclopentyl)oxy]-1,3-benzoxazol-2-yl}azetidin-3-yl)-3-[(1-methyl-1H-pyrazol-4-yl)amino]pyrazine-2-carboxamide; 3-[(1-methyl-1H-pyrazol-4-yl)amino]-6-(1-{6-[(1-methyl-1H-pyrazol-3-yl)methoxy]-1,3-benzoxazol-2-yl}azetidin-3-yl)pyrazine-2-carboxamide; 3-[(1-methyl-1H-pyrazol-4-yl)amino]-6-[1-(6-{2-oxo-2-[(2R)-2-(trifluoromethyl)pyrrolidin-1-yl]ethyl}-1,3-benzoxazol-2-yl)azetidin-3-yl]pyrazine-2-carboxamide; 3-[(1-methyl-1H-pyrazol-4-yl)amino]-6-(1-{6-[(5-methyl-1,3-thiazol-2-yl)methoxy]-1,3-benzoxazol-2-yl}azetidin-3-yl)pyrazine-2-carboxamide; 6-(1-{6-[2-(dimethylamino)-2-oxoethoxy]-1,3-benzoxazol-2-yl}azetidin-3-yl)-3-[(1-methyl-1H-pyrazol-4-yl)amino]pyridine-2-carboxamide; 6-{1-[6-(3-hydroxy-3-methylbutoxy)-1,3-benzoxazol-2-yl]azetidin-3-yl}-3-[(1-methyl-1H-pyrazol-4-yl)amino]pyridine-2-carboxamide; 6-[1-(6-hydroxy-1,3-benzoxazol-2-yl)azetidin-3-yl]-3-[(1-methyl-1H-pyrazol-4-yl)amino]pyridine-2-carboxamide; 6-{1-[6-(2-hydroxy-2-methylpropoxy)-1,3-benzoxazol-2-yl]azetidin-3-yl}-3-[(1-methyl-1H-pyrazol-4-yl)amino]pyridine-2-carboxamide; 6-(1-{6-[(5-methyl-1,3,4-oxadiazol-2-yl)methoxy]-1,3-benzoxazol-2-yl}azetidin-3-yl)-3-[(1-methyl-1H-pyrazol-4-yl)amino]pyrazine-2-carboxamide; 6-{1-[6-(2-cyclopropyl-2-oxoethoxy)-1,3-benzoxazol-2-yl]azetidin-3-yl}-3-[(1-methyl-1H-pyrazol-4-yl)amino]pyrazine-2-carboxamide; 6-{1-[6-(4-hydroxyphenyl)-1,3-benzoxazol-2-yl]azetidin-3-yl}-3-[(1-methyl-1H-pyrazol-4-yl)amino]pyrazine-2-carboxamide; 6-(1-{6-[4-(2-hydroxyethoxy)phenyl]-1,3-benzoxazol-2-yl}azetidin-3-yl)-3-[(1-methyl-1H-pyrazol-4-yl)amino]pyrazine-2-carboxamide; 6-(1-{6-[1-(2-hydroxyethyl)-1H-pyrazol-4-yl]-1,3-benzoxazol-2-yl}azetidin-3-yl)-3-[(1-methyl-1H-pyrazol-4-yl)amino]pyrazine-2-carboxamide; 6-(1-{6-[1-(2-methoxyethyl)-1H-pyrazol-4-yl]-1,3-benzoxazol-2-yl}azetidin-3-yl)-3-[(1-methyl-1H-pyrazol-4-yl)amino]pyrazine-2-carboxamide; 3-[(1-methyl-1H-pyrazol-4-yl)amino]-6-{1-[6-(1-methyl-1H-pyrazol-4-yl)-1,3-benzoxazol-2-yl]azetidin-3-yl}pyrazine-2-carboxamide; 3-[(1-methyl-1H-pyrazol-4-yl)amino]-6-(1-{6-[(1H-pyrazol-4-yl)methoxy]-1,3-benzoxazol-2-yl}azetidin-3-yl)pyrazine-2-carboxamide; 3-[(1-tert-butyl-1H-pyrazol-4-yl)amino]-6-{1-[6-(3-hydroxy-3-methylbutoxy)-1,3-benzoxazol-2-yl]azetidin-3-yl}pyrazine-2-carboxamide; 4-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylbutan-2-yl glycinate; 4-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylbutan-2-yl [(tert-butoxycarbonyl)amino]acetate; 6-{1-[6-(3-hydroxy-3-methylbutoxy)-1,3-benzoxazol-2-yl]azetidin-3-yl}-3-{[1-(2-hydroxy-2-methylpropyl)-1H-pyrazol-4-yl]amino}pyrazine-2-carboxamide; 6-{1-[6-(3-hydroxy-3-methylbutoxy)-1,3-benzoxazol-2-yl]azetidin-3-yl}-3-[(1-{[2-(trimethylsilyl)ethoxy]methyl}-1H-pyrazol-4-yl)amino]pyrazine-2-carboxamide; 6-{1-[6-(3-hydroxy-3-methylbutoxy)-1,3-benzoxazol-2-yl]azetidin-3-yl}-3-[(1H-pyrazol-4-yl)amino]pyrazine-2-carboxamide; 3-[(1-methyl-1H-pyrazol-4-yl)amino]-6-(1-{6-[(1-methyl-1H-pyrazol-4-yl)methoxy]-1,3-benzoxazol-2-yl}azetidin-3-yl)pyrazine-2-carboxamide; 6-(1-{6-[(1-methyl-1H-imidazol-4-yl)methoxy]-1,3-benzoxazol-2-yl}azetidin-3-yl)-3-[(1-methyl-1H-pyrazol-4-yl)amino]pyrazine-2-carboxamide; 3-[(1-methyl-1H-pyrazol-4-yl)amino]-6-{1-[6-(2-methyl-1,3-thiazol-5-yl)-1,3-benzoxazol-2-yl]azetidin-3-yl}pyrazine-2-carboxamide; 6-(1-{6-[4-(2-methoxyethoxy)phenyl]-1,3-benzoxazol-2-yl}azetidin-3-yl)-3-[(1-methyl-1H-pyrazol-4-yl)amino]pyrazine-2-carboxamide; 6-{1-[6-(2-ethoxypropan-2-yl)-1,3-benzoxazol-2-yl]azetidin-3-yl}-3-[(1-methyl-1H-pyrazol-4-yl)amino]pyrazine-2-carboxamide; 6-(1-{6-[(5-methoxypyrimidin-2-yl)oxy]-1,3-benzoxazol-2-yl}azetidin-3-yl)-3-[(1-methyl-1H-pyrazol-4-yl)amino]pyrazine-2-carboxamide; 3-[(1-methyl-1H-pyrazol-4-yl)amino]-6-(1-{6-[(pyridazin-3-yl)oxy]-1,3-benzoxazol-2-yl}azetidin-3-yl)pyridine-2-carboxamide; 6-[1-(6-{[1-(2-hydroxyethyl)-1H-pyrazol-4-yl]methoxy}-1,3-benzoxazol-2-yl)azetidin-3-yl]-3-[(1-methyl-1H-pyrazol-4-yl)amino]pyrazine-2-carboxamide; ethyl [4-({[2-(3-{6-carbamoyl-5-[(1-methyl-1H-pyrazol-4-yl)amino]pyrazin-2-yl}azetidin-1-yl)-1,3-benzoxazol-6-yl]oxy}methyl)-1H-pyrazol-1-yl]acetate; 6-{1-[6-(1-methyl-1H-imidazol-4-yl)-1,3-benzoxazol-2-yl]azetidin-3-yl}-3-[(1-methyl-1H-pyrazol-4-yl)amino]pyrazine-2-carboxamide; 1-{[2-(3-{6-carbamoyl-5-[(1-methyl-1H-pyrazol-4-yl)amino]pyridin-2-yl}azetidin-1-yl)-1,3-benzoxazol-6-yl]oxy}-2-methylpropan-2-yl dihydrogen phosphate; 6-{1-[6-(2-cyano-2-methylpropoxy)-1,3-benzoxazol-2-yl]azetidin-3-yl}-3-[(1-methyl-1H-pyrazol-4-yl)amino]pyrazine-2-carboxamide; 1-{[2-(3-{6-carbamoyl-5-[(1-methyl-1H-pyrazol-4-yl)amino]pyridin-2-yl}azetidin-1-yl)-1,3-benzoxazol-6-yl]oxy}-2-methylpropan-2-yl glycinate; 4-{[2-(3-{6-carbamoyl-5-[(1-methyl-1H-pyrazol-4-yl)amino]pyridin-2-yl}azetidin-1-yl)-1,3-benzoxazol-6-yl]oxy}-2-methylbutan-2-yl dihydrogen phosphate; 4-{[2-(3-{6-carbamoyl-5-[(1-methyl-1H-pyrazol-4-yl)amino]pyridin-2-yl}azetidin-1-yl)-1,3-benzoxazol-6-yl]oxy}-2-methylbutan-2-yl glycinate; 6-{1-[6-(1-hydroxy-2-methylpropan-2-yl)-1,3-benzoxazol-2-yl]azetidin-3-yl}-3-[(1-methyl-1H-pyrazol-4-yl)amino]pyrazine-2-carboxamide; ethyl 2-[2-(3-{6-carbamoyl-5-[(1-methyl-1H-pyrazol-4-yl)amino]pyrazin-2-yl}azetidin-1-yl)-1,3-benzoxazol-6-yl]-2-methylpropanoate; 1-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylpropan-2-yl dihydrogen phosphate; and 1-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylpropan-2-yl glycinate.

Exemplary compounds of Formula (I) include, but are not limited to, the compounds shown in Table 1 below, and pharmaceutically acceptable salts thereof. It is to be understood that when there is a discrepancy between the name of the compound found herein and the structure found in Table 1, the structure in Table 1 shall prevail.

TABLE 1
Exemplary Compounds

| Ex. | Structure |
|---|---|
| 0 | 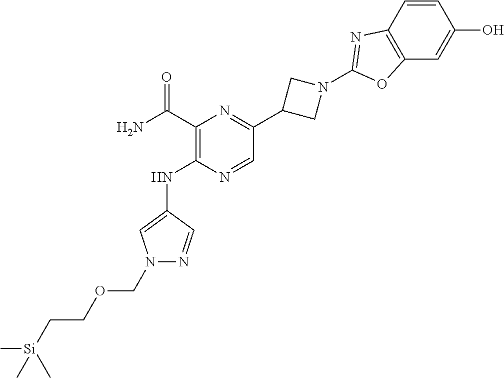 [SMILES: C[Si](C)(C)CCOCn1cc(Nc2ncc(C3CN(c4nc5ccc(O)cc5o4)C3)nc2C(N)=O)cn1] |
| 1 | 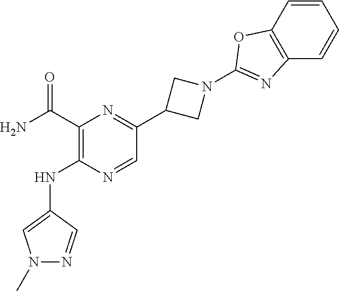 [SMILES: Cn1cc(Nc2ncc(C3CN(c4nc5ccccc5o4)C3)nc2C(N)=O)cn1] |
| 2 | 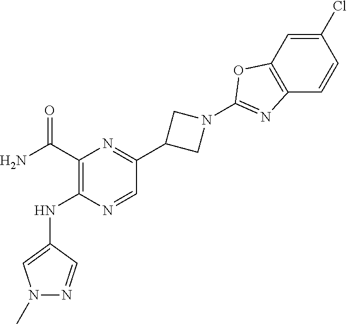 [SMILES: Cn1cc(Nc2ncc(C3CN(c4nc5ccc(Cl)cc5o4)C3)nc2C(N)=O)cn1] |
| 3 | 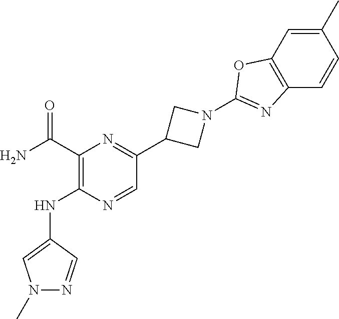 |
| 4 | 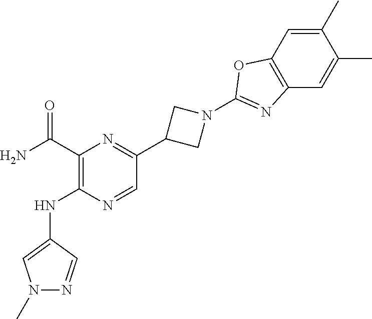 |
| 5 | 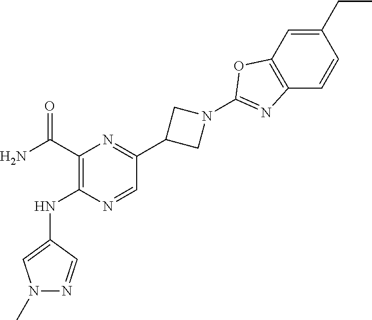 [SMILES: CCc1ccc2nc(N3CC(c4cnc(Nc5cnn(C)c5)c(C(N)=O)n4)C3)oc2c1] |
| 6 | 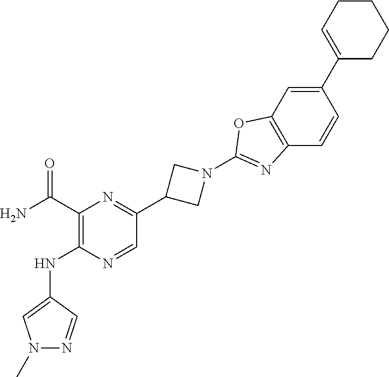 [SMILES: Cn1cc(Nc2ncc(C3CN(c4nc5ccc(C6=CCCCC6)cc5o4)C3)nc2C(N)=O)cn1] |
| 6A | 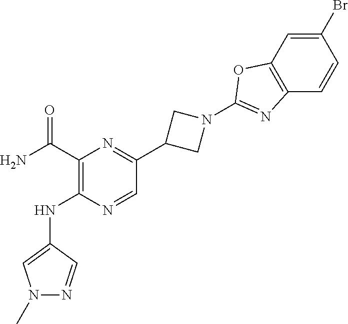 [SMILES: Cn1cc(Nc2ncc(C3CN(c4nc5ccc(Br)cc5o4)C3)nc2C(N)=O)cn1] |
| 7 | 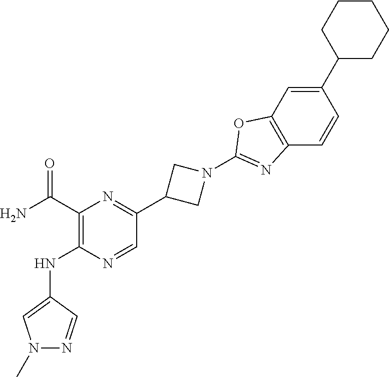 [SMILES: Cn1cc(Nc2ncc(C3CN(c4nc5ccc(C6CCCCC6)cc5o4)C3)nc2C(N)=O)cn1] |
| 8 | 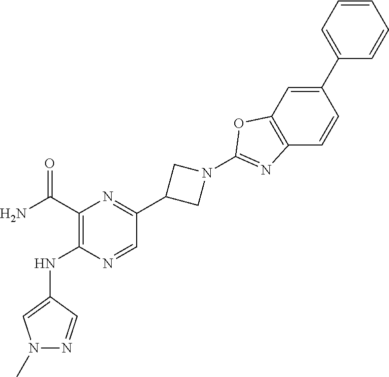 [SMILES: Cn1cc(Nc2ncc(C3CN(c4nc5ccc(-c6ccccc6)cc5o4)C3)nc2C(N)=O)cn1] |
| 9 | 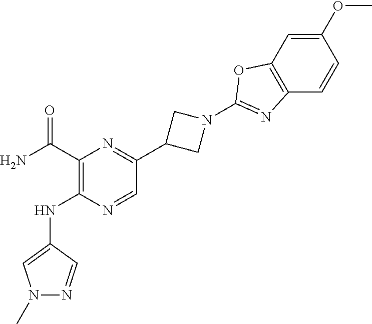 |
| 10 | 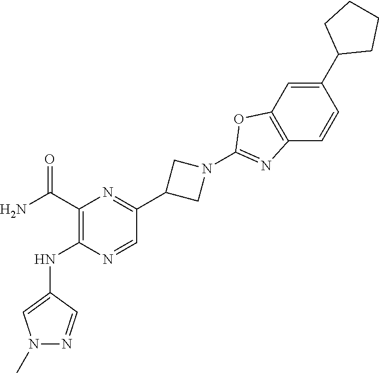 [SMILES: Cn1cc(Nc2ncc(C3CN(c4nc5ccc(C6CCCC6)cc5o4)C3)nc2C(N)=O)cn1] |
| 10A | 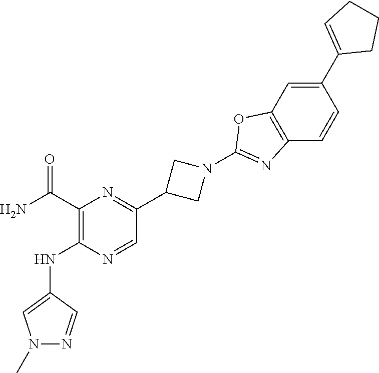 [SMILES: Cn1cc(Nc2ncc(C3CN(c4nc5ccc(C6=CCCC6)cc5o4)C3)nc2C(N)=O)cn1] |
| 11 | 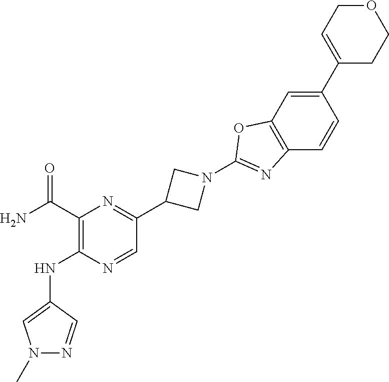 [SMILES: Cn1cc(Nc2ncc(C3CN(c4nc5ccc(C6=CCOCC6)cc5o4)C3)nc2C(N)=O)cn1] |
| 12 | 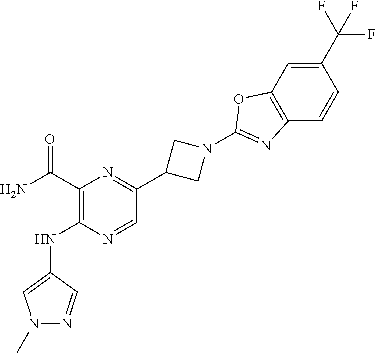 [SMILES: Cn1cc(Nc2ncc(C3CN(c4nc5ccc(C(F)(F)F)cc5o4)C3)nc2C(N)=O)cn1] |
| 13 | 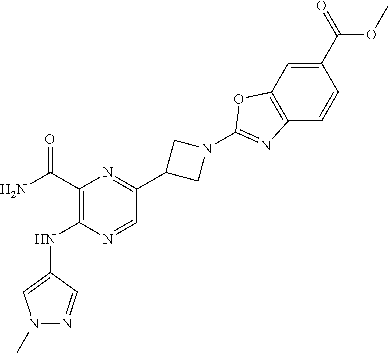 |
| 14 | 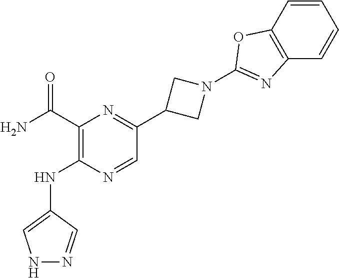 [SMILES: NC(=O)c1nc(C2CN(c3nc4ccccc4o3)C2)cnc1Nc1cn[nH]c1] |
| 15 | 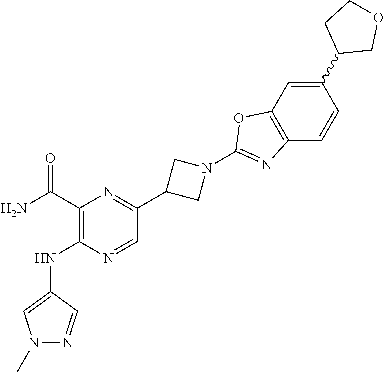 [SMILES: Cn1cc(Nc2ncc(C3CN(c4nc5ccc(C6CCOC6)cc5o4)C3)nc2C(N)=O)cn1] |
| 15A | 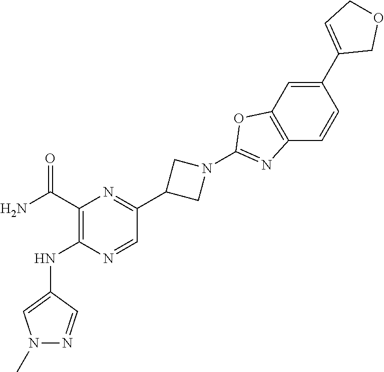 [SMILES: Cn1cc(Nc2ncc(C3CN(c4nc5ccc(C6=CCOC6)cc5o4)C3)nc2C(N)=O)cn1] |
| 16 | 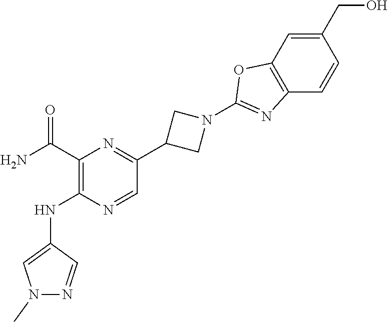 [SMILES: Cn1cc(Nc2ncc(C3CN(c4nc5ccc(CO)cc5o4)C3)nc2C(N)=O)cn1] |
| 17 | 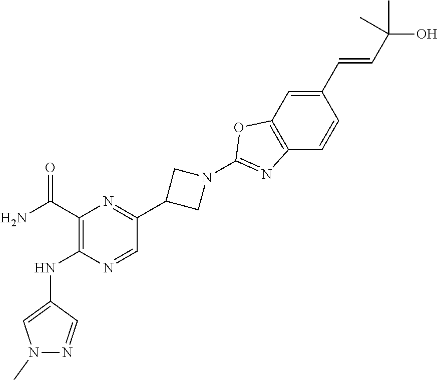 [SMILES: Cn1cc(Nc2ncc(C3CN(c4nc5ccc(/C=C/C(C)(C)O)cc5o4)C3)nc2C(N)=O)cn1] |
| 18 | 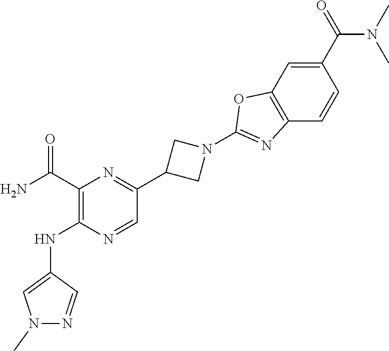 |
| 18A | 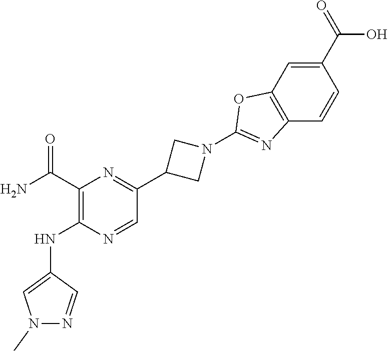 [SMILES: Cn1cc(Nc2ncc(C3CN(c4nc5ccc(C(=O)O)cc5o4)C3)nc2C(N)=O)cn1] |
| 19 | 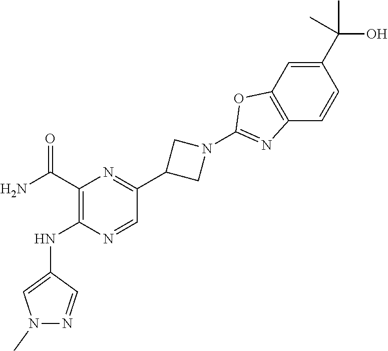 [SMILES: Cn1cc(Nc2ncc(C3CN(c4nc5ccc(C(C)(C)O)cc5o4)C3)nc2C(N)=O)cn1] |
| 20 | 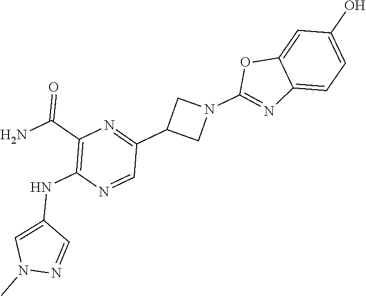 [SMILES: Cn1cc(Nc2ncc(C3CN(c4nc5ccc(O)cc5o4)C3)nc2C(N)=O)cn1] |
| 21 | 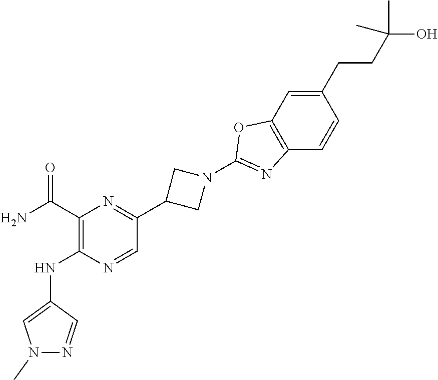 [SMILES: Cn1cc(Nc2ncc(C3CN(c4nc5ccc(CCC(C)(C)O)cc5o4)C3)nc2C(N)=O)cn1] |
| 22 | 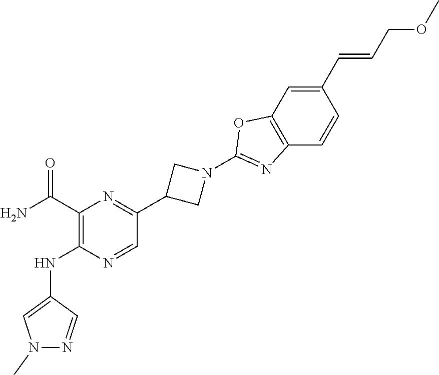 |
| 23 | 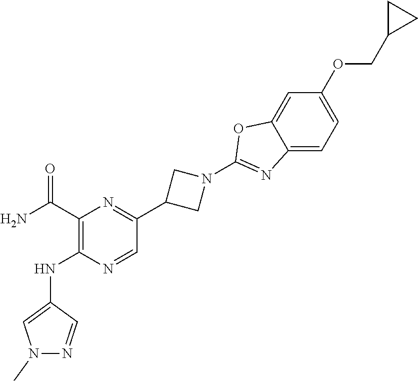 [SMILES: Cn1cc(Nc2ncc(C3CN(c4nc5ccc(OCC6CC6)cc5o4)C3)nc2C(N)=O)cn1] |
| 24 | 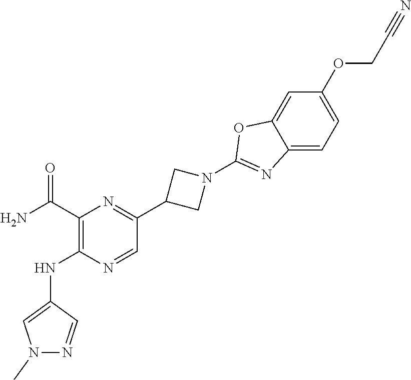 [SMILES: Cn1cc(Nc2ncc(C3CN(c4nc5ccc(OCC#N)cc5o4)C3)nc2C(N)=O)cn1] |
| 25 | 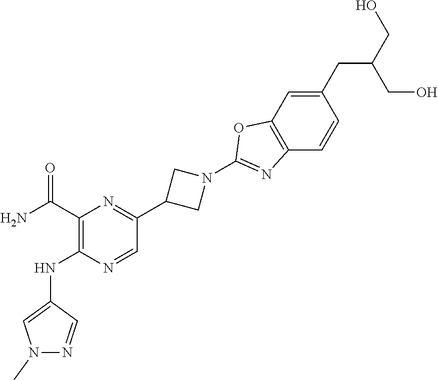 [SMILES: Cn1cc(Nc2ncc(C3CN(c4nc5ccc(CC(CO)CO)cc5o4)C3)nc2C(N)=O)cn1] |
| 26 | 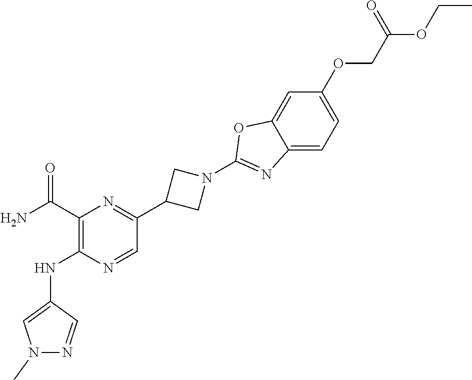 [SMILES: CCOC(=O)COc1ccc2nc(N3CC(c4cnc(Nc5cnn(C)c5)c(C(N)=O)n4)C3)oc2c1] |
| 27 | 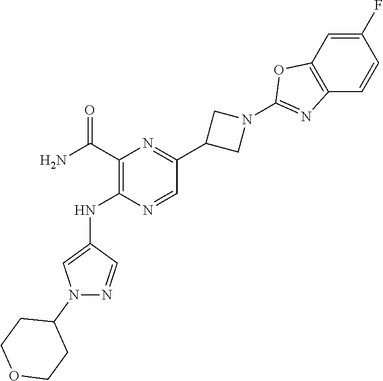 [SMILES: NC(=O)c1nc(C2CN(c3nc4ccc(F)cc4o3)C2)cnc1Nc1cnn(C2CCOCC2)c1] |
| 28 | 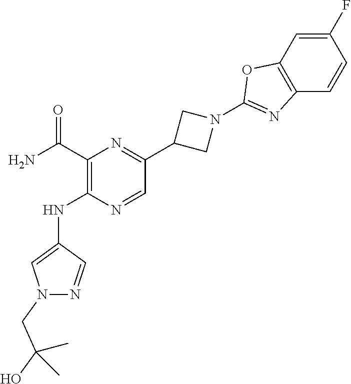 [SMILES: CC(C)(O)Cn1cc(Nc2ncc(C3CN(c4nc5ccc(F)cc5o4)C3)nc2C(N)=O)cn1] |
| 29 | 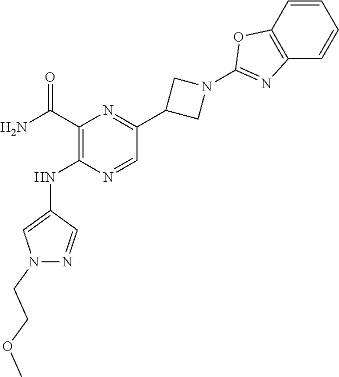 [SMILES: COCCn1cc(Nc2ncc(C3CN(c4nc5ccccc5o4)C3)nc2C(N)=O)cn1] |
| 30 | 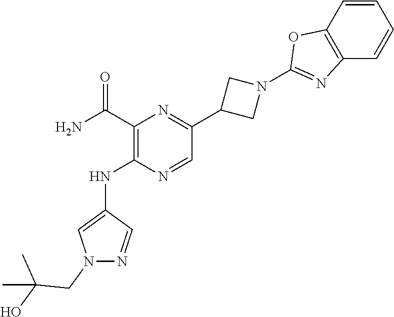 [SMILES: CC(C)(O)Cn1cc(Nc2ncc(C3CN(c4nc5ccccc5o4)C3)nc2C(N)=O)cn1] |
| 31 | 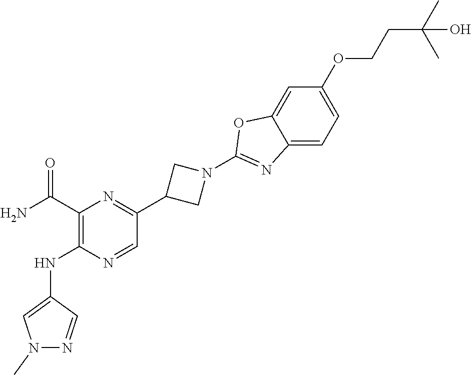 [SMILES: Cn1cc(Nc2ncc(C3CN(c4nc5ccc(OCCC(C)(C)O)cc5o4)C3)nc2C(N)=O)cn1] |
| 32 | 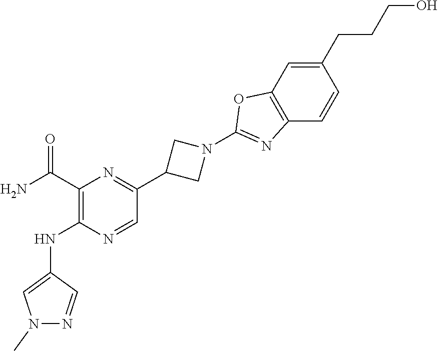 [SMILES: Cn1cc(Nc2ncc(C3CN(c4nc5ccc(CCCO)cc5o4)C3)nc2C(N)=O)cn1] |
| 32A | 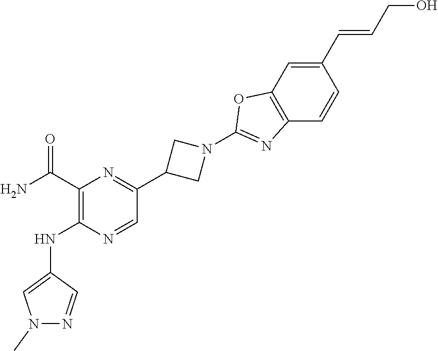 [SMILES: Cn1cc(Nc2ncc(C3CN(c4nc5ccc(/C=C/CO)cc5o4)C3)nc2C(N)=O)cn1] |
| 33 | 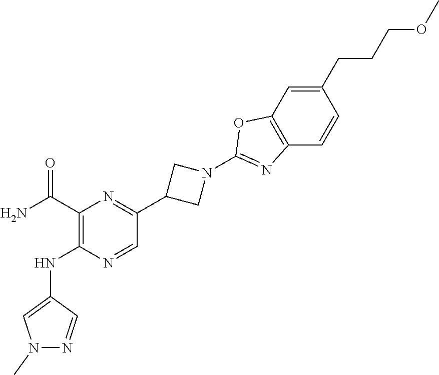 |
| 34 | 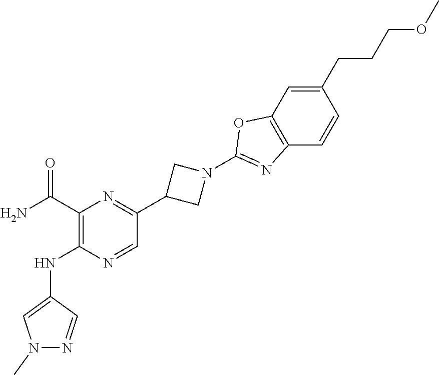 |
| 35 | 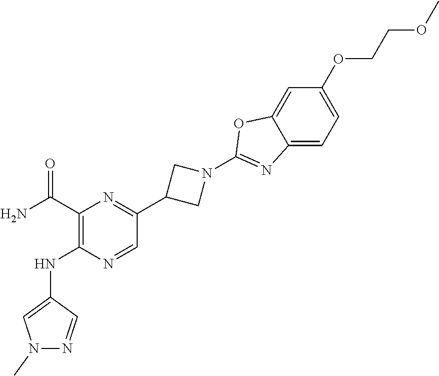 |
| 36 | 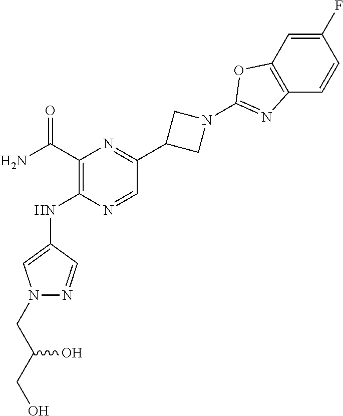 [SMILES: NC(=O)c1nc(C2CN(c3nc4ccc(F)cc4o3)C2)cnc1Nc1cnn(CC(O)CO)c1] |
| 36C | 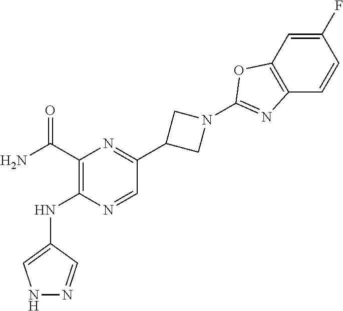 [SMILES: NC(=O)c1nc(C2CN(c3nc4ccc(F)cc4o3)C2)cnc1Nc1cn[nH]c1] |
| 36D | 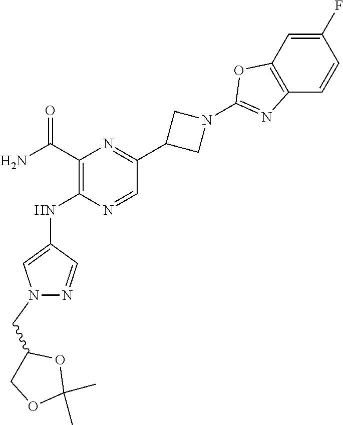 [SMILES: CC1(C)OCC(Cn2cc(Nc3ncc(C4CN(c5nc6ccc(F)cc6o5)C4)nc3C(N)=O)cn2)O1] |
| 37 | 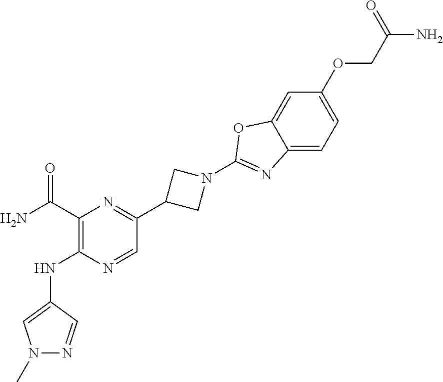 [SMILES: Cn1cc(Nc2ncc(C3CN(c4nc5ccc(OCC(N)=O)cc5o4)C3)nc2C(N)=O)cn1] |
| 38 | 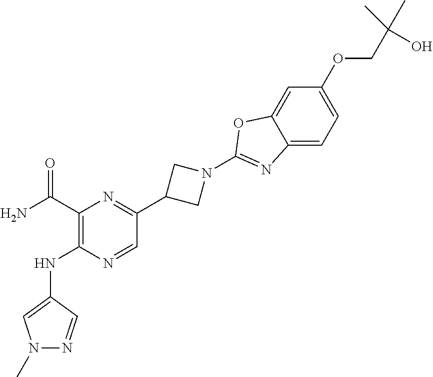 [SMILES: Cn1cc(Nc2ncc(C3CN(c4nc5ccc(OCC(C)(C)O)cc5o4)C3)nc2C(N)=O)cn1] |
| 39 | 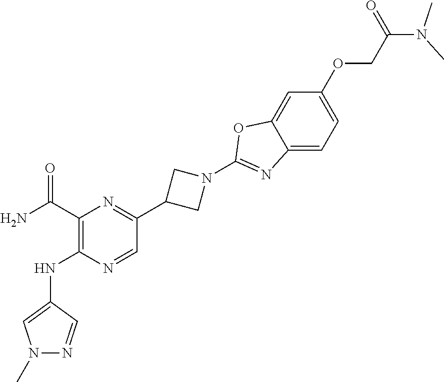 |
| 40 | 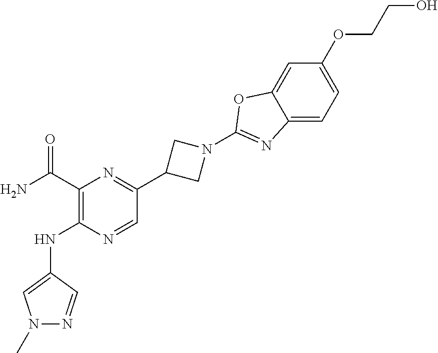 [SMILES: Cn1cc(Nc2ncc(C3CN(c4nc5ccc(OCCO)cc5o4)C3)nc2C(N)=O)cn1] |
| 41 | 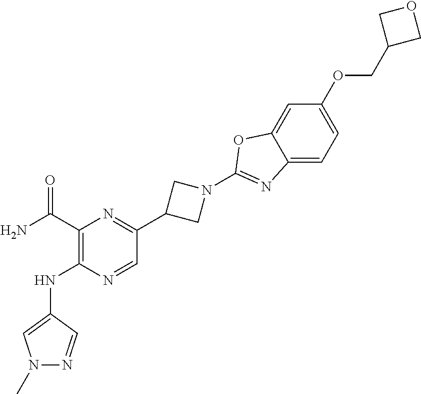 [SMILES: Cn1cc(Nc2ncc(C3CN(c4nc5ccc(OCC6COC6)cc5o4)C3)nc2C(N)=O)cn1] |
| 42 | 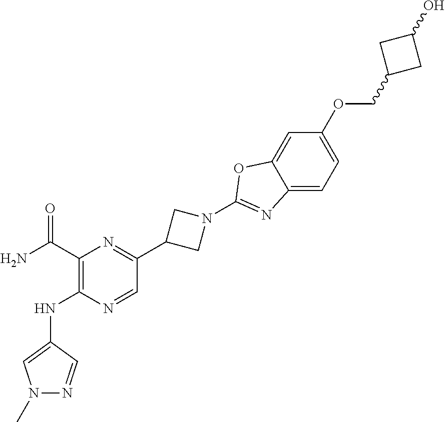 [SMILES: Cn1cc(Nc2ncc(C3CN(c4nc5ccc(OCC6CC(O)C6)cc5o4)C3)nc2C(N)=O)cn1] |
| 43 | 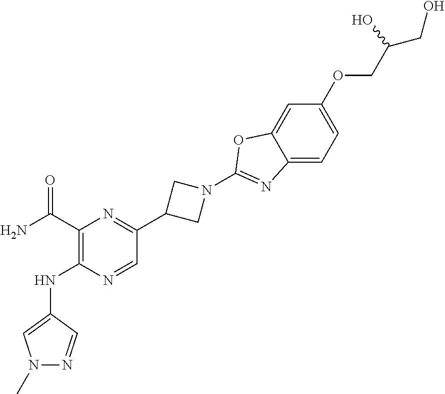 [SMILES: Cn1cc(Nc2ncc(C3CN(c4nc5ccc(OCC(O)CO)cc5o4)C3)nc2C(N)=O)cn1] |
| 44 | 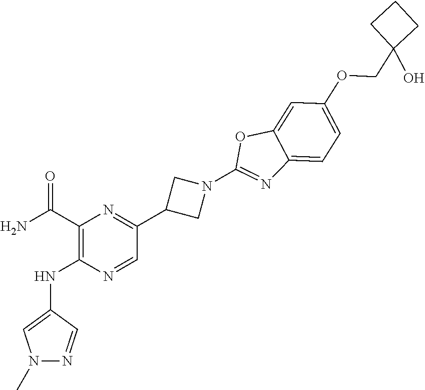 [SMILES: Cn1cc(Nc2ncc(C3CN(c4nc5ccc(OCC6(O)CCC6)cc5o4)C3)nc2C(N)=O)cn1] |
| 45 | 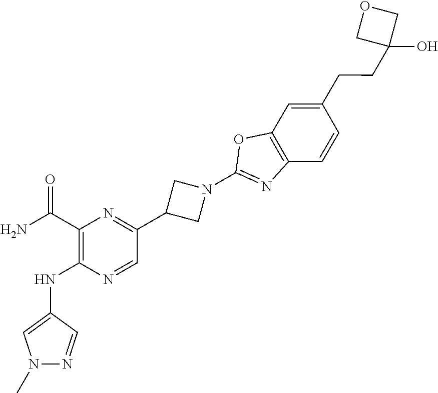 [SMILES: Cn1cc(Nc2ncc(C3CN(c4nc5ccc(CCC6(O)COC6)cc5o4)C3)nc2C(N)=O)cn1] |
| 46 | 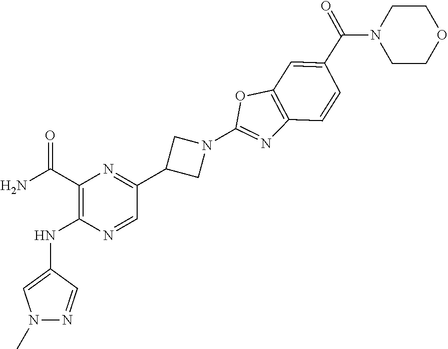 [SMILES: Cn1cc(Nc2ncc(C3CN(c4nc5ccc(C(=O)N6CCOCC6)cc5o4)C3)nc2C(N)=O)cn1] |
| 47 | 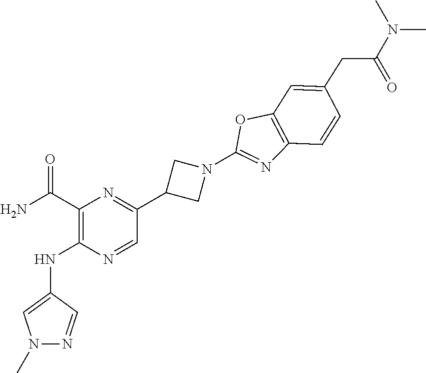 |
| 48 | 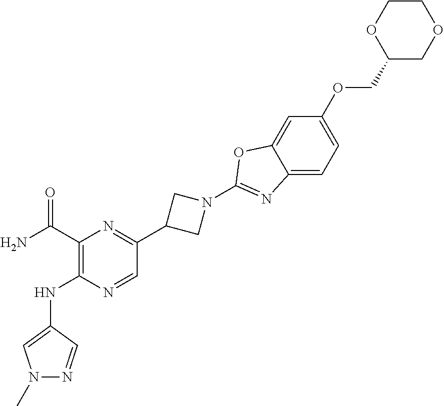 [SMILES: Cn1cc(Nc2ncc(C3CN(c4nc5ccc(OC[C@H]6COCCO6)cc5o4)C3)nc2C(N)=O)cn1] |
| 49 | 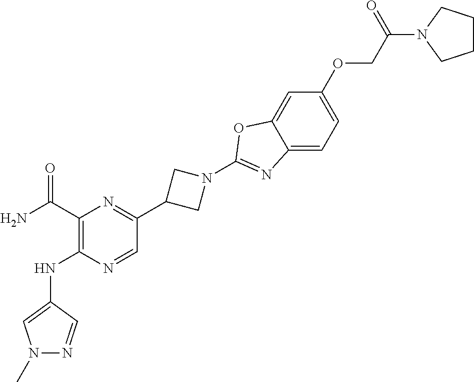 [SMILES: Cn1cc(Nc2ncc(C3CN(c4nc5ccc(OCC(=O)N6CCCC6)cc5o4)C3)nc2C(N)=O)cn1] |
| 50 | 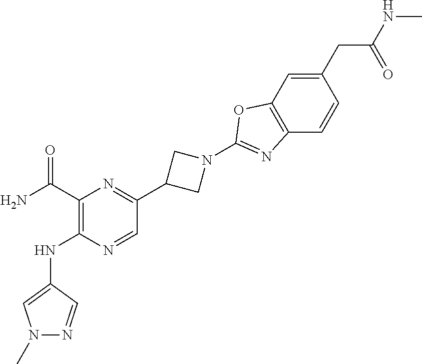 |
| 51 | 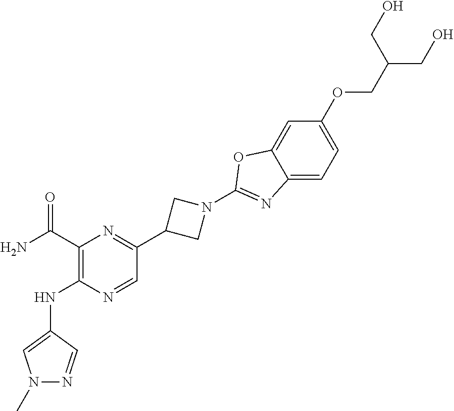 [SMILES: Cn1cc(Nc2ncc(C3CN(c4nc5ccc(OCC(CO)CO)cc5o4)C3)nc2C(N)=O)cn1] |
| 51A | 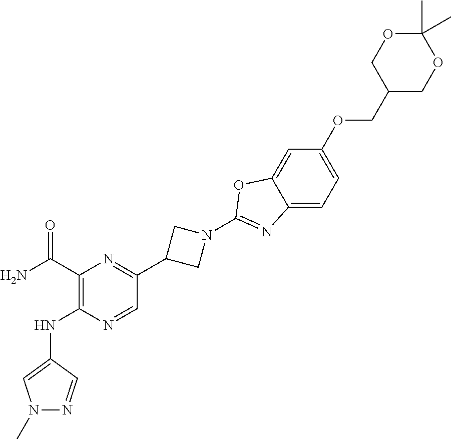 |
| 52 | 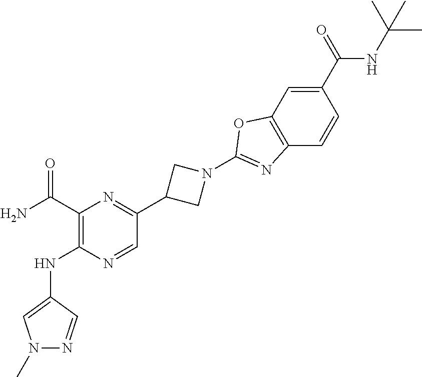 [SMILES: Cn1cc(Nc2ncc(C3CN(c4nc5ccc(C(=O)NC(C)(C)C)cc5o4)C3)nc2C(N)=O)cn1] |
| 53 | 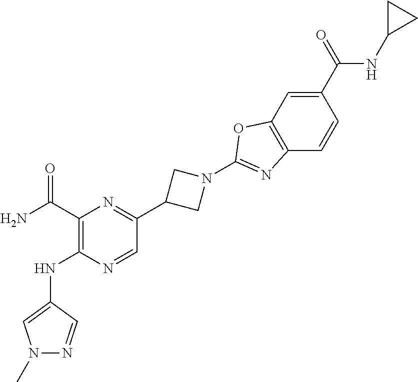 [SMILES: Cn1cc(Nc2ncc(C3CN(c4nc5ccc(C(=O)NC6CC6)cc5o4)C3)nc2C(N)=O)cn1] |
| 54 | 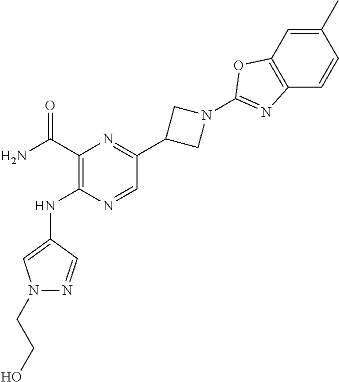 [SMILES: Cc1ccc2nc(N3CC(c4cnc(Nc5cnn(CCO)c5)c(C(N)=O)n4)C3)oc2c1] |
| 55 | 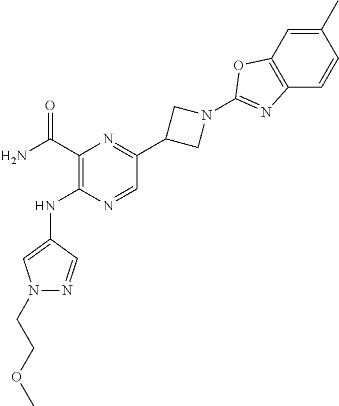 [SMILES: COCCn1cc(Nc2ncc(C3CN(c4nc5ccc(C)cc5o4)C3)nc2C(N)=O)cn1] |
| 56 | 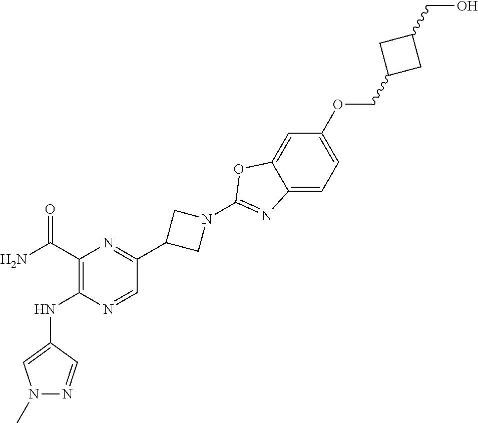 [SMILES: Cn1cc(Nc2ncc(C3CN(c4nc5ccc(OCC6CC(CO)C6)cc5o4)C3)nc2C(N)=O)cn1] |
| 57 | 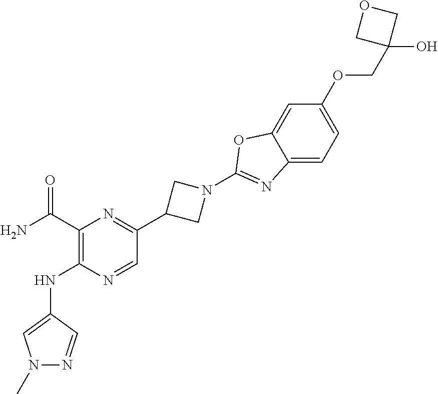 [SMILES: Cn1cc(Nc2ncc(C3CN(c4nc5ccc(OCC6(O)COC6)cc5o4)C3)nc2C(N)=O)cn1] |
| 58 | 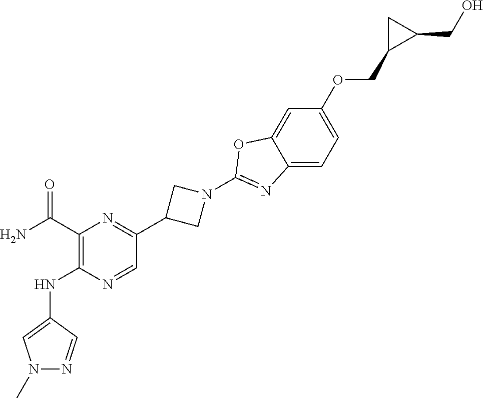 [SMILES: Cn1cc(Nc2ncc(C3CN(c4nc5ccc(OC[C@H]6C[C@H]6CO)cc5o4)C3)nc2C(N)=O)cn1] |
| 59 | 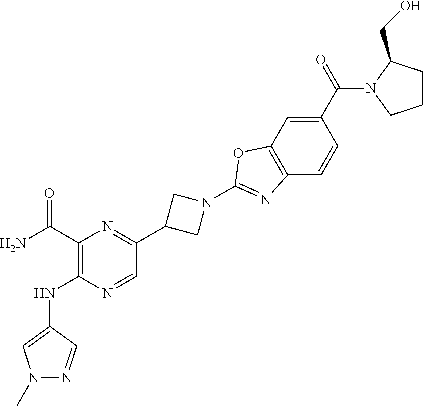 [SMILES: Cn1cc(Nc2ncc(C3CN(c4nc5ccc(C(=O)N6CCC[C@@H]6CO)cc5o4)C3)nc2C(N)=O)cn1] |
| 60 | 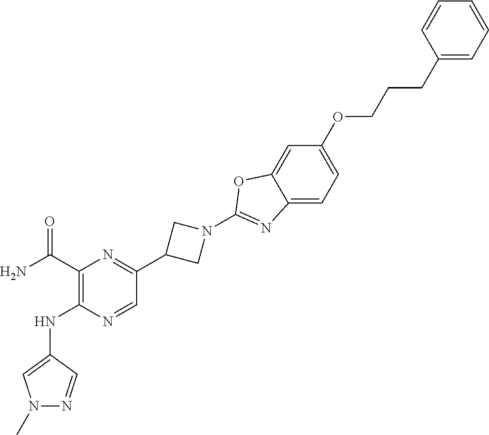 [SMILES: Cn1cc(Nc2ncc(C3CN(c4nc5ccc(OCCCc6ccccc6)cc5o4)C3)nc2C(N)=O)cn1] |
| 61 | 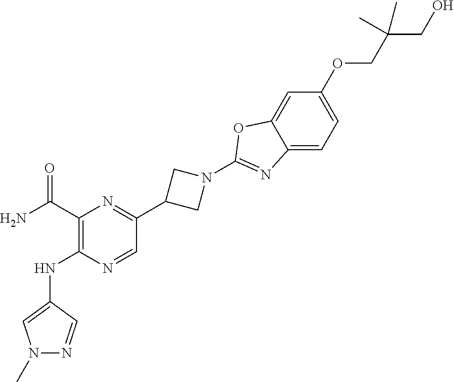 [SMILES: Cn1cc(Nc2ncc(C3CN(c4nc5ccc(OCC(C)(C)CO)cc5o4)C3)nc2C(N)=O)cn1] |
| 62 | 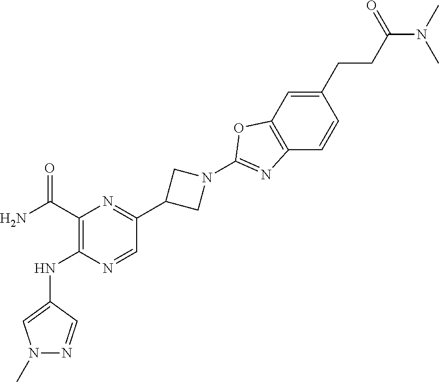 |
| 63 | 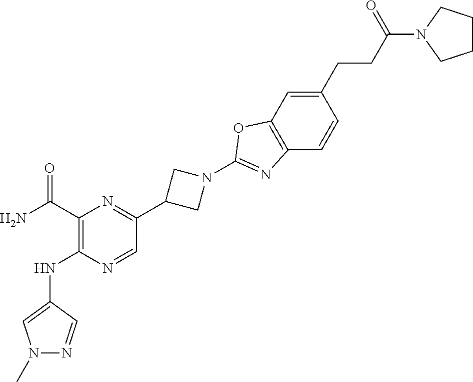 [SMILES: Cn1cc(Nc2ncc(C3CN(c4nc5ccc(CCC(=O)N6CCCC6)cc5o4)C3)nc2C(N)=O)cn1] |
| 64 | 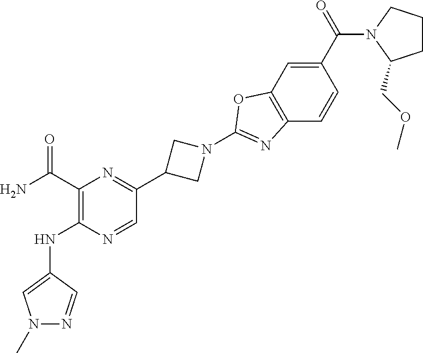 [SMILES: COC[C@H]1CCCN1C(=O)c1ccc2nc(N3CC(c4cnc(Nc5cnn(C)c5)c(C(N)=O)n4)C3)oc2c1] |
| 65 | 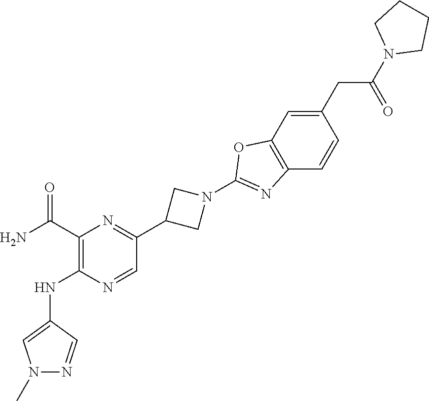 [SMILES: Cn1cc(Nc2ncc(C3CN(c4nc5ccc(CC(=O)N6CCCC6)cc5o4)C3)nc2C(N)=O)cn1] |
| 65C | 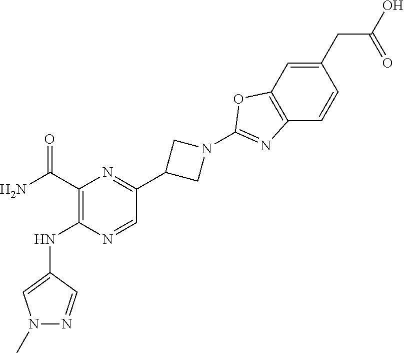 [SMILES: Cn1cc(Nc2ncc(C3CN(c4nc5ccc(CC(=O)O)cc5o4)C3)nc2C(N)=O)cn1] |
| 66 | 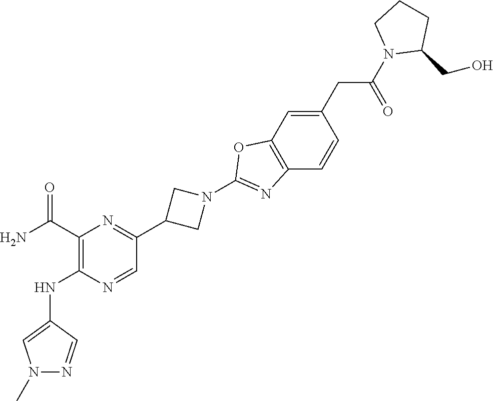 [SMILES: Cn1cc(Nc2ncc(C3CN(c4nc5ccc(CC(=O)N6CCC[C@H]6CO)cc5o4)C3)nc2C(N)=O)cn1] |
| 67 | 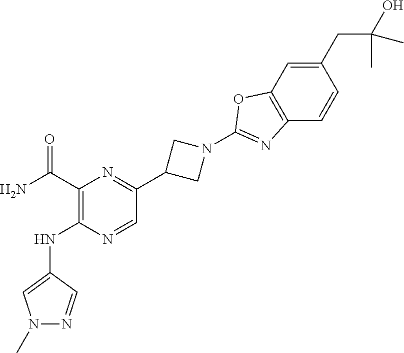 [SMILES: Cn1cc(Nc2ncc(C3CN(c4nc5ccc(CC(C)(C)O)cc5o4)C3)nc2C(N)=O)cn1] |
| 68 | 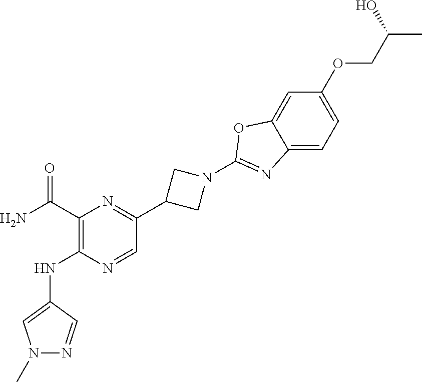 [SMILES: C[C@@H](O)COc1ccc2nc(N3CC(c4cnc(Nc5cnn(C)c5)c(C(N)=O)n4)C3)oc2c1] |
| 69 | 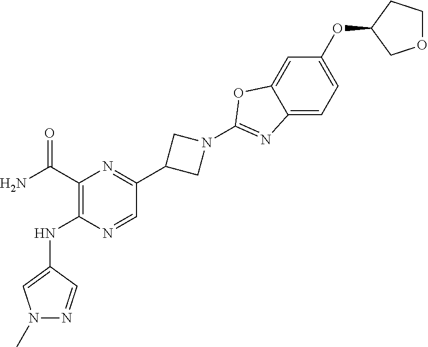 [SMILES: Cn1cc(Nc2ncc(C3CN(c4nc5ccc(O[C@H]6CCOC6)cc5o4)C3)nc2C(N)=O)cn1] |
| 70 | 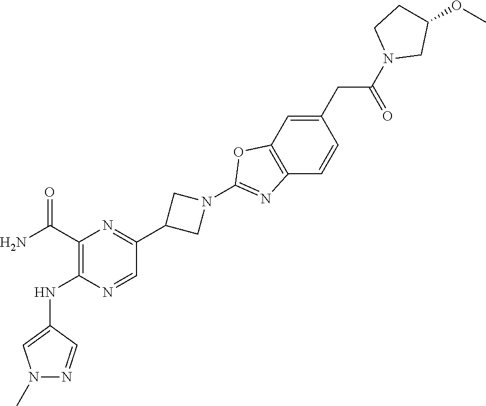 |
| 71 | 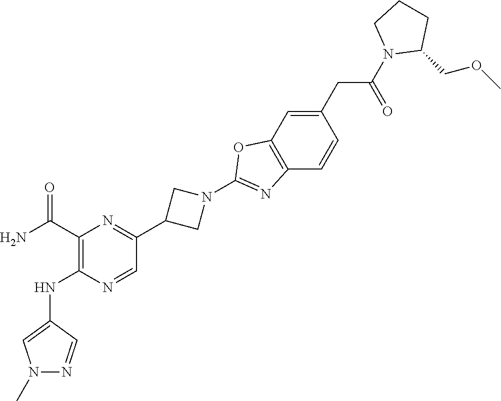 |
| 72 | 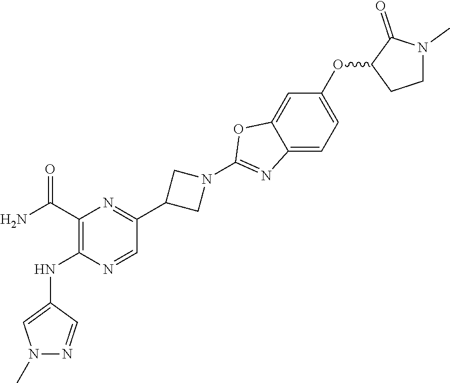 |
| 73 | 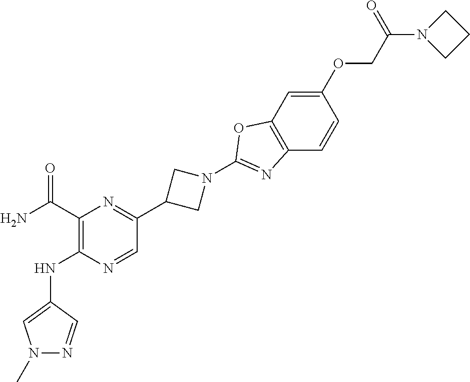 [SMILES: Cn1cc(Nc2ncc(C3CN(c4nc5ccc(OCC(=O)N6CCC6)cc5o4)C3)nc2C(N)=O)cn1] |
| 73A | 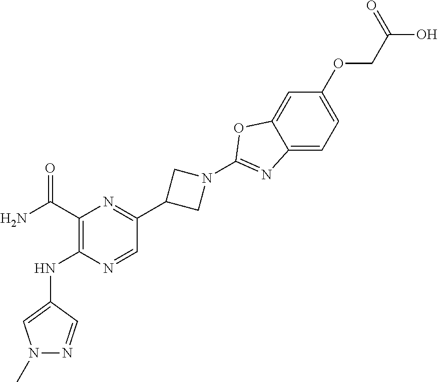 [SMILES: Cn1cc(Nc2ncc(C3CN(c4nc5ccc(OCC(=O)O)cc5o4)C3)nc2C(N)=O)cn1] |
| 74 | 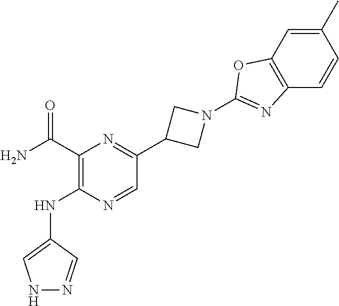 [SMILES: Cc1ccc2nc(N3CC(c4cnc(Nc5cn[nH]c5)c(C(N)=O)n4)C3)oc2c1] |
| 75 | 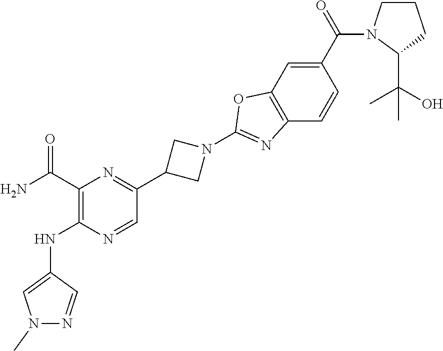 [SMILES: Cn1cc(Nc2ncc(C3CN(c4nc5ccc(C(=O)N6CCC[C@@H]6C(C)(C)O)cc5o4)C3)nc2C(N)=O)cn1] |
| 76 | 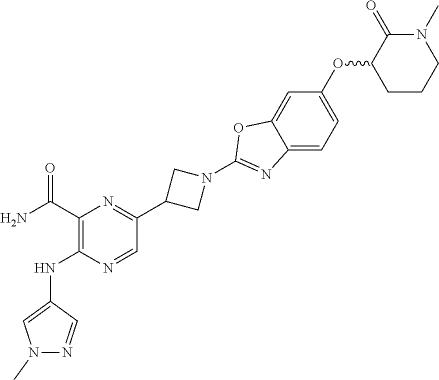 |
| 77 | 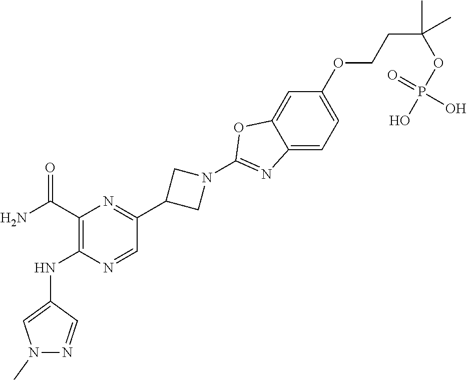 [SMILES: Cn1cc(Nc2ncc(C3CN(c4nc5ccc(OCCC(C)(C)OP(=O)(O)O)cc5o4)C3)nc2C(N)=O)cn1] |
| 77A | 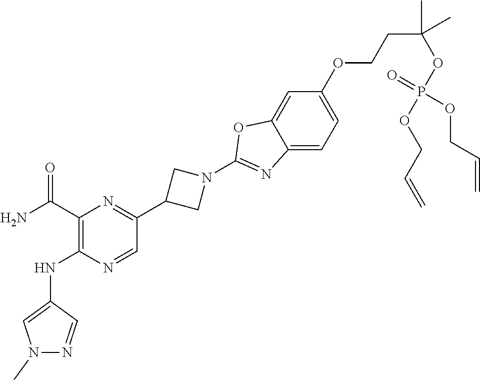 [SMILES: C=CCOP(=O)(OCC=C)OC(C)(C)CCOc1ccc2nc(N3CC(c4cnc(Nc5cnn(C)c5)c(C(N)=O)n4)C3)oc2c1] |
| 78 | 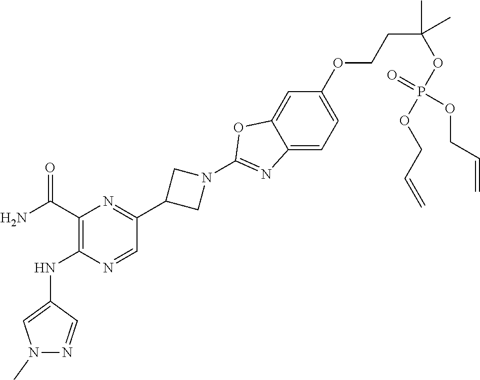 [SMILES: C=CCOP(=O)(OCC=C)OC(C)(C)CCOc1ccc2nc(N3CC(c4cnc(Nc5cnn(C)c5)c(C(N)=O)n4)C3)oc2c1] |
| 79 | 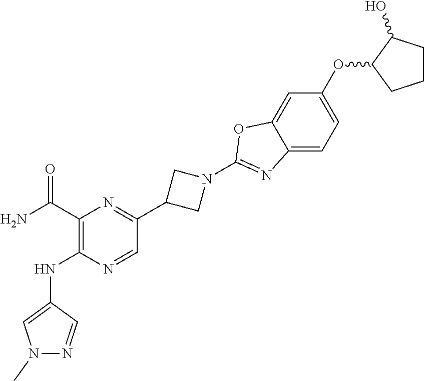 [SMILES: Cn1cc(Nc2ncc(C3CN(c4nc5ccc(OC6CCCC6O)cc5o4)C3)nc2C(N)=O)cn1] |
| 80 | 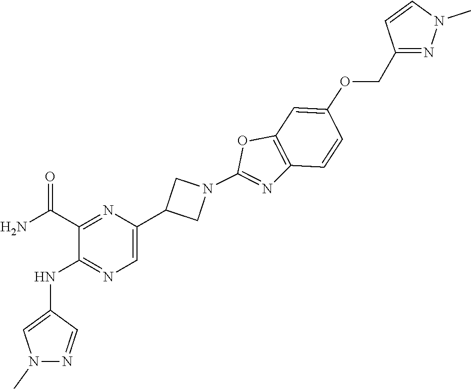 |
| 81 | 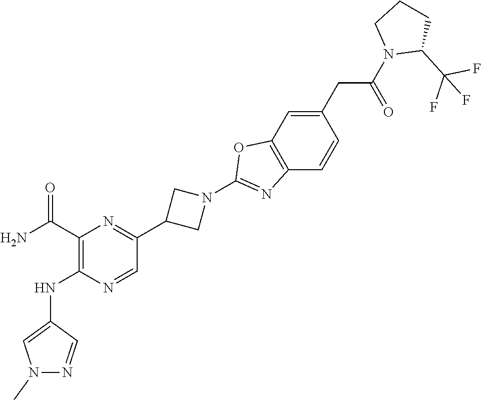 [SMILES: Cn1cc(Nc2ncc(C3CN(c4nc5ccc(CC(=O)N6CCC[C@@H]6C(F)(F)F)cc5o4)C3)nc2C(N)=O)cn1] |
| 82 | 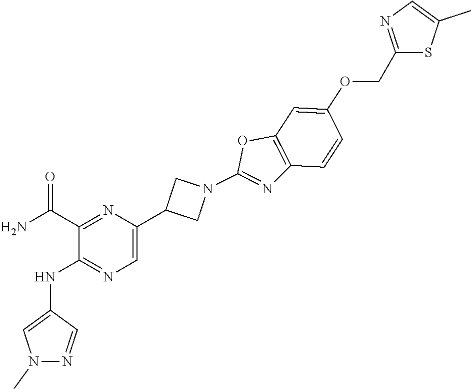 |
| 83 | 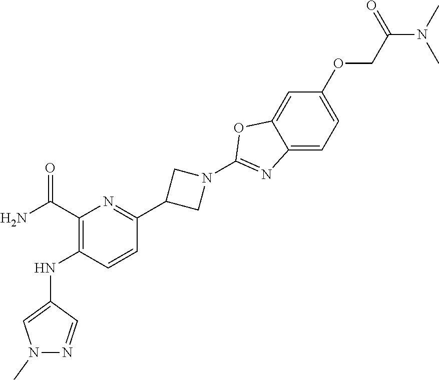 |
| 84 | 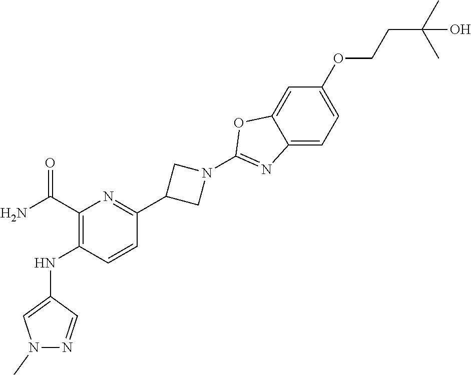 [SMILES: Cn1cc(Nc2ccc(C3CN(c4nc5ccc(OCCC(C)(C)O)cc5o4)C3)nc2C(N)=O)cn1] |
| 84A | 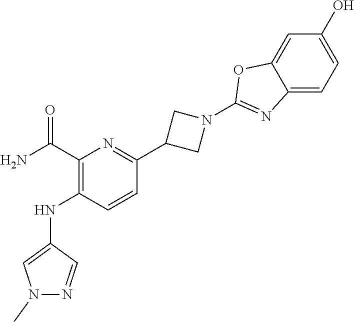 [SMILES: Cn1cc(Nc2ccc(C3CN(c4nc5ccc(O)cc5o4)C3)nc2C(N)=O)cn1] |
| 85 | 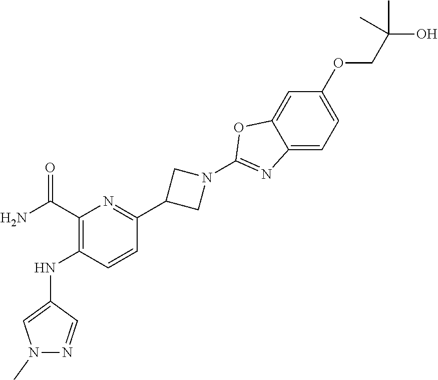 [SMILES: Cn1cc(Nc2ccc(C3CN(c4nc5ccc(OCC(C)(C)O)cc5o4)C3)nc2C(N)=O)cn1] |
| 86 | 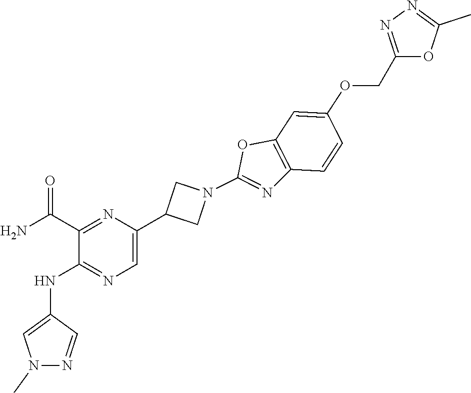 |
| 87 | 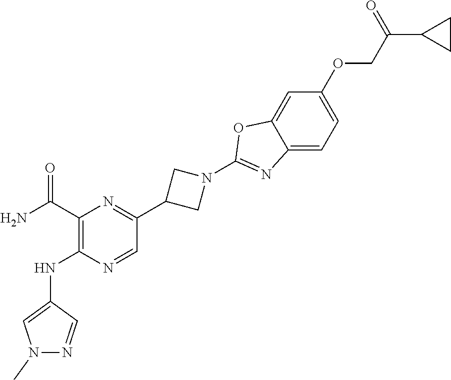 [SMILES: Cn1cc(Nc2ncc(C3CN(c4nc5ccc(OCC(=O)C6CC6)cc5o4)C3)nc2C(N)=O)cn1] |
| 88 | 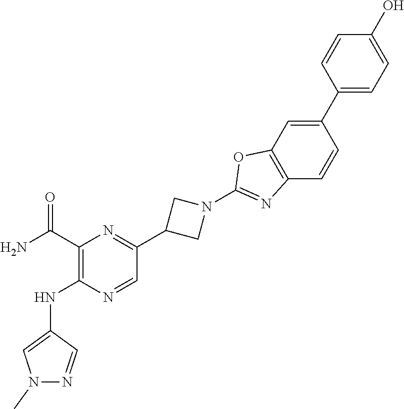 [SMILES: Cn1cc(Nc2ncc(C3CN(c4nc5ccc(-c6ccc(O)cc6)cc5o4)C3)nc2C(N)=O)cn1] |
| 89 | 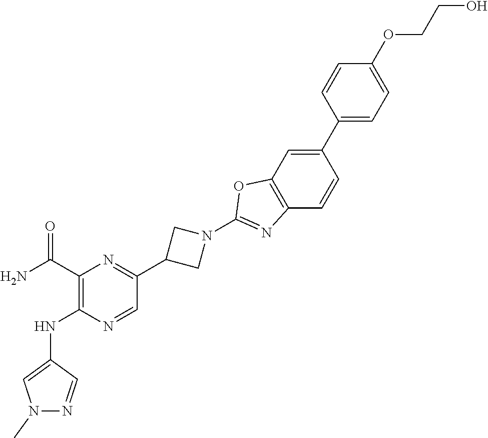 [SMILES: Cn1cc(Nc2ncc(C3CN(c4nc5ccc(-c6ccc(OCCO)cc6)cc5o4)C3)nc2C(N)=O)cn1] |
| 90 | 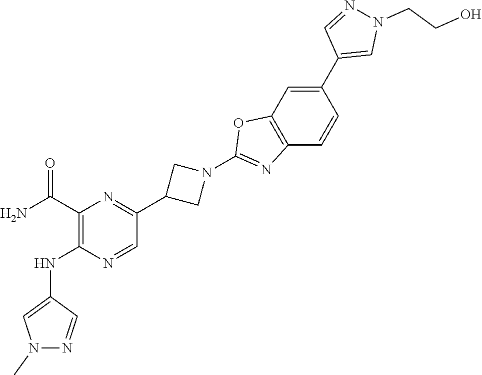 [SMILES: Cn1cc(Nc2ncc(C3CN(c4nc5ccc(-c6cnn(CCO)c6)cc5o4)C3)nc2C(N)=O)cn1] |
| 91 | 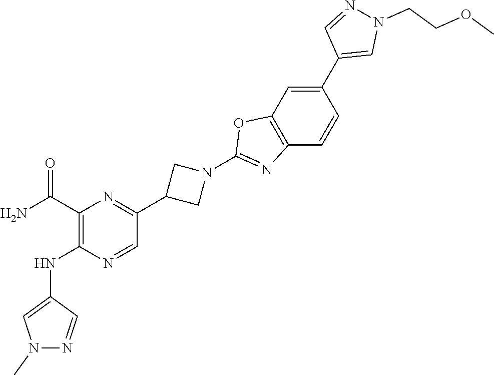 |
| 92 | 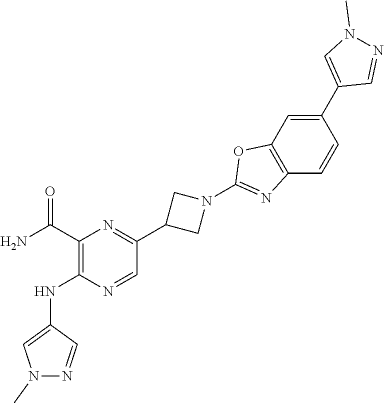 [SMILES: Cn1cc(Nc2ncc(C3CN(c4nc5ccc(-c6cnn(C)c6)cc5o4)C3)nc2C(N)=O)cn1] |
| 93 | 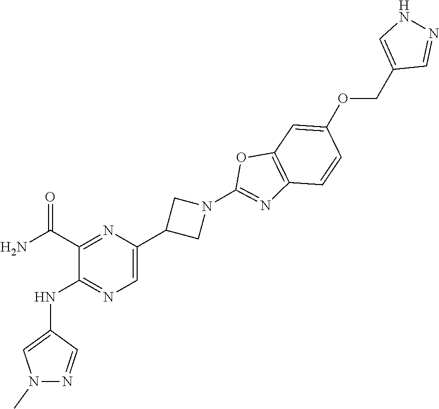 [SMILES: Cn1cc(Nc2ncc(C3CN(c4nc5ccc(OCc6cn[nH]c6)cc5o4)C3)nc2C(N)=O)cn1] |
| 94 | 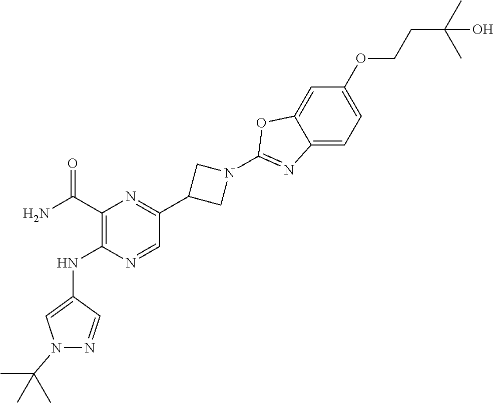 [SMILES: CC(C)(O)CCOc1ccc2nc(N3CC(c4cnc(Nc5cnn(C(C)(C)C)c5)c(C(N)=O)n4)C3)oc2c1] |
| 95 | 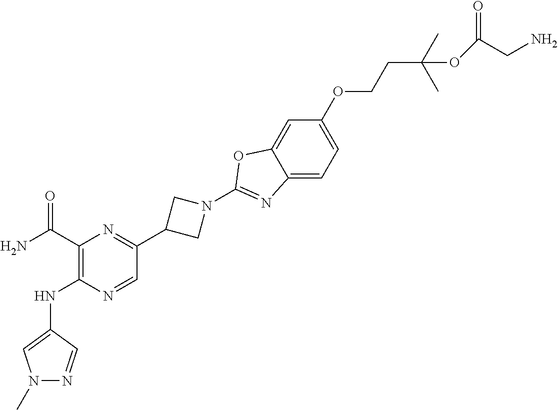 [SMILES: Cn1cc(Nc2ncc(C3CN(c4nc5ccc(OCCC(C)(C)OC(=O)CN)cc5o4)C3)nc2C(N)=O)cn1] |
| 95A | 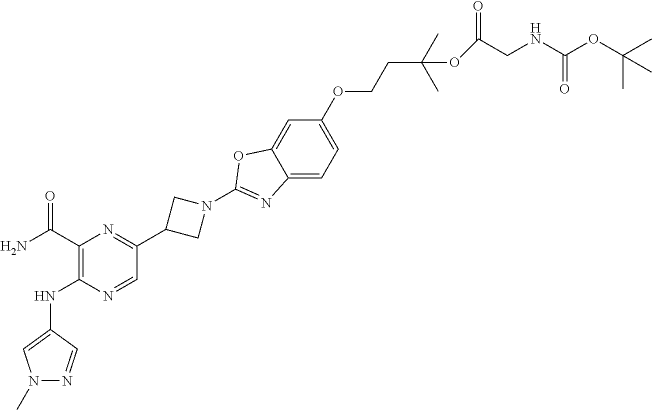 [SMILES: Cn1cc(Nc2ncc(C3CN(c4nc5ccc(OCCC(C)(C)OC(=O)CNC(=O)OC(C)(C)C)cc5o4)C3)nc2C(N)=O)cn1] |
| 96 | 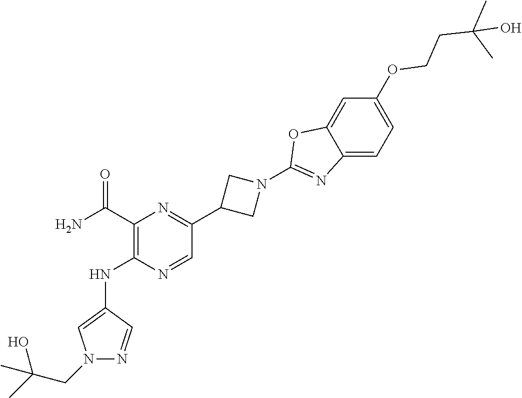 [SMILES: CC(C)(O)CCOc1ccc2nc(N3CC(c4cnc(Nc5cnn(CC(C)(C)O)c5)c(C(N)=O)n4)C3)oc2c1] |
| 96A | 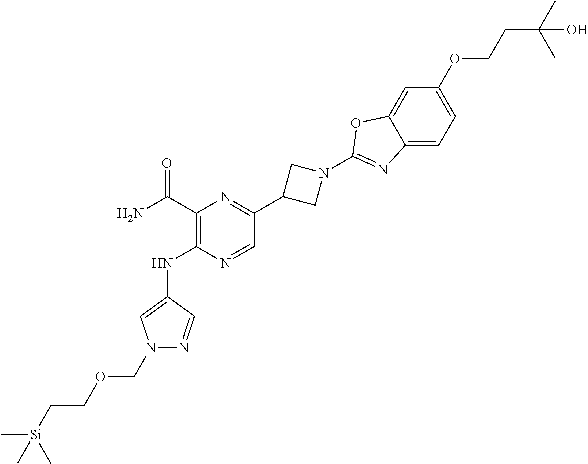 [SMILES: CC(C)(O)CCOc1ccc2nc(N3CC(c4cnc(Nc5cnn(COCC[Si](C)(C)C)c5)c(C(N)=O)n4)C3)oc2c1] |
| 96B | 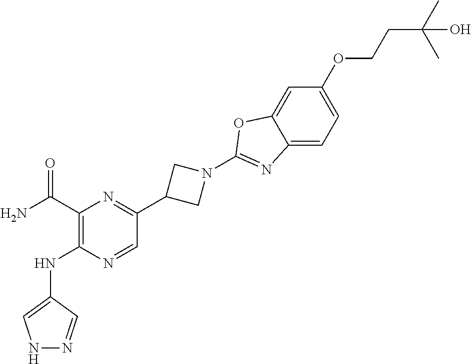 [SMILES: CC(C)(O)CCOc1ccc2nc(N3CC(c4cnc(Nc5cn[nH]c5)c(C(N)=O)n4)C3)oc2c1] |
| 97 | 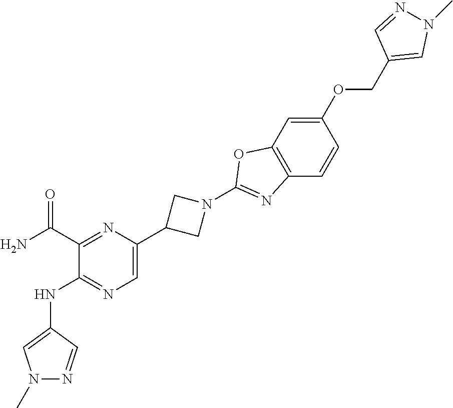 |
| 98 | 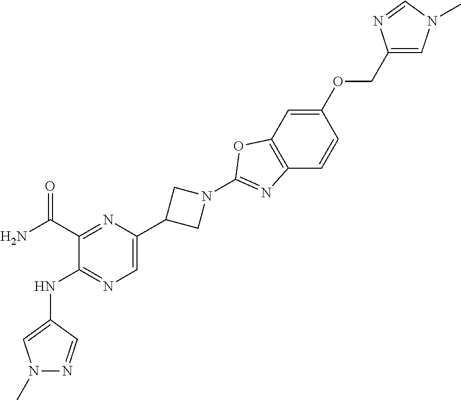 |
| 99 | 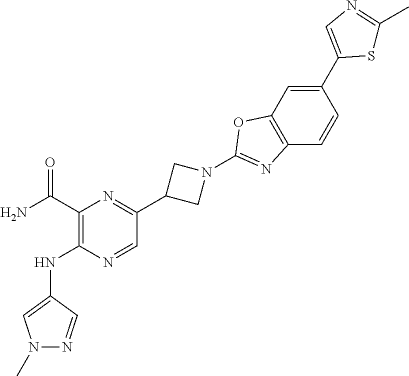 |
| 100 | 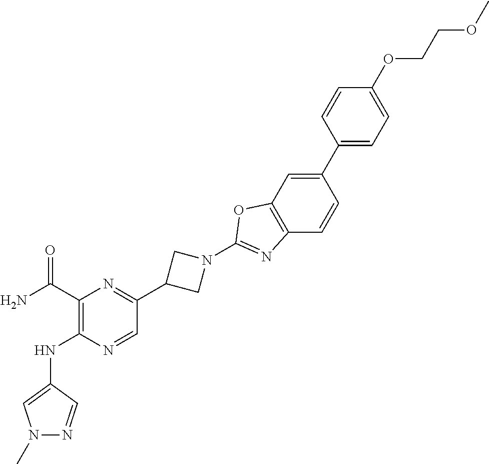 |
| 101 | 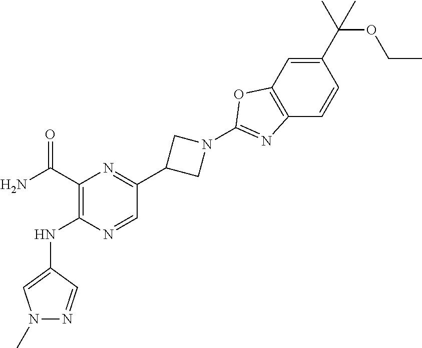 [SMILES: CCOC(C)(C)c1ccc2nc(N3CC(c4cnc(Nc5cnn(C)c5)c(C(N)=O)n4)C3)oc2c1] |
| 102 | 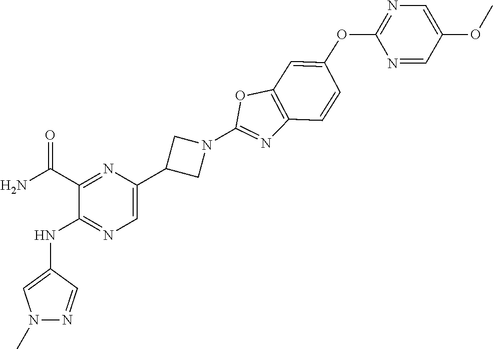 |
| 103 | 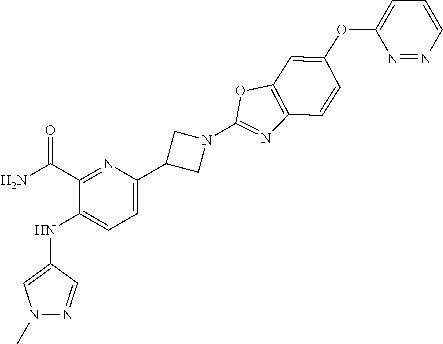 [SMILES: Cn1cc(Nc2ccc(C3CN(c4nc5ccc(Oc6cccnn6)cc5o4)C3)nc2C(N)=O)cn1] |
| 104 | 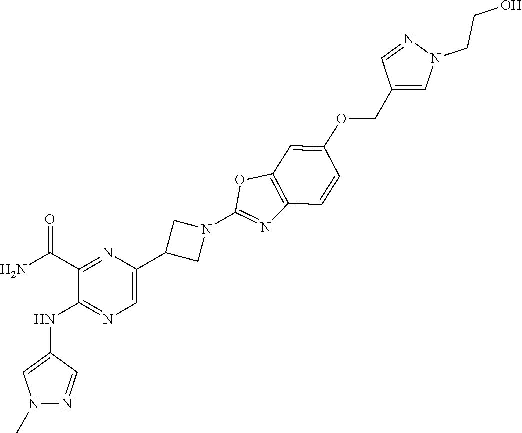 [SMILES: Cn1cc(Nc2ncc(C3CN(c4nc5ccc(OCc6cnn(CCO)c6)cc5o4)C3)nc2C(N)=O)cn1] |
| 104C | 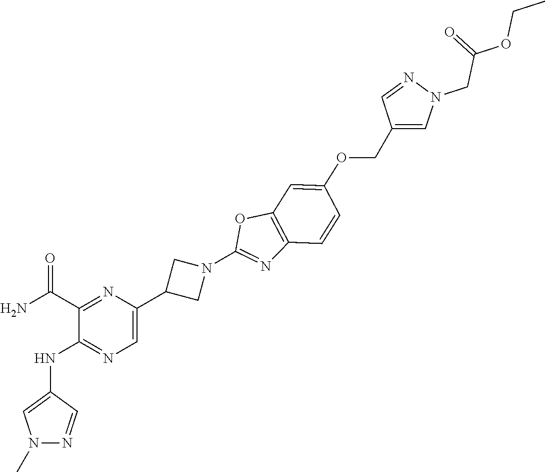 [SMILES: CCOC(=O)Cn1cc(COc2ccc3nc(N4CC(c5cnc(Nc6cnn(C)c6)c(C(N)=O)n5)C4)oc3c2)cn1] |
| 105 | 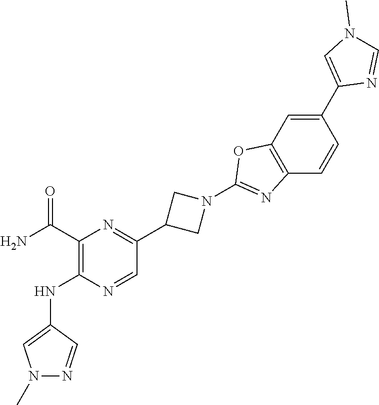 [SMILES: Cn1cnc(-c2ccc3nc(N4CC(c5cnc(Nc6cnn(C)c6)c(C(N)=O)n5)C4)oc3c2)c1] |
| 106 | 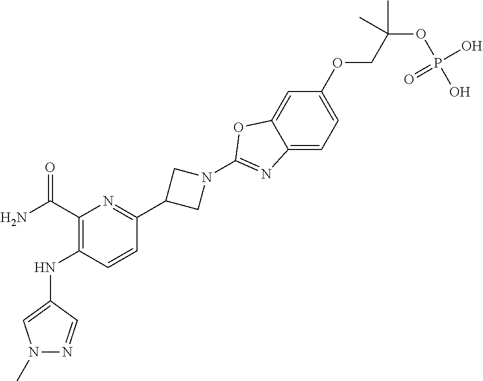 [SMILES: Cn1cc(Nc2ccc(C3CN(c4nc5ccc(OCC(C)(C)OP(=O)(O)O)cc5o4)C3)nc2C(N)=O)cn1] |
| 107 | 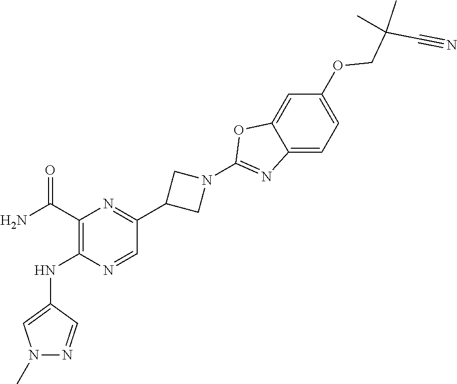 [SMILES: Cn1cc(Nc2ncc(C3CN(c4nc5ccc(OCC(C)(C)C#N)cc5o4)C3)nc2C(N)=O)cn1] |
| 108 | 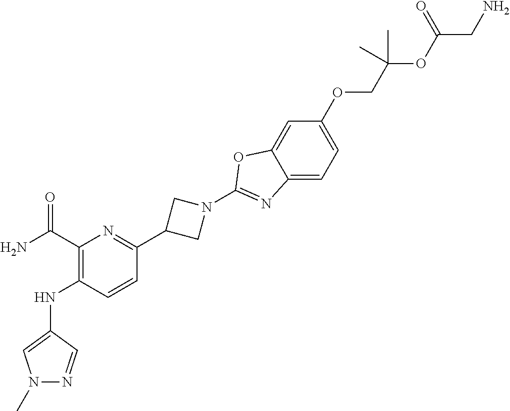 [SMILES: Cn1cc(Nc2ccc(C3CN(c4nc5ccc(OCC(C)(C)OC(=O)CN)cc5o4)C3)nc2C(N)=O)cn1] |
| 109 | 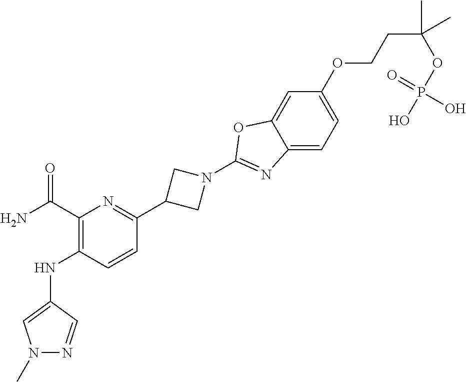 [SMILES: Cn1cc(Nc2ccc(C3CN(c4nc5ccc(OCCC(C)(C)OP(=O)(O)O)cc5o4)C3)nc2C(N)=O)cn1] |
| 110 | 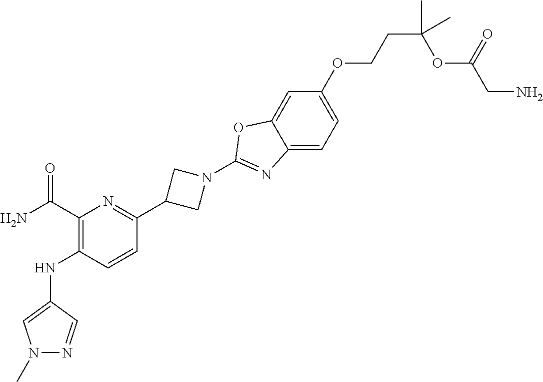 [SMILES: Cn1cc(Nc2ccc(C3CN(c4nc5ccc(OCCC(C)(C)OC(=O)CN)cc5o4)C3)nc2C(N)=O)cn1] |
| 111 | 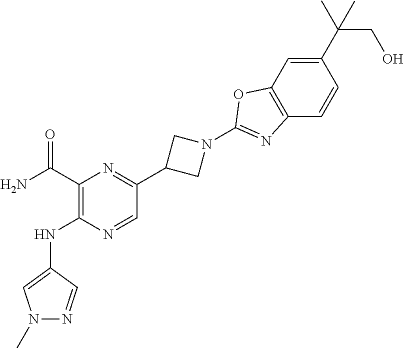 [SMILES: Cn1cc(Nc2ncc(C3CN(c4nc5ccc(C(C)(C)CO)cc5o4)C3)nc2C(N)=O)cn1] |
| 111F | 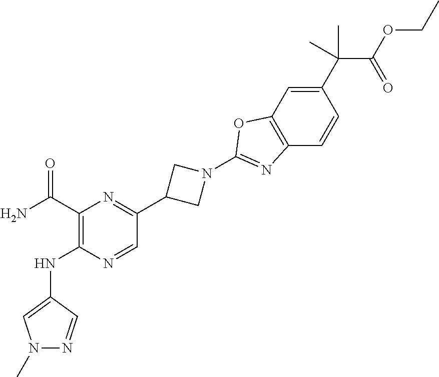 [SMILES: CCOC(=O)C(C)(C)c1ccc2nc(N3CC(c4cnc(Nc5cnn(C)c5)c(C(N)=O)n4)C3)oc2c1] |
| 112 | 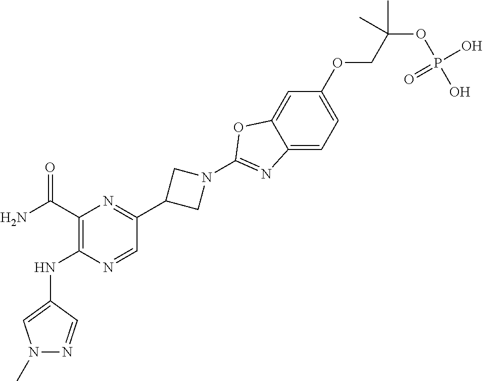 [SMILES: Cn1cc(Nc2ncc(C3CN(c4nc5ccc(OCC(C)(C)OP(=O)(O)O)cc5o4)C3)nc2C(N)=O)cn1] |
| 113 | 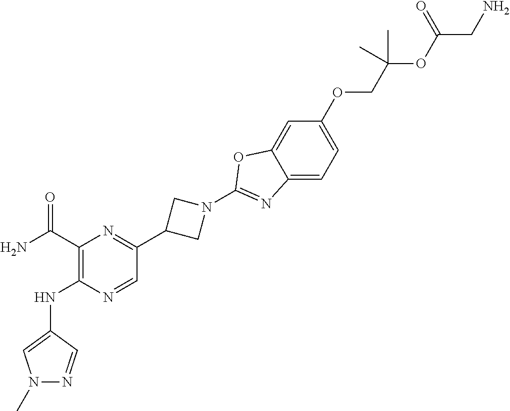 [SMILES: Cn1cc(Nc2ncc(C3CN(c4nc5ccc(OCC(C)(C)OC(=O)CN)cc5o4)C3)nc2C(N)=O)cn1] |

Single stereochemical isomers, enantiomers, diastereomers, racemates, tautomers, prodrugs, hydrates, solvates and pharmaceutically acceptable salts of the above exemplified compounds are also within the scope of the present invention.

Compounds of Formula (I) or Formula (II) may be used in the form of pharmaceutically acceptable salts.

Compounds of Formula (I) or Formula (II) may contain either a basic or an acidic functionality, or both, and may be converted to a pharmaceutically acceptable salt, when desired, by using a suitable acid or base. The salts may be prepared in situ during the final isolation and purification of the compounds of the invention.

Acid addition salts can be prepared by reacting the purified compound in its free-based form, if possible, with a suitable organic or inorganic acid and isolating the salt thus formed. Examples of pharmaceutically acceptable acid addition salts include, without limitations, salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid.

Base addition salts can be prepared by reacting the purified compound in its acid form with a suitable organic or inorganic base and isolating the salt thus formed. Such salts include, without limitations, alkali metal (e.g., sodium, lithium, and potassium), alkaline earth metal (e.g., magnesium and calcium), ammonium and N⁺(C₁₋₄alkyl)₄ salts.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, and valerate salts.

Novel compounds according to the present invention, or enantiomers, diastereomers, racemates, tautomers, prodrugs, hydrates, solvates or pharmaceutically acceptable salts therof exhibit the effect of effectively inhibiting Mer kinase.

Novel compounds according to the present invention, or enantiomers, diastereomers, racemates, tautomers, prodrugs, hydrates, solvates, or pharmaceutically acceptable salts thereof can be used for the prevention or treatment of cancer or immune-related disease.

### General Synthesis

The compounds of the present disclosure can be better understood in connection with the following synthetic schemes and methods which illustrate a means by which the compounds can be prepared. The compounds of this disclosure can be prepared by a variety of synthetic procedures. Representative procedures are shown in, but are not limited to, Schemes 1-5. In Schemes 1-5, the variables R¹, R², R³, L, and W are as described in the Summary.

As shown in Scheme 1, intermediate 1-7 may be prepared from compounds 1-1. Chloronitriles 1-2 may be treated with aminopyrazoles 1-1 where R⁶ is R¹ or a nitrogen protecting group, at elevated temperature to afford compound 1-3. Metal halides 1-4 may be prepared from the corresponding halide, for example the chloride, bromide, or iodide and treatment with a metal, including for example, zinc. Chlorides 1-3 may be coupled with metal halides of formula 1-4 using a suitable palladium catalyst, including, for example, tris(dibenzylideneacetone)dipalladium(0) with tri(furan-2-yl)phosphine ligand, to afford coupled product 1-5. Hydrolysis of nitrile 1-5 with a suitable base, including for example K₂CO₃, at elevated temperature affords amide 1-6. Deprotection of the boc group with acid affords 1-7.

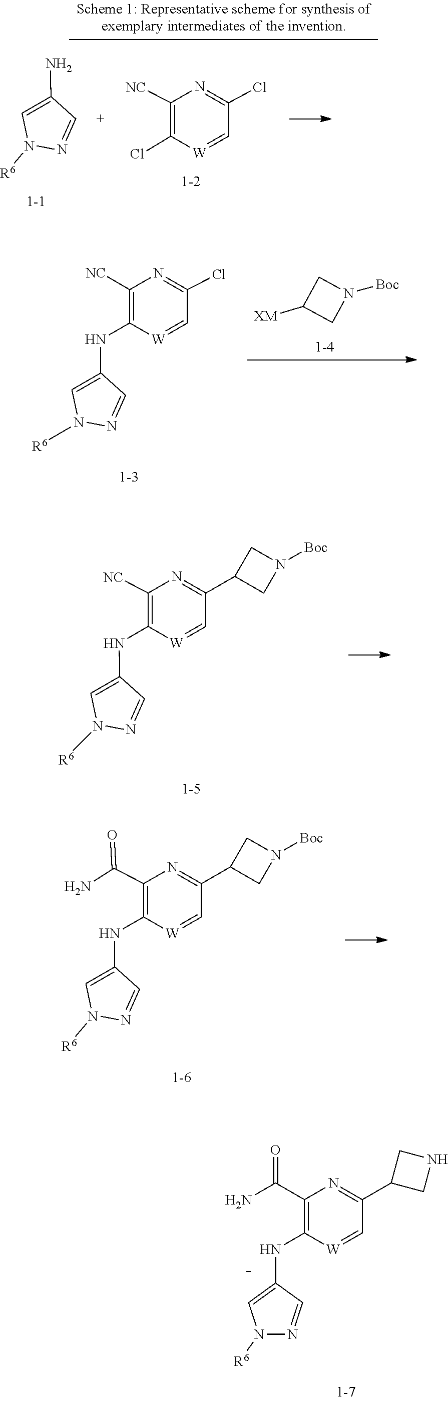

(In the scheme 1, M is Zn, and X is halo, e.g., chloro, bromo or iodo.)

As shown in Scheme 2, Intermediate 2-3 may be prepared from compound 2-1. Treatment of ester 2-1 where R is alkyl, benzyl, or other suitable carboxylate protecting group with metal halides of formula 1-4 using a suitable palladium catalyst, including, for example, tris(dibenzylideneacetone)dipalladium(0) with tri(furan-2-yl)phosphine ligand, affords coupled product 2-2. Deprotection of the boc group with acid affords 2-3.

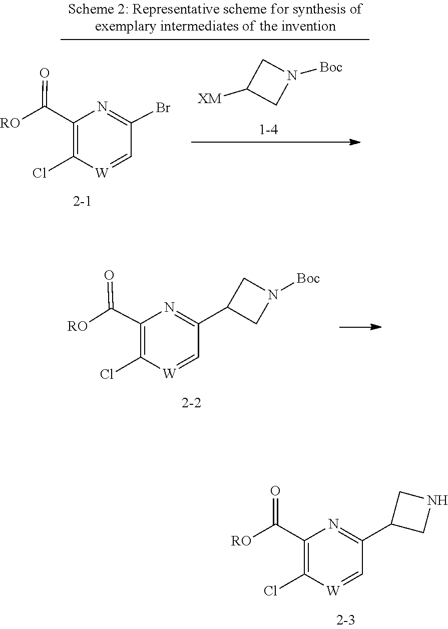

As shown in Scheme 3, compounds of formula 3-8 may be prepared from compound 3-1. Compounds of formula 3-2 where R⁶ is R¹ or a nitrogen protecting group including for example (trimethylsilyl)ethyl, may be prepared from 4-nitro-1H-pyrazole (1-1) via alkylation, acylation or other methods known to one skilled in the art. The nitro group of 3-2 may be reduced with palladium on carbon, for example, to afford amines 1-1 which may undergo an addition elimination reaction with 3-3 where X is Cl, Br, or I, using catalytic base including for example N,N-diisopropylethylamine at elevated temperature to afford 3-4. Halide 3-4 maybe be coupled with metal halides of formula 1-4 as discussed above to afford coupled product 3-5. Amidation of ester 3-5 with ammonia affords amide 1-6, and deprotection of the boc group with acid affords 1-7. Addition of oxazole 3-6 to 1-7 affords 3-8 where R⁶ is R¹ or 3-7 where R⁶ is a protecting group. Protected pyrazole 3-7 may be converted to 3-8 by deprotection of the protecting group to afford 3-8 where R¹ is hydrogen, or followed by alkylation using conditions known to one skilled in the art to afford 3-8 where R¹ is alkyl or heterocyclyl.

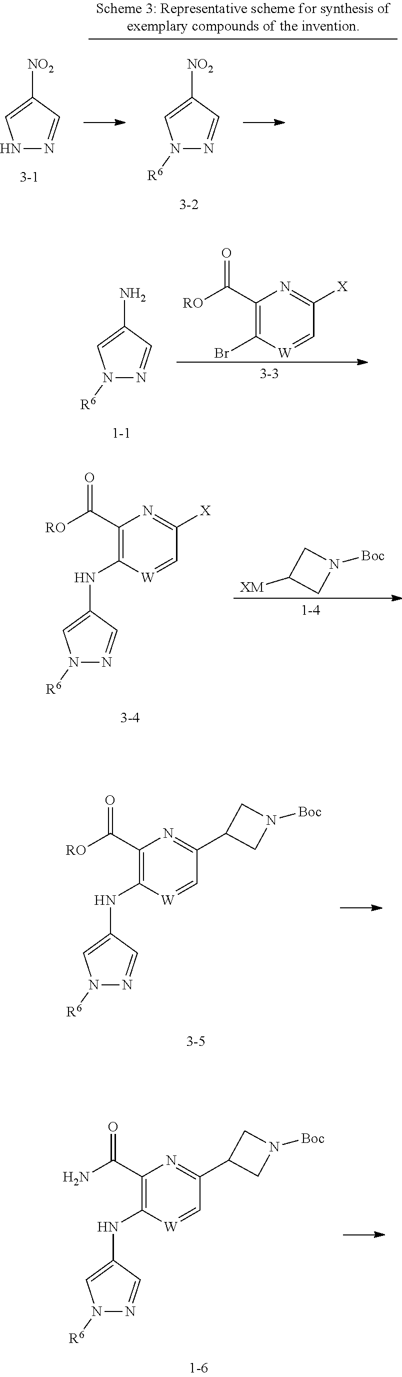

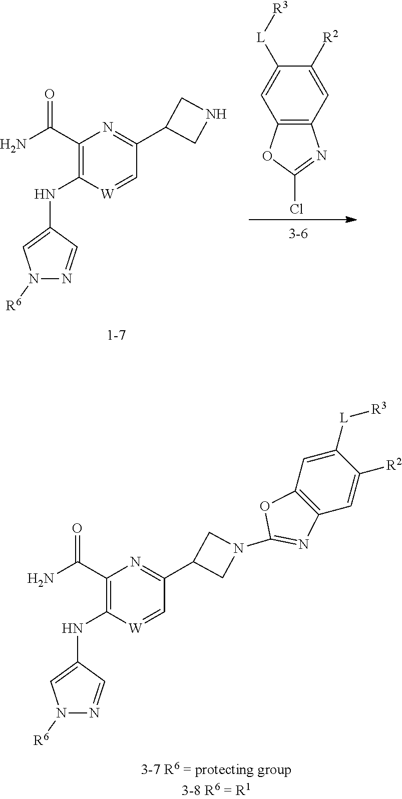

As shown in Scheme 4, compounds of formula 3-8 may be prepared from compounds of formula 2-3. Azetidine 2-3 may be treated with chlorooxazole 3-6 in the presence of a base, including for example, potassium carbonate, to afford 4-1. Addition of amine 1-1 where R⁶ is R¹ or a protecting group, with 4-1 using a base including for example potassium carbonate, affords 4-2. Amidation of ester 4-2 with ammonia affords 3-8 where R⁶ is R¹ or 3-7 where R⁶ is a protecting group. Protected pyrazole 3-7 may be converted to 3-8 as discussed above for Scheme 3.

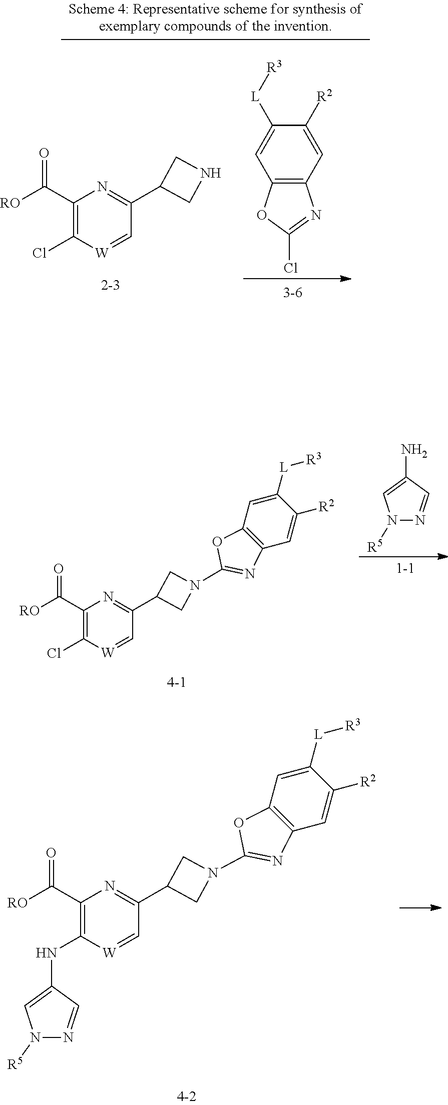

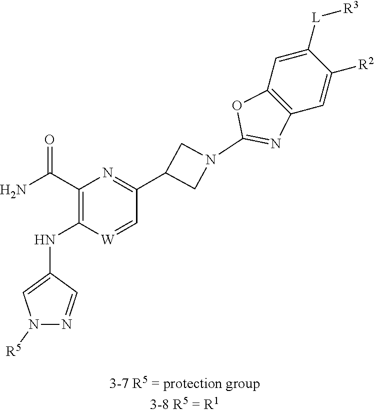

As shown in Scheme 5, compounds of formula 3-8 may be prepared from compounds of formula 5-1. Compound 5-1 which is compound 1-7 where R⁶ is R¹, may undergo addition to 3-6 to afford compounds of formula 3-8. Alternatively, compound 5-1 may undergo addition to 5-2, where R⁷ is halide, hydroxy, or protected hydroxy, to afford compounds of formula 5-3. When R⁷ of 5-3 is a halide, 5-3 may be coupled with 5-4 where R¹² is H (or the boronic ester equivalents where R¹² is alkyl), under Suzuki coupling conditions known to those skilled in the art to afford 3-8. The reaction typically requires the use of a base and a catalyst. Examples of bases include, but are not limited to, potassium carbonate, potassium tert-butoxide, sodium carbonate, cesium carbonate, and cesium fluoride. Examples of catalysts include, but are not limited to, [1,1′-bis(diphenylphosphino)ferrocene]dichloropalladium(II) tetrakis(triphenylphosphine)palladium(0), [1,1′-bis(diphenylphosphino)ferrocene]dichloropalladium(II) dichloromethane, bis(triphenylphosphine)palladium(II) dichloride, and tris(dibenzylideneacetone)dipalladium(0). The reaction may be conducted in a solvent such as, but not limited to, water, dioxane, 1,2-dimethoxyethane, N,N-dimethylformamide, toluene, ethanol, tetrahydrofuran and the like, or mixtures thereof. The reaction may be conducted at ambient or elevated temperatures, and optionally in a microwave oven. When R⁷ of 5-3 is hydroxy, 5-3 may be alkylated with 5-5 where LG¹ is a leaving group, e.g., halogen or sulfonate, and T is alkylene, under nucleophilic substitution reaction conditions to give compounds of formula 3-8. The nucleophilic substitution reaction is performed in the presence of a suitable base including, for example, potassium tert-butoxide in a suitable solvent including, for example, dimethyl sulfoxide.

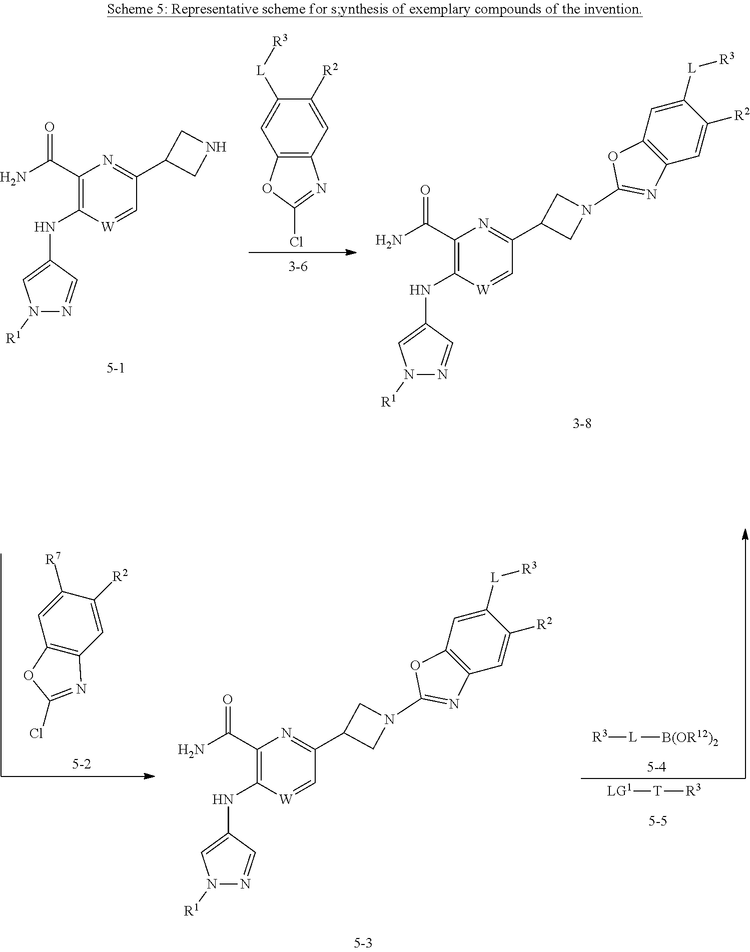

It can be appreciated that the synthetic schemes and specific examples as illustrated in the Examples section are illustrative and are not to be read as limiting the scope of the present disclosure as it is defined in the appended claims. All alternatives, modifications, and equivalents of the synthetic methods and specific examples are included within the scope of the claims.

### Pharmaceutical Compositions

This disclosure also provides for pharmaceutical compositions comprising a therapeutically effective amount of a compound disclosed herein, or a pharmaceutically acceptable salt thereof together with a pharmaceutically acceptable carrier.

The term “pharmaceutically acceptable carrier” as used herein, means a non-toxic, inert solid, semi-solid or liquid filler, diluent, encapsulating material or formulation auxiliary of any type.

The carriers that are used in the present invention may be those that are conventionally used in the art, and examples thereof include, but are not limited to, sugar, starch, microcrystalline cellulose, lactose (lactose hydrate), glucose, di-mannitol, alginate, alkaline earth metal salts, clay, polyethylene glycol, anhydrous dibasic calcium phosphate, or mixtures thereof.

The pharmaceutical composition may be a unit dosage form. In such form the preparation is subdivided into unit doses containing appropriate quantities of the active component.

The dose to be administered to a subject may be determined by the efficacy of the particular compound employed and the condition of the subject, as well as the body weight or surface area of the subject to be treated. The size of the dose also will be determined by the existence, nature, and extent of any adverse side-effects that accompany the administration of a particular compound in a particular subject. In determining the effective amount of the compound to be administered in the treatment or prophylaxis of the disorder being treated, the physician can evaluate factors such as the circulating plasma levels of the compound, compound toxicities, and/or the progression of the disease, etc.

In one embodiment, the composition comprises a compound of any one of formulae (I, or II), or a pharmaceutically acceptable salt thereof, and a pharmaceutically acceptable excipient.

Further, according to another embodiment of the present invention, the excipient includes binders, disintegrants, lubricants, pH-adjusting agents, antioxidants, and the like.

Examples of the binders that may be used in the present invention include, but are not limited to, starch, microcrystalline cellulose, highly dispersed silica, mannitol, di-mannitol, sucrose, lactose hydrate, polyethylene glycol, polyvinylpyrrolidone (povidone), polyvinylpyrrolidone copolymer (copovidone), hypromellose, hydroxypropyl cellulose, natural gum, synthetic gum, copovidone, gelatin, or mixtures thereof.

Examples of the disintegrants that may be used in the present invention include, but are not limited to, starches or modified starches such as sodium starch glycolate, maize starch, potato starch or pregelatinized starch; clays such as bentonite, montmorillonite, or veegum; celluloses such as microcrystalline cellulose, hydroxypropylcellulose or carboxymethylcellulose; algins such as sodium alginate or alginic acid; crosslinked celluloses such as croscarmellose sodium; gums such as guar gum or xanthan gum; crosslinked polymers such as crosslinked polyvinylpyrrolidone (crospovidone); effervescent formulations such as sodium bicarbonate or citric acid; or mixtures thereof.

Examples of the lubricants that may be used in the present invention include, but are not limited to, talc, stearic acid, magnesium stearate, calcium stearate, sodium lauryl sulfate, hydrogenated vegetable oil, sodium benzoate, sodium stearyl fumarate, glyceryl behenate, glyceryl monooleate, glyceryl monostearate, glyceryl palmitostearate, colloidal silicon dioxide, or mixtures thereof.

Examples of the pH-adjusting agents that may be used in the present invention include, but are not limited to, acidifying agents such as acetic acid, adipic acid, ascorbic acid, sodium ascorbate, sodium etherate, malic acid, succinic acid, tartaric acid, fumaric acid or citric acid, and basifying agents such as precipitated calcium carbonate, ammonia water, meglumine, sodium carbonate, magnesium oxide, magnesium carbonate, sodium citrate, or tribasic calcium phosphate.

Examples of the antioxidants that may be used in the present invention include, but are not limited to, dibutyl hydroxytoluene, butylated hydroxyanisole, tocopherol acetate, tocopherol, propyl gallate, sodium hydrogen sulfite, sodium pyrosulfite, and the like.

In one embodiment, the composition comprises a compound of formula (I), or a pharmaceutically acceptable salt thereof, and a pharmaceutically acceptable excipient.

In one embodiment, the composition comprises a compound of formula (II), or a pharmaceutically acceptable salt thereof, and a pharmaceutically acceptable excipient.

In one embodiment, the present invention provides the composition comprises, as active ingredients, the compounds of Formula (I) or (II), the stereoisomer thereof, the enantiomer thereof, or the pharmaceutically acceptable salt thereof and are used for prevention or treatment of a disease which is influenced by inhibition of Mer kinase.

The present invention provides the disease which is influenced by inhibition of Mer kinase is cancer or immune-related diseases.

The cancer is selected from the group consisting of: glioma, gliosarcoma, anaplastic astrocytoma, medulloblastoma, lung cancer, small cell lung carcinoma, cervical carcinoma, colon cancer, rectal cancer, chordoma, throat cancer, Kaposi's sarcoma, lymphangiosarcoma, lymphangioendotheliosarcoma, colorectal cancer, endometrium cancer, ovarian cancer, breast cancer, pancreatic cancer, prostate cancer, renal cell carcinoma, hepatic carcinoma, bile duct carcinoma, choriocarcinoma, seminoma, testicular tumor, Wilms' tumor, Ewing's tumor, bladder carcinoma, angiosarcoma, endotheliosarcoma, adenocarcinoma, sweat gland carcinoma, sebaceous gland sarcoma, papillary sarcoma, papillary adenosarcoma, cystadenosarcoma, bronchogenic carcinoma, medullary carcinoma, mastocytoma, mesotheliorma, synovioma, melanoma, leiomyosarcoma, rhabdomyosarcoma, neuroblastoma, retinoblastoma, oligodentroglioma, acoustic neuroma, hemangioblastoma, meningioma, pinealoma, ependymoma, craniopharyngioma, epithelial carcinoma, embryonal carcinoma, squamous cell carcinoma, basal cell carcinoma, fibrosarcoma, myxoma, myxosarcoma, liposarcorna, chondrosarcoma, osteogenic sarcoma, leukemia and metastatic lesions secondary to these primary tumors.

The immune-related disease is selected from the group consisting of infection and sepsis.

The term “treatment” is used to refer to both prevention of diseases and treatment of pre-existing conditions.

The therapeutic amount varies according to the specific disease and can be determined by the person skilled in the art without undue effort.

In addition, the subject in the prevention or treatment method of the present invention includes mammals, particularly humans.

The dose varies depending on the specific compound used, the specific disease, the patient status, etc. A therapeutic dose is typically sufficient considerably to reduce the undesired cell population in the target tissue while the viability of the patient is maintained. The treatment is generally continued until a considerable reduction has occurred, for example an at least about 50% reduction in the cell burden, and may be continued until essentially no more undesired cells are detected in the body.

### Method for Prevention or Treatment of Immune-Related Diseases or Cancer

The present invention provides a method of treating or preventing immune-related diseases or cancer, the method comprising administering to a mammals including humans in need thereof compositions comprising, as active ingredients, the compounds of Formula (I) or (II), isomers thereof enantiomers, diastereomers, racemates, tautomers, prodrugs, hydrates, solvates or pharmaceutically acceptable salts thereof.

The composition that is used in the inventive method for preventing or treating immune-related diseases or cancer includes the pharmaceutical composition described in the specification

The present invention provides use of compositions comprising, as active ingredients, the compounds of Formula (I) or (II), the stereoisomer thereof, the enantiomer thereof, or the pharmaceutically acceptable salt thereof for preparation of medicaments for preventing or treating cancer or immune-related diseases.

### Methods for Preparing of Novel Mer Kinase Inhibitors

The compounds of this invention can be prepared in accordance with one or more of schemes discussed below.

These methods can be used either directly or with obvious variations to trained chemists to prepare key intermediates and certain compounds of this invention.

Further benefits of Applicants' disclosure will be apparent to one skilled in the art from reading this patent application.

The following Examples may be used for illustrative purposes and should not be deemed to narrow the scope of the present disclosure.

### Advantageous Effects

The compounds, or an enantiomer, a diastereomer, a racemate, a tautomer, a prodrug, a hydrate, a solvate or a pharmaceutically acceptable salt thereof of the present disclosure exhibit the effects of modulating Mer kinase and Axl kinase.

### MODE FOR INVENTION

### Examples

### General

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

Common abbreviations well known to those skilled in the art which are used throughout include: DMSO for dimethyl sulfoxide; HATU for 1-[bis(dimethylamino)methylene]-1H-1,2,3-triazolo[4,5-b]pyridinium 3-oxid hexafluorophosphate or N,N,N-tetramethyl-3-oxo-H-3a,5-[1,2,3]triazolo[4,5-b]pyridine-1-carboximidamidium hexafluoridophosphate(1-) or N,N,N,N-tetramethyl-1-oxo-3H-1λ⁵-[1,2,3]triazolo[4,5-b]pyridine-3-carboximidamidium hexafluoridophosphate(1-); and TFA for trifluoroacetic acid.

Other common abbreviations well known to those skilled in the art which are used throughout include: ATP for adenosine triphosphate; BSA for bovine serum albumin; EDTA for ethylenediaminetetraacetic acid; DMEM for Dulbecco's Modified Eagle's Medium; DTT for dithiothreitol; FBS for fetal bovine serum; GI₅₀ for half-maximal growth inhibitory concentration; FRET for fluorescence energy transfer; GT for glutamate-tyrosine; HEPES for (4-(2-hydroxyethyl)-1-piperazineethanesulfonic acid); HTRF for homogeneous time-resolved fluorescence; IC₅₀ for half maximal inhibitory concentration; RPMI for Roswell Park Memorial Institute; SAR for structure-activity relationship; and TK for tyrosine kinase.

Yet other abbreviations well known to those skilled in the art which are used throughout include: APCI for atmospheric-pressure chemical ionization; atm for atmospheres of gas pressure; ESI for electrospray ionization; g for gram; h for hour or hours; HPLC for high performance liquid chromatography; LC/MS or LCMS for liquid chromatography-mass spectrometry; μL for microliter; μm for micrometer; mg for milligram; min for minute; mL for milliliter; mmol for millimoles; MS for mass spectrum; NMR for nuclear magnetic resonance; psi for pounds per square inch; rt for room temperature; and SFC for supercritical fluid chromatography.

All reagents were of commercial grade and were used as received without further purification, unless otherwise stated. Commercially available anhydrous solvents were used for reactions conducted under inert atmosphere. Reagent grade solvents were used in all other cases, unless otherwise specified. Column chromatography was performed on silica gel 60 (35-70 μm). Thin layer chromatography was carried out using pre-coated silica gel F-254 plates (thickness 0.25 mm). ¹H NMR spectra were recorded on an Agilent 400 MHz NMR spectrometer, a Varian or Bruker 500 MHz spectrometer or a 501 MHz spectrometer. Chemical shifts (6) for ¹H NMR spectra were reported in parts per million (ppm) relative to tetramethylsilane (δ 0.00) or the appropriate residual solvent peak, i.e. CHCl₃ (δ 7.27), as internal reference. Multiplicities were given as singlet (s), doublet (d), doublet of doublets of doublets (ddd), doublet of doublets of doublets of doublets (dddd), doublet of doublets of quartets (ddq), doublet of doublets of triplets (ddt), doublet of quartets (dq), doublet of triplets of doublets (dtd), heptet (hept), triplet (t), triplet of doublets of doublets (tdd), triplet of quartets (tq), quartet (q), quartet of doublets (qd), quartet of triplets (qt), quintuplet (quin), multiplet (m) and broad (br). Electrospray Ionization MS spectra were obtained on a Thermo Vanquish UPLC coupled to a Thermo MSQ Plus mass spectrometer using a Waters Xselect HSS T3 C18 2.5 μm column (2.1 mm×50 mm). A gradient of water containing 0.1% formic acid and 10 mM ammonium acetate (A) and acetonitrile with 0.1% formic acid (B) was used (0-2.3 min linear gradient 100-20% A). Microwave heating was performed with a Biotage® Initiator.

Flash chromatography purifications were performed on a Biotage® Isolera One Flash, Teledyne ISCO CombiFlash® RF+ or an Analogix IntelliFlash 280 system. Reverse phase HPLC purifications were performed on a Waters Prep LC 2487, Gilson PLC 2020, Gilson GX-281 or a Shimadzu LC-8A system using mobile phases of either A: 0.075% trifluoroacetic acid/water and B: CH₃CN, A: 0.04% HCl/H₂O and B: CH₃CN, or A: 10 mM aqueous NH₄HCO₃ and B: CH₃CN.

Super critical fluid chromatography purifications were performed on a That SFC 80 system. The mobile phase comprised of supercritical CO₂ supplied by a Dewar of bone-dry non-certified CO₂ pressurized to 350 psi with a methanol flow rate of 80 mL/min. The column was at ambient temperature and the backpressure regulator was set to maintain 100 bar. The sample was dissolved in methanol at a concentration of 50 mg/mL and was loaded into the modifier stream in 2 mL (100 mg) injections. The mobile phase was held isocratically at 20% co-solvent/CO₂.

### Intermediate 1

### 6-(azetidin-3-yl)-3-[(1-methyl-1H-pyrazol-4-yl)amino]pyrazine-2-carboxamide

### Intermediate 1A

### 6-chloro-3-[(1-methyl-1H-pyrazol-4-yl)amino]pyrazine-2-carbonitrile

To a solution of 3,6-dichloropyrazine-2-carbonitrile (11.25 g, 64.7 mmol) in acetonitrile (100 mL) was added 1-methyl-1H-pyrazol-4-amine (8.16 g, 84 mmol) at 20° C., and the mixture was heated at 85° C. for 5 hours. Three additional reactions were set up as described above. After cooling, all four reaction mixtures were combined, diluted with ethyl acetate, and washed with water. The organic layer was dried over Na₂SO₄, filtered and concentrated. The residue was purified by flash chromatography on silica gel eluting with 50/1 to 3/1 petroleum ether/ethyl acetate to afford the title compound (40 g). ¹H NMR (400 MHz, dimethyl sulfoxide-d₆) δ ppm 3.82 (s, 3H), 7.61 (s, 1H), 7.94 (d, J=2.19 Hz, 1H), 8.52-8.58 (m, 1H), 9.85 (s, 1H).

### Intermediate 1B

### [1-(tert-butoxycarbonyl)azetidin-3-yl](iodido)zinc

To a vial of zinc (3.46 g, 53 mmol) in tetrahydrofuran (40 mL) under nitrogen was added 1,2-dibromoethane (0.73 g, 3.89 mmol) and the suspension was stirred at 65° C. for 5 minutes. After cooling, chlorotrimethylsilane (0.422 g, 3.89 mmol) was added and the mixture was stirred at 20° C. for 1 hour. A solution of tert-butyl 3-iodoazetidine-1-carboxylate (10 g, 35.3 mmol) in tetrahydrofuran (40 mL) was added and the mixture was stirred for 1.5 hours at 20° C. This solution was used directly without purification.

### Intermediate 1C

### tert-butyl 3-{6-cyano-5-[(1-methyl-1H-pyrazol-4-yl)amino]pyrazin-2-yl}azetidine-1-carboxylate

A flask containing Intermediate 1A (5 g, 21.3 mmol) was flushed with nitrogen for 1 hour and tetrahydrofuran (100 mL) was added. A separate flask with tri(furan-2-yl)phosphine (0.99 g, 4.26 mmol) and tris(dibenzylideneacetone)dipalladium(0) (1.95 g, 2.13 mmol) was flushed with nitrogen for 1 hour and tetrahydrofuran (25 mL) was added. This palladium catalyst solution was transferred into an Intermediate 1B solution (145 mL, 63.9 mmol). This combined catalyst-zinc reagent solution was cannulated into the flask containing Intermediate 1A at 20° C. and the mixture was heated at 65° C. for 4 hours. One additional reaction was set up as described above. After cooling, both reaction mixtures were combined, filtered, and the filtrate diluted with ethyl acetate and washed with water. The organic layer was dried over Na₂SO₄, filtered and concentrated. The residue was purified by flash chromatography on silica gel eluting with 50/1 to 5/1 ethyl acetate/methanol to afford the title compound (5 g). ¹H NMR (400 MHz, dimethyl sulfoxide-d₆) δ ppm 1.40 (s, 9H), 3.81 (s, 3H), 3.91 (br s, 3H), 4.15 (br s, 2H), 7.62 (s, 1H), 7.96 (s, 1H), 8.37 (s, 1H), 9.60 (s, 1H).

### Intermediate 1D

### tert-butyl 3-{6-carbamoyl-5-[(1-methyl-1H-pyrazol-4-yl)amino]pyrazin-2-yl}azetidine-1-carboxylate

To a solution of Intermediate 1C (1.667 g, 4.69 mmol) in dioxane (20 mL) and water (5 mL) was added K₂CO₃ (1.945 g, 14.07 mmol) and the mixture was heated at 100° C. for 12 hours. Two additional reactions were set up as described above. After cooling, all three reaction mixtures were combined, diluted with water, and extracted with ethyl acetate. The combined organic layers were dried over Na₂SO₄, filtered, and concentrated to afford the title compound (5 g). ¹H NMR (400 MHz, dimethyl sulfoxide-d₆) δ ppm 1.40 (s, 9H), 3.81 (s, 3H), 3.89-3.97 (m, 1H), 4.04-4.24 (m, 4H), 7.59-7.63 (m, 1H), 7.94-8.01 (m, 1H), 8.04 (s, 1H), 8.27 (br s, 1H), 8.31 (s, 1H), 10.67 (s, 1H).

### Intermediate 1E

### 6-(azetidin-3-yl)-3-[(1-methyl-1H-pyrazol-4-yl)amino]pyrazine-2-carboxamide

To a solution of Intermediate 1D (1.25 g, 3.35 mmol) in dichloromethane (20 mL) was added trifluoroacetic acid (4 mL, 3.35 mmol) at 20° C. and the mixture was stirred at 20° C. for 12 hours. Three additional reactions were set up as described above. All four reaction mixtures were combined and concentrated to afford the title compound (4 g) as the trifluoroacetate salt, which was used without further purification. ¹H NMR (400 MHz, dimethyl sulfoxide-d₆) δ ppm 3.81 (s, 3H), 4.05-4.22 (m, 3H), 4.23-4.39 (m, 2H), 7.61 (s, 1H), 8.04 (s, 1H), 8.09 (br s, 1H), 8.26 (s, 1H), 8.71 (br s, 1H), 8.90 (br s, 1H), 10.80 (s, 1H).

### Intermediate 2

### methyl 6-(azetidin-3-yl)-3-chloropyrazine-2-carboxylate

### Intermediate 2A

### methyl 6-[1-(tert-butoxycarbonyl)azetidin-3-yl]-3-chloropyrazine-2-carboxylate

A flask containing methyl 6-bromo-3-chloropyrazine-2-carboxylate (2.5 g, 9.94 mmol) was flushed with nitrogen for 1 hour and tetrahydrofuran (50 mL) was added. Separately, a flask containing tri(furan-2-yl)phosphine (0.462 g, 1.988 mmol) and tris(dibenzylideneacetone)dipalladium(0) (0.910 g, 0.994 mmol) was flushed with nitrogen for 1 hour and tetrahydrofuran (12.5 mL) was added. This palladium catalyst solution was transferred into a previously prepared solution of Intermediate 1B (45.2 mL, 19.88 mmol). This combined catalyst-zinc reagent solution was cannulated into the methyl 6-bromo-3-chloropyrazine-2-carboxylate solution at 20° C. and the mixture was heated at 65° C. for 1.5 hours. Three additional reactions were set up as described above. After cooling, all four reaction mixtures were combined, filtered, and the filtrate was diluted with ethyl acetate and washed with water. The organic layer was dried over Na₂SO₄, filtered and concentrated. The residue was purified by flash chromatography on silica gel eluting with 80/1 to 20/1 petroleum ether/ethyl acetate to afford the title compound (7.2 g). ¹H NMR (400 MHz, CDCl₃) δ ppm 1.47 (d, J=0.61 Hz, 9H), 3.95-4.02 (m, 1H), 4.04 (d, J=0.73 Hz, 3H), 4.17 (dd, J=8.38, 6.05 Hz, 2H), 4.30-4.38 (m, 2H), 8.44 (s, 1H).

### Intermediate 2B

### methyl 6-(azetidin-3-yl)-3-chloropyrazine-2-carboxylate

To a solution of Intermediate 2A (2.4 g, 7.32 mmol) in dichloromethane (40 mL) was added trifluoroacetic acid (8 mL, 7.32 mmol) dropwise at 20° C., and the mixture was stirred at 20° C. for 12 hours. Two additional reactions were set up as described above. All three reaction mixtures were combined and concentrated to afford the title compound (5.8 g), which was used without further purification as the trifluoroacetate salt. ¹H NMR (400 MHz, CD₃OD) δ ppm 4.03 (s, 3H), 4.31-4.38 (m, 2H), 4.40-4.46 (m, 3H), 8.52 (s, 1H).

### Intermediate 3

### methyl 6-[1-(1,3-benzoxazol-2-yl)azetidin-3-yl]-3-chloropyrazine-2-carboxylate

To a solution of Intermediate 2B (13.5 g, 39.5 mmol) in N,N-dimethylformamide (135 mL) was added K₂CO₃ (21.84 g, 158 mmol) and 2-chlorobenzoxazole (5.52 mL, 47.4 mmol), and the mixture was stirred at 20° C. for 12 hours. One additional reaction was set up as described above. The two reaction mixtures were combined, poured into water, and extracted with ethyl acetate. The organic layers were washed with water, dried over Na₂SO₄, filtered and concentrated. The residue was purified by silica gel chromatography eluting with 50/1 to 0/1 petroleum ether/ethyl acetate to afford the title compound (23 g). ¹H NMR (400 MHz, dimethyl sulfoxide-d₆) δ ppm 3.92 (s, 3H), 4.34-4.45 (m, 3H), 4.55-4.63 (m, 2H), 7.02-7.09 (m, 1H), 7.17 (td, J=7.66, 0.99 Hz, 1H), 7.33 (d, J=7.50 Hz, 1H), 7.43 (d, J=7.94 Hz, 1H), 8.78 (s, 1H).

### Intermediate 4

### 6-(azetidin-3-yl)-3-[(1-methyl-1H-pyrazol-4-yl)amino]pyridine-2-carboxamide

### Intermediate 4A

### methyl 3-bromo-6-chloropyridine-2-carboxylate

To a solution of 3-bromo-6-chloropicolinic acid (10 g, 42.3 mmol) in methanol (100 mL) was added concentrated sulfuric acid (10 mL, 188 mmol) and the mixture was stirred at 70° C. for 16 hours. The reaction was repeated another nine times and the combined reaction mixtures were concentrated and purified by silica gel chromatography, eluting with 20/1 to 5/1 petroleum ether/ethyl acetate to afford the title compound (90 g). ¹H NMR (400 MHz, CDCl₃) δ ppm 4.00 (s, 3H), 7.33 (d, J=8.44 Hz, 1H), 7.94 (d, J=8.44 Hz, 1H).

### Intermediate 4B

### methyl 6-chloro-3-[(1-methyl-1H-pyrazol-4-yl)amino]pyridine-2-carboxylate

To a solution of Intermediate 4A (15 g, 59.9 mmol) in dioxane (150 mL) was added 1-methyl-1H-pyrazol-4-amine hydrochloride (8.80 g, 65.9 mmol), (9,9-dimethyl-9H-xanthene-4,5-diyl)bis(diphenylphosphine) (3.47 g, 5.99 mmol), tris(dibenzylideneacetone)dipalladium(0) (2.74 g, 2.99 mmol) and Cs₂CO₃ (58.5 g, 180 mmol) under nitrogen. The mixture was stirred at 80° C. for 16 hours. The reaction was repeated another five times and the combined mixtures were diluted with tetrahydrofuran, filtered, and the filtrate concentrated. The residue was purified by silica gel chromatography, eluting with 50/1 to 0/1 petroleum ether/tetrahydrofuran to afford the title compound (50 g). ¹H NMR (400 MHz, dimethyl sulfoxide-d₆) δ ppm 3.83 (s, 3H), 3.87 (s, 3H), 7.32-7.39 (m, 1H), 7.41-7.48 (m, 2H), 7.83 (s, 1H), 8.81 (s, 1H).

### Intermediate 4C

### methyl 6-[1-(tert-butoxycarbonyl)azetidin-3-yl]-3-[(1-methyl-1H-pyrazol-4-yl)amino]pyridine-2-carboxylate

To a solution of Intermediate 4B (25 g, 94 mmol) in dimethyl acetamide (250 mL) was added 1,1′-bis(diphenylphosphino)ferrocenedichloro palladium(II) dichloromethane complex (3.83 g, 4.69 mmol), Intermediate 1B solution (562 mL, 281 mmol) and copper (I) iodide (1.785 g, 9.37 mmol) under nitrogen and the mixture was stirred at 80° C. for 12 hours. The reaction was repeated one time and the combined mixtures were quenched with saturated ammonium chloride and extracted with ethyl acetate. The organic layers were dried over Na₂SO₄, filtered, concentrated and purified by silica gel chromatography, eluting with 50/1 to 0/1 petroleum ether/ethyl acetate to afford the title compound (38 g). ¹H NMR (400 MHz, dimethyl sulfoxide-d₆) δ ppm 1.39 (s, 9H), 3.78-3.85 (m, 4H), 3.87 (s, 3H), 3.91-4.00 (m, 2H), 4.14 (br s, 2H), 7.26-7.33 (m, 1H), 7.34-7.41 (m, 1H), 7.44 (s, 1H), 7.80 (s, 1H), 8.71 (s, 1H).

### Intermediate 4D

### tert-butyl 3-{6-carbamoyl-5-[(1-methyl-1H-pyrazol-4-yl)amino]pyridin-2-yl}azetidine-1-carboxylate

To a solution of Intermediate 4C (9.5 g, 24.52 mmol) in methanol (80 mL) was added anhydrous ammonia (80 mL, 800 mmol), and the mixture was stirred at 80° C. for 16 hours. After cooling to ambient temperature, the reaction was repeated three times and the combined reaction mixtures were concentrated, filtered, and the residue dried in vacuo to afford the title compound (35 g). ¹H NMR (400 MHz, dimethyl sulfoxide-d₆) δ ppm 1.39 (s, 9H), 3.82 (s, 3H), 4.00 (dd, J=8.13, 6.05 Hz, 2H), 4.15 (br s, 2H), 7.28 (s, 2H), 7.42 (s, 1H), 7.67 (br d, J=2.20 Hz, 1H), 7.78 (s, 1H), 8.05 (br d, J=2.32 Hz, 1H), 9.76 (s, 1H).

### Intermediate 4E

### 6-(azetidin-3-yl)-3-[(1-methyl-1H-pyrazol-4-yl)amino]pyridine-2-carboxamide

To a solution of Intermediate 4D (7 g, 18.80 mmol) in dichloromethane (100 mL) was added trifluoroacetic acid (14 mL, 182 mmol), and the mixture was stirred at 20° C. for 16 hours. The reaction was repeated five times and the combined reaction mixtures were concentrated, stirred with ethyl acetate (30 mL) and petroleum ether (60 mL) for 1 hour, filtered and dried to afford the title compound as a trifluoroacetate salt (29.2 g). ¹H NMR (400 MHz, CD₃OD) δ ppm 3.89 (s, 3H), 4.06-4.19 (m, 1H), 4.28-4.43 (m, 4H), 7.20 (d, J=8.60 Hz, 1H), 7.31 (d, J=8.60 Hz, 1H), 7.44 (s, 1H), 7.65 (s, 1H).

### Example 0

### 6-[1-(6-hydroxy-1,3-benzoxazol-2-yl)azetidin-3-yl]-3-[(1-{[2-(trimethylsilyl)ethoxy]methyl}-1H-pyrazol-4-yl)amino]pyrazine-2-carboxamide

### Example 0A

### 4-nitro-1-{[2-(trimethylsilyl)ethoxy]methyl}-1H-pyrazole

To a solution of 4-nitro-1H-pyrazole (15 g, 133 mmol) in tetrahydrofuran (150 mL) was added sodium hydride (10.61 g, 265 mmol) in portions at 0° C. under nitrogen. The mixture was stirred at 20° C. for 30 minutes, 2-(trimethylsilyl)ethoxymethyl chloride (28.2 mL, 159 mmol) was added dropwise, and the mixture was stirred at 20° C. for 3 hours. Nineteen additional reactions were set up as described above. All twenty reaction mixtures were combined, quenched with brine and extracted with ethyl acetate. The organic layers were dried over Na₂SO₄, filtered, concentrated, and purified by silica gel chromatography eluting with 100/0 to 90/10 petroleum ether/ethyl acetate to afford the title compound (450 g). ¹H NMR (400 MHz, CD₃OD) δ ppm −0.10-0.02 (m, 9H), 0.87-0.95 (m, 2H), 3.62-3.69 (m, 2H), 5.49 (s, 2H), 8.17 (s, 1H), 8.74 (s, 1H).

### Example 0B

### 1-{[2-(trimethylsilyl)ethoxy]methyl}-1H-pyrazol-4-amine

To a solution of Example 0A (28 g, 115 mmol) in ethyl acetate (600 mL) was added wet palladium on carbon (3.67 g, 3.45 mmol) and the mixture was stirred under hydrogen at 20° C. for 5 hours. Nine additional reactions were set up as described above. All ten reaction mixtures were combined, filtered through diatomaceous earth, and concentrated to afford the title compound (230 g). ¹H NMR (400 MHz, CDCl₃) δ ppm −0.02 (s, 9H), 0.87-0.93 (m, 2H), 2.96 (br s, 2H), 3.53 (br d, J=8.38 Hz, 2H), 5.30 (s, 2H), 7.17 (d, J=0.66 Hz, 1H), 7.21 (s, 1H).

### Example 0C

### methyl 6-bromo-3-[(1-{[2-(trimethylsilyl)ethoxy]methyl}-1H-pyrazol-4-yl)amino]pyrazine-2-carboxylate

A mixture of methyl 3,6-dibromopyrazine-2-carboxylate (37 g, 125 mmol), Example 0B (32 g, 150 mmol) and N,N-diisopropylethylamine (54.6 mL, 313 mmol) in methanol (400 mL) was stirred at 80° C. for 2 hours. Four additional reactions were set up as described above. After cooling to 20° C., all five reaction mixtures were combined, diluted with water, and stirred for 2 hours. The resulting precipitate was filtered and dried in vacuo to afford the title compound (222.7 g). ¹H NMR (400 MHz, dimethyl sulfoxide-d₆) δ ppm −0.06-−0.04 (m, 9H), 0.75-0.91 (m, 2H), 3.44-3.60 (m, 2H), 3.92 (s, 3H), 5.38 (s, 2H), 7.81 (s, 1H), 8.21 (s, 1H), 8.59 (s, 1H), 9.77 (s, 1H).

### Example 0D

### methyl 6-[1-(tert-butoxycarbonyl)azetidin-3-yl]-3-[(1-{[2-(trimethylsilyl)ethoxy]methyl}-1H-pyrazol-4-yl)amino]pyrazine-2-carboxylate

A flask containing Intermediate OC (18.5 g, 43.2 mmol) was flushed with nitrogen for 1 hour, and tetrahydrofuran (200 mL) was added. A separate flask was charged with tri(furan-2-yl)phosphine (0.551 g, 2.375 mmol) and tris(dibenzylideneacetone)dipalladium(0) (0.593 g, 0.648 mmol), flushed with nitrogen for 1 hour, and then tetrahydrofuran (20 mL) was added. This catalyst solution was transferred into a previously prepared solution of Intermediate 1B (294 mL, 130 mmol). This catalyst-zinc reagent solution was cannulated into the flask containing the Example 0C solution at 20° C., and the mixture was heated at 65° C. for 1 hour. Eleven additional reactions were set up as described above. All twelve reaction mixtures were combined, cooled to 20° C., filtered, diluted with ethyl acetate, and washed with water. The organic layer was dried over Na₂SO₄, filtered, concentrated, and purified by silica gel chromatography eluting with 80/1 to 0/1 petroleum ether/ethyl acetate to afford the title compound (150 g). ¹H NMR (400 MHz, CD₃OD) δ ppm −0.02 (s, 9H), 0.89 (br t, J=8.05 Hz, 2H), 1.48 (s, 9H), 3.58 (t, J=8.05 Hz, 2H), 3.92-3.99 (m, 1H), 4.01 (s, 3H), 4.12 (br d, J=7.28 Hz, 2H), 4.21-4.31 (m, 2H), 5.42 (s, 2H), 7.77 (s, 1H), 8.28 (s, 1H), 8.36 (s, 1H).

### Example 0E

### tert-butyl 3-{6-carbamoyl-5-[(1-{[2-(trimethylsilyl)ethoxy]methyl}-1H-pyrazol-4-yl)amino]pyrazin-2-yl}azetidine-1-carboxylate

To a solution of Example 0D (32 g, 63.4 mmol) in methanol (100 mL) was added 7 M ammonia in methanol (725 mL, 5073 mmol) at 25° C., and the mixture was heated at 65° C. for 12 hours. After cooling to ambient temperature, the mixture was filtered, and the precipitate dried in vacuo to afford the title compound (31 g). ¹H NMR (400 MHz, dimethyl sulfoxide-d₆) δ ppm −0.04 (s, 9H), 0.83 (t, J=8.01 Hz, 2H), 1.41 (s, 9H), 3.52 (t, J=8.07 Hz, 2H), 3.91-3.99 (m, 1H), 4.04-4.12 (m, 2H), 4.15 (br d, J=6.11 Hz, 2H), 5.38 (s, 2H), 7.74 (s, 1H), 7.98 (br s, 1H), 8.23 (s, 1H), 8.27 (br s, 1H), 8.34 (s, 1H), 10.72-10.76 (m, 1H), 10.73 (s, 1H).

### Example 0F

### 6-(azetidin-3-yl)-3-[(1-{[2-(trimethylsilyl)ethoxy]methyl}-1H-pyrazol-4-yl)amino]pyrazine-2-carboxamide

To a solution of Example 0E (15 g, 30.6 mmol) in dichloromethane (400 mL) was added zinc(II) bromide (138 g, 613 mmol) in portions at 0° C. and the mixture was stirred at 20° C. for 12 hours. Ten additional reactions were set up as described above. All eleven reaction mixtures were combined, and water was added. The mixture was diluted with tetrahydrofuran and ethyl acetate and washed with saturated Na₂CO₃ solution. The organic layer was dried over Na₂SO₄, filtered and concentrated. The residue was purified by silica gel chromatography eluting with 100/1 to 10/1 tetrahydrofuran/methanol, followed by 20/1 to 10/1 methanol (saturated with NH₃)/tetrahydrofuran to afford the title compound (30.3 g). 1H NMR (400 MHz, CD₃OD) δ ppm −0.02 (s, 9H), 0.89 (t, J=8.05 Hz, 2H), 3.58 (t, J=8.05 Hz, 2H), 3.84-3.92 (m, 2H), 3.99-4.13 (m, 3H), 5.40 (s, 2H), 7.72 (s, 1H), 8.24 (d, J=4.85 Hz, 2H); LCMS (ESI+): m/z 390.1 (M+H)⁺.

### Example 0G

### 6-[1-(6-hydroxy-1,3-benzoxazol-2-yl)azetidin-3-yl]-3-[(1-{[2-(trimethylsilyl)ethoxy]methyl}-1H-pyrazol-4-yl)amino]pyrazine-2-carboxamide

To a mixture of Example 0F (1 g, 2.054 mmol) and K₂CO₃ (0.710 g, 5.13 mmol) in N,N-dimethylformamide (3 mL) was added 2-chloro-1,3-benzoxazol-6-ol (0.348 g, 2.054 mmol) at 20° C. and the mixture was stirred for 5 hours. Two additional reactions were set up as described above. All three reaction mixtures were combined and quenched with water and ethyl acetate. The organic layer was dried over Na₂SO₄, filtered, concentrated, and purified by silica gel chromatography eluting with 10/1 to 1/1 petroleum ether/tetrahydrofuran to afford the title compound (1.2 g). ¹H NMR (400 MHz, CD₃OD) δ ppm −0.03 (s, 9H), 0.88 (t, J=8.05 Hz, 2H), 3.58 (t, J=8.05 Hz, 2H), 4.18-4.29 (m, 1H), 4.44 (t, J=7.06 Hz, 2H), 4.52-4.60 (m, 2H), 5.40 (s, 2H), 5.49 (s, 1H), 6.67 (dd, J=8.60, 2.21 Hz, 1H), 6.80 (d, J=1.98 Hz, 1H), 7.10 (d, J=8.38 Hz, 1H), 7.72 (s, 1H), 8.25 (s, 1H), 8.33 (s, 1H); LCMS (ESI+): m/z 523.3 (M+H)⁺.

### Example 1

### 6-[1-(1,3-benzoxazol-2-yl)azetidin-3-yl]-3-[(1-methyl-1H-pyrazol-4-yl)amino]pyrazine-2-carboxamide

To a mixture of Intermediate 1 (31 mg, 0.080 mmol) and K₂CO₃ (27.7 mg, 0.20 mmol) was added N,N-dimethylformamide (0.5 mL) and 2-chlorobenzo[d]oxazole (10 μL, 0.088 mmol) and the mixture was stirred at ambient temperature for 2 hours. Water and ethyl acetate were added, and the mixture was allowed to stand overnight at ambient temperature. The precipitate was filtered and dried in vacuo to provide the title compound (17 mg). 1H NMR (400 MHz, dimethyl sulfoxide-d₆) δ ppm 10.68 (s, 1H), 8.40-8.36 (m, 1H), 8.35 (s, 1H), 8.01 (s, 1H), 7.85 (d, J=2.3 Hz, 1H), 7.57 (d, J=0.8 Hz, 1H), 7.39 (d, J=7.8 Hz, 1H), 7.28 (d, J=7.7 Hz, 1H), 7.13 (td, J=7.6, 1.1 Hz, 1H), 7.01 (td, J=7.8, 1.3 Hz, 1H), 4.45 (dd, J=7.6, 1.4 Hz, 4H), 4.22 (p, J=7.5 Hz, 1H), 3.79 (s, 3H). LCMS (ESI+): m/z 391.1 (M+H)⁺.

### Example 2

### 6-[1-(6-chloro-1,3-benzoxazol-2-yl)azetidin-3-yl]-3-[(1-methyl-1H-pyrazol-4-yl)amino]pyrazine-2-carboxamide

The title compound was prepared as described in Example 1 using 2,6-dichlorobenzo[d]oxazole in place of 2-chlorobenzo[d]oxazole. ¹H NMR (400 MHz, dimethyl sulfoxide-d₆) δ ppm 10.67 (s, 1H), 8.38 (s, 1H), 8.34 (s, 1H), 8.00 (d, J=0.7 Hz, 1H), 7.85 (d, J=2.2 Hz, 1H), 7.58-7.54 (m, 2H), 7.26 (d, J=8.3 Hz, 1H), 7.17 (dd, J=8.4, 2.0 Hz, 1H), 4.45 (d, J=7.8 Hz, 4H), 4.22 (p, J=7.6 Hz, 1H), 3.78 (s, 3H). LCMS (ESI+): m/z 425.3 (M+H)⁺.

### Example 3

### 6-[1-(6-methyl-1,3-benzoxazol-2-yl)azetidin-3-yl]-3-[(1-methyl-1H-pyrazol-4-yl)amino]pyrazine-2-carboxamide

### Example 3A

### 6-methyl-1,3-benzoxazole-2-thiol

A mixture of 2-amino-5-methylphenol (5.0 g, 40.6 mmol), tetramethylthiuram disulfide (5.86 g, 24.4 mmol) and water (100 mL) was heated at 80° C. for 4 hours. The mixture was cooled to ambient temperature and adjusted to pH 3 by addition of 1 N hydrochloric acid. The precipitate was collected by filtration and rinsed with water to afford the title compound (7.33 g). LCMS (ESI+): m/z 166.1 (M+H)⁺.

### Example 3B

### 2-chloro-6-methyl-1,3-benzoxazole

To a solution of Example 3A (7.3 g, 44.2 mmol) in dichloromethane (100 mL) at 0° C. was added thionyl chloride (13.4 g, 113 mmol) dropwise, followed by N,N-dimethylformamide (4.1 mL, 53 mmol). The mixture was warmed to ambient temperature and stirred overnight. The mixture was concentrated, and the residue was treated with ethyl acetate and saturated NaHCO₃. After stirring for 30 minutes, the aqueous layer was extracted with ethyl acetate, dried over Na₂SO₄, filtered and concentrated. The residue was purified by flash chromatography on silica gel eluting with 100/0 to 96/4 heptanes/ethyl acetate to afford the title compound (4.6 g). LCMS (ESI+): m/z 168.3 (M+H)⁺.

### Example 3C

### 6-[1-(6-methyl-1,3-benzoxazol-2-yl)azetidin-3-yl]-3-[(1-methyl-1H-pyrazol-4-yl)amino]pyrazine-2-carboxamide

To a mixture of Intermediate 1 (347 mg, 0.895 mmol) and K₂CO₃ (309 mg, 2.24 mmol) was added a solution of Example 3B (150 mg, 0.895 mmol) in N,N-dimethylformamide (3 mL) and the mixture was stirred overnight at ambient temperature. The mixture was partitioned between water and ethyl acetate, and the precipitate was washed with water and ethyl acetate and dried to afford the title compound (268 mg). ¹H NMR (501 MHz, dimethyl sulfoxide-d₆) δ ppm 10.68 (s, 1H), 8.36 (app s, 2H), 8.02 (s, 1H), 7.90-7.82 (m, 1H), 7.58 (d, J=0.8 Hz, 1H), 7.22 (s, 1H), 7.16 (d, J=7.8 Hz, 1H), 6.99-6.90 (m, 1H), 4.43 (d, J=7.7 Hz, 4H), 4.22 (p, J=7.6 Hz, 1H), 3.80 (s, 3H), 2.33 (s, 3H). LCMS (ESI+): m/z 405.3 (M+H)⁺

### Example 4

### 6-[1-(5,6-dimethyl-1,3-benzoxazol-2-yl)azetidin-3-yl]-3-[(1-methyl-1H-pyrazol-4-yl)amino]pyrazine-2-carboxamide

The title compound was prepared as described in Examples 3B and 3C using 5,6-dimethylbenzo[d]oxazole-2-thiol in place of Example 3A. ¹H NMR (400 MHz, dimethyl sulfoxide-d₆) δ ppm 10.66 (s, 1H), 8.34 (s, 1H), 8.33 (s, 1H), 8.00 (s, 1H), 7.84 (s, 1H), 7.56 (d, J=0.8 Hz, 1H), 7.17 (s, 1H), 7.06 (s, 1H), 4.41 (d, J=7.6 Hz, 4H), 4.24-4.15 (m, 1H), 3.78 (s, 3H), 2.21 (s, 3H), 2.20 (s, 3H). LCMS (ESI+): m/z 419.3 (M+H)⁺.

### Example 5

### 6-[1-(6-ethyl-1,3-benzoxazol-2-yl)azetidin-3-yl]-3-[(1-methyl-1H-pyrazol-4-yl)amino]pyrazine-2-carboxamide

The title compound was prepared as described in Examples 3A-C using 2-amino-5-ethylphenol in place of 2-amino-5-methylphenol. ¹H NMR (400 MHz, dimethyl sulfoxide-d₆) δ ppm 10.67 (s, 1H), 8.34 (s, 2H), 8.00 (s, 1H), 7.88-7.80 (m, 1H), 7.57 (s, 1H), 7.26-7.21 (m, 1H), 7.17 (d, J=7.9 Hz, 1H), 6.97 (dd, J=7.9, 1.6 Hz, 1H), 4.42 (d, J=7.6 Hz, 4H), 4.21 (p, J=7.6 Hz, 1H), 3.78 (s, 3H), 2.61 (q, J=7.6 Hz, 2H), 1.15 (t, J=7.6 Hz, 3H). LCMS (ESI+): m/z 419.4 (M+H)⁺.

### Example 6

### 6-{1-[6-(cyclohex-1-en-1-yl)-1,3-benzoxazol-2-yl]azetidin-3-yl}-3-[(1-methyl-1H-pyrazol-4-yl)amino]pyrazine-2-carboxamide

### Example 6A

### 6-[1-(6-bromo-1,3-benzoxazol-2-yl)azetidin-3-yl]-3-[(1-methyl-1H-pyrazol-4-yl)amino]pyrazine-2-carboxamide

The title compound was prepared as described in Examples 3A-C using 2-amino-5-bromophenol in place of 2-amino-5-methylphenol. ¹H NMR (501 MHz, dimethyl sulfoxide-d₆) δ ppm 10.68 (s, 1H), 8.43-8.37 (m, 1H), 8.35 (s, 1H), 8.02 (s, 1H), 7.87-7.81 (m, 1H), 7.69 (d, J=1.8 Hz, 1H), 7.58 (d, J=0.8 Hz, 1H), 7.31 (dd, J=8.3, 1.9 Hz, 1H), 7.23 (d, J=8.3 Hz, 1H), 4.46 (d, J=7.7 Hz, 4H), 4.24 (p, J=7.7 Hz, 1H), 3.79 (s, 3H). LCMS (ESI+): m/z 469, 471 (M+H)⁺.

### Example 6B

### 6-{1-[6-(cyclohex-1-en-1-yl)-1,3-benzoxazol-2-yl]azetidin-3-yl}-3-[(1-methyl-1H-pyrazol-4-yl)amino]pyrazine-2-carboxamide

A mixture of Example 6A (50 mg, 0.107 mmol), 2-(cyclohex-1-en-1-yl)-4,4,5,5-tetramethyl-1,3,2-dioxaborolane (33 mg, 0.160 mmol), [1,1′-bis(diphenylphosphino)ferrocene]palladium(II) dichloride (7.8 mg, 10.65 mmol) and Na₂CO₃ (25 mg, 0.234 mmol) was degassed with nitrogen. Dioxane (0.6 mL) and nitrogen-sparged water (0.15 mL) were added, and the mixture was heated at 90° C. for 16 hours. The mixture was cooled to ambient temperature, diluted with water, and extracted with ethyl acetate. The organic layers were dried over Na₂SO₄, filtered, concentrated, and purified by flash chromatography on silica gel eluting with 100/0 to 93/7 dichloromethane/methanol to afford the title compound (35 mg). ¹H NMR (501 MHz, dimethyl sulfoxide-d₆) δ ppm 10.68 (s, 1H), 8.38 (s, 1H), 8.36 (s, 1H), 8.02 (d, J=0.8 Hz, 1H), 7.86 (d, J=2.3 Hz, 1H), 7.58 (d, J=0.8 Hz, 1H), 7.43 (d, J=1.3 Hz, 1H), 7.23-7.18 (m, 2H), 6.11-6.08 (m, 1H), 4.45 (d, J=7.7 Hz, 4H), 4.23 (p, J=7.6 Hz, 1H), 3.80 (s, 3H), 2.36 (tt, J=6.9, 2.8 Hz, 2H), 2.15 (h, J=3.6 Hz, 2H), 1.74-1.66 (m, 2H), 1.61-1.54 (m, 2H). LCMS (ESI+): m/z 471.2 (M+H)⁺.

### Example 7

### 6-[1-(6-cyclohexyl-1,3-benzoxazol-2-yl)azetidin-3-yl]-3-[(1-methyl-1H-pyrazol-4-yl)amino]pyrazine-2-carboxamide

To a solution of Example 6 (17.7 mg, 0.038 mmol) in 1,2-dichloroethane (1 mL) was added 5% palladium on carbon (17 mg, 0.071 mmol) and the mixture was heated at 55° C. for 22 hours under a hydrogen atmosphere (50 psi). The mixture was filtered, concentrated, and purified by flash chromatography on silica gel eluting with 100/0 to 94/6 dichloromethane/methanol to afford the title compound (11 mg). ¹H NMR (501 MHz, dimethyl sulfoxide-d₆) δ ppm 10.67 (s, 1H), 8.37-8.34 (m, 2H), 8.01 (d, J=0.8 Hz, 1H), 7.85 (d, J=2.2 Hz, 1H), 7.58 (d, J=0.8 Hz, 1H), 7.25 (d, J=1.6 Hz, 1H), 7.18 (d, J=8.0 Hz, 1H), 7.00 (dd, J=8.1, 1.6 Hz, 1H), 4.47-4.39 (m, 4H), 4.22 (p, J=7.6 Hz, 1H), 3.79 (s, 3H), 2.48 (p, J=1.8 Hz, 1H), 1.77 (d, J=10.4 Hz, 4H), 1.67 (d, J=13.0 Hz, 1H), 1.44-1.29 (m, 4H), 1.22 (t, J=12.1 Hz, 1H). LCMS (ESI+): m/z 473.4 (M+H)⁺.

### Example 8

### 3-[(1-methyl-1H-pyrazol-4-yl)amino]-6-[1-(6-phenyl-1,3-benzoxazol-2-yl)azetidin-3-yl]pyrazine-2-carboxamide

The title compound was prepared as described in Example 6 using phenylboronic acid in place of 2-(cyclohex-1-en-1-yl)-4,4,5,5-tetramethyl-1,3,2-dioxaborolane. ¹H NMR (400 MHz, dimethyl sulfoxide-d₆) δ ppm 10.67 (s, 1H), 8.38 (s, 1H), 8.36 (s, 1H), 8.01 (s, 1H), 7.85 (d, J=2.2 Hz, 1H), 7.71 (d, J=1.7 Hz, 1H), 7.64 (dd, J=7.7, 1.5 Hz, 2H), 7.57 (s, 1H), 7.46 (dd, J=8.2, 1.7 Hz, 1H), 7.41 (t, J=7.7 Hz, 2H), 7.34 (d, J=8.2 Hz, 1H), 7.29 (t, J=7.4 Hz, 1H), 4.48 (d, J=7.6 Hz, 4H), 4.24 (p, J=7.6 Hz, 1H), 3.78 (s, 3H). LCMS (ESI+): m/z 467.3 (M+H)⁺.

### Example 9

### 6-[1-(6-methoxy-1,3-benzoxazol-2-yl)azetidin-3-yl]-3-[(1-methyl-1H-pyrazol-4-yl)amino]pyrazine-2-carboxamide

The title compound was prepared as described in Examples 3A-C using 2-amino-5-methoxyphenol in place of 2-amino-5-methylphenol. ¹H NMR (501 MHz, dimethyl sulfoxide-d₆) δ ppm 10.67 (s, 1H), 8.39-8.31 (m, 2H), 8.02 (s, 1H), 7.93-7.81 (m, 1H), 7.58 (s, 1H), 7.18 (d, J=8.6 Hz, 1H), 7.10 (d, J=2.4 Hz, 1H), 6.75 (dd, J=8.6, 2.5 Hz, 1H), 4.42 (d, J=7.7 Hz, 4H), 4.21 (p, J=7.6 Hz, 1H), 3.79 (s, 3H), 3.73 (s, 3H). LCMS (ESI+): m/z 421.2 (M+H)⁺.

### Example 10

### 6-[1-(6-cyclopentyl-1,3-benzoxazol-2-yl)azetidin-3-yl]-3-[(1-methyl-1H-pyrazol-4-yl)amino]pyrazine-2-carboxamide

### Example 10A

### 6-{1-[6-(cyclopent-1-en-1-yl)-1,3-benzoxazol-2-yl]azetidin-3-yl}-3-[(1-methyl-1H-pyrazol-4-yl)amino]pyrazine-2-carboxamide

The title compound was prepared as described in Example 6 using cyclopent-1-en-1-ylboronic acid in place of 2-(cyclohex-1-en-1-yl)-4,4,5,5-tetramethyl-1,3,2-dioxaborolane. ¹H NMR (400 MHz, dimethyl sulfoxide-d₆) δ ppm 10.67 (s, 1H), 8.39-8.36 (m, 1H), 8.34 (s, 1H), 8.00 (d, J=0.8 Hz, 1H), 7.88-7.81 (m, 1H), 7.57 (d, J=0.8 Hz, 1H), 7.49 (d, J=1.6 Hz, 1H), 7.25 (dd, J=8.2, 1.6 Hz, 1H), 7.20 (d, J=8.1 Hz, 1H), 6.21-6.14 (m, 1H), 4.44 (d, J=7.7 Hz, 4H), 4.22 (p, J=7.6 Hz, 1H), 3.78 (s, 3H), 2.67-2.58 (m, 2H), 2.49-2.41 (m, 2H), 1.92 (p, J=7.5 Hz, 2H). LCMS (ESI+): m/z 457.4 (M+H)⁺.

### Example 10B

### 6-{1-[6-(cyclopent-1-en-1-yl)-1,3-benzoxazol-2-yl]azetidin-3-yl}-3-[(1-methyl-1H-pyrazol-4-yl)amino]pyrazine-2-carboxamide

The title compound was prepared as described in Example 7 using Example 10A in place of Example 6. ¹H NMR (400 MHz, dimethyl sulfoxide-d₆) δ ppm 10.67 (s, 1H), 8.36-8.32 (m, 2H), 8.00 (d, J=0.8 Hz, 1H), 7.87-7.80 (m, 1H), 7.57 (d, J=0.8 Hz, 1H), 7.27 (d, J=1.6 Hz, 1H), 7.16 (d, J=8.0 Hz, 1H), 7.02 (dd, J=8.3, 1.7 Hz, 1H), 4.42 (d, J=7.7 Hz, 4H), 4.21 (p, J=7.6 Hz, 1H), 3.78 (s, 3H), 3.02-2.92 (m, 1H), 1.98 (q, J=12.2, 9.0 Hz, 2H), 1.79-1.67 (m, 2H), 1.67-1.43 (m, 4H). LCMS (ESI+): m/z 459.3 (M+H)⁺.

### Example 11

### 6-{1-[6-(3,6-dihydro-2H-pyran-4-yl)-1,3-benzoxazol-2-yl]azetidin-3-yl}-3-[(1-methyl-1H-pyrazol-4-yl)amino]pyrazine-2-carboxamide

The title compound was prepared described in Example 6 using 2-(3,6-dihydro-2H-pyran-4-yl)-4,4,5,5-tetramethyl-1,3,2-dioxaborolane in place of 2-(cyclohex-1-en-1-yl)-4,4,5,5-tetramethyl-1,3,2-dioxaborolane. ¹H NMR (400 MHz, dimethyl sulfoxide-d₆) δ ppm 10.67 (s, 1H), 8.38-8.36 (m, 1H), 8.35 (s, 1H), 8.01 (d, J=0.8 Hz, 1H), 7.87-7.83 (m, 1H), 7.57 (d, J=0.8 Hz, 1H), 7.49 (d, J=1.6 Hz, 1H), 7.25 (dd, J=8.3, 1.7 Hz, 1H), 7.22 (d, J=8.2 Hz, 1H), 6.21-6.15 (m, 1H), 4.45 (d, J=7.7 Hz, 4H), 4.27-4.15 (m, 3H), 3.81-3.77 (m, 5H), 2.44-2.39 (m, 2H). LCMS (ESI+): m/z 473.3 (M+H)⁺.

### Example 12

### 3-[(1-methyl-1H-pyrazol-4-yl)amino]-6-{1-[6-(trifluoromethyl)-1,3-benzoxazol-2-yl]azetidin-3-yl}pyrazine-2-carboxamide

### Example 12A

### 6-(trifluoromethyl)-1,3-benzoxazole-2-thiol

To a solution of potassium hydroxide (317 mg, 5.65 mmol) in ethanol (5 mL) and water (2 mL) was added carbon disulfide (0.187 mL, 3.11 mmol), and the mixture was stirred for 15 minutes. 2-Amino-5-(trifluoromethyl)phenol (500 mg, 2.82 mmol) was added, the mixture heated at reflux for 3 hours, and allowed to stand at ambient temperature overnight. The mixture was diluted with water and adjusted to pH 3-4 using 1 N hydrochloric acid. The precipitate was filtered and purified by flash chromatography on silica gel eluting with 25/75 to 40/60 ethyl acetate/heptanes to afford the title compound (205 mg). LCMS (ESI−): m/z 217.9 (M−H)⁻.

### Example 12B

### 3-[(1-methyl-1H-pyrazol-4-yl)amino]-6-{1-[6-(trifluoromethyl)-1,3-benzoxazol-2-yl]azetidin-3-yl}pyrazine-2-carboxamide

The title compound was prepared as described in Examples 3B and 3C using Example 12A in place of Example 3A. ¹H NMR (501 MHz, dimethyl sulfoxide-d₆) δ ppm 10.70 (s, 1H), 8.45-8.40 (m, 1H), 8.36 (s, 1H), 8.02 (d, J=0.7 Hz, 1H), 7.87 (s, 1H), 7.82 (d, J=1.7 Hz, 1H), 7.58 (d, J=0.7 Hz, 1H), 7.53-7.49 (m, 1H), 7.43 (d, J=8.2 Hz, 1H), 4.51 (d, J=7.7 Hz, 4H), 4.27 (p, J=7.7 Hz, 1H), 3.80 (s, 3H). LCMS (ESI+): m/z 459.3 (M+H)⁺.

### Example 13

### methyl 2-(3-{6-carbamoyl-5-[(1-methyl-1H-pyrazol-4-yl)amino]pyrazin-2-yl}azetidin-1-yl)-1,3-benzoxazole-6-carboxylate

The title compound was prepared as described in Examples 3A-C using methyl 4-amino-3-hydroxybenzoate in place of 2-amino-5-methylphenol. ¹H NMR (400 MHz, dimethyl sulfoxide-d₆) δ ppm 10.73 (s, 1H), 8.48-8.43 (m, 1H), 8.38 (s, 1H), 8.04 (s, 1H), 7.91 (d, J=1.6 Hz, 1H), 7.90-7.87 (m, 1H), 7.85 (dd, J=8.3, 1.6 Hz, 1H), 7.61 (d, J=0.8 Hz, 1H), 7.37 (d, J=8.2 Hz, 1H), 4.54 (d, J=7.7 Hz, 4H), 4.28 (p, J=7.6 Hz, 1H), 3.84 (s, 3H), 3.82 (s, 3H). LCMS (ESI+): m/z 449.3 (M+H)⁺.

### Example 14

### 6-[1-(1,3-benzoxazol-2-yl)azetidin-3-yl]-3-[(1H-pyrazol-4-yl)amino]pyrazine-2-carboxamide

The title compound was prepared as described in Examples 27A-C using 2-chlorobenzoxazole in place of Example 36A and tert-butyl 4-amino-1H-pyrazole-1-carboxylate in place of 1-(tetrahydro-2H-pyran-4-yl)-1H-pyrazol-4-amine. ¹H NMR (500 MHz, dimethyl sulfoxide-d₆) δ ppm 14.18 (br s, 1H), 12.61 (s, 1H), 10.71 (s, 1H), 8.40 (s, 2H), 8.12-7.62 (br, 1H), 7.88 (d, 2.4 Hz, 1H), 7.45-7.39 (m, 1H), 7.32 (dd, J=7.8, 1.2 Hz, 1H), 7.17 (td, J=7.7, 1.1 Hz, 1H), 7.05 (td, J=7.7, 1.2 Hz, 1H), 4.52-4.45 (m, 4H), 4.26 (p, J=7.7 Hz, 1H). LCMS (ESI+): m/z 377.3 (M+H)⁺.

### Example 15

### 3-[(1-methyl-1H-pyrazol-4-yl)amino]-6-{1-[6-(oxolan-3-yl)-1,3-benzoxazol-2-yl]azetidin-3-yl}pyrazine-2-carboxamide

### Example 15A

### 6-{1-[6-(2,5-dihydrofuran-3-yl)-1,3-benzoxazol-2-yl]azetidin-3-yl}-3-[(1-methyl-1H-pyrazol-4-yl)amino]pyrazine-2-carboxamide

The title compound was prepared as described in Example 6 using 2-(2,5-dihydrofuran-3-yl)-4,4,5,5-tetramethyl-1,3,2-dioxaborolane in place of 2-(cyclohex-1-en-1-yl)-4,4,5,5-tetramethyl-1,3,2-dioxaborolane. ¹H NMR (400 MHz, dimethyl sulfoxide-d₆) δ ppm 10.67 (s, 1H), 8.39-8.36 (m, 1H), 8.35 (s, 1H), 8.00 (d, J=0.8 Hz, 1H), 7.88-7.81 (m, 1H), 7.57 (d, J=0.8 Hz, 1H), 7.52 (d, J=1.6 Hz, 1H), 7.24 (d, J=8.1 Hz, 1H), 7.18 (dd, J=8.1, 1.6 Hz, 1H), 6.40-6.34 (m, 1H), 4.86 (td, J=4.8, 2.0 Hz, 2H), 4.69 (td, J=4.9, 2.0 Hz, 2H), 4.45 (d, J=7.7 Hz, 4H), 4.23 (p, J=7.6 Hz, 1H), 3.78 (s, 3H). LCMS (ESI+): m/z 459.4 (M+H)⁺.

### Example 15B

### 3-[(1-methyl-1H-pyrazol-4-yl)amino]-6-{1-[6-(oxolan-3-yl)-1,3-benzoxazol-2-yl]azetidin-3-yl}pyrazine-2-carboxamide

The title compound was prepared as described in Example 7 using Example 15A in place of Example 6. ¹H NMR (501 MHz, dimethyl sulfoxide-d₆) δ ppm 10.68 (s, 1H), 8.40-8.32 (m, 2H), 8.02 (s, 1H), 7.85 (d, J=2.3 Hz, 1H), 7.58 (d, J=0.8 Hz, 1H), 7.33 (d, J=1.6 Hz, 1H), 7.21 (d, J=8.0 Hz, 1H), 7.06 (dd, J=8.2, 1.7 Hz, 1H), 4.44 (d, J=7.7 Hz, 4H), 4.23 (p, J=7.7 Hz, 1H), 4.01 (t, J=7.7 Hz, 1H), 3.93 (td, J=8.3, 4.4 Hz, 1H), 3.80 (s, 3H), 3.80-3.73 (m, 1H), 3.51 (t, J=8.0 Hz, 1H), 3.41 (dd, J=15.9, 8.0 Hz, 1H), 2.28 (dtd, J=12.1, 7.6, 4.4 Hz, 1H), 1.91 (dq, J=12.2, 8.2 Hz, 1H). LCMS (ESI+): m/z 461.4 (M+H)⁺.

### Example 16

### 6-{1-[6-(hydroxymethyl)-1,3-benzoxazol-2-yl]azetidin-3-yl}-3-[(1-methyl-1H-pyrazol-4-yl)amino]pyrazine-2-carboxamide

To an ice-cooled suspension of Example 13 (30 mg, 0.067 mmol) in tetrahydrofuran (1 mL) was added dropwise diisobutylaluminum hydride (1M in tetrahydrofuran, 0.3 mL, 0.3 mmol), and the mixture was stirred at ambient temperature for 2 hours. The mixture was quenched with methanol (1 mL) and water (0.2 mL), stirred for 30 minutes and concentrated. The residue was partitioned between water and ethyl acetate, and the organic layers were washed with brine, dried over Na₂SO₄, filtered, and concentrated. The residue was purified by silica gel chromatography eluting with 100/0 to 90/10 dichloromethane/methanol to afford the title compound (13 mg). ¹H NMR (501 MHz, dimethyl sulfoxide-d₆) δ ppm 10.68 (s, 1H), 8.39-8.35 (m, 2H), 8.02 (d, J=0.8 Hz, 1H), 7.86 (d, J=2.2 Hz, 1H), 7.58 (d, J=0.8 Hz, 1H), 7.33 (dd, J=1.5, 0.7 Hz, 1H), 7.22 (d, J=7.9 Hz, 1H), 7.16-6.99 (m, 1H), 5.15 (t, J=5.7 Hz, 1H), 4.50 (d, J=5.5 Hz, 2H), 4.45 (d, J=7.6 Hz, 4H), 4.23 (p, J=7.7 Hz, 1H), 3.80 (s, 3H). LCMS (DCI+): m/z 421.3 (M+H)⁺.

### Example 17

### 6-(1-{6-[(1E)-3-hydroxy-3-methylbut-1-en-1-yl]-1,3-benzoxazol-2-yl}azetidin-3-yl)-3-[(1-methyl-1H-pyrazol-4-yl)amino]pyrazine-2-carboxamide

A mixture of Example 6A (75 mg, 0.16 mmol), (E)-2-methyl-4-(4,4,5,5-tetramethyl-1,3,2-dioxaborolane-2-yl)but-3-en-2-ol (46 mg, 0.22 mmol), K₂CO₃ (44 mg, 0.32 mmol), (1S,3R,5R,7S)-1,3,5,7-tetramethyl-8-phenyl-2,4,6-trioxa-8-phosphaadamantane (2.3 mg, 8.0 μmol) and tris(dibenzylideneacetone)dipalladium(0) (3.7 mg, 4.0 μmol) was sparged with nitrogen, and water (200 μL) and dioxane (600 μL) were added. The mixture was heated at 65° C. for 16 hours, cooled and partitioned between water and ethyl acetate. The organic extracts were dried over Na₂SO₄, concentrated, and purified by flash chromatography on silica gel eluting with 100/0 to 90/10 methanol/dichloromethane to afford the title compound (50 mg). ¹H NMR (501 MHz, dimethyl sulfoxide-d₆) δ ppm 10.69 (s, 1H), 8.39 (d, J=2.2 Hz, 1H), 8.36 (s, 1H), 8.02 (d, J=0.8 Hz, 1H), 7.87 (d, J=2.3 Hz, 1H), 7.58 (d, J=0.8 Hz, 1H), 7.47 (d, J=1.2 Hz, 1H), 7.22-7.18 (m, 2H), 6.50 (d, J=16.0 Hz, 1H), 6.31 (d, J=16.1 Hz, 1H), 4.65 (s, 1H), 4.46 (d, J=7.8 Hz, 4H), 4.24 (p, J=7.6 Hz, 1H), 3.80 (s, 3H), 1.25 (s, 6H). LCMS (ESI+): m/z 474.9 (M+H)⁺.

### Example 18

### 2-(3-{6-carbamoyl-5-[(1-methyl-1H-pyrazol-4-yl)amino]pyrazin-2-yl}azetidin-1-yl)-N,N-dimethyl-1,3-benzoxazole-6-carboxamide

### Example 18A

### 2-(3-f{6-carbamoyl-5-[(1-methyl-1H-pyrazol-4-yl)amino]pyrazin-2-yl}azetidin-1-yl)-1,3-benzoxazole-6-carboxylic acid

A suspension of Example 13 (150 mg, 0.334 mmol) in tetrahydrofuran (1.5 mL) and methanol (0.5 mL) was treated with 2 N lithium hydroxide (1 mL), and the mixture was stirred at ambient temperature for 3 hours and at 50° C. for 1 hour. The mixture was concentrated, diluted with water, and adjusted to pH 4 with 1 N hydrochloric acid. The precipitate was collected by filtration, rinsed with water, and purified by silica gel chromatography eluting with 100/0 to 88/12 dichloromethane/methanol to afford the title compound (15 mg). ¹H NMR (501 MHz, dimethyl sulfoxide-d₆) δ ppm 10.70 (s, 1H), 8.43 (d, J=2.3 Hz, 1H), 8.36 (s, 1H), 8.02 (s, 1H), 7.91-7.84 (m, 2H), 7.81 (dd, J=8.2, 1.6 Hz, 1H), 7.58 (s, 1H), 7.32 (d, J=8.2 Hz, 1H), 4.51 (d, J=7.7).

### Example 18B

### 2-(3-{6-carbamoyl-5-[(1-methyl-1H-pyrazol-4-yl)amino]pyrazin-2-yl}azetidin-1-yl)-N,N-dimethyl-1,3-benzoxazole-6-carboxamide

To a mixture of Example 18A (100 mg, 0.23 mmol), dimethylamine hydrochloride (28 mg, 0.35 mmol), N¹-((ethylimino)methylene)-N,N-dimethylpropane-1,3-diamine hydrochloride (88 mg, 0.46 mmol) and 1H-benzo[d][1,2,3]triazol-1-ol hydrate (42 mg, 0.28 mmol) in N,N-dimethylformamide (1.2 mL) was added dropwise N-ethyl-N-isopropylpropan-2-amine (200 μL, 1.15 mmol), and the mixture was stirred at ambient temperature for 3 hours. The mixture was partitioned between water and ethyl acetate and the extracts were dried over Na₂SO₄, filtered and concentrated. The residue was purified by flash chromatography on silica gel eluting with 100/0 to 92/8 dichloromethane/methanol to afford the title compound (34 mg). ¹H NMR (400 MHz, dimethyl sulfoxide-d₆) δ ppm 10.68 (s, 1H), 8.39 (s, 1H), 8.35 (s, 1H), 8.01 (d, J=0.8 Hz, 1H), 7.85 (s, 1H), 7.57 (d, J=0.8 Hz, 1H), 7.46 (d, J=1.5 Hz, 1H), 7.28 (d, J=8.1 Hz, 1H), 7.20 (dd, J=8.0, 1.5 Hz, 1H), 4.47 (d, J=7.8 Hz, 4H), 4.24 (p, J=7.6 Hz, 1H), 3.78 (s, 3H), 2.93 (s, 6H). LCMS (ESI+): m/z 462.02 (M+H)⁺.

### Example 19

### 6-{1-[6-(2-hydroxypropan-2-yl)-1,3-benzoxazol-2-yl]azetidin-3-yl}-3-[(1-methyl-1H-pyrazol-4-yl)amino]pyrazine-2-carboxamide

To an ice-cooled suspension of Example 13 (43 mg, 0.096 mmol) in tetrahydrofuran (1 mL) was added dropwise methylmagnesium bromide (3 M in diethyl ether, 0.15 mL, 0.45 mmol), and the mixture was stirred at ambient temperature for 16 hours. The mixture was diluted with water and extracted with ethyl acetate. The organic extracts were dried over Na₂SO₄, filtered, concentrated, and purified by silica gel chromatography eluting with 100/0 to 90/10 dichloromethane/methanol, followed by trituration with minimal ethyl acetate to afford the title compound (14 mg). ¹H NMR (400 MHz, dimethyl sulfoxide-d₆) δ ppm 10.66 (s, 1H), 8.36-8.33 (m, 2H), 8.00 (s, 1H), 7.84 (s, 1H), 7.57 (d, J=0.8 Hz, 1H), 7.45 (d, J=1.7 Hz, 1H), 7.24 (dd, J=8.2, 1.7 Hz, 1H), 7.18 (d, J=8.2 Hz, 1H), 4.98 (s, 1H), 4.43 (d, J=7.7 Hz, 4H), 4.21 (p, J=7.6 Hz, 1H), 3.78 (s, 3H), 1.41 (s, 6H). LCMS (ESI+): m/z 448.98 (M+H)⁺.

### Example 20

### 6-[I1-(6-hydroxy-1,3-benzoxazol-2-yl)azetidin-3-yl]-3-[(1-methyl-1H-pyrazol-4-yl)amino]pyrazine-2-carboxamide

The title compound was obtained as described in Example 1 using 2-chlorobenzo[d]oxazol-6-ol in place of 2-chlorobenzo[d]oxazole. ¹H NMR (501 MHz, dimethyl sulfoxide-d₆) δ ppm 10.70 (s, 1H), 9.25 (s, 1H), 8.38 (d, J=4.3 Hz, 2H), 8.04 (s, 1H), 7.89 (d, J=2.3 Hz, 1H), 7.60 (s, 1H), 7.11 (d, J=8.4 Hz, 1H), 6.85 (d, J=2.3 Hz, 1H), 6.63 (dd, J=8.4, 2.3 Hz, 1H), 4.43 (d, J=7.7 Hz, 4H), 4.23 (t, J=7.7 Hz, 1H), 3.81 (s, 3H). LCMS (ESI+): m/z 407.0 (M+H)⁺.

### Example 21

### 6-{1-[6-(3-hydroxy-3-methylbutyl)-1,3-benzoxazol-2-yl]azetidin-3-yl}-3-[(1-methyl-1H-pyrazol-4-yl)amino]pyrazine-2-carboxamide

The title compound was prepared as described in Example 7 using Example 17 in place of Example 6. ¹H NMR (400 MHz, dimethyl sulfoxide-d₆) δ ppm 10.67 (s, 1H), 8.38-8.32 (m, 2H), 8.00 (d, J=0.7 Hz, 1H), 7.84 (d, J=2.4 Hz, 1H), 7.57 (d, J=0.8 Hz, 1H), 7.20 (d, J=1.5 Hz, 1H), 7.16 (d, J=7.9 Hz, 1H), 6.95 (dd, J=8.0, 1.6 Hz, 1H), 4.42 (d, J=7.6 Hz, 4H), 4.25-4.16 (m, 2H), 3.78 (s, 3H), 2.66-2.58 (m, 2H), 1.64-1.57 (m, 2H), 1.10 (s, 6H). LCMS (ESI+): m/z 477.2 (M+H)⁺.

### Example 22

### 6-(1-{6-[(1E)-3-methoxyprop-1-en-1-yl]-1,3-benzoxazol-2-yl}azetidin-3-yl)-3-[(1-methyl-1H-pyrazol-4-yl)amino]pyrazine-2-carboxamide

The title compound was prepared as described in Example 17 using (E)-2-(3-methoxyprop-1-en-1-yl)-4,4,5,5-tetramethyl-1,3,2-dioxaborolane in place of (E)-2-methyl-4-(4,4,5,5-tetramethyl-1,3,2-dioxaborolane-2-yl)but-3-en-2-ol. ¹H NMR (400 MHz, dimethyl sulfoxide-d₆) δ ppm 10.68 (s, 1H), 8.40-8.36 (m, 1H), 8.35 (s, 1H), 8.01 (d, J=0.8 Hz, 1H), 7.85 (d, J=2.1 Hz, 1H), 7.57 (d, J=0.8 Hz, 1H), 7.54 (s, 1H), 7.25-7.17 (m, 2H), 6.59 (dt, J=16.0, 1.5 Hz, 1H), 6.25 (dt, J=15.9, 5.9 Hz, 1H), 4.45 (d, J=7.7 Hz, 4H), 4.22 (p, J=7.6 Hz, 1H), 3.99 (dd, J=6.0, 1.4 Hz, 2H), 3.78 (s, 3H), 3.24 (s, 3H). LCMS (ESI+): m/z 461.2 (M+H)⁺.

### Example 23

### 6-{1-[6-(cyclopropylmethoxy)-1,3-benzoxazol-2-yl]azetidin-3-yl}-3-[(1-methyl-1H-pyrazol-4-yl)amino]pyrazine-2-carboxamide

A mixture of Example 20 (0.050 g, 0.123 mmol) and sodium hydride (5.4 mg, 0.135 mmol, 60% dispersion in mineral oil) in N,N-dimethylformamide (0.5 mL) was stirred at ambient temperature for 15 minutes. (Iodomethyl)cyclopropane (2.7 mg, 0.0148 mmol) was added, and the mixture was stirred at ambient temperature for 1.5 hours. The mixture was quenched with water and diluted with ethyl acetate. The aqueous layer was washed with ethyl acetate, and the organic layers were washed with water, brine, dried over Na₂SO₄, and concentrated. The residue was purified by silica gel chromatography eluting with 100:0 to 100:10 dichloromethane/methanol to afford the title compound (21 mg). ¹H NMR (501 MHz, dimethyl sulfoxide-d₆) δ ppm 10.69 (s, 1H), 8.38 (s, 2H), 8.04 (d, J=0.8 Hz, 1H), 7.88 (d, J=2.2 Hz, 1H), 7.60 (d, J=0.8 Hz, 1H), 7.18 (d, J=8.6 Hz, 1H), 7.09 (d, J=2.4 Hz, 1H), 6.76 (dd, J=8.6, 2.4 Hz, 1H), 4.43 (d, J=7.7 Hz, 4H), 4.23 (t, J=7.7 Hz, 1H), 3.81 (s, 3H), 3.79 (d, J=6.9 Hz, 2H), 1.22 (d, J=13.6 Hz, 1H), 0.59-0.49 (m, 2H), 0.35-0.23 (m, 2H). LCMS (ESI+): m/z 461.5 (M+H)⁺.

### Example 24

### 6-{1-[6-(cyanomethoxy)-1,3-benzoxazol-2-yl]azetidin-3-yl}-3-[(1-methyl-1H-pyrazol-4-yl)amino]pyrazine-2-carboxamide

The title compound was prepared as described in Example 23 using 2-iodoacetonitrile in place of (iodomethyl)cyclopropane. ¹H NMR (501 MHz, dimethyl sulfoxide-d₆) δ ppm 10.70 (s, 1H), 8.39 (d, J=11.6 Hz, 2H), 8.04 (d, J=0.8 Hz, 1H), 7.87 (d, J=2.4 Hz, 1H), 7.60 (d, J=0.7 Hz, 1H), 7.34-7.23 (m, 2H), 6.92 (dd, J=8.6, 2.5 Hz, 1H), 5.14 (s, 2H), 4.46 (dd, J=7.7, 1.7 Hz, 4H), 4.26 (q, J=7.8 Hz, 1H), 3.81 (s, 3H). LCMS (ESI+): m/z 446.3 (M+H)⁺.

### Example 25

### 6-(1-{6-[3-hydroxy-2-(hydroxymethyl)propyl]-1,3-benzoxazol-2-yl}azetidin-3-yl)-3-[(1-methyl-1H-pyrazol-4-yl)amino]pyrazine-2-carboxamide

A mixture of Example 6A (50 mg, 0.107 mmol), potassium trifluoro(oxetan-3-ylmethyl)borate (24.65 mg, 0.139 mmol), dicyclohexyl(2′,4′,6′-triisopropyl-[1,1′-biphenyl]-2-yl)phosphine (5.08 mg, 10.65 μmol), cesium carbonate (104 mg, 0.32 mmol) and diacetoxypalladium (1.196 mg, 5.33 μmol) was sparged with nitrogen for 30 minutes. Water (0.1 mL) and cyclopentyl methyl ether (1 mL) were added and the mixture was heated at 85° C. for 16 hours. The cooled mixture was partitioned between water and ethyl acetate, and the organic extracts were dried over Na₂SO₄, concentrated, and purified by silica gel chromatography eluting with 100/0 to 88/12 dichloromethane/methanol to afford the title compound (5.7 mg). ¹H NMR (400 MHz, dimethyl sulfoxide-d₆) δ ppm 10.67 (s, 1H), 8.36 (s, 1H), 8.35 (s, 1H), 8.01 (d, J=0.8 Hz, 1H), 7.84 (d, J=2.2 Hz, 1H), 7.57 (d, J=0.8 Hz, 1H), 7.20 (d, J=1.5 Hz, 1H), 7.16 (d, J=7.9 Hz, 1H), 6.95 (dd, J=8.0, 1.6 Hz, 1H), 4.42 (dd, J=7.7, 1.6 Hz, 4H), 4.34 (t, J=5.1 Hz, 2H), 4.21 (p, J=7.6 Hz, 1H), 3.78 (s, 3H), 3.39-3.24 (m, 4H), 2.57 (d, J=7.1 Hz, 2H), 1.70 (p, J=6.3 Hz, 1H). LCMS (ESI+): m/z 479.02 (M+H)⁺.

### Example 26

### ethyl {[2-(3-{6-carbamoyl-5-[(1-methyl-1H-pyrazol-4-yl)amino]pyrazin-2-yl}azetidin-1-yl)-1,3-benzoxazol-6-yl]oxy}acetate

A mixture of Example 20 (0.036 g, 0.089 mmol) and potassium carbonate (0.018 g, 0.133 mmol) in N,N-dimethylformamide (0.5 mL) was stirred at ambient temperature for 15 minutes. Ethyl iodoacetate (0.025 g, 0.115 mmol) was added and the mixture was heated at 90° C. for 5 hours. The mixture was cooled, quenched with water, and diluted with ethyl acetate. The aqueous layer was washed with ethyl acetate, and the organic layers were washed with water, brine, dried over Na₂SO₄, and concentrated. The residue was purified by silica gel chromatography eluting with 100/0 to 100/10 dichloromethane/methanol to afford the title compound (26 mg). ¹H NMR (400 MHz, dimethyl sulfoxide-d₆) δ ppm 10.57 (s, 1H), 8.35 (s, 1H), 8.00 (s, 1H), 7.58 (d, J=0.8 Hz, 1H), 7.19 (d, J=8.6 Hz, 1H), 7.08 (d, J=2.4 Hz, 1H), 6.77 (dd, J=8.6, 2.5 Hz, 1H), 4.71 (s, 2H), 4.50-4.31 (m, 4H), 4.23 (d, J=8.5 Hz, 1H), 4.14 (q, J=7.1 Hz, 2H), 3.79 (s, 3H), 1.18 (t, J=7.1 Hz, 3H). LCMS (ESI+): m/z 493.4 (M+H)⁺.

### Example 27

### 6-[1-(6-fluoro-1,3-benzoxazol-2-yl)azetidin-3-yl]-3-{[1-(oxan-4-yl)-1H-pyrazol-4-yl]amino}pyrazine-2-carboxamide

### Example 27A

### methyl 3-chloro-6-[1-(6-fluoro-1,3-benzoxazol-2-yl)azetidin-3-yl]pyrazine-2-carboxylate

To a mixture of Intermediate 2B (500 mg, 1.46 mmol) and potassium carbonate (506 mg, 3.66 mmol) was added dimethylformamide (2 mL) followed by Example 36A (276 mg, 1.61 mmol). The mixture was stirred at ambient temperature overnight, diluted with water and extracted with ethyl acetate. The organic phases were washed with brine, dried over Na₂SO₄ and concentrated. The residue was purified by flash chromatography on silica gel eluting with 90/10 to 40/60 heptane/ethyl acetate to afford the title compound (0.41 g). LCMS (ESI+): m/z 363.1 (M+H)⁺.

### Example 27B

### methyl 6-[1-(6-fluoro-1,3-benzoxazol-2-yl)azetidin-3-yl]-3-{[1-(oxan-4-yl)-1H-pyrazol-4-yl]amino}pyrazine-2-carboxylate

A mixture of Example 27A (50 mg, 0.14 mmol), 1-(tetrahydro-2H-pyran-4-yl)-1H-pyrazol-4-amine (23 mg, 0.14 mmol), K₂CO₃ (191 mg, 1.38 mmol), diacetoxypalladium (1.5 mg, 0.0069 mmol) and (9,9-dimethyl-9H-xanthene-4,5-diyl) bis(diphenylphosphine) (8.0 mg, 0.014 mmol) was degassed with nitrogen for 30 minutes. Dioxane (0.7 mL) was added, and the vial was degassed with nitrogen for an additional 5 minutes. The mixture was heated at 80° C. overnight, cooled, diluted with water, and extracted with ethyl acetate. The extracts were dried over Na₂SO₄, concentrated, and purified by flash chromatography on silica gel eluting with 100/0 to 95/5 dichloromethane/methanol to afford the title compound (34 mg). LCMS (ESI+): m/z 494.3 (M+H)⁺.

### Example 27C

### 6-[1-(6-fluoro-1,3-benzoxazol-2-yl)azetidin-3-yl]-3-{[1-(oxan-4-yl)-1H-pyrazol-4-yl]amino}pyrazine-2-carboxamide

A mixture of Example 27B (34 mg, 0.069 mmol) and 7 M ammonia in methanol (1.0 mL, 7.0 mmol) was heated at 55° C. for 3 hours. After cooling, the precipitate was collected by filtration and rinsed with methanol to afford the title compound (19 mg). ¹H NMR (501 MHz, dimethyl sulfoxide-d₆) δ ppm 10.72 (s, 1H), 8.41 (d, J=2.2 Hz, 1H), 8.38 (s, 1H), 8.11 (d, J=0.7 Hz, 1H), 7.88 (d, J=2.2 Hz, 1H), 7.67 (d, J=0.7 Hz, 1H), 7.44 (dd, J=8.5, 2.5 Hz, 1H), 7.28 (dd, J=8.6, 4.9 Hz, 1H), 7.02 (ddd, J=10.2, 8.6, 2.5 Hz, 1H), 4.49-4.43 (m, 4H), 4.36 (tt, J=10.2, 5.0 Hz, 1H), 4.25 (p, J=7.6 Hz, 1H), 3.98-3.92 (m, 2H), 3.45 (td, J=11.4, 3.1 Hz, 2H), 1.98-1.87 (m, 4H). LCMS (ESI+): m/z 479.4 (M+H)⁺.

### Example 28

### 6-[1-(6-fluoro-1,3-benzoxazol-2-yl)azetidin-3-yl]-3-{[1-(2-hydroxy-2-methylpropyl)-1H-pyrazol-4-yl]amino}pyrazine-2-carboxamide

The title compound was prepared as described in Example 27B using 1-(4-amino-1H-pyrazol-1-yl)-2-methylpropan-2-ol in place of 1-(tetrahydro-2H-pyran-4-yl)-1H-pyrazol-4-amine. ¹H NMR (501 MHz, dimethyl sulfoxide-d₆) δ ppm 10.75 (s, 1H), 8.41 (d, J=2.3 Hz, 1H), 8.39 (s, 1H), 8.06 (s, 1H), 7.90-7.86 (m, 1H), 7.62 (s, 1H), 7.44 (dd, J=8.5, 2.5 Hz, 1H), 7.28 (dd, J=8.6, 4.9 Hz, 1H), 7.02 (ddd, J=10.9, 8.7, 2.6 Hz, 1H), 4.66 (s, 1H), 4.50-4.43 (m, 4H), 4.24 (p, J=7.7 Hz, 1H), 3.98 (s, 2H), 1.05 (s, 6H). LCMS (ESI+): m/z 467.2 (M+H)⁺.

### Example 29

### 6-[1-(1,3-benzoxazol-2-yl)azetidin-3-yl]-3-{[1-(2-methoxyethyl)-1H-pyrazol-4-yl]amino}pyrazine-2-carboxamide

To a solution of Example 14 (100 mg, 0.266 mmol) in N,N-dimethylformamide (1 mL) was added Cs₂CO₃ (104 mg, 0.319 mmol) and 1-bromo-2-methoxyethane (44.3 mg, 0.319 mmol) and the mixture was stirred at 50° C. for 5 hours. The mixture was filtered, and the filtrate purified by prep-HPLC to afford the title compound (62 mg). ¹H NMR (400 MHz, dimethyl sulfoxide-d₆) δ ppm 3.22 (s, 3H), 3.67 (t, J=5.48 Hz, 2H), 4.19-4.31 (m, 1H), 4.19-4.30 (m, 3H), 4.46-4.52 (m, 4H), 7.00-7.10 (m, 1H), 7.18 (t, J=7.45 Hz, 1H), 7.32 (d, J=7.89 Hz, 1H), 7.44 (d, J=7.89 Hz, 1H), 7.64 (s, 1H), 7.92 (s, 1H), 8.08 (s, 1H), 8.40 (s, 1H), 8.45 (br s, 1H), 10.75 (s, 1H).

### Example 30

### 6-[1-(1,3-benzoxazol-2-yl)azetidin-3-yl]-3-{[1-(2-hydroxy-2-methylpropyl)-1H-pyrazol-4-yl]amino}pyrazine-2-carboxamide

To a solution of Example 14 (200 mg, 0.53 mmol) and 2,2-dimethyloxirane (115 mg, 1.59 mmol) in N,N-dimethylformamide (3 mL) was added cesium carbonate (346 mg, 1.06 mmol) and the mixture was stirred at 120° C. for 3 hours. The mixture was quenched with water and extracted with ethyl acetate. The organic extracts were washed with brine, dried over Na₂SO₄, concentrated, and purified by prep-HPLC to afford the title compound (86 mg). ¹H NMR (400 MHz, CD₃OD) δ ppm 1.18 (s, 6H), 4.07 (s, 2H), 4.31-4.42 (m, 1H), 4.60-4.69 (m, 2H), 4.70-4.78 (m, 2H), 7.21-7.27 (m, 1H), 7.33 (t, J=7.28 Hz, 1H), 7.37-7.42 (m, 1H), 7.48 (d, J=8.16 Hz, 1H), 7.70 (s, 1H), 8.12 (s, 1H), 8.33 (s, 1H). LCMS (ESI+): m/z 449.1 (M+H)⁺.

### Example 31

### 6-{1-[6-(3-hydroxy-3-methylbutoxy)-1,3-benzoxazol-2-yl]azetidin-3-yl}-3-[(1-methyl-1H-pyrazol-4-yl)amino]pyrazine-2-carboxamide

A mixture of Example 20 (15.0 g, 36.9 mmol) and sodium hydride (2.21 g, 55.4 mmol, 60% dispersion in mineral oil) in N,N-dimethylformamide (130 mL) was stirred at ambient temperature for 15 minutes. 4-Bromo-2-methylbutane-2-ol (8.22 g mg, 44.3 mmol) in N,N-dimethylformamide (20 mL) was added, and the mixture was stirred at 70° C. for 16 hours. The mixture was cooled to ambient temperature, quenched with cold water, and diluted with ethyl acetate. The organic layer was washed with water, and the aqueous layer was washed with ethyl acetate. The combined organic layers were dried over Na₂SO₄ and concentrated. The residue was purified by silica gel chromatography, eluting with 100:0 to 100:20 dichloromethane/methanol to afford the title compound (12 g). ¹H NMR (501 MHz, dimethyl sulfoxide-d₆) δ ppm 10.70 (s, 1H), 8.38 (s, 2H), 8.04 (s, 1H), 7.88 (d, J=2.2 Hz, 1H), 7.60 (d, J=0.8 Hz, 1H), 7.19 (d, J=8.5 Hz, 1H), 7.10 (d, J=2.4 Hz, 1H), 6.76 (dd, J=8.6, 2.4 Hz, 1H), 4.49-4.39 (m, 4H), 4.36 (s, 1H), 4.23 (p, J=7.7 Hz, 1H), 4.06 (t, J=7.2 Hz, 2H), 3.81 (s, 3H), 1.83 (t, J=7.2 Hz, 2H), 1.16 (s, 6H). LCMS (ESI+): m/z 493.3 (M+H)⁺.

### Example 32

### 6-{1-[6-(3-hydroxypropyl)-1,3-benzoxazol-2-yl]azetidin-3-yl}-3-[(1-methyl-1H-pyrazol-4-yl)amino]pyrazine-2-carboxamide

### Example 32A

### 6-(1-{6-[(1E)-3-hydroxyprop-1-en-1-yl]-1,3-benzoxazol-2-yl}azetidin-3-yl)-3-[(1-methyl-1H-pyrazol-4-yl)amino]pyrazine-2-carboxamide

The title compound was prepared as described in Example 17 using (E)-3-(4,4,5,5-tetramethyl-1,3,2-dioxaborolan-2-yl)prop-2-en-1-ol in place of (E)-2-methyl-4-(4,4,5,5-tetramethyl-1,3,2-dioxaborolane-2-yl)but-3-en-2-ol. ¹H NMR (400 MHz, dimethyl sulfoxide-d₆) δ ppm 10.68 (s, 1H), 8.40-8.36 (m, 1H), 8.35 (s, 1H), 8.01 (d, J=0.8 Hz, 1H), 7.85 (d, J=2.1 Hz, 1H), 7.57 (d, J=0.8 Hz, 1H), 7.54 (s, 1H), 7.25-7.17 (m, 2H), 6.59 (dt, J=16.0, 1.5 Hz, 1H), 6.25 (dt, J=15.9, 5.9 Hz, 1H), 4.45 (d, J=7.7 Hz, 4H), 4.22 (p, J=7.6 Hz, 1H), 3.99 (dd, J=6.0, 1.4 Hz, 2H), 3.78 (s, 3H), 3.24 (s, 3H). LCMS (ESI+): m/z 447.3 (M+H)⁺.

### Example 32B

### 6-{1-[6-(3-hydroxypropyl)-1,3-benzoxazol-2-yl]azetidin-3-yl}-3-[(1-methyl-1H-pyrazol-4-yl)amino]pyrazine-2-carboxamide

The title compound was prepared as described in Example 7 using Example 32A in place of Example 6. ¹H NMR (400 MHz, dimethyl sulfoxide-d₆) δ ppm 10.66 (s, 1H), 8.37-8.32 (m, 2H), 8.00 (d, J=0.8 Hz, 1H), 7.84 (d, J=2.2 Hz, 1H), 7.56 (d, J=0.8 Hz, 1H), 7.22 (d, J=1.5 Hz, 1H), 7.16 (d, J=8.0 Hz, 1H), 6.96 (dd, J=8.0, 1.6 Hz, 1H), 4.47-4.38 (m, 5H), 4.21 (p, J=7.6 Hz, 1H), 3.78 (s, 3H), 3.40-3.35 (m, 2H), 2.65-2.57 (m, 2H), 1.73-1.62 (m, 2H). LCMS (ESI+): m/z 449.3 (M+H)⁺.

### Example 33

### 6-{1-[6-(3-methoxypropyl)-1,3-benzoxazol-2-yl]azetidin-3-yl}-3-[(1-methyl-1H-pyrazol-4-yl)amino]pyrazine-2-carboxamide

### Example 33A

### 6-(1-{6-[(1E)-3-methoxyprop-1-en-1-yl]-1,3-benzoxazol-2-yl}azetidin-3-yl)-3-[(1-methyl-1H-pyrazol-4-yl)amino]pyrazine-2-carboxamide

The title compound was prepared as described in Example 17 using (E)-2-(3-methoxyprop-1-en-1-yl)-4,4,5,5-tetramethyl-1,3,2-dioxaborolane in place of (E)-2-methyl-4-(4,4,5,5-tetramethyl-1,3,2-dioxaborolane-2-yl)but-3-en-2-ol. ¹H NMR (400 MHz, dimethyl sulfoxide-d₆) δ ppm 10.68 (s, 1H), 8.40-8.36 (m, 1H), 8.35 (s, 1H), 8.01 (d, J=0.8 Hz, 1H), 7.85 (d, J=2.1 Hz, 1H), 7.57 (d, J=0.8 Hz, 1H), 7.54 (s, 1H), 7.25-7.17 (m, 2H), 6.59 (dt, J=16.0, 1.5 Hz, 1H), 6.25 (dt, J=15.9, 5.9 Hz, 1H), 4.45 (d, J=7.7 Hz, 4H), 4.22 (p, J=7.6 Hz, 1H), 3.99 (dd, J=6.0, 1.4 Hz, 2H), 3.78 (s, 3H), 3.24 (s, 3H). LCMS (ESI+): m/z 461.2 (M+H)⁺.

### Example 33B

### 6-{1-[6-(3-methoxypropyl)-1,3-benzoxazol-2-yl]azetidin-3-yl}-3-[(1-methyl-1H-pyrazol-4-yl)amino]pyrazine-2-carboxamide

The title compound was prepared as described in Example 7 using Example 33A in place of Example 6. ¹H NMR (500 MHz, dimethyl sulfoxide-d₆) δ ppm 10.71 (s, 1H), 8.41-8.39 (d, J=2.4 Hz, 1H), 8.39 (s, 1H), 8.05 (s, 1H), 7.89 (d, J=2.4 Hz, 1H), 7.61 (s, 1H), 7.27 (d, J=1.6 Hz, 1H), 7.21 (d, J=7.9 Hz, 1H), 7.00 (dd, J=8.0, 1.6 Hz, 1H), 4.46 (dd, J=7.7, 1.6 Hz, 4H), 4.25 (p, J=7.6 Hz, 1H), 3.82 (s, 3H), 3.34-3.28 (m, 2H), 3.23 (s, 3H), 2.66 (dd, J=8.8, 6.6 Hz, 2H), 1.84-1.75 (m, 2H). LCMS (ESI+): m/z 463.3 (M+H)⁺.

### Example 34

### 6-[1-(1,3-benzoxazol-2-yl)azetidin-3-yl]-3-({1-[2-(dimethylamino)-2-oxoethyl]-1H-pyrazol-4-yl}amino)pyrazine-2-carboxamide

The title compound was prepared as described in Example 29 using 2-bromo-N,N-dimethylacetamide in place of 1-bromo-2-methoxyethane. ¹H NMR (400 MHz, dimethyl sulfoxide-d₆) δ ppm 2.85 (s, 3H), 3.02 (s, 3H), 4.23-4.32 (m, 1H), 4.47-4.53 (m, 4H), 5.08 (s, 2H), 7.04-7.09 (m, 1H), 7.19 (t, J=7.64 Hz, 1H), 7.33 (d, J=7.70 Hz, 1H), 7.45 (d, J=7.83 Hz, 1H), 7.62 (s, 1H), 7.91 (s, 1H), 8.05 (s, 1H), 8.39 (s, 1H), 8.44 (br s, 1H), 10.76-10.81 (m, 1H), 10.76-10.81 (m, 1H), 10.78 (s, 1H). LCMS (ESI+): m/z 462.3 (M+H)⁺.

### Example 35

### 6-{1-[6-(2-methoxyethoxy)-1,3-benzoxazol-2-yl]azetidin-3-yl}-3-[(1-methyl-1H-pyrazol-4-yl)amino]pyrazine-2-carboxamide

The title compound was prepared as described in Example 31 using 2-bromoethyl methyl ether in place of 4-bromo-2-methylbutane-2-ol. ¹H NMR (400 MHz, dimethyl sulfoxide-d₆) δ ppm 10.70 (s, 1H), 8.38 (d, J=2.8 Hz, 2H), 8.06-8.00 (m, 1H), 7.88 (d, J=2.3 Hz, 1H), 7.60 (d, J=0.8 Hz, 1H), 7.19 (d, J=8.5 Hz, 1H), 7.13 (d, J=2.4 Hz, 1H), 6.78 (dd, J=8.6, 2.5 Hz, 1H), 4.48-4.39 (m, 4H), 4.24 (q, J=7.6 Hz, 1H), 4.11-4.03 (m, 2H), 3.82 (s, 3H), 3.68-3.61 (m, 2H), 3.31 (s, 3H). LCMS (ESI+): m/z 465.3 (M+H)⁺.

### Example 36

### 3-{[1-(2,3-dihydroxypropyl)-1H-pyrazol-4-yl]amino}-6-[1-(6-fluoro-1,3-benzoxazol-2-yl)azetidin-3-yl]pyrazine-2-carboxamide

### Example 36A

### 2-chloro-6-fluoro-1,3-benzoxazole

The title compound was prepared as described in Examples 3A and 3B using 2-amino-5-fluorophenol in place of 2-amino-5-methylphenol. LCMS (ESI+): m/z 172.2 (M+H)⁺.

### Example 36B

### methyl 3-chloro-6-(1-(6-fluorobenzo[d]oxazol-2-yl)azetidin-3-yl)pyrazine-2-carboxylate

The title compound was prepared as described in Example 1 using Example 36A in place of 2-chlorobenzo[d]oxazole and Intermediate 2B in place of Intermediate 1. LCMS (ESI+): m/z 363.1 (M+H)⁺.

### Example 36C

### 6-[1-(6-fluoro-1,3-benzoxazol-2-yl)azetidin-3-yl]-3-[(1H-pyrazol-4-yl)amino]pyrazine-2-carboxamide

The title compound was prepared as described in Example 27B using tert-butyl 4-amino-1H-pyrazole-1-carboxylate in place of 1-(tetrahydro-2H-pyran-4-yl)-1H-pyrazol-4-amine. ¹H NMR (400 MHz, dimethyl sulfoxide-d₆) δ ppm 12.57 (s, 1H), 10.67 (s, 1H), 8.36 (s, 1H), 8.35 (s, 1H), 8.00-7.70 (br m, 2H), 7.84 (d, J=2.2 Hz, 1H), 7.41 (dd, J=8.5, 2.5 Hz, 1H), 7.25 (dd, J=8.6, 5.0 Hz, 1H), 6.99 (ddd, J=10.2, 8.6, 2.6 Hz, 1H), 4.43 (dd, J=7.7, 1.2 Hz, 4H), 4.21 (p, J=7.6 Hz, 1H). LCMS (ESI+): m/z 395.2 (M+H)⁺.

### Example 36D

### 3-({1-[(2,2-dimethyl-1,3-dioxolan-4-yl)methyl]-1H-pyrazol-4-yl}amino)-6-[1-(6-fluoro-1,3-benzoxazol-2-yl)azetidin-3-yl]pyrazine-2-carboxamide

The title compound was prepared as described in Example 29 using Example 36C in place of Example 14 and 4-(bromomethyl)-2,2-dimethyl-1,3-dioxolane in place of 1-bromo-2-methoxyethane. ¹H NMR (400 MHz, dimethyl sulfoxide-d₆) δ ppm 10.70 (s, 1H), 8.40-8.37 (m, 1H), 8.35 (s, 1H), 8.07 (d, J=0.7 Hz, 1H), 7.85 (d, J=2.3 Hz, 1H), 7.62 (d, J=0.8 Hz, 1H), 7.41 (dd, J=8.5, 2.5 Hz, 1H), 7.25 (dd, J=8.6, 4.9 Hz, 1H), 6.99 (ddd, J=10.1, 8.6, 2.6 Hz, 1H), 4.48-4.40 (m, 4H), 4.38-4.31 (m, 1H), 4.27-4.12 (m, 3H), 3.97 (dd, J=8.4, 6.3 Hz, 1H), 3.67 (dd, J=8.5, 5.9 Hz, 1H), 1.28 (s, 3H), 1.22 (s, 3H). LCMS (ESI+): m/z 509.2 (M+H)⁺.

### Example 36E

### 3-{[1-(2,3-dihydroxypropyl)-1H-pyrazol-4-yl]amino}-6-[1-(6-fluoro-1,3-benzoxazol-2-yl)azetidin-3-yl]pyrazine-2-carboxamide

A mixture of Example 36D (27 mg, 0.053 mmol), 4-methylbenzenesulfonic acid hydrate (14 mg, 0.074 mmol), tetrahydrofuran (0.53 mL) and water (0.53 mL) was stirred at 20° C. for 1 hour and at 50° C. for 4 hours. The cooled mixture was diluted with water and saturated NaHCO₃ and allowed to stand for 15 minutes. The precipitate was collected by filtration and rinsed with water and ethyl acetate to afford the title compound (15 mg). ¹H NMR (400 MHz, dimethyl sulfoxide-d₆) δ ppm 10.70 (s, 1H), 8.41-8.37 (m, 1H), 8.35 (s, 1H), 8.03 (d, J=0.8 Hz, 1H), 7.88-7.81 (m, 1H), 7.59 (d, J=0.8 Hz, 1H), 7.41 (dd, J=8.5, 2.5 Hz, 1H), 7.25 (dd, J=8.6, 4.9 Hz, 1H), 6.99 (ddd, J=10.2, 8.6, 2.5 Hz, 1H), 4.91 (d, J=5.3 Hz, 1H), 4.67 (t, J=5.6 Hz, 1H), 4.44 (dd, J=7.7, 1.7 Hz, 4H), 4.21 (p, J=7.8 Hz, 1H), 4.15 (dd, J=13.9, 4.1 Hz, 1H), 3.92 (dd, J=13.8, 7.6 Hz, 1H), 3.80-3.71 (m, 1H), 3.28-3.21 (m, 2H). LCMS (ESI+): m/z 469.3 (M+H)⁺.

### Example 37

### 6-{1-[6-(2-amino-2-oxoethoxy)-1,3-benzoxazol-2-yl]azetidin-3-yl}-3-[(1-methyl-1H-pyrazol-4-yl)amino]pyrazine-2-carboxamide

The title compound was prepared as described in Example 31 using 2-bromoacetamide in place of 4-bromo-2-methylbutane-2-ol. ¹H NMR (400 MHz, dimethyl sulfoxide-d₆) δ ppm 10.67 (s, 1H), 8.34 (s, 2H), 8.00 (d, J=0.8 Hz, 1H), 7.92-7.79 (m, 1H), 7.57 (d, J=0.8 Hz, 1H), 7.42 (d, J=47.9 Hz, 2H), 7.18 (d, J=8.6 Hz, 1H), 7.10 (d, J=2.4 Hz, 1H), 6.79 (dd, J=8.6, 2.4 Hz, 1H), 4.41 (dd, J=7.7, 1.5 Hz, 4H), 4.37 (s, 2H), 4.29-4.13 (m, 1H), 3.78 (s, 3H). LCMS (ESI+): m/z 464.3 (M+H)⁺.

### Example 38

### 6-{1-[6-(2-hydroxy-2-methylpropoxy)-1,3-benzoxazol-2-yl]azetidin-3-yl}-3-[(1-methyl-1H-pyrazol-4-yl)amino]pyrazine-2-carboxamide

The title compound was prepared as described in Example 31 using 1-bromo-2-methylpropan-2-ol in place of 4-bromo-2-methylbutane-2-ol. ¹H NMR (400 MHz, dimethyl sulfoxide-d₆) δ ppm 10.67 (s, 1H), 8.34 (d, J=1.7 Hz, 2H), 8.01 (d, J=0.7 Hz, 1H), 7.85 (d, J=2.2 Hz, 1H), 7.57 (d, J=0.8 Hz, 1H), 7.15 (d, J=8.6 Hz, 1H), 7.07 (d, J=2.3 Hz, 1H), 6.82-6.68 (m, 1H), 4.57 (s, 1H), 4.41 (d, J=7.8 Hz, 4H), 4.20 (p, J=7.5 Hz, 1H), 3.78 (s, 3H), 3.66 (s, 2H), 1.17 (s, 6H). LCMS (ESI+): m/z 479.4 (M+H)⁺.

### Example 39

### 6-(1-{6-[2-(dimethylamino)-2-oxoethoxy]-1,3-benzoxazol-2-yl}azetidin-3-yl)-3-[(1-methyl-1H-pyrazol-4-yl)amino]pyrazine-2-carboxamide

The title compound was prepared as described in Example 31 using 2-bromo-N,N-dimethylacetamide in place of 4-bromo-2-methylbutane-2-ol. ¹H NMR (501 MHz, dimethyl sulfoxide-d₆) δ ppm 10.70 (s, 1H), 8.38 (s, 2H), 8.04 (d, J=0.8 Hz, 1H), 7.88 (d, J=2.4 Hz, 1H), 7.60 (d, J=0.8 Hz, 1H), 7.19 (d, J=8.6 Hz, 1H), 7.11 (s, 1H), 6.78 (dd, J=8.6, 2.5 Hz, 1H), 4.77 (s, 2H), 4.51-4.37 (m, 4H), 4.24 (p, J=7.6 Hz, 1H), 3.82 (s, 3H), 3.00 (s, 3H), 2.84 (s, 3H). LCMS (ESI+): m/z 492.4 (M+H)⁺.

### Example 40

### 6-{1-[6-(2-hydroxyethoxy)-1,3-benzoxazol-2-yl]azetidin-3-yl}-3-[(1-methyl-1H-pyrazol-4-yl)amino]pyrazine-2-carboxamide

The title compound was prepared as described in Example 31 using 2-bromoethanol in place of 4-bromo-2-methylbutane-2-ol. ¹H NMR (400 MHz, dimethyl sulfoxide-d₆) δ ppm 10.70 (s, 1H), 8.38 (s, 2H), 8.04 (d, J=0.8 Hz, 1H), 7.88 (s, 1H), 7.60 (d, J=0.8 Hz, 1H), 7.19 (d, J=8.6 Hz, 1H), 7.12 (d, J=2.4 Hz, 1H), 6.86-6.71 (m, 1H), 4.84 (t, J=5.5 Hz, 1H), 4.44 (dd, J=7.6, 1.3 Hz, 4H), 4.24 (p, J=7.6 Hz, 1H), 4.02-3.86 (m, 2H), 3.82 (s, 3H), 3.71 (q, J=5.2 Hz, 2H). LCMS (ESI+): m/z 451.2 (M+H)⁺.

### Example 41

### 3-[(1-methyl-1H-pyrazol-4-yl)amino]-6-(1-{6-[(oxetan-3-yl)methoxy]-1,3-benzoxazol-2-yl}azetidin-3-yl)pyrazine-2-carboxamide

The title compound was prepared as described in Example 31 using 3-(bromomethyl)oxetane in place of 4-bromo-2-methylbutane-2-ol. ¹H NMR (500 MHz, dimethyl sulfoxide-d₆) δ ppm 10.71 (s, 1H), 8.39 (s, 2H), 8.05 (d, J=0.8 Hz, 1H), 7.95-7.79 (m, 1H), 7.61 (d, J=0.8 Hz, 1H), 7.27-7.06 (m, 2H), 6.81 (dd, J=8.6, 2.4 Hz, 1H), 4.71 (dd, J=7.9, 6.0 Hz, 2H), 4.52-4.36 (m, 6H), 4.25 (q, J=7.7 Hz, 1H), 4.19 (d, J=6.7 Hz, 2H), 3.38 (s, 1H). LCMS (ESI+): m/z 477.4 (M+H)⁺.

### Example 42

### 6-(1-{6-[(3-hydroxycyclobutyl)methoxy]-1,3-benzoxazol-2-yl}azetidin-3-yl)-3-[(1-methyl-1H-pyrazol-4-yl)amino]pyrazine-2-carboxamide

A mixture of Example 20 (0.05 g, 0.123 mmol) and cesium carbonate (0.08 g, 0.246 mmol) in N,N-dimethylformamide (0.5 mL) was stirred at ambient temperature for 15 minutes. (3-Hydroxycyclobutyl)methyl 4-methylbenzenesulfonate (0.038 g, 0.148 mmol) was added and the mixture was heated at 90° C. for 16 hours. The mixture was cooled, quenched with water, and diluted with ethyl acetate. The aqueous layer was washed with ethyl acetate, and the organic layers were washed with water and brine, dried over Na₂SO₄ and concentrated. The residue was purified by silica gel chromatography eluting with 100:0 to 100:10 dichloromethane/methanol to afford the title compound (23 mg). ¹H NMR (500 MHz, dimethyl sulfoxide-d₆) δ ppm 10.71 (s, 1H), 8.39 (d, J=3.5 Hz, 2H), 8.05 (s, 1H), 7.89 (d, J=2.4 Hz, 1H), 7.61 (d, J=0.8 Hz, 1H), 7.19 (d, J=8.5 Hz, 1H), 7.10 (d, J=2.4 Hz, 1H), 6.81-6.62 (m, 1H), 5.04 (d, J=6.4 Hz, 1H), 4.50-4.39 (m, 4H), 4.24 (p, J=7.6 Hz, 1H), 4.07-3.92 (m, 1H), 3.90 (d, J=6.4 Hz, 2H), 3.82 (s, 3H), 2.31 (dddd, J=11.0, 9.4, 5.0, 2.6 Hz, 2H), 2.10 (dtd, J=13.5, 6.6, 2.6 Hz, 1H), 1.69-1.58 (m, 2H). LCMS (ESI+): m/z 491.5 (M+H)⁺.

### Example 43

### 6-{1-[6-(2,3-dihydroxypropoxy)-1,3-benzoxazol-2-yl]azetidin-3-yl}-3-[(1-methyl-1H-pyrazol-4-yl)amino]pyrazine-2-carboxamide

A mixture of Example 20 (0.05 g, 0.123 mmol) and sodium hydride (7.4 mg, 0.135 mmol, 60% dispersion in mineral oil) in N,N-dimethylformamide (0.5 mL) was stirred at ambient temperature for 15 minutes. 4-(Bromomethyl)-2,2-dimethyl-1,3-dioxalane (0.029 g, 0.148 mmol) was added and the mixture was stirred at 70° C. for 16 hours. The mixture was quenched with water and diluted with ethyl acetate. The aqueous layer was washed with ethyl acetate, and the organic layers were washed with water and brine, dried over Na₂SO₄ and concentrated. To the residue was added tetrahydrofuran (0.5 mL) and 4-methylbenzenesulfonic acid (21.2 mg, 0.123 mmol), and the mixture was heated to 50° C. for 4 hours. The mixture was cooled, concentrated, and purified by silica gel chromatography eluting with 100:0 to 100:10 dichloromethane/methanol to afford the title compound (21 mg). ¹H NMR (501 MHz, dimethyl sulfoxide-d₆) δ ppm 10.70 (s, 1H), 8.38 (d, J=3.7 Hz, 2H), 8.04 (d, J=0.8 Hz, 1H), 7.88 (s, 1H), 7.60 (d, J=0.7 Hz, 1H), 7.19 (d, J=8.5 Hz, 1H), 7.10 (d, J=2.4 Hz, 1H), 6.77 (dd, J=8.6, 2.4 Hz, 1H), 4.92 (d, J=5.0 Hz, 1H), 4.64 (t, J=5.7 Hz, 1H), 4.50-4.37 (m, 4H), 4.23 (p, J=7.7 Hz, 1H), 3.97 (dd, J=9.9, 4.2 Hz, 1H), 3.85 (dd, J=9.8, 6.0 Hz, 1H), 3.81 (s, 3H), 3.77 (dt, J=10.9, 5.4 Hz, 1H), 3.44 (td, J=5.6, 1.2 Hz, 2H). LCMS (ESI+): m/z 481.4 (M+H)⁺.

### Example 44

### 6-(1-{6-[(1-hydroxycyclobutyl)methoxy]-1,3-benzoxazol-2-yl}azetidin-3-yl)-3-[(1-methyl-1H-pyrazol-4-yl)amino]pyrazine-2-carboxamide

The title compound was prepared as described in Example 31 using 1-(bromomethyl)cyclobutan-1-ol in place of 4-bromo-2-methylbutane-2-ol. ¹H NMR (501 MHz, dimethyl sulfoxide-d₆) δ ppm 10.70 (s, 1H), 8.38 (s, 2H), 8.04 (d, J=0.8 Hz, 1H), 7.88 (s, 1H), 7.60 (d, J=0.8 Hz, 1H), 7.19 (d, J=8.5 Hz, 1H), 7.14 (d, J=2.4 Hz, 1H), 6.80 (dd, J=8.5, 2.4 Hz, 1H), 5.20 (s, 1H), 4.44 (dd, J=7.7, 1.4 Hz, 4H), 4.23 (p, J=7.6 Hz, 1H), 3.88 (s, 2H), 3.82 (s, 3H), 2.11 (tt, J=8.9, 3.1 Hz, 2H), 2.00 (d, J=11.5 Hz, 2H), 1.75-1.64 (m, 1H), 1.56 (dt, J=11.1, 8.8 Hz, 1H). LCMS (ESI+): m/z 491.2 (M+H)⁺.

### Example 45

### 6-(1-{6-[2-(3-hydroxyoxetan-3-yl)ethyl]-1,3-benzoxazol-2-yl}azetidin-3-yl)-3-[(1-methyl-1H-pyrazol-4-yl)amino]pyrazine-2-carboxamide

### Example 45A

### 6-(1-{6-[(3-hydroxyoxetan-3-yl)ethynyl]-1,3-benzoxazol-2-yl}azetidin-3-yl)-3-[(1-methyl-1H-pyrazol-4-yl)amino]pyrazine-2-carboxamide

A mixture of Example 6A (50 mg, 0.107 mmol), 3-ethynyloxetan-3-ol (20.90 mg, 0.213 mmol), copper(I) iodide (2.0 mg, 11 μmol), and tetrakis(triphenylphosphine)palladium(0) (6.2 mg, 5.3 μmol) was sparged with nitrogen. N,N-Dimethylformamide (0.5 mL), and triethylamine (0.04 mL, 0.287 mmol) were added, and nitrogen was bubbled through the mixture for 5 minutes. The mixture was heated at 90° C. for 16 hours and partitioned between water and ethyl acetate. The organic extracts were dried over Na₂SO₄, concentrated and purified by flash chromatography on silica gel eluting with 100/0 to 94/6 dichloromethane/methanol to afford the title compound (22 mg). LCMS (ESI+): m/z 487.4 (M+H)⁺.

### Example 45B

### 6-(1-{6-[2-(3-hydroxyoxetan-3-yl)ethyl]-1,3-benzoxazol-2-yl}azetidin-3-yl)-3-[(1-methyl-1H-pyrazol-4-yl)amino]pyrazine-2-carboxamide

The title compound was prepared as described in Example 7 using Example 45A in place of Example 6. ¹H NMR (400 MHz, dimethyl sulfoxide-d₆) δ ppm 10.67 (s, 1H), 8.38-8.32 (m, 2H), 8.01 (d, J=0.8 Hz, 1H), 7.85 (d, J=2.4 Hz, 1H), 7.57 (d, J=0.9 Hz, 1H), 7.27 (d, J=1.6 Hz, 1H), 7.17 (d, J=7.9 Hz, 1H), 7.01 (dd, J=8.0, 1.6 Hz, 1H), 5.61 (s, 1H), 4.43 (d, J=7.7 Hz, 4H), 4.36 (d, J=6.2 Hz, 2H), 4.27 (d, J=6.2 Hz, 2H), 4.22 (q, J=7.6 Hz, 1H), 3.78 (s, 3H), 2.69-2.61 (m, 2H), 2.01-1.88 (m, 2H). LCMS (ESI+): m/z 491.3 (M+H)⁺.

### Example 46

### 3-[(1-methyl-1H-pyrazol-4-yl)amino]-6-{1-[6-(morpholine-4-carbonyl)-1,3-benzoxazol-2-yl]azetidin-3-yl}pyrazine-2-carboxamide

The title compound was prepared as described in Example 18B using morpholine in place of dimethylamine hydrochloride. ¹H NMR (400 MHz, dimethyl sulfoxide-d₆) δ ppm 10.68 (s, 1H), 8.39 (s, 1H), 8.35 (s, 1H), 8.01 (s, 1H), 7.86 (s, 1H), 7.57 (s, 1H), 7.50-7.44 (m, 1H), 7.30 (d, J=8.0 Hz, 1H), 7.21 (dd, J=8.1, 1.5 Hz, 1H), 4.47 (d, J=7.7 Hz, 4H), 4.24 (p, J=7.7 Hz, 1H), 3.78 (s, 3H), 3.61-3.37 (m, 8H). LCMS (ESI+): m/z 504.3 (M+H)⁺.

### Example 47

### 6-(1-{6-[2-(dimethylamino)-2-oxoethyl]-1,3-benzoxazol-2-yl}azetidin-3-yl)-3-[(1-methyl-1H-pyrazol-4-yl)amino]pyrazine-2-carboxamide

The title compound was prepared as described in Example 18B using Example 65C in place of Example 18A. ¹H NMR (400 MHz, dimethyl sulfoxide-d₆) δ ppm 10.67 (s, 1H), 8.37-8.32 (m, 2H), 8.01 (s, 1H), 7.84 (s, 1H), 7.57 (d, J=0.8 Hz, 1H), 7.23 (d, J=1.6 Hz, 1H), 7.19 (d, J=8.0 Hz, 1H), 6.98 (dd, J=7.9, 1.6 Hz, 1H), 4.50-4.37 (m, 4H), 4.22 (p, J=7.6 Hz, 1H), 3.78 (s, 3H), 3.68 (s, 2H), 2.97 (s, 3H), 2.79 (s, 3H). LCMS (ESI+): m/z 476.3 (M+H)⁺.

### Example 48

### 6-[1-(6-{[(2R)-1,4-dioxan-2-yl]methoxy}-1,3-benzoxazol-2-yl)azetidin-3-yl]-3-[(1-methyl-1H-pyrazol-4-yl)amino]pyrazine-2-carboxamide

The title compound was prepared as described in Example 31 using [(2R)-1,4-dioxan-2-yl]methyl 4-methylbenzene-1-sulfonate in place of 4-bromo-2-methylbutane-2-ol. ¹H NMR (400 MHz, dimethyl sulfoxide-d₆) δ ppm 10.67 (s, 1H), 8.34 (s, 2H), 8.00 (d, J=0.8 Hz, 1H), 7.85 (d, J=2.2 Hz, 1H), 7.57 (d, J=0.8 Hz, 1H), 7.16 (d, J=8.5 Hz, 1H), 7.10 (d, J=2.4 Hz, 1H), 6.74 (dd, J=8.6, 2.4 Hz, 1H), 4.45-4.34 (m, 4H), 4.20 (p, J=7.6 Hz, 1H), 3.94-3.86 (m, 2H), 3.85-3.78 (m, 2H), 3.78 (s, 3H), 3.76-3.69 (m, 1H), 3.66-3.54 (m, 2H), 3.52-3.42 (m, 1H), 3.40-3.32 (m, 1H). LCMS (ESI+): m/z 507.3 (M+H)⁺.

### Example 49

### 3-[(1-methyl-1H-pyrazol-4-yl)amino]-6-(1-{6-[2-oxo-2-(pyrrolidin-1-yl)ethoxy]-1,3-benzoxazol-2-yl}azetidin-3-yl)pyrazine-2-carboxamide

The title compound was prepared as described in Example 31 using 2-bromo-1-(pyrrolidin-1-yl)ethan-1-one in place of 4-bromo-2-methylbutane-2-ol. ¹H NMR (400 MHz, dimethyl sulfoxide-d₆) δ ppm 10.67 (s, 1H), 8.34 (s, 2H), 8.00 (d, J=0.8 Hz, 1H), 7.84 (d, J=2.4 Hz, 1H), 7.57 (d, J=0.8 Hz, 1H), 7.16 (d, J=8.6 Hz, 1H), 7.08 (d, J=2.4 Hz, 1H), 6.74 (dd, J=8.6, 2.4 Hz, 1H), 4.65 (s, 2H), 4.47-4.35 (m, 4H), 4.20 (p, J=7.6 Hz, 1H), 3.78 (s, 3H), 3.43 (t, J=6.7 Hz, 2H), 1.94-1.82 (m, 2H), 1.80-1.62 (m, 2H). LCMS (ESI+): m/z 518.4 (M+H)⁺.

### Example 50

### 6-(1-{6-[2-(methylamino)-2-oxoethyl]-1,3-benzoxazol-2-yl}azetidin-3-yl)-3-[(1-methyl-1H-pyrazol-4-yl)amino]pyrazine-2-carboxamide

The title compound was prepared as described in Example 18B using Example 65C in place of Example 18A and methyl amine in place of dimethylamine hydrochloride. ¹H NMR (400 MHz, dimethyl sulfoxide-d₆) δ ppm 10.67 (s, 1H), 8.35 (br s, 2H), 8.01 (s, 1H), 7.88-7.82 (m, 2H), 7.57 (d, J=0.7 Hz, 1H), 7.27 (d, J=1.6 Hz, 1H), 7.18 (d, J=7.9 Hz, 1H), 7.01 (dd, J=8.0, 1.6 Hz, 1H), 4.43 (d, J=7.6 Hz, 4H), 4.26-4.16 (m, 1H), 3.78 (s, 3H), 3.38 (s, 2H), 2.53 (d, J=4.6 Hz, 3H). LCMS (ESI+): m/z 362.5 (M+H)⁺.

### Example 51

### 6-(1-{6-[3-hydroxy-2-(hydroxymethyl)propoxy]-1,3-benzoxazol-2-yl}azetidin-3-yl)-3-[(1-methyl-1H-pyrazol-4-yl)amino]pyrazine-2-carboxamide

### Example 51A

### 6-(1-{6-[(2,2-dimethyl-1,3-dioxan-5-yl)methoxy]-1,3-benzoxazol-2-yl}azetidin-3-yl)-3-[(1-methyl-1H-pyrazol-4-yl)amino]pyrazine-2-carboxamide

The title compound was prepared as described in Example 31 using 5-(bromomethyl)-2,2-dimethyl-1,3-dioxane in place of 4-bromo-2-methylbutane-2-ol. ¹H NMR (400 MHz, dimethyl sulfoxide-d₆) δ ppm 10.67 (s, 1H), 8.35 (s, 2H), 8.00 (d, J=0.8 Hz, 1H), 7.85 (s, 1H), 7.57 (d, J=0.8 Hz, 1H), 7.16 (d, J=8.5 Hz, 1H), 7.10 (d, J=2.4 Hz, 1H), 6.76 (dd, J=8.6, 2.4 Hz, 1H), 4.41 (d, J=7.8 Hz, 4H), 4.21 (q, J=7.6 Hz, 1H), 4.03-3.88 (m, 4H), 3.78 (s, 3H), 3.71 (dd, J=11.8, 6.0 Hz, 2H), 2.01 (s, 1H), 1.31 (d, J=11.1 Hz, 6H). LCMS (ESI+): m/z 535.2 (M+H)⁺.

### Example 51B

### 6-(1-{6-[3-hydroxy-2-(hydroxymethyl)propoxy]-1,3-benzoxazol-2-yl}azetidin-3-yl)-3-[(1-methyl-1H-pyrazol-4-yl)amino]pyrazine-2-carboxamide

To a solution of Example 51A (0.020 g, 0.037 mmol) in tetrahydrofuran (374 μL) was added para-toluenesulfonic acid (8.38 mg, 0.049 mmol) and the mixture was stirred at 50° C. for 2 hours. The mixture was concentrated, quenched with water, and acidified with dilute acetic acid. The mixture was washed with ethyl acetate, and the organic layer was dried over Na₂SO₄, concentrated and purified by flash chromatography eluting with 100/0 to 100/10 dichloromethane/methanol to provide the title compound (13 mg). ¹H NMR (400 MHz, dimethyl sulfoxide-d₆) δ ppm 10.67 (s, 1H), 8.35 (s, 2H), 8.00 (d, J=0.8 Hz, 1H), 7.85 (d, J=2.3 Hz, 1H), 7.57 (d, J=0.8 Hz, 1H), 7.15 (d, J=8.5 Hz, 1H), 7.06 (d, J=2.3 Hz, 1H), 6.73 (dd, J=8.6, 2.4 Hz, 1H), 4.48 (t, J=5.2 Hz, 2H), 4.41 (d, J=7.7 Hz, 4H), 4.20 (p, J=7.7 Hz, 1H), 3.92 (d, J=5.8 Hz, 2H), 3.78 (s, 3H), 3.48 (q, J=5.1 Hz, 4H), 1.95-1.88 (m, 1H). LCMS (ESI+): m/z 495.4 (M+H)⁺.

### Example 52

### N-tert-butyl-2-(3-{6-carbamoyl-5-[(1-methyl-1H-pyrazol-4-yl)amino]pyrazin-2-yl}azetidin-1-yl)-1,3-benzoxazole-6-carboxamide

The title compound was prepared as described in Example 18B using 2-methylpropan-2-amine in place of dimethylamine hydrochloride. ¹H NMR (500 MHz, dimethyl sulfoxide-d₆) δ ppm 10.74 (s, 1H), 8.46 (d, J=2.3 Hz, 1H), 8.40 (s, 1H), 8.06 (d, J=0.8 Hz, 1H), 7.92 (d, J=2.3 Hz, 1H), 7.88 (dd, J=1.6, 0.5 Hz, 1H), 7.72 (dd, J=8.2, 1.7 Hz, 1H), 7.65 (s, 1H), 7.62 (d, J=0.8 Hz, 1H), 7.31 (dd, J=8.2, 0.5 Hz, 1H), 4.57-4.50 (m, 4H), 4.33-4.25 (m, 1H), 3.84 (s, 3H), 1.40 (s, 9H). LCMS (ESI+): m/z 490.4 (M+H)⁺.

### Example 53

### 2-(3-{6-carbamoyl-5-[(1-methyl-1H-pyrazol-4-yl)amino]pyrazin-2-yl}azetidin-1-yl)-N-cyclopropyl-1,3-benzoxazole-6-carboxamide

The title compound was prepared as described in Example 18B using cyclopropanamine in place of dimethylamine hydrochloride. ¹H NMR (400 MHz, dimethyl sulfoxide-d₆) δ ppm 10.68 (s, 1H), 8.40 (s, 1H), 8.35 (s, 1H), 8.29 (d, J=4.1 Hz, 1H), 8.01 (d, J=0.7 Hz, 1H), 7.85 (s, 1H), 7.81 (d, J=1.7 Hz, 1H), 7.68 (dd, J=8.2, 1.7 Hz, 1H), 7.57 (d, J=0.8 Hz, 1H), 7.27 (d, J=8.2 Hz, 1H), 4.53-4.43 (m, 4H), 4.24 (p, J=7.6 Hz, 1H), 3.78 (s, 3H), 2.84-2.75 (m, 1H), 0.70-0.62 (m, 2H), 0.56-0.50 (m, 2H). LCMS (ESI+): m/z 474.3 (M+H)⁺.

### Example 54

### 3-{[1-(2-hydroxyethyl)-1H-pyrazol-4-yl]amino}-6-[1-(6-methyl-1,3-benzoxazol-2-yl)azetidin-3-yl]pyrazine-2-carboxamide

### Example 54A

### methyl 3-chloro-6-[1-(6-methyl-1,3-benzoxazol-2-yl)azetidin-3-yl]pyrazine-2-carboxylate

The title compound was prepared as described in Example 1 using Example 3B in place of 2-chlorobenzo[d]oxazole and Intermediate 2 in place of Intermediate 1. LCMS (ESI+): m/z 359.1 (M+H)⁺.

### Example 54B

### 6-[1-(6-methyl-1,3-benzoxazol-2-yl)azetidin-3-yl]-3-[(1H-pyrazol-4-yl)amino]pyrazine-2-carboxamide

The title compound was prepared as described in Examples 27B and 27C using tert-butyl 4-amino-1H-pyrazole-1-carboxylate in place of 1-(tetrahydro-2H-pyran-4-yl)-1H-pyrazol-4-amine. LCMS (ESI+): m/z 391.2 (M+H)⁺.

### Example 54C

### 3-{[1-(2-hydroxyethyl)-1H-pyrazol-4-yl]amino}-6-[1-(6-methyl-1,3-benzoxazol-2-yl)azetidin-3-yl]pyrazine-2-carboxamide

The title compound was prepared as described in Example 29 using Example 54B in place of Example 14 and 2-bromoethanol in place of 1-bromo-2-methoxyethane. ¹H NMR (500 MHz, dimethyl sulfoxide-d₆) δ ppm 10.75 (s, 1H), 8.43-8.40 (m, 2H), 8.10 (d, J=0.8 Hz, 1H), 7.91 (d, J=2.3 Hz, 1H), 7.65 (d, J=0.8 Hz, 1H), 7.26 (dt, J=1.5, 0.7 Hz, 1H), 7.20 (d, J=7.9 Hz, 1H), 6.99 (ddd, J=7.9, 1.6, 0.8 Hz, 1H), 4.89 (t, J=5.3 Hz, 1H), 4.51-4.43 (m, 4H), 4.31-4.20 (m, 1H), 4.13 (t, J=5.6 Hz, 2H), 3.74 (q, J=5.5 Hz, 2H), 2.37 (s, 3H). LCMS (ESI+): m/z 435.2 (M+H)⁺.

### Example 55

### 3-{[1-(2-methoxyethyl)-1H-pyrazol-4-yl]amino}-6-[1-(6-methyl-1,3-benzoxazol-2-yl)azetidin-3-yl]pyrazine-2-carboxamide

The title compound was prepared as described in Example 29 using Example 54B in place of Example 14. ¹H NMR (500 MHz, dimethyl sulfoxide-d₆) δ ppm 10.75 (s, 1H), 8.43-8.40 (m, 2H), 8.09 (d, J=0.8 Hz, 1H), 7.91 (d, J=2.3 Hz, 1H), 7.66 (d, J=0.8 Hz, 1H), 7.26-7.25 (m, 1H), 7.20 (d, J=7.9 Hz, 1H), 6.99 (ddd, J=7.9, 1.6, 0.8 Hz, 1H), 4.51-4.44 (m, 4H), 4.29-4.21 (m, 3H), 3.68 (dd, J=5.6, 5.0 Hz, 2H), 3.24 (s, 3H), 2.37 (s, 3H). LCMS (ESI+): m/z 449.3 (M+H)⁺.

### Example 56

### 6-[1-(6-{[3-(hydroxymethyl)cyclobutyl]methoxy}-1,3-benzoxazol-2-yl)azetidin-3-yl]-3-[(1-methyl-1H-pyrazol-4-yl)amino]pyrazine-2-carboxamide

The title compound was prepared as described in Example 31 using (3-(hydroxymethyl)cyclobutyl)methyl 4-methylbenzenesulfonate in place of 4-bromo-2-methylbutane-2-ol. ¹H NMR (400 MHz, dimethyl sulfoxide-d₆) δ ppm 10.70 (s, 1H), 8.38 (s, 2H), 8.04 (d, J=0.8 Hz, 1H), 7.88 (d, J=2.4 Hz, 1H), 7.60 (d, J=0.7 Hz, 1H), 7.18 (dd, J=8.6, 2.4 Hz, 1H), 7.10 (dd, J=9.5, 2.4 Hz, 1H), 6.76 (ddd, J=8.6, 7.9, 2.4 Hz, 1H), 4.52-4.34 (m, 5H), 4.23 (p, J=7.6 Hz, 1H), 3.96 (d, J=7.0 Hz, 1H), 3.86 (d, J=6.7 Hz, 1H), 3.42 (dd, J=6.7, 5.3 Hz, 1H), 3.35 (s, 1H), 2.60 (dq, J=22.9, 8.2, 7.8 Hz, 1H), 2.44-2.24 (m, 1H), 2.13-2.01 (m, 1H), 1.90-1.79 (m, 2H), 1.55 (dt, J=11.6, 8.9 Hz, 1H). LCMS (ESI+): m/z 505.5 (M+H)⁺.

### Example 57

### 6-(1-{6-[(3-hydroxyoxetan-3-yl)methoxy]-1,3-benzoxazol-2-yl}azetidin-3-yl)-3-[(1-methyl-1H-pyrazol-4-yl)amino]pyrazine-2-carboxamide

The title compound was prepared as described in Example 31 using (3-hydroxyoxetan-3-yl)methyl 4-methylbenzenesulfonate in place of 4-bromo-2-methylbutane-2-ol. ¹H NMR (400 MHz, dimethyl sulfoxide-d₆) δ ppm 10.71 (s, 1H), 8.38 (s, 2H), 8.05 (dd, J=5.0, 0.8 Hz, 1H), 7.92-7.82 (m, 1H), 7.62 (dd, J=11.9, 0.8 Hz, 1H), 7.22 (dd, J=8.5, 2.4 Hz, 1H), 7.18 (d, J=2.3 Hz, 1H), 6.84 (ddd, J=8.6, 2.5, 1.6 Hz, 1H), 6.00 (d, J=0.7 Hz, 1H), 4.52-4.42 (m, 10H), 4.24 (p, J=7.6 Hz, 1H), 3.82 (d, J=1.8 Hz, 3H). LCMS (ESI+): m/z 193.3 (M+H)⁺.

### Example 58

### 6-[1-(6-{1[(1S,2R)-2-(hydroxymethyl)cyclopropyl]methoxy}-1,3-benzoxazol-2-yl)azetidin-3-yl]-3-[(1-methyl-1H-pyrazol-4-yl)amino]pyrazine-2-carboxamide

The title compound was prepared as described in Example 31 using ((1R,2S)-2-(hydroxymethyl)cyclopropyl)methyl 4-methylbenzenesulfonate in place of 4-bromo-2-methylbutane-2-ol. ¹H NMR (400 MHz, dimethyl sulfoxide-d₆) δ ppm 10.70 (s, 1H), 8.38 (s, 2H), 8.04 (s, 1H), 7.88 (s, 1H), 7.60 (d, J=0.8 Hz, 1H), 7.19 (d, J=8.5 Hz, 1H), 7.12 (d, J=2.4 Hz, 1H), 6.78 (dd, J=8.5, 2.4 Hz, 1H), 4.44 (d, J=7.7 Hz, 4H), 4.23 (p, J=7.6 Hz, 1H), 4.11 (dd, J=10.6, 6.6 Hz, 1H), 4.00 (dd, J=10.6, 7.4 Hz, 1H), 3.82 (s, 3H), 3.78 (d, J=7.7 Hz, 2H), 1.54-1.33 (m, 2H), 0.95 (td, J=8.4, 4.8 Hz, 1H), 0.49 (q, J=5.4 Hz, 1H). LCMS (ESI+): m/z 491.4 (M+H)⁺.

### Example 59

### 6-(1-{6-[(2R)-2-(hydroxymethyl)pyrrolidine-1-carbonyl]-1,3-benzoxazol-2-yl}azetidin-3-yl)-3-[(1-methyl-1H-pyrazol-4-yl)amino]pyrazine-2-carboxamide

The title compound was prepared as described in Example 18B using R-pyrrolidine-2-ylmethanol in place of dimethylamine hydrochloride. ¹H NMR (400 MHz, dimethyl sulfoxide-d₆) δ ppm 10.68 (s, 1H), 8.40 (s, 1H), 8.35 (s, 1H), 8.01 (d, J=0.8 Hz, 1H), 7.87-7.82 (m, 1H), 7.57 (d, J=0.8 Hz, 1H), 7.55-7.53 (m, 1H), 7.35-7.26 (m, 2H), 4.78-4.68 (m, 1H), 4.52-4.44 (m, 4H), 4.24 (p, J=7.6 Hz, 1H), 4.17-4.06 (m, 1H), 3.79 (s, 3H), 3.64-3.40 (m, 3H), 1.98-1.77 (m, 4H), 1.73-1.57 (m, 1H). LCMS (ESI+): m/z 518.2 (M+H)⁺.

### Example 60

### 3-[(1-methyl-1H-pyrazol-4-yl)amino]-6-{1-[6-(3-phenylpropoxy)-1,3-benzoxazol-2-yl]azetidin-3-yl}pyrazine-2-carboxamide

The title compound was obtained as described in the Example 31 using 1-bromo-3-phenylpropane in place of 4-bromo-2-methylbutane-2-ol. ¹H NMR (400 MHz, dimethyl sulfoxide-d₆) δ ppm 10.70 (s, 1H), 8.38 (s, 2H), 8.04 (d, J=0.8 Hz, 1H), 7.88 (d, J=2.4 Hz, 1H), 7.60 (d, J=0.8 Hz, 1H), 7.33-7.13 (m, 6H), 7.11 (d, J=2.4 Hz, 1H), 6.77 (dd, J=8.6, 2.4 Hz, 1H), 4.49-4.36 (m, 4H), 4.23 (p, J=7.6 Hz, 1H), 3.95 (t, J=6.3 Hz, 2H), 3.82 (s, 3H), 2.74 (dd, J=8.7, 6.7 Hz, 2H), 2.11-1.87 (m, 2H). LCMS (ESI+): m/z 525.2 (M+H)⁺.

### Example 61

### 6-{1-[6-(3-hydroxy-2,2-dimethylpropoxy)-1,3-benzoxazol-2-yl]azetidin-3-yl}-3-[(1-methyl-1H-pyrazol-4-yl)amino]pyrazine-2-carboxamide

The title compound was obtained as described in the Example 31 using 3-bromo-2,2-dimethyl-1-propanol in place of 4-bromo-2-methylbutane-2-ol. ¹H NMR (400 MHz, dimethyl sulfoxide-d₆) δ ppm 10.70 (s, 1H), 8.38 (s, 2H), 8.04 (d, J=0.8 Hz, 1H), 7.88 (d, J=2.3 Hz, 1H), 7.60 (d, J=0.8 Hz, 1H), 7.18 (d, J=8.5 Hz, 1H), 7.08 (d, J=2.3 Hz, 1H), 6.76 (dd, J=8.5, 2.4 Hz, 1H), 4.58 (t, J=5.3 Hz, 1H), 4.44 (d, J=7.7 Hz, 4H), 4.23 (p, J=7.5 Hz, 1H), 3.82 (s, 3H), 3.68 (s, 2H), 3.28 (d, J=5.3 Hz, 2H), 0.92 (s, 6H). LCMS (ESI+): m/z 493.2 (M+H)⁺.

### Example 62

### 6-(1-{6-[3-(dimethylamino)-3-oxopropyl]-1,3-benzoxazol-2-yl}azetidin-3-yl)-3-[(1-methyl-1H-pyrazol-4-yl)amino]pyrazine-2-carboxamide

### Example 62A

### tert-butyl (2E)-3-[2-(3-{6-carbamoyl-5-[(1-methyl-1H-pyrazol-4-yl)amino]pyrazin-2-yl}azetidin-1-yl)-1,3-benzoxazol-6-yl]prop-2-enoate

A mixture of Example 6A (500 mg, 1.065 mmol) and bis(triphenylphosphine)palladium(II) dichloride (37.4 mg, 0.053 mmol) was sparged with nitrogen and N,N-dimethylformamide (2.66 mL), triethylamine (0.418 mL, 3.0 mmol), and tert-butyl acrylate (0.31 mL, 2.13 mmol) were added. The mixture was sparged for 5 minutes and heated for 18 hours at 90° C. The cooled mixture was partitioned between water and ethyl acetate and the organic layers were dried over Na₂SO₄, filtered, concentrated and purified by silica gel chromatography eluting with 100/0 to 95/5 dichloromethane/methanol to afford the title compound (340 mg). LCMS (ESI+): m/z 517.23 (M+H)⁺.

### Example 62B

### tert-butyl 3-[2-(3-{6-carbamoyl-5-[(1-methyl-1H-pyrazol-4-yl)amino]pyrazin-2-yl}azetidin-1-yl)-1,3-benzoxazol-6-yl]propanoate

The title compound was prepared as described in Example 7 using Example 62A in place Example 6. LCMS (ESI+): m/z 519.06 (M+H)⁺.

### Example 62C

### 3-[2-(3-f{6-carbamoyl-5-[(1-methyl-1H-pyrazol-4-yl)amino]pyrazin-2-yl}azetidin-1-yl)-1,3-benzoxazol-6-yl]propanoic acid

A mixture of Example 62B (184 mg, 0.366 mmol), dichloromethane (3.5 mL) and trifluoroacetic acid (0.27 mL) was stirred at ambient temperature for 18 hours. Additional trifluoroacetic acid (1 mL) was added and the mixture stirred an additional 2.5 hours. The mixture was concentrated, treated with cyclopentyl methyl ether (10 mL) and concentrated. Additional cyclopentyl methyl ether (10 mL) was added. The slurry was allowed to stand overnight and filtered to afford the title compound (185 mg) as the trifluoroacetic acid salt. LCMS (ESI+): m/z 463.04 (M+H)⁺.

### Example 62D

### 6-(1-{6-[3-(dimethylamino)-3-oxopropyl]-1,3-benzoxazol-2-yl}azetidin-3-yl)-3-[(1-methyl-1H-pyrazol-4-yl)amino]pyrazine-2-carboxamide

The title compound was prepared as described in Example 18B using Example 62C in place of Example 18A. ¹H NMR (400 MHz, dimethyl sulfoxide-d₆) δ ppm 10.67 (s, 1H), 8.36-8.32 (m, 2H), 8.01 (d, J=0.7 Hz, 1H), 7.84 (d, J=2.3 Hz, 1H), 7.57 (d, J=0.8 Hz, 1H), 7.28 (d, J=1.5 Hz, 1H), 7.16 (d, J=8.0 Hz, 1H), 7.00 (dd, J=8.0, 1.6 Hz, 1H), 4.42 (d, J=7.6 Hz, 4H), 4.21 (p, J=7.6 Hz, 1H), 3.78 (s, 3H), 2.89 (s, 3H), 2.81 (dd, J=8.5, 6.9 Hz, 2H), 2.78 (s, 3H), 2.56 (dd, J=8.6, 6.9 Hz, 2H). LCMS (ESI+): m/z 490.18 (M+H)⁺.

### Example 63

### 3-[(1-methyl-1H-pyrazol-4-yl)amino]-6-(1-{6-[3-oxo-3-(pyrrolidin-1-yl)propyl]-1,3-benzoxazol-2-yl}azetidin-3-yl)pyrazine-2-carboxamide

The title compound was prepared as described in Example 18B using Example 62C in place of Example 18A and pyrrolidine in place of dimethylamine hydrochloride. ¹H NMR (400 MHz, dimethyl sulfoxide-d₆) δ ppm 10.67 (s, 1H), 8.37-8.32 (m, 2H), 8.00 (s, 1H), 7.84 (d, J=2.3 Hz, 1H), 7.57 (d, J=0.8 Hz, 1H), 7.28 (d, J=1.5 Hz, 1H), 7.16 (d, J=8.0 Hz, 1H), 7.00 (dd, J=8.0, 1.6 Hz, 1H), 4.42 (d, J=7.6 Hz, 4H), 4.21 (p, J=7.6 Hz, 1H), 3.78 (s, 3H), 3.34-3.21 (m, 4H), 2.82 (t, J=7.7 Hz, 2H), 2.52-2.47 (m, 2H), 1.84-1.65 (m, 4H). LCMS (ESI+): m/z 516.21 (M+H)⁺.

### Example 64

### 6-(1-{6-[(2R)-2-(methoxymethyl)pyrrolidine-1-carbonyl]-1,3-benzoxazol-2-yl}azetidin-3-yl)-3-[(1-methyl-1H-pyrazol-4-yl)amino]pyrazine-2-carboxamide

The title compound was prepared as described in Example 18B using (R)-2-(methoxymethyl)pyrrolidine in place of dimethylamine hydrochloride. ¹H NMR (400 MHz, dimethyl sulfoxide-d₆) δ ppm 10.69 (s, 1H), 8.40 (d, J=2.2 Hz, 1H), 8.35 (s, 1H), 8.01 (d, J=0.8 Hz, 1H), 7.86 (d, J=2.3 Hz, 1H), 7.57 (d, J=0.8 Hz, 1H), 7.53 (t, J=1.1 Hz, 1H), 7.32-7.25 (m, 2H), 4.48 (d, J=7.8 Hz, 4H), 4.29-4.15 (m, 2H), 3.79 (s, 3H), 3.63-3.18 (m, 7H), 2.01-1.91 (m, 1H), 1.89-1.61 (m, 3H). LCMS (ESI+): m/z 532.47 (M+H)⁺.

### Example 65

### 3-[(1-methyl-1H-pyrazol-4-yl)amino]-6-(1-{6-[2-oxo-2-(pyrrolidin-1-yl)ethyl]-1,3-benzoxazol-2-yl}azetidin-3-yl)pyrazine-2-carboxamide

### Example 65A

### 6-(1-{6-[(E)-2-ethoxyethenyl]-1,3-benzoxazol-2-yl}azetidin-3-yl)-3-[(1-methyl-1H-pyrazol-4-yl)amino]pyrazine-2-carboxamide

The title compound was prepared as described in Example 17 using (E)-2-(2-ethoxyvinyl)-4,4,5,5-tetramethyl-1,3,2-dioxaborolane in place of (E)-2-methyl-4-(4,4,5,5-tetramethyl-1,3,2-dioxaborolane-2-yl)but-3-en-2-ol. LCMS (ESI+): m/z 461.4 (M+H)⁺.

### Example 65B

### 3-[(1-methyl-1H-pyrazol-4-yl)amino]-6-{1-[6-(2-oxoethyl)-1,3-benzoxazol-2-yl]azetidin-3-yl}pyrazine-2-carboxamide

A solution of Example 65A (59 mg, 0.128 mmol) in dichloromethane (0.5 mL) was treated with trifluoroacetic acid (0.5 mL, 6.49 mmol), stirred for 30 minutes at ambient temperature and diluted with dichloromethane (5 mL) and saturated NaHCO₃ (15 mL). The aqueous phase was extracted with dichloromethane and the organic layers were dried over Na₂SO₄, filtered, and concentrated to afford the title compound (52 mg). LCMS (ESI−): m/z 430.9 (M−H)⁻.

### Example 65C

### [2-(3-{6-carbamoyl-5-[(1-methyl-1H-pyrazol-4-yl)amino]pyrazin-2-yl}azetidin-1-yl)-1,3-benzoxazol-6-yl]acetic acid

To a suspension of Example 65B (300 mg, 0.694 mmol) in tetrahydrofuran (5 mL) and water (5 mL) was added 2-methylbut-2-ene (0.735 mL, 6.94 mmol) followed by sodium chlorite (502 mg, 5.55 mmol) and sodium dihydrogenphosphate dihydrate (974 mg, 6.24 mmol). The mixture was stirred at 20° C. for 3 hours and diluted with 1 N sodium hydroxide (10 mL) and dichloromethane (15 mL). The organic layer was extracted with water and the combined aqueous layers were adjusted to pH 2 by addition of 1 N hydrochloric acid. The precipitate was collected by filtration and the filtrate extracted with dichloromethane. The precipitate and the organic extracts were combined and concentrated. The residue was purified by silica gel chromatography eluting with 100/0 to 88/12 methanol/dichloromethane to afford the title compound (148 mg). ¹H NMR (400 MHz, dimethyl sulfoxide-d₆) δ ppm 12.23 (s, 1H), 10.67 (s, 1H), 8.37-8.33 (m, 2H), 8.01 (d, J=0.7 Hz, 1H), 7.84 (d, J=2.3 Hz, 1H), 7.57 (d, J=0.8 Hz, 1H), 7.29 (d, J=1.6 Hz, 1H), 7.20 (d, J=8.0 Hz, 1H), 7.02 (dd, J=8.0, 1.6 Hz, 1H), 4.44 (d, J=7.7 Hz, 4H), 4.27-4.17 (m, 1H), 3.78 (s, 3H), 3.57 (s, 2H). LCMS (ESI+): m/z 449.30 (M+H)⁺.

### Example 65D

### 3-[(1-methyl-1H-pyrazol-4-yl)amino]-6-(1-{6-[2-oxo-2-(pyrrolidin-1-yl)ethyl]-1,3-benzoxazol-2-yl}azetidin-3-yl)pyrazine-2-carboxamide

The title compound was prepared as described in Example 18B using Example 65C in place of Example 18A and pyrrolidine in place of dimethylamine hydrochloride. ¹H NMR (400 MHz, dimethyl sulfoxide-d₆) δ ppm 10.68 (s, 1H), 8.35 (d, J=3.4 Hz, 2H), 8.01 (d, J=0.8 Hz, 1H), 7.84 (d, J=2.3 Hz, 1H), 7.57 (d, J=0.8 Hz, 1H), 7.25 (d, J=1.6 Hz, 1H), 7.19 (d, J=8.0 Hz, 1H), 7.00 (dd, J=8.0, 1.6 Hz, 1H), 4.43 (d, J=7.7 Hz, 4H), 4.22 (p, J=7.7 Hz, 1H), 3.79 (s, 3H), 3.62 (s, 2H), 3.43 (t, J=6.8 Hz, 2H), 3.27-3.23 (m, 2H), 1.87-1.78 (m, 2H), 1.77-1.68 (m, 2H). LCMS (ESI+): m/z 502.38 (M+H)⁺.

### Example 66

### 6-[1-(6-{2-[(2S)-2-(hydroxymethyl)pyrrolidin-1-yl]-2-oxoethyl}-1,3-benzoxazol-2-yl)azetidin-3-yl]-3-[(1-methyl-1H-pyrazol-4-yl)amino]pyrazine-2-carboxamide

To a mixture of Example 65C (60 mg, 0.134 mmol) and 2-(3H-[1,2,3]triazolo[4,5-b]pyridin-3-yl)-1,1,3,3-tetramethylisouronium hexafluorophosphate(V) (102 mg, 0.268 mmol) was added N,N-dimethylformamide (0.5 mL), N-ethyl-N-isopropylpropan-2-amine (0.12 mL, 0.694 mmol) and (S)-pyrrolidin-2-ylmethanol (0.02 mL, 0.20 mmol) and the mixture was stirred at ambient temperature for 18 hours. The mixture was partitioned between water and ethyl acetate. The organic layers were dried over Na₂SO₄, filtered, concentrated and purified by silica gel chromatography eluting with 100/0 to 92/8 dichloromethane/methanol to afford the title compound (340 mg). ¹H NMR (500 MHz, dimethyl sulfoxide-d₆) δ ppm 10.73 (s, 1H), 8.44-8.41 (m, 1H), 8.40 (s, 1H), 8.06 (s, 1H), 7.92-7.89 (m, 1H), 7.62 (s, 1H), 7.29 (dd, J=5.3, 1.5 Hz, 1H), 7.24 (dd, J=8.0, 3.5 Hz, 1H), 7.04 (dt, J=8.1, 2.1 Hz, 1H), 5.00 (t, J=5.7 Hz, 0.3H), 4.76 (t, J=5.6 Hz, 0.7H), 4.48 (dd, J=7.7, 2.4 Hz, 4H), 4.27 (p, J=7.6 Hz, 1H), 4.04 (d, J=6.4 Hz, 0.3H), 3.96 (dt, J=8.5, 4.5 Hz, 0.7H), 3.83 (s, 3H), 3.67 (s, 2H), 3.56-3.42 (m, 2H), 3.40-3.23 (m, 2H), 1.98-1.75 (m, 4H). LCMS (ESI+): m/z 532.18 (M+H)⁺.

### Example 67

### 6-{1-[6-(2-hydroxy-2-methylpropyl)-1,3-benzoxazol-2-yl]azetidin-3-yl}-3-[(1-methyl-1H-pyrazol-4-yl)amino]pyrazine-2-carboxamide

### Example 67A

### methyl [2-(3-{6-carbamoyl-5-[(1-methyl-1H-pyrazol-4-yl)amino]pyrazin-2-yl}azetidin-1-yl)-1,3-benzoxazol-6-yl]acetate

A mixture of Example 65C (200 mg, 0.446 mmol), methanol (5 mL) and thionyl chloride (2 drops) was stirred for 18 hours. Additional thionyl chloride (2 drops) was added and the mixture was stirred for an additional 2.5 hours. The mixture was concentrated and partitioned between half saturated NaHCO₃ and ethyl acetate. The organic layers were dried over Na₂SO₄, filtered, and concentrated to afford the title compound (180 mg) that was used without further purification. ¹H NMR (400 MHz, dimethyl sulfoxide-d₆) δ ppm 10.68 (s, 1H), 8.36 (s, 1H), 8.35 (s, 1H), 8.01 (d, J=0.8 Hz, 1H), 7.84 (d, J=2.2 Hz, 1H), 7.57 (d, J=0.8 Hz, 1H), 7.30 (d, J=1.6 Hz, 1H), 7.20 (d, J=8.0 Hz, 1H), 7.02 (dd, J=8.0, 1.6 Hz, 1H), 4.46-4.41 (m, 4H), 4.22 (p, J=7.6 Hz, 1H), 3.78 (s, 3H), 3.68 (s, 2H), 3.57 (s, 3H). LCMS (ESI+): m/z 463.11 (M+H)⁺.

### Example 67B

### 6-{1-[6-(2-hydroxy-2-methylpropyl)-1,3-benzoxazol-2-yl]azetidin-3-yl}-3-[(1-methyl-1H-pyrazol-4-yl)amino]pyrazine-2-carboxamide

The title compound was prepared as described in Example 19 using Example 67A in place of Example 13. ¹H NMR (400 MHz, dimethyl sulfoxide-d₆) δ ppm 10.68 (s, 1H), 8.37-8.34 (m, 2H), 8.01 (s, 1H), 7.86-7.83 (m, 1H), 7.57 (d, J=0.7 Hz, 1H), 7.22 (d, J=1.5 Hz, 1H), 7.15 (d, J=7.9 Hz, 1H), 6.96 (dd, J=7.9, 1.6 Hz, 1H), 4.43 (dd, J=7.7, 1.7 Hz, 4H), 4.24 (s, 1H), 4.21 (p, J=7.6 Hz, 1H), 3.79 (s, 3H), 2.65 (s, 2H), 1.02 (s, 6H). LCMS (ESI+): m/z 463.06 (M+H)⁺.

### Example 68

### 6-(1-{6-[(2R)-2-hydroxypropoxy]-1,3-benzoxazol-2-yl}azetidin-3-yl)-3-[(1-methyl-1H-pyrazol-4-yl)amino]pyrazine-2-carboxamide

A mixture of Example 20 (0.05 g, 0.123 mmol) and cesium carbonate (0.06 g, 0.185 mmol) in N,N-dimethylformamide (0.5 mL) was stirred at ambient temperature for 15 minutes. (R)-Propylene oxide (15 mg, 0.27 mmol) was added and the mixture was heated at 70° C. for 10 hours. The mixture was cooled, quenched with water, and diluted with ethyl acetate. The aqueous layer was washed with ethyl acetate, and the organic layers were washed with water, washed with brine, dried over Na₂SO₄, and concentrated. The residue was purified by silica gel chromatography eluting with 100:0 to 100:10 dichloromethane/methanol to afford the title compound (31 mg). ¹H NMR (400 MHz, dimethyl sulfoxide-d₆) δ ppm 10.71 (s, 1H), 8.39 (d, J=9.3 Hz, 2H), 8.04 (s, 1H), 7.89 (d, J=2.3 Hz, 1H), 7.61 (s, 1H), 7.21 (d, J=8.6 Hz, 1H), 7.14 (d, J=2.4 Hz, 1H), 6.80 (dd, J=8.6, 2.4 Hz, 1H), 4.47 (m, 5H), 4.27-4.23 (m, 1H), 3.98-3.89 (m, 1H), 3.82 (s, 3H), 3.77 (dt, J=9.6, 4.5 Hz, 1H), 1.15 (d, J=6.3 Hz, 3H). LCMS (ESI+): m/z 465.2 (M+H)⁺.

### Example 69

### 3-[(1-methyl-1H-pyrazol-4-yl)amino]-6-[1-(6-{[(3S)-oxolan-3-yl]oxy}-1,3-benzoxazol-2-yl)azetidin-3-yl]pyrazine-2-carboxamide

The title compound was prepared as described in Example 31 using (R)-tetrahydrofuran-3-yl 4-methylbenzenesulfonate in place of 4-bromo-2-methylbutane-2-ol. ¹H NMR (400 MHz, dimethyl sulfoxide-d₆) δ ppm 10.70 (s, 1H), 8.38 (s, 2H), 8.04 (d, J=0.8 Hz, 1H), 7.97-7.82 (m, 1H), 7.60 (d, J=0.8 Hz, 1H), 7.20 (d, J=8.5 Hz, 1H), 7.12 (d, J=2.4 Hz, 1H), 6.75 (dd, J=8.5, 2.4 Hz, 1H), 4.99 (d, J=1.8 Hz, 1H), 4.44 (dd, J=7.6, 1.8 Hz, 4H), 4.25 (d, J=7.6 Hz, 1H), 3.90-3.83 (m, 2H), 3.82 (s, 3H), 3.80-3.70 (m, 2H), 2.26-2.13 (m, 1H), 1.96 (dt, J=12.6, 5.6 Hz, 1H). LCMS (ESI+): m/z 477.4 (M+H)⁺.

### Example 70

### 6-[1-(6-{2-[(3S)-3-methoxypyrrolidin-1-yl]-2-oxoethyl}-1,3-benzoxazol-2-yl)azetidin-3-yl]-3-[(1-methyl-1H-pyrazol-4-yl)amino]pyrazine-2-carboxamide

The title compound was prepared as described in Example 66 using (S)-3-methoxypyrrolidine hydrochloride in place of (S)-pyrrolidin-2-ylmethanol. ¹H NMR (400 MHz, dimethyl sulfoxide-d₆, T=90° C.) δ ppm 10.51 (s, 1H), 8.34 (s, 1H), 8.02 (br s, 1H), 7.93 (d, J=0.8 Hz, 1H), 7.50 (br s, 1H), 7.54 (d, J=0.8 Hz, 1H), 7.24 (d, J=1.6 Hz, 1H), 7.19 (d, J=8.0 Hz, 1H), 7.01 (dd, J=8.1, 1.6 Hz, 1H), 4.50 (t, J=8.3 Hz, 2H), 4.42 (dd, J=7.9, 6.4 Hz, 2H), 4.21 (tt, J=8.6, 6.4 Hz, 1H), 4.01-3.88 (m, 1H), 3.79 (s, 3H), 3.62 (s, 2H), 3.59-3.25 (m, 4H), 3.21 (s, 3H), 2.00-1.81 (m, 2H). LCMS (ESI+): m/z 490.4 (M+H)⁺.

### Example 71

### 6-[1-(6-{2-[(2R)-2-(methoxymethyl)pyrrolidin-1-yl]-2-oxoethyl}-1,3-benzoxazol-2-yl)azetidin-3-yl]-3-[(1-methyl-1H-pyrazol-4-yl)amino]pyrazine-2-carboxamide

The title compound was prepared as described in Example 66 using (R)-2-(methoxymethyl)pyrrolidine in place of (S)-pyrrolidin-2-ylmethanol. ¹H NMR (400 MHz, dimethyl sulfoxide-d₆) δ ppm 10.68 (s, 1H), 8.36 (d, J=2.7 Hz, 1H), 8.35 (s, 1H), 8.01 (d, J=0.7 Hz, 1H), 7.84 (d, J=2.1 Hz, 1H), 7.57 (d, J=0.8 Hz, 1H), 7.23 (d, J=1.5 Hz, 1H), 7.19 (dd, J=7.9, 2.3 Hz, 1H), 6.99 (dt, J=8.1, 2.1 Hz, 1H), 4.44 (d, J=7.8 Hz, 4H), 4.22 (p, J=7.6 Hz, 1H), 4.05-3.98 (m, 1H), 3.79 (s, 3H), 3.62 (d, J=4.9 Hz, 1H), 3.45-3.40 (m, 1H), 3.38 (dd, J=9.0, 3.4 Hz, 1H), 3.29-3.17 (m, 7H), 1.93-1.71 (m, 4H). LCMS (ESI+): m/z 546.4 (M+H)⁺.

### Example 72

### 6-(1-{6-[(1-methyl-2-oxopyrrolidin-3-yl)oxy]-1,3-benzoxazol-2-yl}azetidin-3-yl)-3-[(1-methyl-1H-pyrazol-4-yl)amino]pyrazine-2-carboxamide

A solution of Example 20 (0.25 g, 0.615 mmol) in N,N-dimethylformamide (2.6 mL) under nitrogen was treated with cesium carbonate (0.401 g, 1.23 mmol) and potassium iodide (112 mg, 0.677 mmol), and the mixture was stirred at ambient temperature for 20 minutes. 3-Bromo-1-methylpyrrolidin-2-one (120 mg, 0.677 mmol) was added and the mixture was stirred at ambient temperature overnight. The mixture was washed with ethyl acetate, and the organic layers were dried over Na₂SO₄, concentrated, and purified by silica gel chromatography eluting with 0:100 to 30:100 methanol/dichloromethane to provide the title compound (0.17 g). ¹H NMR (600 MHz, dimethyl sulfoxide-d₆) δ ppm 10.71 (s, 1H), 8.39 (d, J=15.7 Hz, 2H), 8.04 (d, J=0.8 Hz, 1H), 7.89 (d, J=2.3 Hz, 1H), 7.60 (d, J=0.8 Hz, 1H), 7.23 (d, J=2.4 Hz, 1H), 7.22-7.17 (m, 1H), 6.85 (dd, J=8.6, 2.4 Hz, 1H), 4.91 (dd, J=7.8, 6.8 Hz, 1H), 4.50-4.38 (m, 4H), 4.29-4.19 (m, 1H), 3.82 (s, 3H), 3.44-3.36 (m, 1H), 3.32-3.30 (m, 1H), 2.79 (d, J=0.5 Hz, 3H), 2.57-2.52 (m, 1H), 1.91 (ddt, J=13.3, 8.8, 6.8 Hz, 1H). LCMS (ESI+): m/z 504.2 (M+H)⁺.

### Example 73

### 6-(1-{6-[2-(azetidin-1-yl)-2-oxoethoxy]-1,3-benzoxazol-2-yl}azetidin-3-yl)-3-[(1-methyl-1H-pyrazol-4-yl)amino]pyrazine-2-carboxamide

### Example 73A

### {[2-(3-f{6-carbamoyl-5-[(1-methyl-1H-pyrazol-4-yl)amino]pyrazin-2-yl}azetidin-1-yl)-1,3-benzoxazol-6-yl]oxy}acetic acid

To a solution of Example 26 (0.553 g, 1.123 mmol) in ethanol (1 mL) was added lithium hydroxide (0.134 g, 5.61 mmol) in tetrahydrofuran (1 mL) and the mixture was heated at 50° C. for 2 hours. The mixture was concentrated, washed with ethyl acetate, and filtered to afford the title compound which was carried to the next step without further purification.

### Example 73B

### 6-(1-{6-[2-(azetidin-1-yl)-2-oxoethoxy]-1,3-benzoxazol-2-yl}azetidin-3-yl)-3-[(1-methyl-1H-pyrazol-4-yl)amino]pyrazine-2-carboxamide

To a mixture of Example 73A (0.045 g, 0.097 mmol), azetidine hydrochloride (0.01 g, 0.107 mmol), 1-ethyl-3-(3-dimethylaminopropyl)carbodiimide hydrochloride (0.037 g, 0.194 mmol) and 1-hydroxybenzotriazole (0.018 mg, 0.116 mmol) was added N,N-dimethylformamide (0.5 mL) followed by N,N-diisopropylethylamine (0.084 mL, 0.484 mmol), and the mixture was stirred at ambient temperature for 16 hours. The mixture was diluted with ethyl acetate, washed with water, washed with brine, dried over Na₂SO₄, and concentrated. Purification by silica gel chromatography eluting with 0:100 to 20:100 methanol/ethyl acetate provided the title compound (0.021 g). ¹H NMR (500 MHz, dimethyl sulfoxide-d₆) δ ppm 10.71 (s, 1H), 8.39 (d, J=10.4 Hz, 2H), 8.04 (d, J=0.8 Hz, 1H), 7.95-7.82 (m, 1H), 7.60 (d, J=0.8 Hz, 1H), 7.21 (d, J=8.5 Hz, 1H), 7.10 (d, J=2.5 Hz, 1H), 6.77 (dd, J=8.6, 2.5 Hz, 1H), 4.55 (s, 2H), 4.51-4.37 (m, 4H), 4.24 (td, J=7.5, 2.6 Hz, 3H), 3.90 (t, J=7.8 Hz, 2H), 3.82 (s, 3H), 2.30-2.16 (m, 2H). LCMS (ESI+): m/z 504.3 (M+H)⁺.

### Example 74

### 6-[1-(6-methyl-1,3-benzoxazol-2-yl)azetidin-3-yl]-3-[(1H-pyrazol-4-yl)amino]pyrazine-2-carboxamide

### Example 74A

### 6-[1-(6-methyl-1,3-benzoxazol-2-yl)azetidin-3-yl]-3-[(1-{[2-(trimethylsilyl)ethoxy]methyl}-1H-pyrazol-4-yl)amino]pyrazine-2-carboxamide

The title compound was prepared as described for Example OG using Example 3B in place of 2-chlorobenzo[d]oxazol-6-ol. LCMS (ESI+): m/z 521.2 (M+H)⁺.

### Example 74B

### 6-[1-(6-methyl-1,3-benzoxazol-2-yl)azetidin-3-yl]-3-[(1H-pyrazol-4-yl)amino]pyrazine-2-carboxamide

To a solution of Example 74A (0.61 g, 1.172 mmol) in tetrahydrofuran (5 mL) was added 1 N tetrabutylammonium fluoride in tetrahydrofuran (7.03 mL, 7.03 mmol) dropwise. The mixture was heated at reflux overnight, diluted with water, and extracted with ethyl acetate. The extracts were washed with water, dried over MgSO₄, and concentrated to afford the title compound (421 mg). ¹H NMR (600 MHz, dimethyl sulfoxide-d₆) δ ppm 12.61 (s, 1H), 10.71 (s, 1H), 8.39 (s, 1H), 8.38 (d, J=2.2 Hz, 1H), 8.04 (s, 1H), 7.89-7.86 (m, 1H), 7.71 (s, 1H), 7.24 (dt, J=1.5, 0.7 Hz, 1H), 7.18 (d, J=7.9 Hz, 1H), 6.98 (ddd, J=7.9, 1.6, 0.7 Hz, 1H), 4.50-4.42 (m, 4H), 4.28-4.20 (m, 1H), 2.35 (s, 3H). LCMS (ESI+): m/z 391.1 (M+H)⁺.

### Example 75

### 6-(1-{6-[(2R)-2-(2-hydroxypropan-2-yl)pyrrolidine-1-carbonyl]-1,3-benzoxazol-2-yl}azetidin-3-yl)-3-[(1-methyl-1H-pyrazol-4-yl)amino]pyrazine-2-carboxamide

The title compound was prepared as described in Example 18B using (R)-2-(pyrrolidin-2-yl)propan-2-ol hydrochloride in place of dimethylamine hydrochloride. ¹H NMR (600 MHz, dimethyl sulfoxide-d₆) δ ppm 10.72 (s, 1H), 8.44 (d, J=2.4 Hz, 1H), 8.38 (s, 1H), 8.05 (d, J=0.8 Hz, 1H), 7.90 (d, J=2.3 Hz, 1H), 7.65 (s, 1H), 7.61 (d, J=0.8 Hz, 1H), 7.42 (d, J=8.1 Hz, 1H), 7.32 (d, J=8.1 Hz, 1H), 4.89 (s, 1H), 4.51 (dd, J=7.6, 5.8 Hz, 4H), 4.34-4.24 (m, 2H), 3.82 (s, 3H), 3.53 (td, J=10.3, 6.8 Hz, 1H), 3.45-3.37 (m, 1H), 1.90 (q, J=7.5 Hz, 2H), 1.81 (s, 1H), 1.56 (q, J=9.7 Hz, 1H), 1.13 (s, 3H), 1.10 (s, 3H). LCMS (ESI+): m/z 546.4 (M+H)⁺.

### Example 76

### 6-(1-{6-[(1-methyl-2-oxopiperidin-3-yl)oxy]-1,3-benzoxazol-2-yl}azetidin-3-yl)-3-[(1-methyl-1H-pyrazol-4-yl)amino]pyrazine-2-carboxamide

The title compound was prepared as described in Example 72 using 3-bromo-1-methylpiperidin-2-one in place of 3-bromo-1-methylpyrrolidin-2-one. ¹H NMR (400 MHz, dimethyl sulfoxide-d₆) δ ppm 10.67 (s, 1H), 8.35 (s, 2H), 8.01 (d, J=0.8 Hz, 1H), 7.84 (d, J=2.3 Hz, 1H), 7.57 (d, J=0.8 Hz, 1H), 7.21-7.11 (m, 2H), 6.80 (dd, J=8.6, 2.4 Hz, 1H), 4.69 (dd, J=7.0, 4.8 Hz, 1H), 4.41 (dd, J=7.5, 1.5 Hz, 4H), 4.20 (p, J=7.6 Hz, 1H), 3.78 (s, 3H), 3.27 (s, 2H), 2.80 (s, 3H), 2.02 (ddd, J=12.1, 8.6, 4.0 Hz, 1H), 1.99-1.72 (m, 3H). LCMS (ESI+): m/z 518.2 (M+H)⁺.

### Example 77

### 4-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylbutan-2-yl dihydrogen phosphate

### Example 77A

### 4-{[2-(3-f{6-carbamoyl-5-[(1-methyl-1H-pyrazol-4-yl)amino]pyrazin-2-yl}azetidin-1-yl)-1,3-benzoxazol-6-yl]oxy}-2-methylbutan-2-yl diprop-2-en-1-yl phosphate

A mixture of Example 31 (1.00 g, 2.03 mmol) in N,N-dimethylformamide (20 mL) and 0.45 M tetrazole solution in acetonitrile (13.54 mL, 6.09 mmol) under nitrogen was treated with diallyl N,N-diisopropylphosphoramidite (1.25 g, 5.08 mmol) and the mixture was stirred at ambient temperature for 2 hours. The mixture was cooled to 0° C., hydrogen peroxide (30% w/w) (2.09 mL, 20.3 mmol) was added, and the mixture was stirred for 16 hours. The mixture was diluted with ethyl acetate, washed with sodium thiosulfate, washed with water, washed with brine, and dried over Na₂SO₄. Concentration and purification by prep-HPLC (100:0 to 100:70 acetonitrile/water) afforded the title compound (1.10 g). ¹H NMR (400 MHz, dimethyl sulfoxide-d₆) δ ppm 10.71 (s, 1H), 8.38 (d, J=3.9 Hz, 2H), 8.04 (s, 1H), 7.89 (d, J=2.4 Hz, 1H), 7.60 (s, 1H), 7.28-7.08 (m, 2H), 6.78 (dd, J=8.6, 2.4 Hz, 1H), 5.92 (ddt, J=17.2, 10.5, 5.3 Hz, 2H), 5.32 (dq, J=17.2, 1.8 Hz, 2H), 5.20 (dq, J=10.4, 1.5 Hz, 2H), 4.56-4.38 (m, 8H), 4.24 (p, J=7.6 Hz, 1H), 4.09 (q, J=6.9 Hz, 2H), 3.82 (s, 3H), 2.23-2.03 (m, 2H), 1.51 (s, 6H). LCMS (ESI+): m/z 653.5 (M+H)⁺.

### Example 77B

### 4-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylbutan-2-yl dihydrogen phosphate

To a mixture of Example 77A (1.1 g, 1.685 mmol) and tetrakis(triphenylphosphine)palladium(0) (0.195 g, 0.169 mmol) in tetrahydrofuran (8.43 mL) under nitrogen was treated with pyrrolidine (0.599 g, 8.43 mmol) and the mixture stirred at ambient temperature for 2 hours. Concentration and purification by prep-HPLC (100:0 to 100:70 acetonitrile/water) afforded the title compound (0.70 g). ¹H NMR (500 MHz, dimethyl sulfoxide-d₆) δ ppm 10.73 (s, 1H), 8.41 (d, J=15.2 Hz, 2H), 8.06 (s, 1H), 7.92 (d, J=2.3 Hz, 1H), 7.62 (d, J=0.7 Hz, 1H), 7.23 (d, J=8.6 Hz, 1H), 7.17 (d, J=2.4 Hz, 1H), 6.80 (dd, J=8.6, 2.4 Hz, 1H), 4.50-4.45 (m, 4H), 4.28 (s, 1H), 4.13 (s, 2H), 3.83 (s, 3H), 2.12 (t, J=7.0 Hz, 2H), 1.46 (s, 6H). LCMS (ESI+): m/z 573.1 (M+H)⁺.

### Example 78

### 6-[1-(6-methyl-1,3-benzoxazol-2-yl)azetidin-3-yl]-3-[(1-methyl-1H-pyrazol-4-yl)amino]pyridine-2-carboxamide

The title compound was prepared as described in Example 1 using 2-chloro-6-methylbenzo[d]oxazole in place of 2-chlorobenzo[d]oxazole and Intermediate 4 in place of Intermediate 1. ¹H NMR (400 MHz, dimethyl sulfoxide-d₆) δ ppm 9.77 (s, 1H), 8.13 (d, J=3.1 Hz, 1H), 7.75 (s, 1H), 7.56 (d, J=3.1 Hz, 1H), 7.39 (d, J=0.8 Hz, 1H), 7.33 (d, J=8.8 Hz, 1H), 7.26 (d, J=8.7 Hz, 1H), 7.20 (d, J=1.7 Hz, 1H), 7.14 (d, J=7.9 Hz, 1H), 6.94 (ddd, J=8.0, 1.6, 0.8 Hz, 1H), 4.43 (t, J=8.2 Hz, 2H), 4.36 (dd, J=7.9, 6.4 Hz, 2H), 4.09 (tt, J=8.7, 6.4 Hz, 1H), 3.78 (s, 3H), 2.31 (s, 3H). LCMS (ESI+): m/z 404.2 (M+H)⁺.

### Example 79

### 6-(1-{6-[(2-hydroxycyclopentyl)oxy]-1,3-benzoxazol-2-yl}azetidin-3-yl)-3-[(1-methyl-1H-pyrazol-4-yl)amino]pyrazine-2-carboxamide

The title compound was prepared as described in Example 68 using 6-oxobicyclo[3.1.0]hexane in place of (R)-propylene oxide. ¹H NMR (600 MHz, dimethyl sulfoxide-d₆) δ ppm 10.71 (s, 1H), 8.39 (d, J=9.7 Hz, 2H), 8.04 (d, J=0.8 Hz, 1H), 7.89 (d, J=2.3 Hz, 1H), 7.60 (d, J=0.8 Hz, 1H), 7.18 (d, J=8.5 Hz, 1H), 7.13 (d, J=2.4 Hz, 1H), 6.76 (dd, J=8.6, 2.4 Hz, 1H), 4.96 (s, 1H), 4.48-4.37 (m, 5H), 4.29-4.19 (m, 1H), 4.05 (s, 1H), 3.82 (s, 3H), 2.08 (ddt, J=13.2, 9.0, 6.4 Hz, 1H), 1.93-1.82 (m, 1H), 1.77-1.65 (m, 2H), 1.62 (dddd, J=21.5, 10.6, 5.6, 3.2 Hz, 1H), 1.52 (dddd, J=13.2, 8.4, 5.2, 3.5 Hz, 1H). LCMS (ESI+): m/z 491.2 (M+H)⁺.

### Example 80

### 3-[(1-methyl-1H-pyrazol-4-yl)amino]-6-(1-{6-[(1-methyl-1H-pyrazol-3-yl)methoxy]-1,3-benzoxazol-2-yl}azetidin-3-yl)pyrazine-2-carboxamide

A mixture of Example 20 (100 mg, 0.246 mmol) and potassium carbonate (102 mg, 0.738 mmol) in N,N-dimethylformamide (2 mL) was stirred at ambient temperature for 15 minutes. 3-(Bromomethyl)-1-methyl-1H-pyrazole hydrobromic acid (63 mg, 0.246 mmol) was added, and the mixture was stirred at ambient temperature overnight. Water was added and the mixture was extracted with ethyl acetate. The organic layers were washed with water, washed with brine, dried over Na₂SO₄, and concentrated. The residue was purified by silica gel chromatography eluting with 100/0 to 100/10 dichloromethane/methanol to afford the title compound (90 mg). ¹H NMR (600 MHz, dimethyl sulfoxide-d₆) δ ppm 10.72 (s, 1H), 8.39 (d, J=14.9 Hz, 2H), 8.05 (d, J=0.8 Hz, 1H), 7.90 (d, J=2.3 Hz, 1H), 7.66 (d, J=2.1 Hz, 1H), 7.61 (d, J=0.8 Hz, 1H), 7.23-7.18 (m, 2H), 6.83 (dd, J=8.6, 2.5 Hz, 1H), 6.30 (d, J=2.2 Hz, 1H), 4.97 (s, 2H), 4.48-4.41 (m, 4H), 4.24 (tt, J=8.6, 7.0 Hz, 1H), 3.82 (d, J=3.9 Hz, 6H). LC/MS (ESI+): m/z 501 (M+H)⁺.

### Example 81

### 3-[(1-methyl-1H-pyrazol-4-yl)amino]-6-[1-(6-{2-oxo-2-[(2R)-2-(trifluoromethyl)pyrrolidin-1-yl]ethyl}-1,3-benzoxazol-2-yl)azetidin-3-yl]pyrazine-2-carboxamide

The title compound was prepared as described in Example 66 using (R)-2-(trifluoromethyl)pyrrolidine in place of (S)-pyrrolidin-2-ylmethanol. ¹H NMR (400 MHz, dimethyl sulfoxide-d₆, 90° C.) δ ppm 10.51 (s, 1H), 8.34 (s, 1H), 8.02 (br s, 1H), 7.92 (s, 1H), 7.54 (s, 1H), 7.50 (br s, 1H), 7.23 (d, J=1.6 Hz, 1H), 7.20 (d, J=8.0 Hz, 1H), 7.01 (dd, J=8.0, 1.6 Hz, 1H), 4.50 (t, J=8.3 Hz, 2H), 4.43 (dd, J=7.9, 6.4 Hz, 2H), 4.22 (tt, J=8.6, 6.4 Hz, 1H), 3.79 (s, 3H), 3.81-3.57 (m, 3H), 3.51-3.40 (m, 1H), 3.15-3.09 (m, 1H), 2.13-1.88 (m, 4H). LC/MS (ESI+): m/z 570.3 (M+H)⁺.

### Example 82

### 3-[(1-methyl-1H-pyrazol-4-yl)amino]-6-(1-{6-[(5-methyl-1,3-thiazol-2-yl)methoxy]-1,3-benzoxazol-2-yl}azetidin-3-yl)pyrazine-2-carboxamide

The title compound was prepared as described in Example 80 using 2-(chloromethyl)-5-methylthiazole in place of 3-(bromomethyl)-1-methyl-1H-pyrazole hydrobromic acid. ¹H NMR (500 MHz, dimethyl sulfoxide-d₆) δ ppm 10.72 (s, 1H), 8.39 (d, J=15.8 Hz, 2H), 8.05 (d, J=0.8 Hz, 1H), 7.90 (d, J=2.2 Hz, 1H), 7.61 (d, J=0.8 Hz, 1H), 7.50 (q, J=1.2 Hz, 1H), 7.25 (d, J=2.5 Hz, 1H), 7.22 (d, J=8.6 Hz, 1H), 6.88 (dd, J=8.5, 2.5 Hz, 1H), 5.33 (s, 2H), 4.52-4.40 (m, 4H), 4.29-4.19 (m, 1H), 3.82 (s, 3H), 2.44 (d, J=1.2 Hz, 3H). LCMS (ESI+): m/z 518 (M+H)⁺.

### Example 83

### 6-(1-{6-[2-(dimethylamino)-2-oxoethoxy]-1,3-benzoxazol-2-yl}azetidin-3-yl)-3-[(1-methyl-1H-pyrazol-4-yl)amino]pyridine-2-carboxamide

The title compound was prepared as described in Example 23 using 2-chloro-N,N-dimethylacetamide in place of (iodomethyl)cyclopropane. ¹H NMR (400 MHz, dimethyl sulfoxide-d₆) δ ppm 9.79 (s, 1H), 8.16 (d, J=3.1 Hz, 1H), 7.78 (s, 1H), 7.59 (d, J=3.0 Hz, 1H), 7.42 (d, J=3.0 Hz, 1H), 7.36 (d, J=8.7 Hz, 1H), 7.29 (d, J=8.7 Hz, 1H), 7.19 (d, J=8.6 Hz, 1H), 7.11 (d, J=2.5 Hz, 1H), 6.78 (dd, J=8.6, 2.5 Hz, 1H), 4.77 (s, 2H), 4.46 (t, J=8.2 Hz, 2H), 4.38 (t, J=7.1 Hz, 2H), 4.19-4.07 (m, 1H), 3.82 (s, 3H), 3.00 (s, 3H), 2.84 (s, 3H). LCMS (ESI+): m/z 491.3 (M+H)⁺.

### Example 84

### 6-{1-[6-(3-hydroxy-3-methylbutoxy)-1,3-benzoxazol-2-yl]azetidin-3-yl}-3-[(1-methyl-1H-pyrazol-4-yl)amino]pyridine-2-carboxamide

### Example 84A

### 6-[1-(6-hydroxy-1,3-benzoxazol-2-yl)azetidin-3-yl]-3-[(1-methyl-1H-pyrazol-4-yl)amino]pyridine-2-carboxamide

To a mixture of Intermediate 4E (500 mg, 1.0 mmol) and potassium carbonate (552 mg, 4.0 mmol) in N,N-dimethylformamide (0.5 mL) was added 2-chlorobenzo[d]oxazol-6-ol (169 mg, 1.0 mmol), and the mixture was stirred for 2 hours. The mixture was diluted with water, washed with ethyl acetate, and dried over Na₂SO₄. Concentration and purification on silica gel using 100/0 to 100/5 ethyl acetate/methanol afforded the title compound (200 mg). ¹H NMR (600 MHz, dimethyl sulfoxide-d₆) δ ppm 9.79 (s, 1H), 9.26 (s, 1H), 8.16 (d, J=3.2 Hz, 1H), 7.84-7.74 (m, 1H), 7.60 (d, J=3.2 Hz, 1H), 7.42 (d, J=0.8 Hz, 1H), 7.36 (d, J=8.7 Hz, 1H), 7.30 (d, J=8.7 Hz, 1H), 7.09 (dd, J=8.4, 0.4 Hz, 1H), 6.83 (dd, J=2.3, 0.4 Hz, 1H), 6.61 (dd, J=8.4, 2.3 Hz, 1H), 4.42 (dd, J=8.6, 7.7 Hz, 2H), 4.35 (dd, J=7.8, 6.5 Hz, 2H), 4.17-4.04 (m, 1H), 3.82 (s, 3H). LCMS (ESI+): m/z 406.2 (M+H)⁺.

### Example 84B

### 6-{1-[6-(3-hydroxy-3-methylbutoxy)-1,3-benzoxazol-2-yl]azetidin-3-yl}-3-[(1-methyl-1H-pyrazol-4-yl)amino]pyridine-2-carboxamide

The title compound was prepared as described in Example 31 using Example 84A in place of Example 20. ¹H NMR (500 MHz, dimethyl sulfoxide-d₆) δ ppm 9.82 (s, 1H), 8.18 (d, J=3.1 Hz, 1H), 7.81 (d, J=0.8 Hz, 1H), 7.63 (d, J=3.1 Hz, 1H), 7.44 (d, J=0.8 Hz, 1H), 7.38 (d, J=8.7 Hz, 1H), 7.31 (d, J=8.7 Hz, 1H), 7.20 (d, J=8.5 Hz, 1H), 7.12 (d, J=2.4 Hz, 1H), 6.77 (dd, J=8.5, 2.4 Hz, 1H), 4.46 (dd, J=8.6, 7.8 Hz, 2H), 4.43-4.35 (m, 3H), 4.19-4.11 (m, 1H), 4.08 (t, J=7.2 Hz, 2H), 1.84 (t, J=7.2 Hz, 2H), 1.18 (s, 6H). LCMS (ESI+): m/z 492.3 (M+H)⁺.

### Example 85

### 6-{1-[6-(2-hydroxy-2-methylpropoxy)-1,3-benzoxazol-2-yl]azetidin-3-yl}-3-[(1-methyl-1H-pyrazol-4-yl)amino]pyridine-2-carboxamide

The title compound was prepared as described in Example 31 using 1-bromo-2-methylpropan-2-ol in place of 4-bromo-2-methylbutan-2-ol and Example 84A in place of Example 20. ¹H NMR (600 MHz, dimethyl sulfoxide-d₆) δ ppm 9.80 (s, 1H), 8.17 (d, J=3.2 Hz, 1H), 7.79 (d, J=0.8 Hz, 1H), 7.61 (d, J=3.1 Hz, 1H), 7.42 (d, J=0.9 Hz, 1H), 7.37 (d, J=8.7 Hz, 1H), 7.30 (d, J=8.7 Hz, 1H), 7.19 (d, J=8.5 Hz, 1H), 7.10 (d, J=2.4 Hz, 1H), 6.78 (dd, J=8.5, 2.4 Hz, 1H), 4.61 (s, 1H), 4.45 (dd, J=8.6, 7.7 Hz, 2H), 4.38 (dd, J=7.8, 6.4 Hz, 2H), 4.12 (tt, J=8.6, 6.5 Hz, 1H), 3.82 (s, 3H), 3.69 (s, 2H), 1.20 (s, 6H). LCMS (ESI+): m/z 478.2 (M+H)⁺.

### Example 86

### 6-(1-{6-[(5-methyl-1,3,4-oxadiazol-2-yl)methoxy]-1,3-benzoxazol-2-yl}azetidin-3-yl)-3-[(1-methyl-1H-pyrazol-4-yl)amino]pyrazine-2-carboxamide

The title compound was prepared as described in Example 80 using 2-(bromomethyl)-5-methyl-1,3,4-oxadiazole in place of 3-(bromomethyl)-1-methyl-1H-pyrazole hydrobromic acid and cesium carbonate in place of potassium carbonate. ¹H NMR (400 MHz, dimethyl sulfoxide-d₆) δ ppm 10.71 (s, 1H), 8.39 (d, J=8.8 Hz, 2H), 8.04 (s, 1H), 7.88 (d, J=2.2 Hz, 1H), 7.61 (d, J=0.8 Hz, 1H), 7.28 (d, J=2.5 Hz, 1H), 7.23 (d, J=8.5 Hz, 1H), 6.90 (dd, J=8.6, 2.5 Hz, 1H), 5.34 (s, 2H), 4.52-4.39 (m, 4H), 4.25 (q, J=7.7 Hz, 1H), 3.82 (s, 3H), 2.53 (s, 3H). LCMS (ESI+): m/z 503 (M+H)⁺.

### Example 87

### 6-{1-[6-(2-cyclopropyl-2-oxoethoxy)-1,3-benzoxazol-2-yl]azetidin-3-yl}-3-[(1-methyl-1H-pyrazol-4-yl)amino]pyrazine-2-carboxamide

The title compound was prepared as described in Example 31 using 2-bromo-1-cyclopropylethanone in place of 4-bromo-2-methylbutan-2-ol. ¹H NMR (400 MHz, dimethyl sulfoxide-d₆) δ ppm 10.70 (s, 1H), 8.38 (s, 2H), 8.04 (s, 1H), 7.88 (s, 1H), 7.60 (s, 1H), 7.20 (d, J=8.5 Hz, 1H), 7.10 (d, J=2.4 Hz, 1H), 6.77 (dd, J=8.6, 2.5 Hz, 1H), 4.92 (s, 2H), 4.53-4.37 (m, 4H), 4.24 (p, J=7.6 Hz, 1H), 3.82 (s, 3H), 2.22 (tt, J=7.7, 4.6 Hz, 1H), 1.04-0.84 (m, 4H). LCMS (ESI+): m/z 489.0 (M+H)⁺.

### Example 88

### 6-{1-[6-(4-hydroxyphenyl)-1,3-benzoxazol-2-yl]azetidin-3-yl}-3-[(1-methyl-1H-pyrazol-4-yl)amino]pyrazine-2-carboxamide

A mixture of Example 6A (410 mg, 0.76 mmol), 4-hydroxyphenylboronic acid (159 mg, 1.15 mmol) and aqueous potassium phosphate (375 mg, 2.02 mmol, 1 mL water) was combined in dioxane (3 mL) in a microwave tube (20 mL). After sparging with nitrogen for 20 minutes, tris(dibenzylideneacetone)dipalladium(0) (20.9 mg, 0.023 mmol) and (1S,3R,5R,7S)-1,3,5,7-tetramethyl-8-phenyl-2,4,6-trioxa-8-phosphaadamantane (14.1 mg, 0.048 mmol) were added. The vial was heated at 65° C. for 18 hours and cooled to ambient temperature. The mixture was filtered, washed with methanol, and dried in vacuo to afford the title compound (358 mg). ¹H NMR (400 MHz, dimethyl sulfoxide-d₆) δ ppm 10.71 (s, 1H), 9.48 (s, 1H), 8.41 (s, 1H), 8.39 (s, 1H), 8.04 (s, 1H), 7.92-7.86 (m, 1H), 7.63 (d, J=1.7 Hz, 1H), 7.61 (s, 1H), 7.52-7.43 (m, 2H), 7.39 (dd, J=8.2, 1.8 Hz, 1H), 7.32 (d, J=8.2 Hz, 1H), 6.87-6.79 (m, 2H), 4.50 (d, J=7.7 Hz, 4H), 4.27 (p, J=7.6 Hz, 1H), 3.82 (s, 3H). LCMS (APCI+) m/z 483.8 (M+H)⁺.

### Example 89

### 6-(1-{6-[4-(2-hydroxyethoxy)phenyl]-1,3-benzoxazol-2-yl}azetidin-3-yl)-3-[(1-methyl-1H-pyrazol-4-yl)amino]pyrazine-2-carboxamide

The title compound was prepared as described in Example 88 using 4-(2-hydroxyethoxy)phenylboronic acid in place of 4-hydroxyphenylboronic acid. The crude reaction mixture was concentrated and purified by RP-HPLC (Sunfire column) using a gradient elution of 5/95 acetonitrile/0.1% trifluoroacetic acid in water to 70/30 over 30 minutes to provide the title compound as the trifluoroacetate salt. This was dissolved in methanol and loaded onto a Silicycle carbonate column (2 g) and the free base eluted from the column with methanol. After concentrating in vacuo, the title compound was obtained. ¹H NMR (600 MHz, dimethyl sulfoxide-d₆) δ ppm 10.73 (s, 1H), 8.45-8.41 (m, 1H), 8.39 (s, 1H), 8.05 (d, J=0.8 Hz, 1H), 7.91 (d, J=2.4 Hz, 1H), 7.68 (dd, J=1.8, 0.6 Hz, 1H), 7.63-7.56 (m, 3H), 7.43 (dd, J=8.2, 1.7 Hz, 1H), 7.34 (dd, J=8.2, 0.5 Hz, 1H), 7.04-6.98 (m, 2H), 4.89 (t, J=5.5 Hz, 1H), 4.54-4.47 (m, 4H), 4.31-4.23 (m, 1H), 4.02 (dd, J=5.4, 4.6 Hz, 2H), 3.82 (s, 3H), 3.74 (q, J=5.2 Hz, 2H). LCMS (APCI+) m/z 527.4 (M+H)⁺.

### Example 90

### 6-(1-{6-[1-(2-hydroxyethyl)-1H-pyrazol-4-yl]-1,3-benzoxazol-2-yl}azetidin-3-yl)-3-[(1-methyl-1H-pyrazol-4-yl)amino]pyrazine-2-carboxamide

The compound was prepared as described in Example 88 using 2-(4-(4,4,5,5-tetramethyl-1,3,2-dioxaborolan-2-yl)-1H-pyrazol-1-yl)ethanol in place of 4-hydroxyphenylboronic acid. Purification was carried out as described in Example 89. ¹H NMR (600 MHz, dimethyl sulfoxide-d₆) δ ppm 10.72 (s, 1H), 8.43 (d, J=2.2 Hz, 1H), 8.39 (s, 1H), 8.10 (d, J=0.8 Hz, 1H), 8.05 (d, J=0.8 Hz, 1H), 7.90 (d, J=2.3 Hz, 1H), 7.86 (d, J=0.8 Hz, 1H), 7.69-7.64 (m, 1H), 7.61 (d, J=0.8 Hz, 1H), 7.39 (dd, J=8.1, 1.6 Hz, 1H), 7.27 (d, J=8.0 Hz, 1H), 4.94 (s, 1H), 4.52-4.45 (m, 4H), 4.30-4.22 (m, 1H), 4.15 (t, J=5.7 Hz, 2H), 3.82 (s, 3H), 3.76 (t, J=5.7 Hz, 2H). LCMS (APCI+) m/z 501.5 (M+H)⁺.

### Example 91

### 6-(1-{6-[1-(2-methoxyethyl)-1H-pyrazol-4-yl]-1,3-benzoxazol-2-yl}azetidin-3-yl)-3-[(1-methyl-1H-pyrazol-4-yl)amino]pyrazine-2-carboxamide

The title compound was prepared as described in Example 88 using 1-(2-methoxyethyl)-1H-pyrazol-4-ylboronic acid in place of 4-hydroxyphenylboronic acid. Purification was carried out as described in Example 89. ¹H NMR (600 MHz, dimethyl sulfoxide-d₆) δ ppm 10.72 (s, 1H), 8.45-8.42 (m, 1H), 8.39 (s, 1H), 8.11 (d, J=0.8 Hz, 1H), 8.05 (d, J=0.8 Hz, 1H), 7.93-7.89 (m, 1H), 7.87 (d, J=0.8 Hz, 1H), 7.65 (dd, J=1.7, 0.6 Hz, 1H), 7.61 (d, J=0.8 Hz, 1H), 7.39 (dd, J=8.1, 1.7 Hz, 1H), 7.27 (dd, J=8.1, 0.5 Hz, 1H), 4.52-4.45 (m, 4H), 4.30-4.22 (m, 3H), 3.82 (s, 3H), 3.71 (dd, J=5.7, 5.0 Hz, 2H), 3.25 (s, 3H). LCMS (APCI+) m/z 515.1 (M+H)⁺.

### Example 92

### 3-[(1-methyl-1H-pyrazol-4-yl)amino]-6-{1-[6-(1-methyl-1H-pyrazol-4-yl)-1,3-benzoxazol-2-yl]azetidin-3-yl}pyrazine-2-carboxamide

The title compound was prepared as described in Example 88 using 1-methyl-4-(4,4,5,5-tetramethyl-1,3,2-dioxaborolan-2-yl)-1H-pyrazole in place of 4-hydroxyphenylboronic acid. Purification was carried out as described in Example 89. ¹H NMR (500 MHz, dimethyl sulfoxide-d₆) δ ppm 10.72 (s, 1H), 8.43 (d, J=2.3 Hz, 1H), 8.39 (s, 1H), 8.08 (d, J=0.8 Hz, 1H), 8.05 (d, J=0.7 Hz, 1H), 7.91 (d, J=2.3 Hz, 1H), 7.84 (d, J=0.8 Hz, 1H), 7.64 (d, J=1.6 Hz, 1H), 7.61 (d, J=0.8 Hz, 1H), 7.38 (dd, J=8.1, 1.6 Hz, 1H), 7.27 (d, J=8.1 Hz, 1H), 4.53-4.44 (m, 4H), 4.31-4.21 (m, 1H), 3.86 (s, 3H), 3.82 (s, 3H). LCMS (APCI+) m/z 471.1 (M+H)⁺.

### Example 93

### 3-[(1-methyl-1H-pyrazol-4-yl)amino]-6-(1-{6-[(1H-pyrazol-4-yl)methoxy]-1,3-benzoxazol-2-yl}azetidin-3-yl)pyrazine-2-carboxamide

### Example 93A

### tert-butyl 4-{[(methanesulfonyl)oxy]methyl}-1H-pyrazole-1-carboxylate

A mixture of tert-butyl 4-(hydroxymethyl)-1H-pyrazole-1-carboxylate (120 mg, 0.605 mmol), N,N-dimethylpyridin-4-amine (7.4 mg, 0.061 mmol), and triethylamine (169 μL, 1.21 mmol) in dichloromethane (3 mL) was cooled to 0° C. and methanesulfonyl chloride (47 μL, 0.605 mmol) was added. After warming to ambient temperature overnight, the mixture was diluted with dichloromethane, washed with saturated NaHCO₃, washed with brine, dried over MgSO₄, filtered, and concentrated. The residue was purified by silica gel chromatography eluting with 100/0 to 100/10 dichloromethane/methanol to afford the title compound (100 mg). ¹H NMR (400 MHz, dimethyl sulfoxide) δ ppm 8.57-8.02 (m, 1H), 7.83 (d, J=0.7 Hz, 1H), 4.67 (s, 2H), 3.31 (s, 3H), 1.54 (s, 9H).

### Example 93B

### tert-butyl 4-({[2-(3-{6-carbamoyl-5-[(1-methyl-1H-pyrazol-4-yl)amino]pyrazin-2-yl}azetidin-1-yl)-1,3-benzoxazol-6-yl]oxy}methyl)-1H-pyrazole-1-carboxylate

A mixture of Example 20 (60 mg, 0.148 mmol), cesium carbonate (120 mg, 0.369 mmol) and Example 93A (44.9 mg, 0.162 mmol) in tetrahydrofuran (2 mL) was stirred at 70° C. for 3 hours. Water was added and the mixture was extracted with ethyl acetate. The organic layers were washed with water, washed with brine, dried over Na₂SO₄, concentrated and purified by silica gel chromatography eluting with 100/0 to 100/10 dichloromethane/methanol to afford the title compound (40 mg). LCMS (ESI+): m/z 587 (M+H)⁺.

### Example 93C

### 3-[(1-methyl-1H-pyrazol-4-yl)amino]-6-(1-{6-[(1H-pyrazol-4-yl)methoxy]-1,3-benzoxazol-2-yl}azetidin-3-yl)pyrazine-2-carboxamide

To a solution of Example 93B (40 mg, 0.068 mmol) in dichloromethane (1 mL) was added trifluoroacetic acid (105 μL, 1.36 mmol), and the mixture was stirred at ambient temperature for 1 hour. The mixture was concentrated, and the residue re-dissolved in methanol and passed through a SilicaPrep Carbonate column. Concentration afforded the title compound (17 mg). ¹H NMR (400 MHz, dimethyl sulfoxide) δ ppm 12.78 (s, 1H), 10.67 (s, 1H), 8.35 (d, J=6.9 Hz, 2H), 8.01 (d, J=0.8 Hz, 1H), 7.93-7.62 (m, 2H), 7.57 (d, J=0.8 Hz, 2H), 7.29-6.95 (m, 2H), 6.77 (dd, J=8.6, 2.4 Hz, 1H), 4.93 (s, 2H), 4.40 (dd, J=7.6, 2.0 Hz, 4H), 4.30-4.11 (m, 1H), 3.78 (s, 3H). LCMS (ESI+): m/z 487 (M+H)⁺.

### Example 94

### 3-[(1-tert-butyl-1H-pyrazol-4-yl)amino]-6-{1-[6-(3-hydroxy-3-methylbutoxy)-1,3-benzoxazol-2-yl]azetidin-3-yl}pyrazine-2-carboxamide

### Example 94A

### methyl 3-chloro-6-[1-(6-hydroxy-1,3-benzoxazol-2-yl)azetidin-3-yl]pyrazine-2-carboxylate

The title compound was prepared as described for Intermediate 3, using 2-chlorobenzo[d]oxazol-6-ol in place of 2-chlorobenzoxazole. ¹H NMR (400 MHz, dimethyl sulfoxide-d₆) δ ppm 9.26 (s, 1H), 8.77 (s, 1H), 7.11 (d, J=8.4 Hz, 1H), 6.84 (d, J=2.3 Hz, 1H), 6.62 (dd, J=8.4, 2.3 Hz, 1H), 4.52 (dd, J=8.3, 7.2 Hz, 2H), 4.45-4.36 (m, 1H), 4.36-4.23 (m, 2H), 3.93 (s, 3H). LCMS (ESI+): m/z 361.3 (M+H)⁺.

### Example 94B

### methyl 3-[(1-tert-butyl-1H-pyrazol-4-yl)amino]-6-[1-(6-hydroxy-1,3-benzoxazol-2-yl)azetidin-3-yl]pyrazine-2-carboxylate

To a solution of Example 94A (0.16 g, 0.444 mmol) in methanol (4.43 mL) was added 1-(tert-butyl)-1H-pyrazol-4-amine (0.105 g, 0.754 mmol) and N,N-diisopropylethylamine (0.093 mL, 0.532 mmol), and the mixture was heated at 70° C. for 4 hours. The mixture was cooled to ambient temperature, quenched with water, and extracted with ethyl acetate. The organic extracts were washed with brine, dried over Na₂SO₄, concentrated, and purified by silica gel chromatography to afford the title compound (0.11 g, 90% purity). This material was used in the next step without further purification. LCMS (ESI+): m/z 464.1 (M+H)⁺.

### Example 94C

### 3-[(1-tert-butyl-1H-pyrazol-4-yl)amino]-6-[1-(6-hydroxy-1,3-benzoxazol-2-yl)azetidin-3-yl]pyrazine-2-carboxamide

To a solution of Example 94B (0.10 g, 0.215 mmol) in methanol (2.15 mL) was added 7 M ammonia in methanol (0.614 mL, 4.30 mmol), and the mixture was heated at 65° C. for 12 hours. After cooling to ambient temperature, the mixture was filtered, and the residue dried in vacuo to afford the title compound (0.085 g). ¹H NMR (500 MHz, dimethyl sulfoxide-d₆) δ ppm 10.71 (s, 1H), 9.25 (s, 1H), 8.39 (d, J=13.9 Hz, 2H), 8.11 (d, J=0.7 Hz, 1H), 7.89 (d, J=2.3 Hz, 1H), 7.68 (d, J=0.7 Hz, 1H), 7.09 (d, J=8.4 Hz, 1H), 6.83 (d, J=2.4 Hz, 1H), 6.61 (dd, J=8.4, 2.3 Hz, 1H), 4.47-4.34 (m, 4H), 4.21 (q, J=7.8 Hz, 1H), 1.51 (s, 9H). LCMS (ESI+): m/z 449.4 (M+H)⁺.

### Example 94D

### 3-[(1-tert-butyl-1H-pyrazol-4-yl)amino]-6-{1-[6-(3-hydroxy-3-methylbutoxy)-1,3-benzoxazol-2-yl]azetidin-3-yl}pyrazine-2-carboxamide

The title compound was prepared as described in Example 31 using Example 94C in place of Example 20. ¹H NMR (600 MHz, dimethyl sulfoxide-d₆) δ ppm 10.72 (s, 1H), 8.41 (d, J=9.0 Hz, 2H), 8.11 (d, J=0.8 Hz, 1H), 7.94-7.85 (m, 1H), 7.69 (d, J=0.8 Hz, 1H), 7.20 (d, J=8.6 Hz, 1H), 7.13 (d, J=2.4 Hz, 1H), 6.77 (dd, J=8.6, 2.4 Hz, 1H), 4.45 (d, J=7.7 Hz, 4H), 4.23 (p, J=7.6 Hz, 1H), 4.06 (t, J=7.2 Hz, 2H), 1.83 (t, J=7.2 Hz, 2H), 1.51 (s, 9H), 1.16 (s, 6H). LCMS (ESI+):536.2 (M+H)⁺.

### Example 95

### 4-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylbutan-2-yl glycinate

### Example 95A

### 4-{[2-(3-f{6-carbamoyl-5-[(1-methyl-1H-pyrazol-4-yl)amino]pyrazin-2-yl}azetidin-1-yl)-1,3-benzoxazol-6-yl]oxy}-2-methylbutan-2-yl [(tert-butoxycarbonyl)amino]acetate

To a mixture of Example 31 (6.81 g, 13.83 mmol), Boc-glycine (7.27 g, 41.5 mmol) and 4-pyrrolopyridine (2.05 g, 13.83 mmol) in dichloromethane (45 mL) was added a solution of N,N′-diisopropylcarbodiimide (5.23 g, 41.5 mmol) in dichloromethane (25 mL) slowly over 1 hour under reflux, and the mixture was refluxed for another 3 hours. (The product mixture included the desired coupling product, along with a product which underwent a second acylation to convert the desired product to an imide which could be converted to the desired product by careful treatment with hydrazine). The reaction mixture was cooled to ambient temperature, hydrazine hydrate was added (50% solution, 12.13 mL, 124 mmol), and the mixture stirred for 2 hours to completely convert the imide to the desired product. The mixture was cooled to 0° C., filtered, and the filtrate was washed with cold 1 N hydrochloric acid, washed with saturated NaHCO₃, and washed with brine. The organic layer was dried, concentrated, and purified by silica gel chromatography (100:0 to 100:10 ethyl acetate/methanol) to provide the title compound (7.5 g). ¹H NMR (600 MHz, dimethyl sulfoxide-d₆) δ ppm 10.71 (s, 1H), 8.38 (d, J=4.4 Hz, 2H), 8.04 (d, J=0.8 Hz, 1H), 7.89 (d, J=2.2 Hz, 1H), 7.60 (d, J=0.8 Hz, 1H), 7.23-7.14 (m, 3H), 6.78 (dd, J=8.5, 2.4 Hz, 1H), 4.48-4.39 (m, 4H), 4.29-4.19 (m, 1H), 4.05 (t, J=7.0 Hz, 2H), 3.82 (s, 3H), 3.58 (d, J=6.2 Hz, 2H), 2.22 (t, J=7.0 Hz, 2H), 1.47 (s, 6H), 1.37 (s, 9H). LCMS (ESI+): m/z 650.4 (M+H)⁺.

### Example 95B

### 4-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylbutan-2-yl glycinate

A solution of Example 95A (7.32 g, 11.27 mmol) in a 1:2 trifluoroacetic acid/dichloromethane (1 mL) was stirred for 2 hours at ambient temperature. The mixture was concentrated, re-dissolved in dichloromethane, washed with saturated aqueous NaHCO₃, washed with brine, and dried over Na₂SO₄. Concentration and purification by silica gel chromatography (100:0 to 100:30 ethyl acetate/methanol) afforded the title compound (5.21 g). ¹H NMR (600 MHz, dimethyl sulfoxide-d₆) δ ppm 10.70 (s, 1H), 8.38 (s, 2H), 8.04 (d, J=0.7 Hz, 1H), 7.89 (d, J=2.4 Hz, 1H), 7.60 (d, J=0.8 Hz, 1H), 7.19 (d, J=8.6 Hz, 1H), 7.14 (d, J=2.4 Hz, 1H), 6.77 (dd, J=8.6, 2.4 Hz, 1H), 4.44 (dd, J=7.7, 1.8 Hz, 4H), 4.29-4.18 (m, 1H), 4.05 (t, J=6.9 Hz, 2H), 3.82 (s, 3H), 3.18 (s, 2H), 2.23 (t, J=6.9 Hz, 2H), 1.48 (s, 6H); only visible peaks. LCMS (ESI+): m/z 550.4 (M+H)⁺.

### Example 96

### 6-{1-[6-(3-hydroxy-3-methylbutoxy)-1,3-benzoxazol-2-yl]azetidin-3-yl}-3-{[1-(2-hydroxy-2-methylpropyl)-1H-pyrazol-4-yl]amino}pyrazine-2-carboxamide

### Example 96A

### 6-{1-[6-(3-hydroxy-3-methylbutoxy)-1,3-benzoxazol-2-yl]azetidin-3-yl}-3-[(1-{[2-(trimethylsilyl)ethoxy]methyl}-1H-pyrazol-4-yl)amino]pyrazine-2-carboxamide

A solution of Example 0 (500 mg, 0.957 mmol) in N,N-dimethylformamide (3.8 mL) under nitrogen was treated with 60% sodium hydride (57.4 mg, 1.435 mmol), and the mixture stirred at ambient temperature for 20 minutes. 4-Bromo-2-methylbutan-2-ol (90%, 213 mg, 1.148 mmol) was added and the mixture was heated to 70° C. overnight. The mixture was cooled to ambient temperature, quenched with water, washed with ethyl acetate, and dried over Na₂SO₄. Concentration and purification by silica gel chromatography (0:100 to 10:100 methanol/dichloromethane) provided the title compound (539 mg). ¹H NMR (400 MHz, dimethyl sulfoxide-d₆) δ ppm 10.77 (s, 1H), 8.40 (d, J=3.3 Hz, 2H), 8.23 (d, J=0.7 Hz, 1H), 7.90 (d, J=2.3 Hz, 1H), 7.74 (d, J=0.7 Hz, 1H), 7.19 (d, J=8.5 Hz, 1H), 7.10 (d, J=2.4 Hz, 1H), 6.76 (dd, J=8.6, 2.4 Hz, 1H), 5.38 (s, 2H), 4.49-4.41 (m, 4H), 4.36 (s, 1H), 4.24 (p, J=7.6 Hz, 1H), 4.06 (t, J=7.2 Hz, 2H), 3.58-3.48 (m, 2H), 1.83 (t, J=7.2 Hz, 2H), 1.16 (s, 6H), 0.88-0.78 (m, 2H), −0.06 (s, 9H). LCMS (ESI+): m/z 609.2 (M+H)⁺.

### Example 96B

### 6-{1-[6-(3-hydroxy-3-methylbutoxy)-1,3-benzoxazol-2-yl]azetidin-3-yl}-3-[(1H-pyrazol-4-yl)amino]pyrazine-2-carboxamide

A solution of Example 96A (573 mg, 0.941 mmol) in tetrahydrofuran (7.76 mL) under nitrogen was treated with tetrabutylammonium fluoride (9.41 mL, 9.41 mmol, 1 M in tetrahydrofuran), and the mixture was stirred at 85° C. for 16 hours. The mixture was cooled, diluted with ethyl acetate, washed with water, washed with brine, dried over Na₂SO₄, and concentrated. The residue was purified by silica gel chromatography (0:100 to 10:100 methanol/dichloromethane) to provide the title compound (343 mg). ¹H NMR (400 MHz, dimethyl sulfoxide-d₆) δ ppm 12.60 (s, 1H), 10.71 (s, 1H), 8.39 (d, J=6.8 Hz, 2H), 8.04 (s, 1H), 7.88 (d, J=2.4 Hz, 1H), 7.70 (s, 1H), 7.19 (d, J=8.5 Hz, 1H), 7.11 (d, J=2.3 Hz, 1H), 6.76 (dd, J=8.5, 2.4 Hz, 1H), 4.44 (dd, J=7.7, 1.2 Hz, 4H), 4.35 (s, 1H), 4.23 (p, J=7.6 Hz, 1H), 4.06 (t, J=7.2 Hz, 2H), 1.83 (t, J=7.2 Hz, 2H), 1.16 (s, 6H). LCMS (ESI+): m/z 479.3 (M+H)⁺.

### Example 96C

### 6-{1-[6-(3-hydroxy-3-methylbutoxy)-1,3-benzoxazol-2-yl]azetidin-3-yl}-3-{[1-(2-hydroxy-2-methylpropyl)-1H-pyrazol-4-yl]amino}pyrazine-2-carboxamide

To a mixture of Example 96B (31 mg, 0.065 mmol) and cesium carbonate (42.2 mg, 0.13 mmol) in N,N-dimethylformamide (0.65 mL) was added 2,2-dimethyloxirane (0.012 mL, 0.13 mmol), and the mixture was heated at 70° C. for 16 hours. The mixture was cooled, diluted with ethyl acetate, washed with water, and washed with brine. The organic layer was dried over Na₂SO₄, concentrated, and purified by silica gel chromatography (0:100 to 20:100 methanol/dichloromethane) to provide the title compound (0.015 g). ¹H NMR (400 MHz, dimethyl sulfoxide-d₆) δ ppm 10.75 (s, 1H), 8.39 (d, J=2.9 Hz, 2H), 8.07 (s, 1H), 7.89 (d, J=2.4 Hz, 1H), 7.62 (s, 1H), 7.20 (d, J=8.5 Hz, 1H), 7.12 (d, J=2.4 Hz, 1H), 6.77 (dd, J=8.6, 2.4 Hz, 1H), 4.45 (d, J=7.7 Hz, 4H), 4.24 (q, J=7.5 Hz, 1H), 4.07 (d, J=7.1 Hz, 2H), 3.98 (s, 2H), 1.83 (t, J=7.2 Hz, 2H), 1.16 (s, 6H), 1.06 (s, 6H). LCMS (ESI+): m/z 551.9 (M+H)⁺.

### Example 97

### 3-[(1-methyl-1H-pyrazol-4-yl)amino]-6-(1-{6-[(1-methyl-1H-pyrazol-4-yl)methoxy]-1,3-benzoxazol-2-yl}azetidin-3-yl)pyrazine-2-carboxamide

The title compound was prepared as described in Example 80 using 4-(bromomethyl)-1-methyl-1H-pyrazole, hydrobromic acid in place of 3-(bromomethyl)-1-methyl-1H-pyrazole hydrobromic acid and cesium carbonate in place of potassium carbonate. ¹H NMR (500 MHz, dimethyl sulfoxide-d₆) δ ppm 10.71 (s, 1H), 8.39 (d, J=12.1 Hz, 2H), 8.05 (d, J=0.8 Hz, 1H), 7.90 (d, J=2.3 Hz, 1H), 7.78 (s, 1H), 7.61 (d, J=0.8 Hz, 1H), 7.48 (d, J=0.8 Hz, 1H), 7.25-7.06 (m, 2H), 6.80 (dd, J=8.5, 2.5 Hz, 1H), 4.93 (s, 2H), 4.57-4.37 (m, 4H), 4.32-4.13 (m, 1H), 3.82 (d, J=2.8 Hz, 6H). LCMS (ESI+): m/z 501 (M+H)⁺

### Example 98

### 6-(1-{6-[(1-methyl-1H-imidazol-4-yl)methoxy]-1,3-benzoxazol-2-yl}azetidin-3-yl)-3-[(1-methyl-1H-pyrazol-4-yl)amino]pyrazine-2-carboxamide

The title compound was prepared as described in Example 80 using 4-(bromomethyl)-1-methyl-1H-imidazole hydrobromic acid in place of 3-(bromomethyl)-1-methyl-1H-pyrazole hydrobromic acid and sodium hydride in place of potassium carbonate. ¹H NMR (600 MHz, dimethyl sulfoxide-d₆) δ ppm 10.71 (s, 1H), 8.39 (d, J=11.2 Hz, 2H), 8.04 (d, J=0.8 Hz, 1H), 7.89 (d, J=2.3 Hz, 1H), 7.61 (d, J=0.8 Hz, 1H), 7.59-7.44 (m, 1H), 7.32-7.07 (m, 3H), 6.82 (dd, J=8.6, 2.4 Hz, 1H), 4.88 (s, 2H), 4.57-4.37 (m, 4H), 4.24 (tt, J=8.7, 7.0 Hz, 1H), 3.82 (s, 3H), 3.63 (s, 3H). LCMS (ESI+): m/z 501 (M+H)⁺.

### Example 99

### 3-[(1-methyl-1H-pyrazol-4-yl)amino]-6-{1-[6-(2-methyl-1,3-thiazol-5-yl)-1,3-benzoxazol-2-yl]azetidin-3-yl}pyrazine-2-carboxamide

The title compound was prepared as described in Example 88 using 2-methyl-5-(4,4,5,5-tetramethyl-1,3,2-dioxaborolan-2-yl)thiazole in place of 4-hydroxyphenylboronic acid. Purification was carried out as described in Example 89. ¹H NMR (400 MHz, dimethyl sulfoxide-d₆) δ ppm 10.72 (s, 1H), 8.46-8.41 (m, 1H), 8.38 (s, 1H), 8.04 (s, 1H), 7.96 (s, 1H), 7.93-7.85 (m, 1H), 7.74 (d, J=1.7 Hz, 1H), 7.61 (s, 1H), 7.39 (dd, J=8.1, 1.8 Hz, 1H), 7.32 (d, J=8.1 Hz, 1H), 4.51 (d, J=7.8 Hz, 4H), 4.27 (p, J=7.6 Hz, 1H), 3.82 (s, 3H), 2.66 (s, 3H). LCMS (ESI+) m/z 487.8 (M+H)⁺.

### Example 100

### 6-(1-{6-[4-(2-methoxyethoxy)phenyl]-1,3-benzoxazol-2-yl}azetidin-3-yl)-3-[(1-methyl-1H-pyrazol-4-yl)amino]pyrazine-2-carboxamide

The title compound was prepared as described in Example 88 using [4-(2-methoxyethoxy)phenyl]boronic acid in place of 4-hydroxyphenylboronic acid. Purification was carried out as described in Example 89. ¹H NMR (500 MHz, dimethyl sulfoxide-d₆) δ ppm 10.73 (s, 1H), 8.43 (d, J=2.3 Hz, 1H), 8.39 (s, 1H), 8.05 (d, J=0.7 Hz, 1H), 7.91 (d, J=2.4 Hz, 1H), 7.69 (dd, J=1.7, 0.5 Hz, 1H), 7.63-7.56 (m, 3H), 7.43 (dd, J=8.2, 1.7 Hz, 1H), 7.34 (dd, J=8.1, 0.6 Hz, 1H), 7.05-6.98 (m, 2H), 4.55-4.46 (m, 4H), 4.32-4.22 (m, 1H), 4.15-4.10 (m, 2H), 3.82 (s, 3H), 3.70-3.65 (m, 2H), 3.32 (s, 3H). LCMS (ESI+) m/z 541.2 (M+H)⁺.

### Example 101

### 6-{1-[6-(2-ethoxypropan-2-yl)-1,3-benzoxazol-2-yl]azetidin-3-yl}-3-[(1-methyl-1H-pyrazol-4-yl)amino]pyrazine-2-carboxamide

To a solution of Example 19 (100 mg, 0.223 mmol) in ethanol (5 mL) was added 4-methylbenzenesulfonic acid hydrate (4.24 mg, 0.022 mmol), and the mixture was stirred at 25° C. for 12 hours. The mixture was adjusted to pH 8 and concentrated. The residue was purified by prep-HPLC to afford the title compound (46.5 mg). ¹H NMR (400 MHz, dimethyl sulfoxide-d₆) δ ppm 1.06 (t, J=6.91 Hz, 3H), 1.47 (s, 6H), 3.13 (q, J=6.85 Hz, 2H), 3.82 (s, 3H), 4.17-4.32 (m, 1H), 4.39-4.53 (m, 4H), 7.10-7.31 (m, 2H), 7.41 (s, 1H), 7.60 (s, 1H), 7.90 (br s, 1H), 8.04 (s, 1H), 8.31-8.52 (m, 2H), 10.72 (s, 1H).

### Example 102

### 6-(1-{6-[(5-methoxypyrimidin-2-yl)oxy]-1,3-benzoxazol-2-yl}azetidin-3-yl)-3-[(1-methyl-1H-pyrazol-4-yl)amino]pyrazine-2-carboxamide

A mixture of Example 20 (60 mg, 0.148 mmol), 2-chloro-5-methoxypyrimidine (25.6 mg, 0.177 mmol), cesium carbonate (144 mg, 0.443 mmol) in dimethyl sulfoxide (2 mL) was heated in microwave at 120° C. for 1 hour. Water was added and the mixture extracted with ethyl acetate. The organic layer was washed with brine, dried over MgSO₄, and concentrated. The residue was purified by silica gel chromatography eluting with 100/0 to 100/10 dichloromethane/methanol to afford the title compound (50 mg). ¹H NMR (500 MHz, dimethyl sulfoxide-d₆) δ ppm 10.72 (s, 1H), 8.44 (d, J=2.3 Hz, 1H), 8.38 (d, J=2.6 Hz, 3H), 8.05 (d, J=0.8 Hz, 1H), 7.90 (d, J=2.3 Hz, 1H), 7.61 (d, J=0.8 Hz, 1H), 7.35 (d, J=2.3 Hz, 1H), 7.30 (d, J=8.5 Hz, 1H), 6.97 (dd, J=8.4, 2.3 Hz, 1H), 4.53-4.42 (m, 4H), 4.26 (ddd, J=15.4, 8.4, 7.0 Hz, 1H), 3.84 (d, J=16.8 Hz, 6H). LCMS (ESI+): m/z 515 (M+H)⁺.

### Example 103

### 3-[(1-methyl-1H-pyrazol-4-yl)amino]-6-(1-{6-[(pyridazin-3-yl)oxy]-1,3-benzoxazol-2-yl}azetidin-3-yl)pyridine-2-carboxamide

The title compound was prepared as described in Example 102 using 3-chloropyridazine in place of 2-chloro-5-methoxypyrimidine and Example 84A in place of Example 20. ¹H NMR (500 MHz, dimethyl sulfoxide) δ ppm 10.74 (s, 1H), 9.01 (dd, J=4.5, 1.3 Hz, 1H), 8.46 (d, J=2.1 Hz, 1H), 8.40 (s, 1H), 8.06 (d, J=0.7 Hz, 1H), 7.91 (d, J=2.3 Hz, 1H), 7.77 (dd, J=9.0, 4.5 Hz, 1H), 7.62 (d, J=0.8 Hz, 1H), 7.53-7.28 (m, 3H), 7.06 (dd, J=8.5, 2.3 Hz, 1H), 4.59-4.43 (m, 4H), 4.37-4.21 (m, 1H), 3.84 (s, 3H). LCMS (ESI+): m/z 485 (M+H)⁺.

### Example 104

### 6-[1-(6-{[1-(2-hydroxyethyl)-1H-pyrazol-4-yl]methoxy}-1,3-benzoxazol-2-yl)azetidin-3-yl]-3-[(1-methyl-1H-pyrazol-4-yl)amino]pyrazine-2-carboxamide

### Example 104A

### ethyl [4-(hydroxymethyl)-1H-pyrazol-1-yl]acetate

A mixture of (1H-pyrazol-4-yl)methanol (1 g, 10.19 mmol), potassium carbonate (2.82 g, 20.39 mmol), ethyl 2-bromoacetate (2.04 g, 12.23 mmol) in acetone (20 mL) was heated at 60° C. overnight. The mixture was filtered, and the filtrate concentrated and purified on silica gel, eluting with 100/0 to 100/10 dichloromethane/methanol to afford the title compound (1 g). ¹H NMR (500 MHz, dimethyl sulfoxide) δ ppm 7.62 (d, J=0.8 Hz, 1H), 7.39 (d, J=0.7 Hz, 1H), 5.01 (s, 2H), 4.87 (t, J=5.5 Hz, 1H), 4.37 (d, J=5.4 Hz, 2H), 4.16 (q, J=7.1 Hz, 2H), 1.22 (t, J=7.1 Hz, 3H).

### Example 104B

### ethyl [4-(chloromethyl)-1H-pyrazol-1-yl]acetate

Example 104A (188 mg, 1.02 mmol) in dichloromethane (3 mL) was cooled to 0° C. and sulfurous dichloride (372 μL, 5.1 mmol) was added. The mixture was warmed to ambient temperature over 4 hours and concentrated to afford the title compound as the hydrochloride salt (233 mg).

### Example 104C

### ethyl [4-({[2-(3-{6-carbamoyl-5-[(1-methyl-1H-pyrazol-4-yl)amino]pyrazin-2-yl}azetidin-1-yl)-1,3-benzoxazol-6-yl]oxy}methyl)-1H-pyrazol-1-yl]acetate

The title compound was prepared as described in Example 80 using Example 104B in place of 3-(bromomethyl)-1-methyl-1H-pyrazole hydrobromic acid and cesium carbonate in place of potassium carbonate. ¹H NMR (500 MHz, dimethyl sulfoxide-d₆) δ ppm 10.71 (s, 1H), 8.40 (d, J=2.4 Hz, 1H), 8.38 (s, 1H), 8.04 (d, J=0.8 Hz, 1H), 7.89 (d, J=2.3 Hz, 1H), 7.84 (d, J=0.7 Hz, 1H), 7.61 (d, J=0.8 Hz, 1H), 7.55 (d, J=0.7 Hz, 1H), 7.20 (dd, J=5.5, 3.0 Hz, 2H), 6.82 (dd, J=8.6, 2.4 Hz, 1H), 5.05 (s, 2H), 4.96 (s, 2H), 4.49-4.40 (m, 4H), 4.29-4.19 (m, 1H), 4.14 (q, J=7.1 Hz, 2H), 3.82 (s, 3H), 1.20 (t, J=7.1 Hz, 3H). LCMS (ESI+): m/z 573 (M+H)⁺.

### Example 104D

### 6-[1-(6-{[1-(2-hydroxyethyl)-1H-pyrazol-4-yl]methoxy}-1,3-benzoxazol-2-yl)azetidin-3-yl]-3-[(1-methyl-1H-pyrazol-4-yl)amino]pyrazine-2-carboxamide

To a solution of Example 104C (104 mg, 0.182 mmol) in tetrahydrofuran (3 mL) was added diisobutylaluminum hydride solution (1453 μL, 1.453 mmol, 1 M in toluene), and the mixture was stirred at ambient temperature for 4 hours. Saturated ammonium chloride solution was added, and the mixture extracted with ethyl acetate. The organic layer was washed with brine, dried over MgSO₄, and purified by silica gel chromatography, eluting with 100/0 to 100/10 dichloromethane/methanol to afford the title compound (20 mg). ¹H NMR (500 MHz, dimethyl sulfoxide-d₆) δ ppm 10.71 (s, 1H), 8.40 (d, J=2.4 Hz, 1H), 8.38 (s, 1H), 8.04 (d, J=0.8 Hz, 1H), 7.89 (d, J=2.4 Hz, 1H), 7.80 (d, J=0.8 Hz, 1H), 7.60 (d, J=0.8 Hz, 1H), 7.51 (d, J=0.8 Hz, 1H), 7.23-7.17 (m, 2H), 6.81 (dd, J=8.6, 2.4 Hz, 1H), 4.93 (s, 2H), 4.89 (t, J=5.2 Hz, 1H), 4.48-4.40 (m, 4H), 4.24 (p, J=7.6 Hz, 1H), 4.12 (t, J=5.7 Hz, 2H), 3.82 (s, 3H), 3.72 (q, J=5.5 Hz, 2H). LCMS (ESI+): m/z 531 (M+H)⁺.

### Example 105

### 6-{1-[6-(1-methyl-1H-imidazol-4-yl)-1,3-benzoxazol-2-yl]azetidin-3-yl}-3-[(1-methyl-1H-pyrazol-4-yl)amino]pyrazine-2-carboxamide

The title compound was prepared as described in Example 88 using (1-methyl-1H-imidazol-4-yl)boronic acid in place of 4-hydroxyphenylboronic acid. Purification was carried out as described in Example 89. ¹H NMR (600 MHz, dimethyl sulfoxide-d₆) δ ppm 10.72 (s, 1H), 8.45-8.41 (m, 1H), 8.39 (s, 1H), 8.08 (d, J=0.8 Hz, 1H), 8.05 (d, J=0.8 Hz, 1H), 7.92-7.89 (m, 1H), 7.84 (d, J=0.8 Hz, 1H), 7.64 (dd, J=1.7, 0.5 Hz, 1H), 7.61 (d, J=0.8 Hz, 1H), 7.38 (dd, J=8.1, 1.6 Hz, 1H), 7.27 (d, J=8.1 Hz, 1H), 4.52-4.45 (m, 4H), 4.30-4.22 (m, 1H), 3.86 (s, 4H), 3.82 (s, 3H). LCMS (ESI+) m/z 471.2 (M+H)⁺.

### Example 106

### 1-{[2-(3-{6-carbamoyl-5-[(1-methyl-1H-pyrazol-4-yl)amino]pyridin-2-yl}azetidin-1-yl)-1,3-benzoxazol-6-yl]oxy}-2-methylpropan-2-yl dihydrogen phosphate

The title compound was prepared as described in Example 77 using Example 85 in place of Example 31. ¹H NMR (500 MHz, dimethyl sulfoxide-d₆) δ ppm 9.81 (s, 1H), 8.18 (d, J=3.0 Hz, 1H), 7.80 (d, J=0.8 Hz, 1H), 7.62 (d, J=2.8 Hz, 1H), 7.43 (d, J=0.9 Hz, 1H), 7.37 (d, J=8.8 Hz, 1H), 7.30 (d, J=8.7 Hz, 1H), 7.20 (d, J=8.6 Hz, 1H), 7.12 (d, J=2.4 Hz, 1H), 6.79 (dd, J=8.5, 2.4 Hz, 1H), 4.47 (t, J=8.2 Hz, 2H), 4.40 (dd, J=7.8, 6.5 Hz, 2H), 4.13 (tt, J=8.7, 6.5 Hz, 1H), 3.83 (s, 3H), 3.70 (s, 2H), 1.20 (s, 6H). LCMS (ESI+): m/z 558.3 (M+H)⁺.

### Example 107

### 6-{1-[6-(2-cyano-2-methylpropoxy)-1,3-benzoxazol-2-yl]azetidin-3-yl}-3-[(1-methyl-1H-pyrazol-4-yl)amino]pyrazine-2-carboxamide

The title compound was prepared as described in Example 31 using 2-cyano-2-methylpropyl 4-methylbenzenesulfonate in place of 4-bromo-2-methylbutane-2-ol. ¹H NMR (500 MHz, dimethyl sulfoxide-d₆) δ ppm 10.71 (s, 1H), 8.39 (d, J=4.2 Hz, 2H), 8.04 (d, J=0.8 Hz, 1H), 7.88 (d, J=2.3 Hz, 1H), 7.60 (d, J=0.8 Hz, 1H), 7.27-7.15 (m, 2H), 6.84 (dd, J=8.5, 2.4 Hz, 1H), 4.45 (dd, J=7.7, 1.2 Hz, 4H), 4.25 (q, J=7.8 Hz, 1H), 3.99 (s, 2H), 3.82 (s, 3H), 1.41 (s, 6H). LCMS (ESI+): m/z 487.9 (M)⁺.

### Example 108

### 1-{[2-(3-{6-carbamoyl-5-[(1-methyl-1H-pyrazol-4-yl)amino]pyridin-2-yl}azetidin-1-yl)-1,3-benzoxazol-6-yl]oxy}-2-methylpropan-2-yl glycinate

The title compound was prepared as described in Example 95 using Example 85 in place of Example 31. ¹H NMR (500 MHz, dimethyl sulfoxide-d₆) δ ppm 9.81 (s, 1H), 8.17 (d, J=3.1 Hz, 1H), 7.79 (s, 1H), 7.62 (d, J=3.1 Hz, 1H), 7.43 (d, J=0.9 Hz, 1H), 7.37 (d, J=8.8 Hz, 1H), 7.30 (d, J=8.7 Hz, 1H), 7.20 (d, J=8.5 Hz, 1H), 7.14 (d, J=2.4 Hz, 1H), 6.80 (dd, J=8.6, 2.4 Hz, 1H), 4.46 (t, J=8.2 Hz, 2H), 4.39 (dd, J=7.9, 6.5 Hz, 2H), 4.14 (dd, J=8.5, 6.4 Hz, 1H), 4.12 (s, 2H), 3.83 (s, 3H), 3.17 (s, 2H), 1.51 (s, 6H). LCMS (ESI+): m/z 535.3 (M+H)⁺.

### Example 109

### 4-{[2-(3-{6-carbamoyl-5-[(1-methyl-1H-pyrazol-4-yl)amino]pyridin-2-yl}azetidin-1-yl)-1,3-benzoxazol-6-yl]oxy}-2-methylbutan-2-yl dihydrogen phosphate

The title compound was prepared as described in Example 77 using compound in Example 84 in place of Example 31. ¹H NMR (500 MHz, dimethyl sulfoxide-d₆) δ ppm 9.81 (s, 1H), 8.24-8.11 (m, 1H), 7.79 (d, J=0.9 Hz, 1H), 7.67-7.55 (m, 1H), 7.42 (d, J=0.9 Hz, 1H), 7.36 (d, J=8.8 Hz, 1H), 7.30 (d, J=8.7 Hz, 1H), 7.22 (d, J=8.5 Hz, 1H), 7.15 (d, J=2.5 Hz, 1H), 6.79 (dd, J=8.6, 2.4 Hz, 1H), 4.47 (t, J=8.3 Hz, 2H), 4.40 (dd, J=7.9, 6.4 Hz, 2H), 4.17-4.12 (m, 1H), 4.10 (t, J=7.0 Hz, 2H), 3.82 (s, 3H), 2.11 (t, J=7.0 Hz, 2H), 1.45 (s, 6H). LCMS (ESI+): m/z 572.3 (M+H)⁺.

### Example 110

### 4-{[2-(3-{6-carbamoyl-5-[(1-methyl-1H-pyrazol-4-yl)amino]pyridin-2-yl}azetidin-1-yl)-1,3-benzoxazol-6-yl]oxy}-2-methylbutan-2-yl glycinate

The title compound was prepared as described in Example 95 using compound in Example 84 in place of Example 31. ¹H NMR (400 MHz, dimethyl sulfoxide-d₆) δ ppm 9.80 (s, 1H), 8.15 (d, J=3.1 Hz, 1H), 7.78 (s, 1H), 7.60 (d, J=3.1 Hz, 1H), 7.42 (d, J=0.8 Hz, 1H), 7.36 (d, J=8.8 Hz, 1H), 7.30 (d, J=8.7 Hz, 1H), 7.19 (d, J=8.5 Hz, 1H), 7.13 (d, J=2.4 Hz, 1H), 6.76 (dd, J=8.5, 2.4 Hz, 1H), 4.54-4.30 (m, 4H), 4.12 (ddd, J=8.5, 6.4, 2.1 Hz, 1H), 4.04 (td, J=7.0, 2.4 Hz, 2H), 3.82 (s, 3H), 3.17 (s, 2H), 2.22 (t, J=6.9 Hz, 2H), 1.48 (s, 6H). LCMS (ESI+): m/z 549.4 (M+H)⁺.

### Example 111

### 6-{1-[6-(1-hydroxy-2-methylpropan-2-yl)-1,3-benzoxazol-2-yl]azetidin-3-yl}-3-[(1-methyl-1H-pyrazol-4-yl)amino]pyrazine-2-carboxamide

### Example 111A

### ethyl 2-(3-methoxy-4-nitrophenyl)-2-methylpropanoate

To a solution of sodium tert-butoxide (5.78 g, 60.2 mmol) in N,N-dimethylformamide (250 mL) was added ethyl 2-(3-methoxy-4-nitrophenyl)acetate (12 g, 50.2 mmol) in portions at 0° C. Methyl iodide (4.7 mL, 75 mmol) was added dropwise at 0° C. and, mixture was stirred at 20° C. for 1 hour. After the mixture was cooled to 0° C., sodium tert-butoxide (5.78 g, 60.2 mmol) and methyl iodide (4.70 mL, 75 mmol) were added, and the mixture was stirred at 20° C. for 12 hours. The mixture was diluted with water and extracted with ethyl acetate. The organic extracts were washed with brine, dried over Na₂SO₄, and concentrated to afford the title compound (10 g), which was used to the next step directly without further purification. ¹H NMR (400 MHz, CDCl₃) δ ppm 1.19 (t, J=7.17 Hz, 3H), 1.59 (s, 6H), 3.95 (s, 3H), 4.10-4.16 (m, 2H), 6.96-7.05 (m, 2H), 7.82 (d, J=8.38 Hz, 1H).

### Example 111B

### ethyl 2-(3-hydroxy-4-nitrophenyl)-2-methylpropanoate

To a solution of Example 111A (10 g, 37.4 mmol) in N,N-dimethylformamide (100 mL) was added lithium chloride (4.76 g, 112 mmol) at 20° C., and the mixture was stirred at 140° C. for 16 hours. The mixture was cooled to ambient temperature, diluted with water, and extracted with ethyl acetate. The organic layer was washed with brine, dried over Na₂SO₄, filtered, concentrated, and purified by silica gel chromatography, eluting with 60/1 to 10/1 petroleum ether/ethyl acetate to afford the title compound (6 g).

### Example 111C

### ethyl 2-(4-amino-3-hydroxyphenyl)-2-methylpropanoate

To a solution of palladium on carbon (2.52 g, 23.69 mmol) in tetrahydrofuran (150 mL) was added Example 111B (6 g, 23.69 mmol) at 20° C., and the mixture was stirred under a hydrogen balloon at 20° C. for 12 hours. The suspension was filtered through a pad of silica gel, and the filter cake was washed with tetrahydrofuran. The filtrates were concentrated to afford the title compound (5 g). ¹H NMR (400 MHz, dimethyl sulfoxide-d₆) δ ppm 1.10 (t, J=7.09 Hz, 3H) 1.39 (s, 6H) 3.94-4.07 (m, 2H) 4.42 (br s, 2H) 6.47-6.54 (m, 2H) 6.61 (s, 1H) 8.94 (br s, 1H).

### Example 111D

### ethyl 2-methyl-2-(2-sulfanyl-1,3-benzoxazol-6-yl)propanoate

To a solution of Example 111C (5 g, 22.39 mmol) in ethanol (100 mL) was added potassium O-ethyl carbonodithioate (10.77 g, 67.2 mmol) and potassium hydroxide (1.885 g, 33.6 mmol) at 20° C., and the mixture was stirred at 80° C. for 12 hours. The mixture was concentrated, poured into water, and acetic acid added to adjust to pH 5. After standing for 12 hours, the precipitate was collected by filtration and dried in vacuo to afford the title compound (5 g). ¹H NMR (400 MHz, CDCl₃) δ ppm 1.18 (t, J=7.15 Hz, 3H), 1.55-1.65 (m, 6H), 4.13 (q, J=7.11 Hz, 2H), 7.11 (d, J=8.34 Hz, 1H), 7.23-7.26 (m, 1H), 7.36 (d, J=1.43 Hz, 1H), 10.95 (br s, 1H).

### Example 111E

### ethyl 2-(2-chloro-1,3-benzoxazol-6-yl)-2-methylpropanoate

To a solution of Example 111D (5 g, 18.84 mmol) in thionyl chloride (20 mL, 274 mmol) was added N,N-dimethylformamide (1.46 mL, 18.84 mmol), and the mixture stirred at 20° C. for 20 minutes. The mixture was slowly poured into saturated Na₂CO₃ and extracted with ethyl acetate. The organic extracts were dried over Na₂SO₄, filtered, concentrated, and purified by silica gel chromatography, eluting with 50/1 to 5/1 petroleum ether/ethyl acetate to afford the title compound (4 g).

### Example 111F

### ethyl 2-[2-(3-{6-carbamoyl-5-[(1-methyl-1H-pyrazol-4-yl)amino]pyrazin-2-yl}azetidin-1-yl)-1,3-benzoxazol-6-yl]-2-methylpropanoate

To a solution of Intermediate 1 (4.8 g, 12.39 mmol) in N,N-dimethylformamide (80 mL) was added potassium carbonate (6.85 g, 49.6 mmol) and Example 111E (3.98 g, 14.87 mmol), and the mixture was stirred at 20° C. for 12 hours. The mixture was diluted with water and extracted with ethyl acetate. The organic extracts were dried over Na₂SO₄, filtered, concentrated, and purified by silica gel chromatography, eluting with 10/1 to 1/1 petroleum ether/tetrahydrofuran to afford the title compound (4 g). ¹H NMR (400 MHz, dimethyl sulfoxide-d₆) δ ppm 1.10 (t, J=7.09 Hz, 3H), 1.51 (s, 6H), 3.82 (s, 3H), 4.05 (q, J=7.15 Hz, 2H), 4.18-4.32 (m, 1H), 4.39-4.52 (m, 4H), 7.10 (dd, J=8.23, 1.55 Hz, 1H), 7.25 (d, J=8.23 Hz, 1H), 7.38 (d, J=1.31 Hz, 1H), 7.61 (s, 1H), 7.91 (br s, 1H), 8.04 (s, 1H), 8.30-8.48 (m, 2H), 10.73 (s, 1H).

### Example 111G

### 6-{1-[6-(1-hydroxy-2-methylpropan-2-yl)-1,3-benzoxazol-2-yl]azetidin-3-yl}-3-[(1-methyl-1H-pyrazol-4-yl)amino]pyrazine-2-carboxamide

To an ice-cooled suspension of Example 111F (4 g, 7.93 mmol) in tetrahydrofuran (80 mL) was added lithium triethylborohydride (31.7 mL, 31.7 mmol), and the mixture was stirred at 20° C. for 4 hours. The mixture was quenched with saturated ammonium chloride, the organic phase separated, and the aqueous layer extracted with ethyl acetate. The organic extracts were dried over Na₂SO₄, filtered, concentrated, and purified by silica gel chromatography, eluting with 10/1 to 1/1 petroleum ether/tetrahydrofuran to afford the title compound (2 g). ¹H NMR (400 MHz, dimethyl sulfoxide-d₆) δ ppm 1.24 (s, 6H), 1.76 (s, 1H), 3.41 (d, J=5.50 Hz, 2H), 3.82 (s, 3H), 4.19-4.30 (m, 1H), 4.41-4.51 (m, 4H), 4.63 (t, J=5.38 Hz, 1H), 7.13-7.25 (m, 2H), 7.42 (s, 1H), 7.60 (s, 1H), 7.89 (br s, 1H), 8.04 (s, 1H), 8.38 (s, 2H), 10.71 (s, 1H).

### Example 112

### 1-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylpropan-2-yl dihydrogen phosphate

The title compound was prepared as described in Example 77 using Example 38 in place of Example 31. ¹H NMR (600 MHz, dimethyl sulfoxide-d₆) δ ppm 10.71 (s, 1H), 8.39 (d, J=10.7 Hz, 2H), 8.04 (d, J=0.8 Hz, 1H), 7.89 (d, J=2.2 Hz, 1H), 7.61 (d, J=0.7 Hz, 1H), 7.22 (d, J=8.5 Hz, 1H), 7.16 (d, J=2.4 Hz, 1H), 6.82 (dd, J=8.6, 2.4 Hz, 1H), 4.47 (d, J=7.7 Hz, 4H), 4.28-4.25 (m, 1H), 3.96 (s, 2H), 3.82 (s, 3H), 1.50 (s, 6H). LCMS (ESI+): m/z 559.4 (M+H)⁺.

### Example 113

### 1-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylpropan-2-yl glycinate

The title compound was prepared as described in Example 95 using Example 38 in place of Example 31. ¹H NMR (400 MHz, dimethyl sulfoxide-d₆) δ ppm 10.70 (s, 1H), 8.38 (d, J=2.2 Hz, 2H), 8.04 (s, 1H), 7.88 (s, 1H), 7.60 (d, J=0.8 Hz, 1H), 7.23-7.08 (m, 2H), 6.80 (dd, J=8.5, 2.4 Hz, 1H), 4.44 (d, J=7.7 Hz, 4H), 4.24 (q, J=7.6 Hz, 1H), 4.11 (s, 2H), 3.82 (s, 3H), 3.15 (s, 2H), 1.51 (s, 6H). LCMS (ESI+): m/z 536.4 (M+H)⁺.

### Determination of Biological Activity

### HTRF Kinase Activity Biochemical Assay

For the SAR (structure-activity relationship) and compound screening, HTRF (Homogeneous Time Resolved Fluorescence) kinase activity assay was employed for all MER, AXL and TYRO3 kinases using Cisbio HTRF® KinEASE™-TK kit (Cisbio, USA). The kit included biotin-labeled TK substrate, streptavidin-XL665, Eu3+-cryptate-labeled TK antibody and HTRF® Detection buffer. There are two main steps in the kinase assay: kinase reaction and detection of phosphorylated substrate. The reaction was carried out in white low volume 384-well plate (Corning, USA) with 25 nL compound in dimethyl sulfoxide in each well. To measure the compound mediated inhibition of kinase activity, 2.5 μL of the recombinant kinases were pre-incubated with test compounds for 30 minutes in the kinase reaction buffer (20 mM HEPES pH 7.4, 2 mM MnCl₂, 10 mM MgCl₂, 100 μM Na₃VO₄, 0.0075% Triton X 100, 0.005% BSA and 1 mM DTT) prior to the addition of 2.5 μL of 1 μM biotin-labeled TK substrates and 10 uM ATP. Then the reaction was stopped after 1 hour incubation at room temperature by adding 5 μL of HTRF® Detection buffer which also contains 0.375 nM Eu3+-cryptate-labeled TK antibody and 0.062 μM streptavidin-XL665(SA-XL665) to allow for detection of the phosphorylated peptide product. After 1 hour incubation at room temperature, the fluorescence intensity was measured with Envision® plate reader (PerkinElmer, USA). Upon excitation at 340 nm by UV, the energy from Eu3+ donor of the antibody is transferred to the FRET acceptor XL665, and XL655 emits light at 665 nm. The level of kinase activity was quantified by the HTRF ratio that calculated from the intensity of emission at 665 nm and emission at 620 nm (fluorescence intensity @665 nm/fluorescence intensity @620 nm×10,000). The recombinant protein of human MER (528-end) was purchased from Carnabio, Japan. The recombinant human AXL (473-end) and TYRO3 (455-end) were purchased from SignalChem, Canada.

TABLE 2
HTRF Kinase Activity Data

| | HTRF | HTRF | HTRF |
|---|---|---|---|
| | MERTK | AXL | TYRO |
| | IC₅₀ | IC₅₀ | IC₅₀ |
| EXAMPLE | (μM) | (μM) | (μM) |
| 0 | 0.034 | 0.060 | 2.68 |
| 1 | 0.009 | 0.007 | 1.03 |
| 2 | <0.001 | 0.002 | 0.056 |
| 3 | <0.001 | 0.004 | 0.092 |
| 4 | 0.008 | 0.019 | 0.45 |
| 5 | <0.001 | 0.002 | 0.083 |
| 6 | <0.001 | 0.00104 | 0.072 |
| 6A | <0.001 | 0.004 | 0.06 |
| 7 | 0.003 | 0.006 | 0.68 |
| 8 | 0.002 | 0.007 | 0.28 |
| 9 | 0.0018 | 0.007 | 0.13 |
| 10 | <0.001 | <0.001 | 0.11 |
| 10A | 0.003 | 0.004 | 0.69 |
| 11 | 0.001 | 0.002 | 0.28 |
| 12 | 0.001 | 0.004 | 0.22 |
| 13 | 0.002 | 0.003 | 0.12 |
| 14 | 0.008 | 0.014 | 0.49 |
| 15 | 0.003 | 0.009 | 0.55 |
| 15A | 0.002 | 0.005 | 0.074 |
| 16 | 0.007 | 0.030 | 0.86 |
| 17 | <0.001 | 0.002 | 0.241 |
| 18 | 0.014 | 0.039 | 2.72 |
| 18A | 3.71 | 5.90 | >12.5 |
| 19 | 0.007 | 0.018 | 1.07 |
| 20 | 0.029 | | 0.98 |
| 21 | <0.001 | 0.001 | 0.005 |
| 22 | <0.001 | <0.001 | 0.090 |
| 23 | <0.001 | <0.001 | 0.008 |
| 24 | 0.002 | 0.003 | 0.13 |
| 25 | 0.007 | 0.010 | 1.10 |
| 26 | <0.001 | 0.003 | 0.094 |
| 27 | 0.001 | 0.003 | 0.082 |
| 28 | 0.001 | 0.006 | 0.15 |
| 29 | 0.005 | 0.010 | 0.34 |
| 30 | 0.003 | 0.011 | 0.36 |
| 31 | 0.001 | 0.003 | 0.10 |
| 32 | 0.001 | 0.003 | 0.19 |
| 32A | <0.001 | 0.002 | 0.14 |
| 33 | 0.001 | 0.002 | 0.13 |
| 34 | 0.008 | 0.019 | 0.62 |
| 35 | 0.001 | 0.002 | 0.24 |
| 36 | 0.001 | 0.009 | 0.17 |
| 36C | 0.001 | 0.003 | >10 |
| 36D | 0.003 | 0.010 | 0.43 |
| 37 | 0.010 | 0.029 | 2.13 |
| 38 | 0.001 | 0.002 | 0.27 |
| 39 | 0.001 | 0.005 | 0.51 |
| 40 | 0.002 | 0.008 | 0.46 |
| 41 | <0.001 | 0.002 | 0.21 |
| 42 | <0.001 | 0.002 | 0.064 |
| 43 | 0.009 | 0.025 | 2.14 |
| 44 | <0.001 | 0.002 | 0.11 |
| 45 | 0.003 | 0.009 | 0.62 |
| 46 | 0.003 | 0.009 | 0.009 |
| 47 | <0.001 | 0.004 | 0.63 |
| 48 | 0.002 | 0.003 | 0.23 |
| 49 | 0.004 | 0.011 | 2.03 |
| 50 | 0.009 | 0.016 | 3.55 |
| 51 | 0.003 | 0.006 | 1.74 |
| 51A | 0.002 | 0.017 | 2.38 |
| 52 | 0.001 | 0.001 | 0.051 |
| 53 | 0.004 | 0.002 | 2.67 |
| 54 | 0.005 | 0.023 | 0.62 |
| 55 | 0.003 | 0.014 | 0.22 |
| 56 | <0.001 | 0.002 | 0.092 |
| 57 | 0.007 | 0.008 | 2.31 |
| 58 | <0.001 | 0.001 | 0.033 |
| 59 | <0.001 | 0.002 | 0.03 |
| 60 | <0.001 | 0.001 | 0.10 |
| 61 | <0.001 | 0.001 | 0.026 |
| 62 | 0.003 | 0.006 | 1.75 |
| 63 | 0.011 | 0.016 | 4.3 |
| 64 | <0.001 | 0.005 | 0.037 |
| 65 | <0.001 | 0.001 | 1.01 |
| 65C | 0.035 | 0.095 | 3.53 |
| 66 | 0.002 | 0.003 | 2.90 |
| 67 | 0.001 | 0.003 | 0.19 |
| 68 | 0.002 | 0.005 | 0.59 |
| 69 | 0.002 | 0.008 | 0.57 |
| 70 | 0.003 | 0.020 | >12.5 |
| 71 | 0.002 | 0.004 | 3.73 |
| 72 | 0.001 | 0.003 | 0.94 |
| 73 | 0.002 | 0.009 | 1.70 |
| 73A | 0.150 | 0.210 | 12.50 |
| 74 | 0.002 | 0.004 | 0.13 |
| 75 | <0.001 | 0.003 | 0.065 |
| 76 | <0.001 | 0.002 | 0.24 |
| 77 | 0.033 | 0.054 | 3.30 |
| 77A | 0.002 | 0.006 | 0.25 |
| 78 | 0.002 | 0.006 | 0.27 |
| 79 | <0.001 | 0.002 | 0.066 |
| 80 | <0.001 | 0.001 | 0.048 |
| 81 | 0.001 | 0.003 | 1.07 |
| 82 | <0.001 | 0.003 | 0.15 |
| 83 | 0.004 | 0.026 | 6.8 |
| 84 | 0.002 | 0.007 | 1.37 |
| 84A | 0.014 | 0.033 | 2.07 |
| 85 | 0.002 | 0.004 | 1.85 |
| 86 | 0.005 | 0.011 | 0.063 |
| 87 | 0.001 | 0.005 | 0.33 |
| 88 | <0.001 | 0.003 | 0.051 |
| 89 | 0.001 | 0.006 | 0.26 |
| 90 | 0.002 | 0.004 | 0.88 |
| 91 | 0.002 | 0.004 | 1.43 |
| 92 | <0.001 | 0.003 | 0.08 |
| 93 | <0.001 | 0.002 | 0.044 |
| 94 | <0.001 | 0.003 | 0.91 |
| 95 | <0.001 | 0.003 | 0.28 |
| 95A | <0.001 | 0.003 | 0.46 |
| 96 | 0.003 | 0.012 | 1.61 |
| 96A | 0.011 | 0.031 | 1.39 |
| 96B | 0.002 | 0.007 | 0.67 |
| 97 | 0.001 | 0.004 | 0.15 |
| 98 | 0.0011 | 0.007 | 1.28 |
| 99 | <0.001 | 0.0016 | 0.024 |
| 100 | 0.001 | 0.004 | 0.12 |
| 101 | 0.008 | 0.012 | 1.49 |
| 102 | <0.001 | 0.002 | 0.10 |
| 103 | <0.001 | 0.003 | 0.71 |
| 104 | 0.008 | 0.059 | 3.50 |
| 104C | 0.004 | 0.027 | 0.62 |
| 105 | <0.001 | 0.005 | 0.053 |
| 106 | 0.0018 | 0.006 | 1.07 |
| 107 | <0.001 | 0.0015 | 0.046 |
| 108 | <0.001 | 0.004 | 0.88 |
| 109 | 0.027 | 0.078 | 2.20 |
| 110 | <0.001 | 0.0018 | 0.28 |
| 111 | 0.005 | 0.006 | 0.64 |
| 111F | 0.018 | 0.043 | 3.23 |
| 112 | 0.052 | 0.069 | 5.86 |
| 113 | <0.001 | 0.001 | 0.16 |

### MC-38 Efficacy Assay

MC-38 cells were maintained in Dulbecco's Modified Eagle Medium (DMEM) (Invitrogen, Carlsbad, CA) supplemented with 10% fetal bovine serum. Female C57BL/6 mice were obtained from Charles River (Wilmington, MA). The body weight upon arrival was 18-20 g. Food and water were available ad libitum. Mice were acclimated to the animal facilities for a period of at least one week prior to commencement of experiments. Animals were tested in the light phase of a 12-hour light: 12-hour dark schedule (lights on at 06:00 hours).

Tumor growth inhibition (TGI) indicates the divergence between the mean tumor volume of a drug-treated group and the mean tumor volume of the control and is expressed as a percentage of the mean volume of the control group. The TGI-value is determined at the last time point before the first animal is removed from a group because it reached its tumor burden limit. Statistically significant differences in mean tumor volume of compound-treated versus vehicle treated were assessed using the Student's t-test.

The stock of MerTK inhibitors were maintained in powder form in a storage desiccator and resuspended for dosing in a solution of 10% EtOH, 30% PEG 400, and 60% Phozol 50. The MerTK inhibitors were dosed once a day for 21 days.

1×10⁵ Viable MC-38 cells were inoculated subcutaneously into the right flank of the mice on Day 0. The injection volume was 0.1 mL composed of a 1:1 mixture of S-MEM and Matrigel (BD, Franklin Lakes, NJ). All tumors were size matched at approximately 150-200 mm³ unless otherwise indicated. Therapy began within 24 hours after size matching the tumors. Mice weighed approximately 20 grams at the onset of therapy. Tumor volume was measured two to three times weekly. Measurements of the length (L) and width (W) of the tumor were taken via electronic caliper and the volume was calculated according to the following equation: V=L×W²/2. Mice were euthanized when tumor volume reached up to 3,000 mm³ or skin ulcerations occurred.

MC-38 cells were grown to passage three in vitro. One hundred thousand cells per mouse were inoculated subcutaneously into the right flank of female C57BL6 mice on Day 0. Tumors were size matched on Day 7. The mean tumor volume at tumor staging was approximately 150 mm³.

TABLE 3
MC-38 Efficacy

| Example | % TGI (dose) | |
|---|---|---|
| 1 | 37% | (10 mg/kg/d) |
| 2 | 83% | (10 mg/kg/d) |
| 3 | 51% | (15 mg/kg/d) |
| 21 | 18% | (10 mg/kg/d) |
| 28 | 17% | (10 mg/kg/d) |
| 30 | 33% | (10 mg/kg/d) |
| 31 | 68% | (10 mg/kg/d) |
| 38 | 64% | (10 mg/kg/d) |
| 39 | 31% | (10 mg/kg/d) |
| 47 | 0% | (30 mg/kg/d) |
| 72 | 44% | (30 mg/kg/d) |
| 77 | 39% | (15 mg/kg/d) |
| 80 | 23% | (10 mg/kg/d) |
| 85 | 37% | (10 mg/kg/d) |

Further benefits of Applicants' invention will be apparent to one skilled in the art from reading this patent application. It is understood that the foregoing detailed description and accompanying examples are merely illustrative and are not to be taken as limitations upon the scope of the present disclosure, which is defined by the appended claims and their equivalents. Various changes and modifications to the described embodiments will be apparent to those skilled in the art. Such changes and modifications, including without limitation those relating to the chemical structures, substituents, derivatives, intermediates, syntheses, formulations, or methods, or any combination of such changes and modifications of use of the present disclosure, may be made without departing from the spirit and scope thereof.

1. A compound of formula (I), or an enantiomer, a diastereomer, a racemate, a tautomer, a prodrug, a hydrate, a solvate or a pharmaceutically acceptable salt thereof: 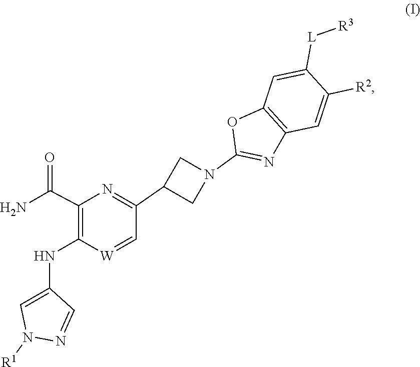
where
W is N or CH;
L is selected from the group consisting of a bond, —CH═CHCR^{a}R^{b}—, —(CR^{a}R^{b})ₙ—O—, —(CR^{a}R^{b})ₙ—C(O)—, —O—, —O—(CR^{a}R^{b})ₙ—, —O—(CR^{a}R^{b})ₙ—O—, —OCH₂C(O)—, and —C(O)—;
each R^{a} is independently selected from the group consisting of hydrogen, C₁-C₄ alkyl, and C₁-C₂ hydroxyalkyl;
each R^{b} is independently selected from the group consisting of hydrogen and C₁-C₄ alkyl;
where R^{a} and R^{b} optionally combine to form a C₃₋₆ cycloalkyl or 3-6 membered heterocycle;
R¹ is selected from the group consisting of H, C₁-C₆ alkyl, and 5-6 membered heterocyclyl; wherein the R¹ C₁-C₆ alkyl is optionally substituted with 1 or 2 R⁴;
R² is selected from the group consisting of hydrogen, fluoro, chloro, bromo, and C₁-C₄ alkyl;
R³ is selected from the group consisting of hydrogen, fluoro, chloro, bromo, —CN, C₁-C₄ alkyl, C₁-C₄ haloalkyl, —OH, —OR^{3a}, —NR^{3b}R^{3c}, —P(O)(OR^{3d})₂, —C(O)CHR^{3e}NR^{3b}R^{3c}, —C(O)OH, —C(O)OR^{3a}, —C(O)NR^{3b}R^{3c}, C₃-C₈ cycloalkyl, C₅-C₇ cycloalkenyl, 3-6 membered heterocyclyl, C₅-C₇ heterocycloalkenyl, C₆ aryl, 5-6 membered heteroaryl and 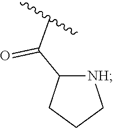 [SMILES: *C(=O)C1CCCN1]
wherein the C₃-C₈ cycloalkyl, C₆ aryl, 3-6 membered heterocyclyl, and 5-6 membered heteroaryl of R³, are optionally substituted with 1 or 2 of R⁵;
R^{3a} is C₁-C₄ alkyl;
R^{3b} and R^{3c} are each independently selected from the group consisting of hydrogen, C₁-C₄ alkyl, C₃-C₆ cycloalkyl, and —CO₂R^{3f};
R^{3d} is selected from the group consisting of hydrogen, C₁-C₄ alkyl, and C₂-C₄ alkenyl;
R^{3e} is selected from the group consisting of hydrogen and C₁-C₄ alkyl; wherein the C₁-C₄ alkyl of R^{3e} is optionally substituted with 1 of R^{3g};
R^{3f} is C₁-C₄ alkyl;
R^{3g} selected from the group consisting of —OH, —SH, —SCH₃, —NH₂, —C(O)OH, —C(O)NH₂, —NHC(NH)NH₂, C₆ aryl, and 5-9 membered heteroaryl; wherein the C₆ aryl of R^{3g} is optionally substituted with 1 of —OH;
R⁴ is selected from the group consisting of —OH, —OR^{4a}, —C(O)NR^{4b}R^{4c}, and 5-6 membered heterocyclyl; wherein the 5-6 membered heterocyclyl of R⁴ is optionally substituted with 1 or 2 of C₁-C₄ alkyl;
R^{4a} is C₁-C₄ alkyl; wherein the C₁-C₄ alkyl of R^{4a} is optionally substituted with 1-Si(R^{4d})₃;
R^{4b} and R^{4c} are each independently selected from the group consisting of hydrogen and C₁-C₄ alkyl;
R^{4d} is C₁-C₄ alkyl;
R⁵ is selected from the group consisting of fluoro, —CN, —OH, —OR^{5a}, =O, —CH₂CO₂R^{5b}, C₁-C₄ alkyl, C₁-C₄ haloalkyl, C₁-C₄ hydroxyalkyl, C₁-C₄ hydroxyalkoxy, and C₂-C₆ alkoxyalkyl;
R^{5a} is C₁-C₄ alkyl; and
R^{5b} is C₁-C₄ alkyl;
n is an integer of 1 to 3.

2. The compound of claim 1, or an enantiomer, a diastereomer, a racemate, a tautomer, a prodrug, a hydrate, a solvate or a pharmaceutically acceptable salt thereof, wherein L is —O—(CR^{a}R^{b})ₙO—.

3. The compound of claim 1, or an enantiomer, a diastereomer, a racemate, a tautomer, a prodrug, a hydrate, a solvate or a pharmaceutically acceptable salt thereof, wherein the compound is represented by the following formula (II) 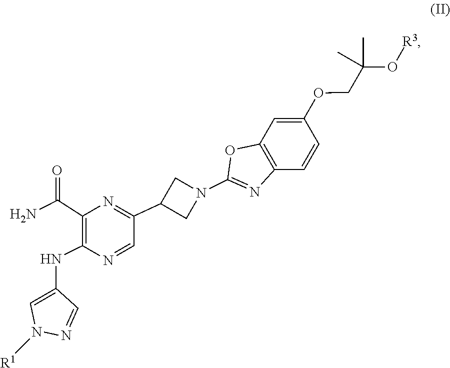
where
R¹ is selected from the group consisting of hydrogen, C₁-C₆ alkyl, and 5-6 membered heterocyclyl; wherein the C₁-C₆ alkyl of R¹ is optionally substituted with 1 or 2 of R⁴;
R³ is selected from the group consisting of hydrogen, C₁-C₄ alkyl, C₁-C₄ haloalkyl, —P(O)(OR^{3d})₂, —C(O)CHR^{3e}NR^{3b}R^{3c}, —C(O)OH, —C(O)OR^{3a}, —C(O)NR^{3b}R^{3c}, C₃-C₈ cycloalkyl, C₅-C₇ cycloalkenyl, 3-6 membered heterocyclyl, C₅-C₇ heterocycloalkenyl, C₆ aryl, and 5-6 membered heteroaryl;
wherein the C₃-C₈ cycloalkyl, C₆ aryl, 3-6 membered heterocyclyl, and 5-6 membered heteroaryl of R³, are optionally substituted with 1 or 2 of R⁵;
R^{3a} is C₁-C₄ alkyl;
R^{3b} and R^{3c} are each independently selected from the group consisting of hydrogen, C₁-C₄ alkyl, C₃-C₆ cycloalkyl, and —CO₂R^{3f};
R^{3d} is selected from the group consisting of hydrogen, C₁-C₄ alkyl, and C₂-C₄ alkenyl;
R^{3e} is selected from the group consisting of hydrogen and C₁-C₄ alkyl; wherein the C₁-C₄ alkyl of R^{3d} is optionally substituted with 1 of R^{3g};
R^{3f} is C₁-C₄ alkyl;
R^{3g} selected from the group consisting of —OH, —SH, —SCH₃, —NH₂, —C(O)OH, —C(O)NH₂, —NHC(NH)NH₂, C₆ aryl, and 5-9 membered heteroaryl; wherein the C₆ aryl of R^{3g} is optionally substituted with 1 of —OH;
R⁴ is selected from the group consisting of —OH, —OR^{4a}, —C(O)NR^{4b}R^{4c}, and 5-6 membered heterocyclyl; wherein the 3-10 membered heterocyclyl of R⁴ is optionally substituted with 1 or 2 of C₁-C₄ alkyl;
R^{4a} is C₁-C₄ alkyl; wherein the C₁-C₄ alkyl of R^{4a} is optionally substituted with 1 of —Si(R^{4d})₃;
R^{4b} and R^{4c} are each independently selected from the group consisting of H and C₁-C₄ alkyl;
R^{4d} is C₁-C₄ alkyl;
R⁵ is selected from the group consisting of fluoro, —CN, —OH, —OR^{5a}, ═O, —CH₂CO₂R^{5b}, C₁-C₄ alkyl, C₁-C₄ haloalkyl, C₁-C₄ hydroxyalkyl, C₁-C₄ hydroxyalkoxy, and C₂-C₆ alkoxyalkyl;
R^{5a} is C₁-C₄ alkyl; and
R^{5b} is C₁-C₄ alkyl;
n is an integer of 1 to 3.

4. The compound of claim 3, or an enantiomer, a diastereomer, a racemate, a tautomer, a prodrug, a hydrate, a solvate or a pharmaceutically acceptable salt thereof, wherein R³ is selected from the group consisting of hydrogen, C₁-C₄ alkyl, —P(O)(OR^{3d})₂, and —C(O)CHR^{3e}NR^{3b}R^{3c}.

5. The compound of claim 4, or an enantiomer, a diastereomer, a racemate, a tautomer, a prodrug, a hydrate, a solvate or a pharmaceutically acceptable salt thereof, wherein R¹ is selected from the group consisting of hydrogen and C₁-C₆ alkyl; wherein the C₁-C₆ alkyl of R¹ is optionally substituted with 1 or 2 of R⁴; and R⁴ is selected from the group consisting of —OH and —OR^{4a}.

6. The compound of claim 5, or an enantiomer, a diastereomer, a racemate, a tautomer, a prodrug, a hydrate, a solvate, or a pharmaceutically acceptable salt thereof, wherein the compound is

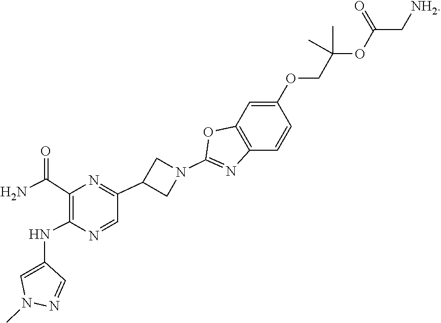 [SMILES: Cn1cc(Nc2ncc(C3CN(c4nc5ccc(OCC(C)(C)OC(=O)CN)cc5o4)C3)nc2C(N)=O)cn1]

7. The compound of claim 5, or an enantiomer, a diastereomer, a racemate, a tautomer, a prodrug, a hydrate, a solvate, or a pharmaceutically acceptable salt thereof which is

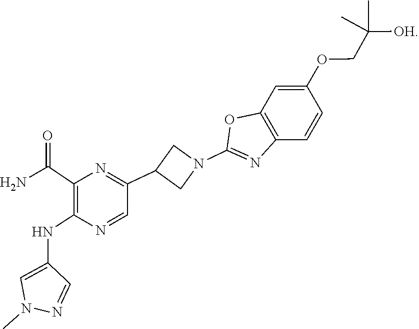 [SMILES: Cn1cc(Nc2ncc(C3CN(c4nc5ccc(OCC(C)(C)O)cc5o4)C3)nc2C(N)=O)cn1]

8. The compound of claim 1, or an enantiomer, a diastereomer, a racemate, a tautomer, a prodrug, a hydrate, a solvate, or a pharmaceutically acceptable salt thereof, wherein
L is a bond; and
R³ is selected from the group consisting of hydrogen, fluoro, chloro, bromo, —CN, C₁-C₄ alkyl, C₁-C₄ haloalkyl, —OH, —OR^{3a}, —NR^{3b}R^{3c}, —C(O)OH, —C(O)OR^{3a}, —C(O)NR^{3b}R^{3c}, C₃-C₈ cycloalkyl, C₅-C₇ cycloalkenyl, 3-6 membered heterocyclyl, C₅-C₇ heterocycloalkenyl, C₆ aryl, and 5-6 membered heteroaryl;
wherein the C₃-C₈ cycloalkyl, C₆ aryl, 3-6 membered heterocyclyl, and 5-6 membered heteroaryl of R³, are optionally substituted with 1 or 2 of R⁵.

9. The compound of claim 8, or an enantiomer, a diastereomer, a racemate, a tautomer, a prodrug, a hydrate, a solvate, or a pharmaceutically acceptable salt thereof, wherein R¹ is C₁-C₆ alkyl; wherein the C₁-C₆ alkyl of R¹ is optionally substituted with 1 or 2 of R⁴; and R⁴ is selected from the group consisting of —OH, —OR^{4a}, —C(O)NR^{4b}R^{4c}, and 5-6 membered heterocyclyl.

10. The compound of claim 9, or an enantiomer, a diastereomer, a racemate, a tautomer, a prodrug, a hydrate, a solvate, or a pharmaceutically acceptable salt thereof, wherein R¹ is methyl.

11. The compound of claim 10, or an enantiomer, a diastereomer, a racemate, a tautomer, a prodrug, a hydrate, a solvate, or a pharmaceutically acceptable salt thereof, wherein R³ is selected from the group consisting of hydrogen, C₁-C₄ alkyl, C₆ aryl, and 5-6 membered heteroaryl;
wherein the C₆ aryl, and 5-6 membered heteroaryl of R³, are optionally substituted with 1 or 2 of R⁵; and
R⁵ is selected from the group consisting of —OH, —OR^{5a}, C₁-C₄ alkyl, C₁-C₄ hydroxyalkyl, C₁-C₄ hydroxyalkoxy, and C₂-C₆ alkoxyalkyl.

12. The compound of claim 1, or an enantiomer, a diastereomer, a racemate, a tautomer, a prodrug, a hydrate, a solvate, or a pharmaceutically acceptable salt thereof, wherein
L is —O—(CR^{a}R^{b})ₙ—; and
R³ is selected from the group consisting of —CN, C₃-C₈ cycloalkyl, 3-6 membered heterocyclyl, C₆ aryl, and 5-6 membered heteroaryl;
wherein the R³ C₃-C₈ cycloalkyl, C₆ aryl, 3-6 membered heterocyclyl, and 5-6 membered heteroaryl, are optionally substituted with 1 or 2 of R⁵; and
R⁵ is selected from the group consisting of —OH, —CH₂CO₂R^{5b}, C₁-C₄ alkyl, and C₁-C₄ hydroxyalkyl.

13. The compound of claim 1, or an enantiomer, a diastereomer, a racemate, a tautomer, a prodrug, a hydrate, a solvate, or a pharmaceutically acceptable salt thereof, selected from the group consisting of
6-[1-(6-hydroxy-1,3-benzoxazol-2-yl)azetidin-3-yl]-3-[(1-{[2-(trimethylsilyl)ethoxy]methyl}-1H-pyrazol-4-yl)amino]pyrazine-2-carboxamide;
6-[1-(1,3-benzoxazol-2-yl)azetidin-3-yl]-3-[(1-methyl-1H-pyrazol-4-yl)amino]pyrazine-2-carboxamide;
6-[1-(6-chloro-1,3-benzoxazol-2-yl)azetidin-3-yl]-3-[(1-methyl-1H-pyrazol-4-yl)amino]pyrazine-2-carboxamide;
6-[1-(6-methyl-1,3-benzoxazol-2-yl)azetidin-3-yl]-3-[(1-methyl-1H-pyrazol-4-yl)amino]pyrazine-2-carboxamide;
6-[1-(5,6-dimethyl-1,3-benzoxazol-2-yl)azetidin-3-yl]-3-[(1-methyl-1H-pyrazol-4-yl)amino]pyrazine-2-carboxamide;
6-[1-(6-ethyl-1,3-benzoxazol-2-yl)azetidin-3-yl]-3-[(1-methyl-1H-pyrazol-4-yl)amino]pyrazine-2-carboxamide;
6-{1-[6-(cyclohex-1-en-1-yl)-1,3-benzoxazol-2-yl]azetidin-3-yl}-3-[(1-methyl-1H-pyrazol-4-yl)amino]pyrazine-2-carboxamide;
6-[1-(6-bromo-1,3-benzoxazol-2-yl)azetidin-3-yl]-3-[(1-methyl-1H-pyrazol-4-yl)amino]pyrazine-2-carboxamide;
6-[1-(6-cyclohexyl-1,3-benzoxazol-2-yl)azetidin-3-yl]-3-[(1-methyl-1H-pyrazol-4-yl)amino]pyrazine-2-carboxamide;
3-[(1-methyl-1H-pyrazol-4-yl)amino]-6-[1-(6-phenyl-1,3-benzoxazol-2-yl)azetidin-3-yl]pyrazine-2-carboxamide;
6-[1-(6-methoxy-1,3-benzoxazol-2-yl)azetidin-3-yl]-3-[(1-methyl-1H-pyrazol-4-yl)amino]pyrazine-2-carboxamide;
6-[1-(6-cyclopentyl-1,3-benzoxazol-2-yl)azetidin-3-yl]-3-[(1-methyl-1H-pyrazol-4-yl)amino]pyrazine-2-carboxamide;
6-{1-[6-(cyclopent-1-en-1-yl)-1,3-benzoxazol-2-yl]azetidin-3-yl}-3-[(1-methyl-1H-pyrazol-4-yl)amino]pyrazine-2-carboxamide;
6-{1-[6-(3,6-dihydro-2H-pyran-4-yl)-1,3-benzoxazol-2-yl]azetidin-3-yl}-3-[(1-methyl-1H-pyrazol-4-yl)amino]pyrazine-2-carboxamide;
3-[(1-methyl-1H-pyrazol-4-yl)amino]-6-{1-[6-(trifluoromethyl)-1,3-benzoxazol-2-yl]azetidin-3-yl}pyrazine-2-carboxamide;
methyl 2-(3-{6-carbamoyl-5-[(1-methyl-1H-pyrazol-4-yl)amino]pyrazin-2-yl}azetidin-1-yl)-1,3-benzoxazole-6-carboxylate;
6-[1-(1,3-benzoxazol-2-yl)azetidin-3-yl]-3-[(1H-pyrazol-4-yl)amino]pyrazine-2-carboxamide;
3-[(1-methyl-1H-pyrazol-4-yl)amino]-6-{1-[6-(oxolan-3-yl)-1,3-benzoxazol-2-yl]azetidin-3-yl}pyrazine-2-carboxamide;
6-{1-[6-(2,5-dihydrofuran-3-yl)-1,3-benzoxazol-2-yl]azetidin-3-yl}-3-[(1-methyl-1H-pyrazol-4-yl)amino]pyrazine-2-carboxamide;
6-{1-[6-(hydroxymethyl)-1,3-benzoxazol-2-yl]azetidin-3-yl}-3-[(1-methyl-1H-pyrazol-4-yl)amino]pyrazine-2-carboxamide;
6-(1-{6-[(1E)-3-hydroxy-3-methylbut-1-en-1-yl]-1,3-benzoxazol-2-yl}azetidin-3-yl)-3-[(1-methyl-1H-pyrazol-4-yl)amino]pyrazine-2-carboxamide;
2-(3-{6-carbamoyl-5-[(1-methyl-1H-pyrazol-4-yl)amino]pyrazin-2-yl}azetidin-1-yl)-N,N-dimethyl-1,3-benzoxazole-6-carboxamide;
2-(3-{6-carbamoyl-5-[(1-methyl-1H-pyrazol-4-yl)amino]pyrazin-2-yl}azetidin-1-yl)-1,3-benzoxazole-6-carboxylic acid;
6-{1-[6-(2-hydroxypropan-2-yl)-1,3-benzoxazol-2-yl]azetidin-3-yl}-3-[(1-methyl-1H-pyrazol-4-yl)amino]pyrazine-2-carboxamide;
6-[1-(6-hydroxy-1,3-benzoxazol-2-yl)azetidin-3-yl]-3-[(1-methyl-1H-pyrazol-4-yl)amino]pyrazine-2-carboxamide;
6-{1-[6-(3-hydroxy-3-methylbutyl)-1,3-benzoxazol-2-yl]azetidin-3-yl}-3-[(1-methyl-1H-pyrazol-4-yl)amino]pyrazine-2-carboxamide;
6-(1-{6-[(1E)-3-methoxyprop-1-en-1-yl]-1,3-benzoxazol-2-yl}azetidin-3-yl)-3-[(1-methyl-1H-pyrazol-4-yl)amino]pyrazine-2-carboxamide;
6-{1-[6-(cyclopropylmethoxy)-1,3-benzoxazol-2-yl]azetidin-3-yl}-3-[(1-methyl-1H-pyrazol-4-yl)amino]pyrazine-2-carboxamide;
6-{1-[6-(cyanomethoxy)-1,3-benzoxazol-2-yl]azetidin-3-yl}-3-[(1-methyl-1H-pyrazol-4-yl)amino]pyrazine-2-carboxamide;
6-(1-{6-[3-hydroxy-2-(hydroxymethyl)propyl]-1,3-benzoxazol-2-yl}azetidin-3-yl)-3-[(1-methyl-1H-pyrazol-4-yl)amino]pyrazine-2-carboxamide;
ethyl {[2-(3-{6-carbamoyl-5-[(1-methyl-1H-pyrazol-4-yl)amino]pyrazin-2-yl}azetidin-1-yl)-1,3-benzoxazol-6-yl]oxy}acetate;
6-[1-(6-fluoro-1,3-benzoxazol-2-yl)azetidin-3-yl]-3-{[1-(oxan-4-yl)-1H-pyrazol-4-yl]amino}pyrazine-2-carboxamide;
6-[1-(6-fluoro-1,3-benzoxazol-2-yl)azetidin-3-yl]-3-{[1-(2-hydroxy-2-methylpropyl)-1H-pyrazol-4-yl]amino}pyrazine-2-carboxamide;
6-[1-(1,3-benzoxazol-2-yl)azetidin-3-yl]-3-{[1-(2-methoxyethyl)-1H-pyrazol-4-yl]amino}pyrazine-2-carboxamide;
6-[1-(1,3-benzoxazol-2-yl)azetidin-3-yl]-3-{[1-(2-hydroxy-2-methylpropyl)-1H-pyrazol-4-yl]amino}pyrazine-2-carboxamide;
6-{1-[6-(3-hydroxy-3-methylbutoxy)-1,3-benzoxazol-2-yl]azetidin-3-yl}-3-[(1-methyl-1H-pyrazol-4-yl)amino]pyrazine-2-carboxamide;
6-{1-[6-(3-hydroxypropyl)-1,3-benzoxazol-2-yl]azetidin-3-yl}-3-[(1-methyl-1H-pyrazol-4-yl)amino]pyrazine-2-carboxamide;
6-(1-{6-[(1E)-3-hydroxyprop-1-en-1-yl]-1,3-benzoxazol-2-yl}azetidin-3-yl)-3-[(1-methyl-1H-pyrazol-4-yl)amino]pyrazine-2-carboxamide;
6-{1-[6-(3-methoxypropyl)-1,3-benzoxazol-2-yl]azetidin-3-yl}-3-[(1-methyl-1H-pyrazol-4-yl)amino]pyrazine-2-carboxamide;
6-[1-(1,3-benzoxazol-2-yl)azetidin-3-yl]-3-({1-[2-(dimethylamino)-2-oxoethyl]-1H-pyrazol-4-yl}amino)pyrazine-2-carboxamide;
6-{1-[6-(2-methoxyethoxy)-1,3-benzoxazol-2-yl]azetidin-3-yl}-3-[(1-methyl-1H-pyrazol-4-yl)amino]pyrazine-2-carboxamide;
3-{[1-(2,3-dihydroxypropyl)-1H-pyrazol-4-yl]amino}-6-[1-(6-fluoro-1,3-benzoxazol-2-yl)azetidin-3-yl]pyrazine-2-carboxamide;
6-[1-(6-fluoro-1,3-benzoxazol-2-yl)azetidin-3-yl]-3-[(1H-pyrazol-4-yl)amino]pyrazine-2-carboxamide;
3-({1-[(2,2-dimethyl-1,3-dioxolan-4-yl)methyl]-1H-pyrazol-4-yl}amino)-6-[1-(6-fluoro-1,3-benzoxazol-2-yl)azetidin-3-yl]pyrazine-2-carboxamide;
6-{1-[6-(2-amino-2-oxoethoxy)-1,3-benzoxazol-2-yl]azetidin-3-yl}-3-[(1-methyl-1H-pyrazol-4-yl)amino]pyrazine-2-carboxamide;
6-{1-[6-(2-hydroxy-2-methylpropoxy)-1,3-benzoxazol-2-yl]azetidin-3-yl}-3-[(1-methyl-1H-pyrazol-4-yl)amino]pyrazine-2-carboxamide;
6-(1-{6-[2-(dimethylamino)-2-oxoethoxy]-1,3-benzoxazol-2-yl}azetidin-3-yl)-3-[(1-methyl-1H-pyrazol-4-yl)amino]pyrazine-2-carboxamide;
6-{1-[6-(2-hydroxyethoxy)-1,3-benzoxazol-2-yl]azetidin-3-yl}-3-[(1-methyl-1H-pyrazol-4-yl)amino]pyrazine-2-carboxamide;
3-[(1-methyl-1H-pyrazol-4-yl)amino]-6-(1-{6-[(oxetan-3-yl)methoxy]-1,3-benzoxazol-2-yl}azetidin-3-yl)pyrazine-2-carboxamide;
6-(1-{6-[(3-hydroxycyclobutyl)methoxy]-1,3-benzoxazol-2-yl}azetidin-3-yl)-3-[(1-methyl-1H-pyrazol-4-yl)amino]pyrazine-2-carboxamide;
6-{1-[6-(2,3-dihydroxypropoxy)-1,3-benzoxazol-2-yl]azetidin-3-yl}-3-[(1-methyl-1H-pyrazol-4-yl)amino]pyrazine-2-carboxamide;
6-(1-{6-[(1-hydroxycyclobutyl)methoxy]-1,3-benzoxazol-2-yl}azetidin-3-yl)-3-[(1-methyl-1H-pyrazol-4-yl)amino]pyrazine-2-carboxamide;
6-(1-{6-[2-(3-hydroxyoxetan-3-yl)ethyl]-1,3-benzoxazol-2-yl}azetidin-3-yl)-3-[(1-methyl-1H-pyrazol-4-yl)amino]pyrazine-2-carboxamide;
3-[(1-methyl-1H-pyrazol-4-yl)amino]-6-{1-[6-(morpholine-4-carbonyl)-1,3-benzoxazol-2-yl]azetidin-3-yl}pyrazine-2-carboxamide;
6-(1-{6-[2-(dimethylamino)-2-oxoethyl]-1,3-benzoxazol-2-yl}azetidin-3-yl)-3-[(1-methyl-1H-pyrazol-4-yl)amino]pyrazine-2-carboxamide;
6-[1-(6-{[(2R)-1,4-dioxan-2-yl]methoxy}-1,3-benzoxazol-2-yl)azetidin-3-yl]-3-[(1-methyl-1H-pyrazol-4-yl)amino]pyrazine-2-carboxamide;
3-[(1-methyl-1H-pyrazol-4-yl)amino]-6-(1-{6-[2-oxo-2-(pyrrolidin-1-yl)ethoxy]-1,3-benzoxazol-2-yl}azetidin-3-yl)pyrazine-2-carboxamide;
6-(1-{6-[2-(methylamino)-2-oxoethyl]-1,3-benzoxazol-2-yl}azetidin-3-yl)-3-[(1-methyl-1H-pyrazol-4-yl)amino]pyrazine-2-carboxamide;
6-(1-{6-[3-hydroxy-2-(hydroxymethyl)propoxy]-1,3-benzoxazol-2-yl}azetidin-3-yl)-3-[(1-methyl-1H-pyrazol-4-yl)amino]pyrazine-2-carboxamide;
6-(1-{6-[(2,2-dimethyl-1,3-dioxan-5-yl)methoxy]-1,3-benzoxazol-2-yl}azetidin-3-yl)-3-[(1-methyl-1H-pyrazol-4-yl)amino]pyrazine-2-carboxamide;
N-tert-butyl-2-(3-{6-carbamoyl-5-[(1-methyl-1H-pyrazol-4-yl)amino]pyrazin-2-yl}azetidin-1-yl)-1,3-benzoxazole-6-carboxamide;
2-(3-{6-carbamoyl-5-[(1-methyl-1H-pyrazol-4-yl)amino]pyrazin-2-yl}azetidin-1-yl)-N-cyclopropyl-1,3-benzoxazole-6-carboxamide;
3-{[1-(2-hydroxyethyl)-1H-pyrazol-4-yl]amino}-6-[1-(6-methyl-1,3-benzoxazol-2-yl)azetidin-3-yl]pyrazine-2-carboxamide;
3-{[1-(2-methoxyethyl)-1H-pyrazol-4-yl]amino}-6-[1-(6-methyl-1,3-benzoxazol-2-yl)azetidin-3-yl]pyrazine-2-carboxamide;
6-[1-(6-{[3-(hydroxymethyl)cyclobutyl]methoxy}-1,3-benzoxazol-2-yl)azetidin-3-yl]-3-[(1-methyl-1H-pyrazol-4-yl)amino]pyrazine-2-carboxamide;
6-(1-{6-[(3-hydroxyoxetan-3-yl)methoxy]-1,3-benzoxazol-2-yl}azetidin-3-yl)-3-[(1-methyl-1H-pyrazol-4-yl)amino]pyrazine-2-carboxamide;
6-[1-(6-{[(1S,2R)-2-(hydroxymethyl)cyclopropyl]methoxy}-1,3-benzoxazol-2-yl)azetidin-3-yl]-3-[(1-methyl-1H-pyrazol-4-yl)amino]pyrazine-2-carboxamide;
6-(1-{6-[(2R)-2-(hydroxymethyl)pyrrolidine-1-carbonyl]-1,3-benzoxazol-2-yl}azetidin-3-yl)-3-[(1-methyl-1H-pyrazol-4-yl)amino]pyrazine-2-carboxamide;
3-[(1-methyl-1H-pyrazol-4-yl)amino]-6-{1-[6-(3-phenylpropoxy)-1,3-benzoxazol-2-yl]azetidin-3-yl}pyrazine-2-carboxamide;
6-{1-[6-(3-hydroxy-2,2-dimethylpropoxy)-1,3-benzoxazol-2-yl]azetidin-3-yl}-3-[(1-methyl-1H-pyrazol-4-yl)amino]pyrazine-2-carboxamide;
6-(1-{6-[3-(dimethylamino)-3-oxopropyl]-1,3-benzoxazol-2-yl}azetidin-3-yl)-3-[(1-methyl-1H-pyrazol-4-yl)amino]pyrazine-2-carboxamide;
3-[(1-methyl-1H-pyrazol-4-yl)amino]-6-(1-{6-[3-oxo-3-(pyrrolidin-1-yl)propyl]-1,3-benzoxazol-2-yl}azetidin-3-yl)pyrazine-2-carboxamide;
6-(1-{6-[(2R)-2-(methoxymethyl)pyrrolidine-1-carbonyl]-1,3-benzoxazol-2-yl}azetidin-3-yl)-3-[(1-methyl-1H-pyrazol-4-yl)amino]pyrazine-2-carboxamide;
3-[(1-methyl-1H-pyrazol-4-yl)amino]-6-(1-{6-[2-oxo-2-(pyrrolidin-1-yl)ethyl]-1,3-benzoxazol-2-yl}azetidin-3-yl)pyrazine-2-carboxamide;
[2-(3-{6-carbamoyl-5-[(1-methyl-1H-pyrazol-4-yl)amino]pyrazin-2-yl}azetidin-1-yl)-1,3-benzoxazol-6-yl]acetic acid;
6-[1-(6-{2-[(2S)-2-(hydroxymethyl)pyrrolidin-1-yl]-2-oxoethyl}-1,3-benzoxazol-2-yl)azetidin-3-yl]-3-[(1-methyl-1H-pyrazol-4-yl)amino]pyrazine-2-carboxamide;
6-{1-[6-(2-hydroxy-2-methylpropyl)-1,3-benzoxazol-2-yl]azetidin-3-yl}-3-[(1-methyl-1H-pyrazol-4-yl)amino]pyrazine-2-carboxamide;
6-(1-{6-[(2R)-2-hydroxypropoxy]-1,3-benzoxazol-2-yl}azetidin-3-yl)-3-[(1-methyl-1H-pyrazol-4-yl)amino]pyrazine-2-carboxamide;
3-[(1-methyl-1H-pyrazol-4-yl)amino]-6-[1-(6-{[(3S)-oxolan-3-yl]oxy}-1,3-benzoxazol-2-yl)azetidin-3-yl]pyrazine-2-carboxamide;
6-[1-(6-{2-[(3S)-3-methoxypyrrolidin-1-yl]-2-oxoethyl}-1,3-benzoxazol-2-yl)azetidin-3-yl]-3-[(1-methyl-1H-pyrazol-4-yl)amino]pyrazine-2-carboxamide;
6-[1-(6-{2-[(2R)-2-(methoxymethyl)pyrrolidin-1-yl]-2-oxoethyl}-1,3-benzoxazol-2-yl)azetidin-3-yl]-3-[(1-methyl-1H-pyrazol-4-yl)amino]pyrazine-2-carboxamide;
6-(1-{6-[(1-methyl-2-oxopyrrolidin-3-yl)oxy]-1,3-benzoxazol-2-yl}azetidin-3-yl)-3-[(1-methyl-1H-pyrazol-4-yl)amino]pyrazine-2-carboxamide;
6-(1-{6-[2-(azetidin-1-yl)-2-oxoethoxy]-1,3-benzoxazol-2-yl}azetidin-3-yl)-3-[(1-methyl-1H-pyrazol-4-yl)amino]pyrazine-2-carboxamide;
{[2-(3-{6-carbamoyl-5-[(1-methyl-1H-pyrazol-4-yl)amino]pyrazin-2-yl}azetidin-1-yl)-1,3-benzoxazol-6-yl]oxy}acetic acid;
6-[1-(6-methyl-1,3-benzoxazol-2-yl)azetidin-3-yl]-3-[(1H-pyrazol-4-yl)amino]pyrazine-2-carboxamide;
6-(1-{6-[(2R)-2-(2-hydroxypropan-2-yl)pyrrolidine-1-carbonyl]-1,3-benzoxazol-2-yl}azetidin-3-yl)-3-[(1-methyl-1H-pyrazol-4-yl)amino]pyrazine-2-carboxamide;
6-(1-{6-[(1-methyl-2-oxopiperidin-3-yl)oxy]-1,3-benzoxazol-2-yl}azetidin-3-yl)-3-[(1-methyl-1H-pyrazol-4-yl)amino]pyrazine-2-carboxamide;
4-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylbutan-2-yl dihydrogen phosphate;
4-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylbutan-2-yl diprop-2-en-1-yl phosphate;
6-[1-(6-methyl-1,3-benzoxazol-2-yl)azetidin-3-yl]-3-[(1-methyl-1H-pyrazol-4-yl)amino]pyridine-2-carboxamide;
6-(1-{6-[(2-hydroxycyclopentyl)oxy]-1,3-benzoxazol-2-yl}azetidin-3-yl)-3-[(1-methyl-1H-pyrazol-4-yl)amino]pyrazine-2-carboxamide;
3-[(1-methyl-1H-pyrazol-4-yl)amino]-6-(1-{6-[(1-methyl-1H-pyrazol-3-yl)methoxy]-1,3-benzoxazol-2-yl}azetidin-3-yl)pyrazine-2-carboxamide;
3-[(1-methyl-1H-pyrazol-4-yl)amino]-6-[1-(6-{2-oxo-2-[(2R)-2-(trifluoromethyl)pyrrolidin-1-yl]ethyl}-1,3-benzoxazol-2-yl)azetidin-3-yl]pyrazine-2-carboxamide;
3-[(1-methyl-1H-pyrazol-4-yl)amino]-6-(1-{6-[(5-methyl-1,3-thiazol-2-yl)methoxy]-1,3-benzoxazol-2-yl}azetidin-3-yl)pyrazine-2-carboxamide;
6-(1-{6-[2-(dimethylamino)-2-oxoethoxy]-1,3-benzoxazol-2-yl}azetidin-3-yl)-3-[(1-methyl-1H-pyrazol-4-yl)amino]pyridine-2-carboxamide;
6-{1-[6-(3-hydroxy-3-methylbutoxy)-1,3-benzoxazol-2-yl]azetidin-3-yl}-3-[(1-methyl-1H-pyrazol-4-yl)amino]pyridine-2-carboxamide;
6-[1-(6-hydroxy-1,3-benzoxazol-2-yl)azetidin-3-yl]-3-[(1-methyl-1H-pyrazol-4-yl)amino]pyridine-2-carboxamide;
6-{1-[6-(2-hydroxy-2-methylpropoxy)-1,3-benzoxazol-2-yl]azetidin-3-yl}-3-[(1-methyl-1H-pyrazol-4-yl)amino]pyridine-2-carboxamide;
6-(1-{6-[(5-methyl-1,3,4-oxadiazol-2-yl)methoxy]-1,3-benzoxazol-2-yl}azetidin-3-yl)-3-[(1-methyl-1H-pyrazol-4-yl)amino]pyrazine-2-carboxamide;
6-{1-[6-(2-cyclopropyl-2-oxoethoxy)-1,3-benzoxazol-2-yl]azetidin-3-yl}-3-[(1-methyl-1H-pyrazol-4-yl)amino]pyrazine-2-carboxamide;
6-{1-[6-(4-hydroxyphenyl)-1,3-benzoxazol-2-yl]azetidin-3-yl}-3-[(1-methyl-1H-pyrazol-4-yl)amino]pyrazine-2-carboxamide;
6-(1-{6-[4-(2-hydroxyethoxy)phenyl]-1,3-benzoxazol-2-yl}azetidin-3-yl)-3-[(1-methyl-1H-pyrazol-4-yl)amino]pyrazine-2-carboxamide;
6-(1-{6-[1-(2-hydroxyethyl)-1H-pyrazol-4-yl]-1,3-benzoxazol-2-yl}azetidin-3-yl)-3-[(1-methyl-1H-pyrazol-4-yl)amino]pyrazine-2-carboxamide;
6-(1-{6-[1-(2-methoxyethyl)-1H-pyrazol-4-yl]-1,3-benzoxazol-2-yl}azetidin-3-yl)-3-[(1-methyl-1H-pyrazol-4-yl)amino]pyrazine-2-carboxamide;
3-[(1-methyl-1H-pyrazol-4-yl)amino]-6-{1-[6-(1-methyl-1H-pyrazol-4-yl)-1,3-benzoxazol-2-yl]azetidin-3-yl}pyrazine-2-carboxamide;
3-[(1-methyl-1H-pyrazol-4-yl)amino]-6-(1-{6-[(1H-pyrazol-4-yl)methoxy]-1,3-benzoxazol-2-yl}azetidin-3-yl)pyrazine-2-carboxamide;
3-[(1-tert-butyl-1H-pyrazol-4-yl)amino]-6-{1-[6-(3-hydroxy-3-methylbutoxy)-1,3-benzoxazol-2-yl]azetidin-3-yl}pyrazine-2-carboxamide;
4-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylbutan-2-yl glycinate;
4-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylbutan-2-yl [(tert-butoxycarbonyl)amino]acetate;
6-{1-[6-(3-hydroxy-3-methylbutoxy)-1,3-benzoxazol-2-yl]azetidin-3-yl}-3-{[1-(2-hydroxy-2-methylpropyl)-1H-pyrazol-4-yl]amino}pyrazine-2-carboxamide;
6-{1-[6-(3-hydroxy-3-methylbutoxy)-1,3-benzoxazol-2-yl]azetidin-3-yl}-3-[(1-{[2-(trimethylsilyl)ethoxy]methyl}-1H-pyrazol-4-yl)amino]pyrazine-2-carboxamide;
6-{1-[6-(3-hydroxy-3-methylbutoxy)-1,3-benzoxazol-2-yl]azetidin-3-yl}-3-[(1H-pyrazol-4-yl)amino]pyrazine-2-carboxamide;
3-[(1-methyl-1H-pyrazol-4-yl)amino]-6-(1-{6-[(1-methyl-1H-pyrazol-4-yl)methoxy]-1,3-benzoxazol-2-yl}azetidin-3-yl)pyrazine-2-carboxamide;
6-(1-{6-[(1-methyl-1H-imidazol-4-yl)methoxy]-1,3-benzoxazol-2-yl}azetidin-3-yl)-3-[(1-methyl-1H-pyrazol-4-yl)amino]pyrazine-2-carboxamide;
3-[(1-methyl-1H-pyrazol-4-yl)amino]-6-{1-[6-(2-methyl-1,3-thiazol-5-yl)-1,3-benzoxazol-2-yl]azetidin-3-yl}pyrazine-2-carboxamide;
6-(1-{6-[4-(2-methoxyethoxy)phenyl]-1,3-benzoxazol-2-yl}azetidin-3-yl)-3-[(1-methyl-1H-pyrazol-4-yl)amino]pyrazine-2-carboxamide;
6-{1-[6-(2-ethoxypropan-2-yl)-1,3-benzoxazol-2-yl]azetidin-3-yl}-3-[(1-methyl-1H-pyrazol-4-yl)amino]pyrazine-2-carboxamide;
6-(1-{6-[(5-methoxypyrimidin-2-yl)oxy]-1,3-benzoxazol-2-yl}azetidin-3-yl)-3-[(1-methyl-1H-pyrazol-4-yl)amino]pyrazine-2-carboxamide;
3-[(1-methyl-1H-pyrazol-4-yl)amino]-6-(1-{6-[(pyridazin-3-yl)oxy]-1,3-benzoxazol-2-yl}azetidin-3-yl)pyridine-2-carboxamide;
6-[1-(6-{[1-(2-hydroxyethyl)-1H-pyrazol-4-yl]methoxy}-1,3-benzoxazol-2-yl)azetidin-3-yl]-3-[(1-methyl-1H-pyrazol-4-yl)amino]pyrazine-2-carboxamide;
ethyl [4-({[2-(3-{6-carbamoyl-5-[(1-methyl-1H-pyrazol-4-yl)amino]pyrazin-2-yl}azetidin-1-yl)-1,3-benzoxazol-6-yl]oxy}methyl)-1H-pyrazol-1-yl]acetate;
6-{1-[6-(1-methyl-1H-imidazol-4-yl)-1,3-benzoxazol-2-yl]azetidin-3-yl}-3-[(1-methyl-1H-pyrazol-4-yl)amino]pyrazine-2-carboxamide;
1-{[2-(3-{6-carbamoyl-5-[(1-methyl-1H-pyrazol-4-yl)amino]pyridin-2-yl}azetidin-1-yl)-1,3-benzoxazol-6-yl]oxy}-2-methylpropan-2-yl dihydrogen phosphate;
6-{1-[6-(2-cyano-2-methylpropoxy)-1,3-benzoxazol-2-yl]azetidin-3-yl}-3-[(1-methyl-1H-pyrazol-4-yl)amino]pyrazine-2-carboxamide;
1-{[2-(3-{6-carbamoyl-5-[(1-methyl-1H-pyrazol-4-yl)amino]pyridin-2-yl}azetidin-1-yl)-1,3-benzoxazol-6-yl]oxy}-2-methylpropan-2-yl glycinate;
4-{[2-(3-{6-carbamoyl-5-[(1-methyl-1H-pyrazol-4-yl)amino]pyridin-2-yl}azetidin-1-yl)-1,3-benzoxazol-6-yl]oxy}-2-methylbutan-2-yl dihydrogen phosphate;
4-{[2-(3-{6-carbamoyl-5-[(1-methyl-1H-pyrazol-4-yl)amino]pyridin-2-yl}azetidin-1-yl)-1,3-benzoxazol-6-yl]oxy}-2-methylbutan-2-yl glycinate;
6-{1-[6-(1-hydroxy-2-methylpropan-2-yl)-1,3-benzoxazol-2-yl]azetidin-3-yl}-3-[(1-methyl-1H-pyrazol-4-yl)amino]pyrazine-2-carboxamide;
ethyl 2-[2-(3-{6-carbamoyl-5-[(1-methyl-1H-pyrazol-4-yl)amino]pyrazin-2-yl}azetidin-1-yl)-1,3-benzoxazol-6-yl]-2-methylpropanoate;
1-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylpropan-2-yl dihydrogen phosphate; and
1-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylpropan-2-yl glycinate.

14. The compound of claim 13, or an enantiomer, a diastereomer, a racemate, a tautomer, a prodrug, a hydrate, a solvate or a pharmaceutically acceptable salt thereof, wherein the compound is 6-{1-[6-(2-hydroxy-2-methylpropoxy)-1,3-benzoxazol-2-yl]azetidin-3-yl}-3-[(1-methyl-1H-pyrazol-4-yl)amino]pyrazine-2-carboxamide.

15. The compound of claim 13, or an enantiomer, a diastereomer, a racemate, a tautomer, a prodrug, a hydrate, a solvate or a pharmaceutically acceptable salt thereof which is 1-{[2-(3-{6-carbamoyl-5-[(1-methyl-1H-pyrazol-4-yl)amino]pyrazin-2-yl}azetidin-1-yl)-1,3-benzoxazol-6-yl]oxy}-2-methylpropan-2-yl glycinate.

16. A pharmaceutical composition comprising a therapeutically effective amount of a compound of Formula (I) according to claim 1, or an enantiomer, a diastereomer, a racemate, a tautomer, a prodrug, a hydrate, a solvate or a pharmaceutically acceptable salt thereof, in combination with a pharmaceutically acceptable carrier.

17.-20. (canceled)

21. A method of treating or preventing a disease which is influenced by inhibition of Mer kinase, the method comprising administering to a mammals including humans in need thereof compositions comprising, as active ingredients, the compounds, or an enantiomer, a diastereomer, a racemate, a tautomer, a prodrug, a hydrate, a solvate or pharmaceutically acceptable salts thereof according to claim 1.

22. The method of claim 21, wherein the disease which is influenced by inhibition of Mer kinase is cancer or immune-related diseases.

23. The method of claim 22, wherein the cancer is selected from the group consisting of: glioma, gliosarcoma, anaplastic astrocytoma, medulloblastoma, lung cancer, small cell lung carcinoma, cervical carcinoma, colon cancer, rectal cancer, chordoma, throat cancer, Kaposi's sarcoma, lymphangiosarcoma, lymphangioendotheliosarcoma, colorectal cancer, endometrium cancer, ovarian cancer, breast cancer, pancreatic cancer, prostate cancer, renal cell carcinoma, hepatic carcinoma, bile duct carcinoma, choriocarcinoma, seminoma, testicular tumor, Wilms' tumor, Ewing's tumor, bladder carcinoma, angiosarcoma, endotheliosarcoma, adenocarcinoma, sweat gland carcinoma, sebaceous gland sarcoma, papillary sarcoma, papillary adenosarcoma, cystadenosarcoma, bronchogenic carcinoma, medullary carcinoma, mastocytoma, mesotheliorma, synovioma, melanoma, leiomyosarcoma, rhabdomyosarcoma, neuroblastoma, retinoblastoma, oligodentroglioma, acoustic neuroma, hemangioblastoma, meningioma, pinealoma, ependymoma, craniopharyngioma, epithelial carcinoma, embryonal carcinoma, squamous cell carcinoma, basal cell carcinoma, fibrosarcoma, myxoma, myxosarcoma, liposarcorna, chondrosarcoma, osteogenic sarcoma, leukemia and metastatic lesions secondary to these primary tumors.

24. The method of claim 22, wherein the immune-related diseases are selected from the group consisting of infection and sepsis.
